(19) United States
(12) Patent Application Publication
(10) Pub. No.: US 2011/0166078 A1
(43) Pub. Date: Jul. 7, 2011

(54) OLIGOSACCHARIDE COMPOUNDS FOR USE IN MOBILISING STEM CELLS

(75) Inventors: Eric Cabannes, Antony (FR); Audrey Caravano, Courbevoie (FR); Daniel Lewandowski, Fontenay Aux Roses (FR); Vincent Motte, Lille (FR); Vanessa Nancy-Portebois, Les Pavillons-Sous-Bois (FR); Maurice Petitou, Paris (FR); Pascal Pierdet, Rosny-Sous-Bois (FR)

(73) Assignee: ENDOTIS PHARMA

(21) Appl. No.: 13/063,826

(22) PCT Filed: Sep. 15, 2009
(86) PCT No.: PCT/EP2009/061970

§ 371 Date: Mar. 14, 2011

(30) Foreign Application Priority Data
Sep. 15, 2008 (EP) 08290866.6
Sep. 15, 2008 (EP) 08290867.4

(51) Int. Cl. A61K 38/19; A61P 35/00; A61K 31/727; C08B 37/10; A61P 7/00

(52) U.S. Cl. 514/19.3; 514/1.1; 514/56; 536/21

(57) ABSTRACT

A compound of the following formula or a salt, solvate or formula (I) and a pharmaceutical composition containing said compound. It concerns also its use in the treatment of cancer and/or of pathological angiogenesis and/or in promoting the mobilisation of stem cells, in particular hematopoietic stem cells.

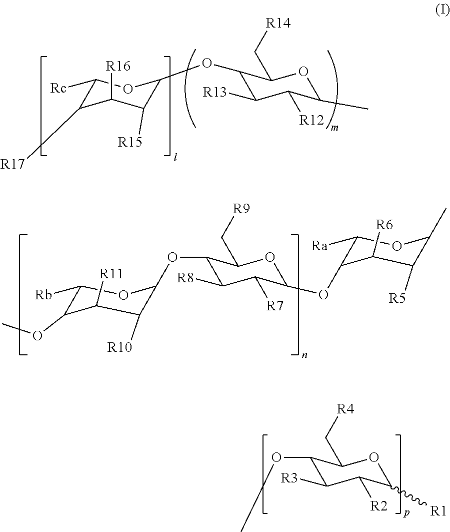

All documents cited herein are incorporated by reference in their entirety.

### TECHNICAL FIELD

The present invention is concerned with oligosaccharide derivatives, their intermediates, uses thereof and processes for their production. In particular, the present invention relates to oligosaccharides that interfere with the interaction of any molecule, such as growth factors, enzymes or chemokines, with heparan sulphate. The oligosaccharides of the present invention are useful in the treatment of cancer, pathological angiogenesis and/or for inducing hematopoietic stem cell (HSC) mobilisation.

### BACKGROUND

Heparan sulphate is a complex polysaccharide of the glycosaminoglycan family. Specifically, heparan sulphate exists as part of a proteoglycan. Heparan sulphate side chains regulate the functions of proteins with clusters of positively charged amino acids by binding to them to alter their activities and concentrations. Proteins, such as VEGF-A, FGF-1, FGF-2, PDGF-β and SDF-1, are under the direct or indirect control of heparan sulphate. Such proteins are involved in several biological phenomena, particularly in angiogenesis and cell trafficking.

Heparan sulfate proteoglycans are integral components of the extracellular matrix that surrounds all mammalian cells. In particular, heparan sulphate proteoglycans are a major component of the basement membrane and the extracellular matrix, which consists of a protein core with multiple complex glycosaminoglycan heparan sulphate side chains. Heparan sulfate proteoglycans play an important role in the self-assembly and integrity of the basement membrane architecture. In addition to providing structural integrity, heparan sulfate proteoglycans act as a storage depot for a variety of heparan sulfate binding proteins, such as growth factors and chemokines.

In order to grow, tumours need nutrient support from the vascular system. Typically, tumours cannot grow beyond a certain size (approximately 2 mm) due to a lack of oxygen and other nutrients. Accordingly, nutrient support is needed. This support is provided by the growth of blood vessels (angiogenesis), which is induced by the secretion of various angiogenic proteins, such as growth factors, enzymes and chemokines.

Studies have been conducted to show that growth factors closely interact with heparan sulphate and their activity has been linked with their affinity for heparan sulphate. For example, it has been shown that if the activity of growth factors is inhibited, the growth of tumours can also be inhibited. Similarly, chemokines have been shown to closely interact with heparan sulphate, and inhibiting their activity also inhibits tumour growth.

Heparanase is a matrix-degrading enzyme that cleaves heparan sulfate side chains. Heparanase is present in the endothelial cell layer that lines the inner surface of blood vessels. Successful penetration of this layer is an important process in tumour growth. Thus, inhibition of heparanase activity stops penetration into the endothelial cell layer and inhibits tumour growth.

Growth factors and chemokines stored in the extracellular matrix are released by the cleavage of heparan sulphate by heparanase, which promotes angiogenesis and therefore tumour growth and metastasis. These angiogenic proteins function by increasing vascular permeability, providing endothelial cell activation and migration, proliferation and, eventually, capillary formation. Thus, heparanase not only liberates heparan sulphate binding proteins, such as growth factors and chemokines, it also contributes to extracellular matrix degradation.

In view of the above, it would be desirable to produce a heparan sulphate mimetics that is capable of binding various angiogenic proteins. In doing so, the activity of these angiogenic proteins would be inhibited, which would, in turn, inhibit the growth of tumours. Compounds intended to mimic the backbone of heparan sulphate have been obtained and they have been shown to have adverse side effects, which include thrombocytopenia, anticoagulant activity and short half lives. Some of these side effects have been seen in the heparan sulphate mimetic PI-88 (Rosenthal, M. A., Annals of Oncology, 2002, 13, 770-776), that inhibits heparanase and acts as a growth factor antagonist, but exhibits undesirable anticoagulant activity.

The present invention, therefore, aims to produce a therapeutic agent adapted to antagonise those angiogenic proteins that are known to be involved in cancer progression and angiogenesis associated with tumour growth. It is a particular aim of the invention to produce a dual-targeting or multi-targeting therapy that interferes with the interaction of at least one angiogenic protein with heparan sulphate, wherein the angiogenic protein, such as growth factors, enzymes and chemokines, is involved in cancer progression and angiogenesis associated with tumour growth and metastasis. An additional aim of the invention is to produce a compound that limits the emergence of drug resistance. A further aim of the present invention is to produce a compound that has negligible, or no, side effects. Anticoagulant activity is a specific example of an undesirable side effect

The trafficking of hematopoietic stem cells (HSCs) between bone marrow and blood also involves growth factors and cytokines that bind to heparan sulphate. Hematopoietic stem cells (HSCs) are found in the bone marrow of bones such as the femur, hip, ribs, and sternum for example. Movement of HSCs between bone marrow (their main site of production) and peripheral blood is a physiological process named mobilisation. The reverse process (from peripheral blood to bone marrow) is called homing.

Hematopoietic stem cells are unique because they have the ability to form multiple cell types (multipotency) and they are able to self-renew. These cells give rise to blood-forming cells, such as monocytes/macrophages, myeloid dendritic cells, granulocytes (i.e. neutrophils, basophils and eosinophils), erythrocytes, megakaryocytes/platelets and mast cells for the myeloid lineage and T-lymphocytes, B-lymphocytes/plasma cells, natural killer cells and lymphoid dendritic cells for the lymphoid lineage.

The ability of HSCs to self-renew has prompted their use in the treatment of several different haematological cancers Hematopoietic stem cells are used to treat these disorders because a small number of stem cells can be used to fully reconstitute the hematopoietic system. To this end HSCs are first collected from blood (from patient or donor) through apheresis, the patient is then treated (usually using a cytotoxic agent), finally HSCs are reinjected to repopulate the bone marrow.

In order to harvest a sufficient number of stem cells, it is necessary to administer a suitable dosage of a particular (or a mix) therapeutic agent that induces the release of stem cells from the bone marrow into the blood. An example of one such therapeutic agent is G-CSF. Granulocyte colony-stimulating factor is presently considered to be the best therapeutic agent to mobilise HSCs and hematopoietic progenitor cells (HPCs) for transplantation in patients after myeloablative treatment. However, broad inter-individual variability of mobilisation efficacy exists and poor HSCs/HPCs mobilisation in response to G-CSF is observed in heavily-treated patients who have cancer and genetic disorders, such as Fanconi's anaemia. Other side effects seen with currently available therapeutic agents that are used to induce the release of stem cells include: bone pain, fever and fluid retention, which can lead to swelling of the ankles or breathlessness.

In an attempt to enhance the effects of G-CSF induced mobilisation, other therapeutic agents have been used in combination with G-CSF. One such example is a therapeutic agent called AMD3100/Mozobil/plerixafor, which is a non-peptide antagonist of the CXCR4 molecule, the receptor of the SDF-1 chemokine. However, some of these additional therapeutic agents have been shown to exhibit undesirable toxic effects.

Once the stem cells have been released into the blood, aphaeresis needs to be performed in order to extract the stem cells from the rest of blood cells.

The present invention aims to produce a therapeutic agent that could be used in the mobilisation of stem cells from the bone marrow into the blood. It is a particular aim of the invention to produce a therapeutic agent that exhibits HSCs/HPCs mobilisation properties alone or in combination with G-CSF and/or other mobilising agents. Another aim of the present invention is to produce a compound that has negligible, or no, side effects. Bone pain, fever, fluid retention and breathlessness are specific examples of undesirable side effects. Another aim of the present invention is to produce a more effective method of mobilising stem cells that does not require the administration of therapeutic agents over several days.

### DISCLOSURE OF THE INVENTION

According to one aspect of the present invention, there is provided a compound or a salt, solvate or prodrug thereof comprising an oligosaccharide that is capable of acting as an inhibitor of at least one angiogenic protein, such as a growth factor, an enzyme and a chemokine, which is effective in the treatment of pathological angiogenesis (e.g. angiogenesis associated with tumour growth, age-related macular degeneration (AMD)) and the treatment of cancer.

The present invention provides a compound of formula (I) or a salt, solvate or prodrug thereof:

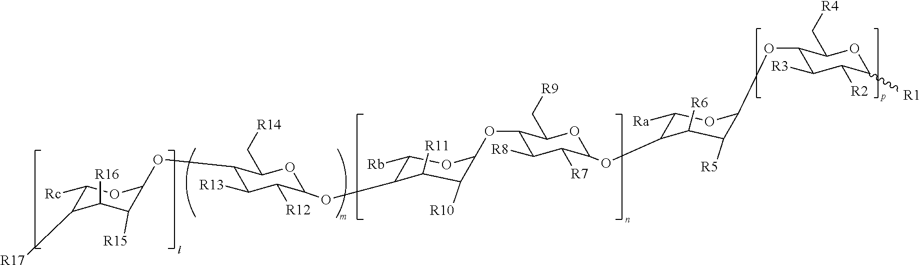

wherein:

Rₐ, R_{b} and R_{c} are selected from the group consisting of: COOH, COO—C₁₋₁₀alkyl, COO—C₃₋₁₀cycloalkyl and COO—C₁₋₁₀alkylC₃₋₁₀aryl, COO—C₃₋₁₀cycloalkylC₃₋₁₀aryl, advantageously Rₐ═R_{b}═R_{c};

R₁ is selected from the group consisting of: hydrogen, C₁₋₁₀alkyl, C₃₋₁₀cycloalkyl, C₂₋₁₀alkenyl, C₃₋₁₀cycloakenyl, C₂₋₁₀alkynyl, O—C₁₋₁₀ alkyl, O—C₃₋₁₀cycloalkyl, O—C₂₋₁₀ alkenyl, O—C₃₋₁₀cycloalkenyl, O—C₂₋₁₀alkynyl, O—C₃₋₁₀aryl, OH and O—SO₃H;

R₂, R₇, R₁₂ are each independently selected from the group consisting of: hydrogen, NH—SO₃H, NH₂, NH—C(O)C₃₋₁₀aryl, NH—C(O)C₁₋₁₀alkylCOOH, NH—C(O)C₃₋₁₀cycloalkylCOOH, NH—C(O)C₃₋₁₀arylSO₃H, NH—C(O)C₃₋₁₀arylCOOH, O—C₁₋₁₀alkyl, O—C₃₋₁₀cycloalkyl, O—C₃₋₁₀ cycloalkylC₃₋₁₀aryl, O—C₁₋₁₀alkylC₃₋₁₀aryl, O—C₁₋₁₀ acyl, O—C₁₋₁₀acylC₃₋₁₀aryl, O—C(O)C₃₋₁₀arylSO₃H, O—C(O)C₃₋₁₀arylCOOH, O—SO₃H, O—C(O)C₃₋₁₀aryl, O—C(O)C₁₋₁₀alkylCOOH, O—C₁₋₁₀alkylOSO₃H, O—C₁₋₁₀alkylNHSO₃H, O—C(O)C₃₋₁₀cycloalkylCOOH, O—C₃₋₁₀cycloalkylOSO₃H, O—C₃₋₁₀cycloalkylNHSO₃H and OH; advantageously NH—C₁₋₁₀acyl, NH—SO₃H, NH₂, NH—C(O)C₃₋₁₀aryl, NH—C(O)C₁₋₁₀alkyl-COOH, NH—C(O)C₃₋₁₀arylSO₃H, NH—C(O)C₃₋₁₀arylCOOH, O—C₁₋₁₀alkyl, O—C₁₋₁₀alkylC₃₁₀aryl, O—C₁₋₁₀acyl, O—SO₃H, O—C(O)C₃₋₁₀aryl, O—C(O)C₃₋₁₀arylSO₃H, O—C(O)C₃₋₁₀arylCOOH, O—C(O)C₁₋₁₀alkylCOOH and OH; still more advantageously NH—C₁₋₁₀acyl, NH—SO₃H, NH₂, NH—C(O)C₃₋₁₀aryl, NH—C(O)C₁₋₁₀alkylCOOH, NH—C(O)C₃₋₁₀arylSO₃H and NH—C(O)C₃₋₁₀arylCOOH, in particular NH—SO₃H and NH₂.

In particular, R₂, R₇ and R₁₂ are each independently selected from the group consisting of: hydrogen, NH—C₁₋₁₀acyl, NH—C₁₋₁₀acylC₃₋₁₀aryl, NH—SO₃H, NH₂, O—C₁₋₁₀alkyl, O—C₁₋₁₀alkylC₃₋₁₀aryl, O—C₁₋₁₀acyl, O—C₁₋₁₀acylC₃₋₁₀aryl, O—SO₃H and OH.

R₃, R₅, R₆, R₈, R₁₀, R₁₁, R₁₃, R₁₅ and R₁₆ are each independently selected from the group consisting of: hydrogen, O—C₁₋₁₀alkyl, O—C₁₋₁₀alkylC₃₋₁₀aryl, O—C₁₋₁₀alkylOSO₃H, O—C₁₋₁₀alkylNHSO₃H, O—C₃₋₁₀cycloalkyl, O—C₃₋₁₀cycloalkylC₃₋₁₀aryl, O—C₃₋₁₀cycloalkylOSO₃H, O—C₃₋₁₀cycloalkylNHSO₃H, O—C₁₋₁₀acyl, O—C₁₋₁₀acylC₃₋₁₀aryl, O—C(O)C₃₋₁₀aryl, O—C(O)C₁₋₁₀alkylCOOH, O—C(O)C₃₋₁₀cycloalkylCOOH, O—SO₃H, O—C(O)C₃₋₁₀arylSO₃H, O—C(O)C₃₋₁₀arylCOOH and OH, advantageously O—C₁₋₁₀alkyl, O—C₁₋₁₀alkylC₃₋₁₀aryl, O—C₁₋₁₀acyl, O—C₁₋₁₀acylC₃₋₁₀aryl, O—C(O)C₃₋₁₀arylSO₃H, O—C(O)C₃₋₁₀arylCOOH, O—SO₃H and OH, still more advantageously O—C₁₋₁₀alkyl, O—C₁₋₁₀alkylC₃₋₁₀aryl, O—SO₃H and OH;

In particular, R₃, R₅, R₆, R₈, R₁₀, R₁₁, R₁₃, R₁₅ and R₁₆ are each independently selected from the group consisting of: hydrogen, O—C₁₋₁₀alkyl, O—C₁₋₁₀alkylC₃₋₁₀aryl, O—C₁₋₁₀acyl, O—C₁₋₁₀acylC₃₋₁₀aryl, O—SO₃H and OH.

R₄, R₉ and R₁₄ are each independently selected from the group consisting of: O—C₁₋₁₀alkyl, O—C₃₋₁₀cycloalkyl, O—C₁₋₁₀alkylC₃₋₁₀aryl, O—C₁₋₁₀alkylOSO₃H, O—C₃₋₁₀cycloalkylC₃₋₁₀aryl, O—C₃₋₁₀cycloalkylOSO₃H, O—C₃₋₁₀cycloalkylNHSO₃H, O—C₁₋₁₀alkylNHSO₃H, O—C₁₋₁₀acyl, O—C₁₋₁₀acylC₃₋₁₀aryl, O—C(O)C₃₋₁₀aryl, O—C(O)C₁₋₁₀alkylCOOH, O—C(O)C₃₋₁₀cycloalkylCOOH, O—SO₃H, OH, O—C(O)C₃₋₁₀arylSO₃H, O—C(O)C₃₋₁₀arylCOOH, NH—C₁₋₁₀acyl, NH—C₁₋₁₀acylC₃₋₁₀aryl, NH—C(O)C₃₋₁₀aryl, NH—C(O)C₁₋₁₀alkylCOOH, NH—C(O)C₃₋₁₀cycloalkylCOOH, NH—SO₃H, NH—C(O)C₃₋₁₀arylSO₃H, NH—C(O)C₃₋₁₀arylCOOH and NH₂, advantageously O—C₁₋₁₀alkyl, O—SO₃H, OH, NH—SO₃H and NH₂, still more advantageously O—SO₃H and OH.

In particular, R₄, R₉ and R₁₄ are each independently selected from the group consisting of: O—C₁₋₁₀alkyl, O—C₁₋₁₀alkylC₃₋₁₀aryl, O—C₁₋₁₀acyl, O—C₁₋₁₀acylC₃₋₁₀aryl, O—SO₃H, OH, NH—C₁₋₁₀acyl, NH—C₁₋₁₀acylC₃₋₁₀aryl, NH—SO₃H and NH₂.

R₁₇ is independently selected from the group consisting of: hydrogen, O—C₁₋₁₀alkyl, O—C₂₋₁₀alkenyl, O—C₃₋₁₀cycloalkenyl, O—C₂₋₁₀alkynyl, O—C₃₋₁₀cycloalkylC₃₋₁₀alkyl, O—C₁₋₁₀alkylC₃₋₁₀aryl, O—C₃₋₁₀cycloalkyl, O—C₁₋₁₀acyl, O—C₁₋₁₀acylC₃₋₁₀aryl, O—SO₃H, O—C(O)C₃₋₁₀aryl, O—C(O)C₁₋₁₀alkylCOOH, O—C(O)C₃₋₁₀cycloalkylCOOH, O—C(O)C₃₋₁₀arylSO₃H, O—C(O)C₃₋₁₀arylCOOH and OH; advantageously O—C₁₋₁₀alkyl, O—C₁₋₁₀alkylC₃₋₁₀aryl, O—SO₃H and —OH, still more advantageously O—C₁₋₁₀alkylC₃₋₁₀aryl, and O—C₁₋₁₀alkyl.

In particular, R₁₇ is independently selected from the group consisting of: hydrogen, O—C₁₋₁₀alkyl, O—C₂₋₁₀alkenyl, O—C₂₋₁₀alkynyl, O—C₁₋₁₀alkylC₃₋₁₀aryl, O—C₁₋₁₀acyl, O—C₁₋₁₀acylC₃₋₁₀aryl, O—SO₃H and OH;

n is an integer selected from 0 to 4;

l, m and p are each an integer independently selected from 0 and 1; in particular m=1

provided at least 20%, advantageously at least 30%, more advantageously at least 50%, of the groups R₂, R₃, R₄, R₅, R₆, R₇, R₈, R₉, R₁₀, R₁₁, R₁₂, R₁₃, R₁₄, R₁₅, R₁₆ and R₁₇ are OSO₃H or NH—SO₃H;

provided at least 20%, advantageously at least 30%, of the groups R₁, R₂, R₃, R₄, R₅, R₆, R₇, R₈, R₉, R₁₀, R₁₁, R₁₂, R₁₃, R₁₄, R₁₅, R₁₆ and R₁₇ are NH—C₁₋₁₀acyl, NH—C₁₋₁₀acylC₃₋₁₀aryl, NH—C(O)C₃₋₁₀aryl, NH—C(O)C₁₋₁₀alkylCOOH, NH—C(O)C₃₋₁₀cycloalkylCOOH, NH—C(O)C₃₋₁₀arylSO₃H, NH—C(O)C₃₋₁₀arylCOOH, O—C₁₋₁₀alkyl, O—C₃₋₁₀cycloalkyl, O—C₁₋₁₀alkylC₃₋₁₀aryl, O—C₃₋₁₀cycloalkylC₃₋₁₀aryl, O—C₁₋₁₀acyl, O—C₁₋₁₀acylC₃₋₁₀aryl, O—C₂₋₁₀alkenyl, O—C₂₋₁₀cycloalkenyl, O—C(O)C₃₋₁₀aryl, O—C(O)C₁₋₁₀alkylCOOH, O—C(O)C₃₋₁₀cycloalkylCOOH, O—C(O)C₃₋₁₀arylSO₃H, O—C(O)C₃₋₁₀arylCOOH, OH or O—C₂₋₁₀alkynyl, advantageously NH—C₁₋₁₀acyl, NH—C₁₋₁₀acylC₃₋₁₀aryl, NH—C(O)C₃₋₁₀aryl, NH—C(O)C₁₋₁₀alkylCOOH, NH—C(O)C₃₋₁₀cycloalkylCOOH, NH—C(O)C₃₋₁₀arylSO₃H, NH—C(O)C₃₋₁₀arylCOOH, O—C₁₋₁₀alkyl, O—C₃₋₁₀cycloalkyl, O—C₁₋₁₀alkylC₃₋₁₀aryl, O—C₃₋₁₀cycloalkylC₃₋₁₀aryl, O—C₁₋₁₀acyl, O—C₁₋₁₀acylC₃₋₁₀aryl, O—C₂₋₁₀alkenyl, O—C₂₋₁₀cycloalkenyl, O—C(O)C₃₋₁₀aryl, O—C(O)C₁₋₁₀alkylCOOH, O—C(O)C₃₋₁₀cycloalkylCOOH, O—C(O)C₃₋₁₀arylSO₃H, O—C(O)C₃₋₁₀arylCOOH, OH or O—C₂₋₁₀alkynyl and

In particular, at least 20% of the groups R₁, R₂, R₃, R₄, R₅, R₆, R₇, R₈, R₉, R₁₀, R₁₁, R₁₂, R₁₃, R₁₄, R₁₅, R₁₆ and R₁₇ are C₁₋₁₀alkyl, C₂₋₁₀alkenyl, C₂₋₁₀alkynyl, NH—C₁₋₁₀acyl, NH—C₁₋₁₀acylC₃₋₁₀aryl, O—C₁₋₁₀alkyl, O—C₁₋₁₀alkylC₃₋₁₀aryl, O—C₁₋₁₀acyl, O—C₁₋₁₀acylC₃₋₁₀aryl, O—C₂₋₁₀alkenyl and O—C₂₋₁₀alkynyl;

wherein any of R₁₋₁₇ are independently optionally substituted with one or more groups independently selected from C₁₋₁₀alkyl, C₃₋₁₀cycloalkyl, C₂₋₁₀alkenyl, C₃₋₁₀cycloalkenyl, O—C₁₋₁₀alkyl, O—C₃₋₁₀cycloalkyl, O—C₂₋₁₀alkenyl, O—C₃₋₁₀cycloalkenyl, O—C₁₋₁₀alkylC₃₋₁₀aryl, O—C₃₋₁₀cycloalkylC₃₋₁₀aryl, C₂₋₁₀alkynyl, C₃₋₁₀aryl, C₃₋₁₀arylSO₃H, C₃₋₁₀arylC₁₋₁₀alkyl, C₃₋₁₀arylC₃₋₁₀cycloalkyl, C₁₋₁₀alkyl C₃₋₁₀aryl, COOH, C₁₋₁₀alkylCOOH, C₃₋₁₀cycloalkylC₃₋₁₀aryl, C₃₋₁₀cycloalkylCOOH, C₃₋₁₀arylCOOH, COOC₁₋₁₀alkyl, COOC₃₋₁₀cycloalkyl, SH, S—C₁₋₁₀alkyl, S—C₃₋₁₀cycloalkyl, SO₂H, SO₂ C₁₋₁₀alkyl, SO₂ C₃₋₁₀cycloalkyl, SO₂C₃₋₁₀aryl, SO₂ C₁₋₁₀alkyl C₃₋₁₀aryl, SO₂ C₃₋₁₀cycloalkylC₃₋₁₀aryl, O—SO₃H, O—P(O)(OH)₂, halo, C₁₋₁₀alkyl halo, C₃₋₁₀cycloalkyl halo, per halo C₁₋₁₀alkyl, per halo C₃₋₁₀cycloalkyl, OH, ═O, NH₂, ═NH, NH—C₁₋₁₀alkyl, N(C₁₋₁₀alkyl)₂, ═NC₁₋₁₀alkyl, NH—C(O)C₁₋₁₀alkyl, NH—C₃₋₁₀cycloalkyl, N(C₃₋₁₀cycloalkyl)₂, N(C₁₋₁₀alkyl)(C₃₋₁₀cycloalkyl), ═N—C₃₋₁₀cycloalkyl, NH—C(O)—C₃₋₁₀cycloalkyl, C(O)NH₂, C(O)NHC₁₋₁₀alkyl, C(O)N(C₁₋₁₀alkyl)₂, C(O)—NH—C₃₋₁₀cycloalkyl, C(O)N(C₃₋₁₀cycloalkyl)₂, C(O)N(C₁₋₁₀alkyl)(C₃₋₁₀cycloalkyl) C(O)NHC₃₋₁₀aryl, NO₂, ONO₂, CN, SO₂, SO₂NH₂, C(O)H and C(O)C₁₋₁₀alkyl, C(O)—C₃₋₁₀cycloalkyl;

provided that when: R₁ is O—CH₂CH═CH₂; R₂, R₇ and R₁₂ are each NH—SO₃H; R₄, R₅, R₉, R₁₀, R₁₄ and R₁₅ are each O—SO₃H; R₃, R₆, R₈, R₁₁, R₁₃ and R₁₆ are each O-benzyl; and R₁₇ is not O-para-methoxybenzyl.

In an advantageous embodiment at least one, more advantageously at least 2, of the groups R₃, R₄, R₅, R₆, R₈, R₉, R₁₀, R₁₁, R₁₃, R₁₄, R₁₅ and R₁₆ does not represent O—SO₃H or OH when R₂, R₇ and R₁₂ represents independently of each other a group NH—SO₃H or NH—C₁₋₁₀acyl;

Some compounds of formula I in which all the groups R₃, R₄, R₅, R₆, R₈, R₉, R₁₀, R₁₁, R₁₃, R₁₄, R₁₅ and R₁₆ represent independently O—SO₃H or OH and all the groups R₂, R₇ and R₁₂ represents independently of each other a group NH—SO₃H or NH—C₁₋₁₀acyl are known in the art.

However, the inventors have surprisingly found that when modifying one of the group R₃, R₄, R₅, R₆, R₈, R₉, R₁₀, R₁₁, R₁₃, R₁₄, R₁₅ and R₁₆ of the compounds of the prior art, in particular in order that one of them represents a group O—C₁₋₁₀alkyl or O—C₁₋₁₀alkylC₃₋₁₀aryl, the compounds thus obtained have modifying properties, in particular more advantageous ones.

The inventors have also discovered that when modifying one of the groups R₂, R₇ and R₁₂ of the compounds of the prior art the compounds thus obtained have modifying properties, in particular more advantageous ones.

In a further aspect of the invention, a pharmaceutical composition is provided wherein the composition comprises a compound or a salt, solvate or prodrug of the following formula (I):

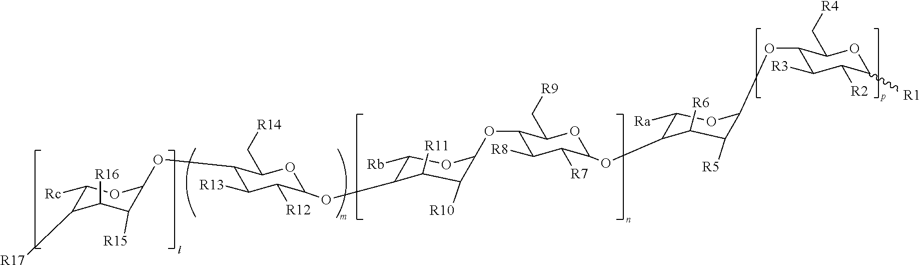

wherein:

Rₐ, R_{b}, R_{c}, R₁₋₁₇, l, m, n, p are as defined above and a compound of formula (I) in which R₁ is O—CH₂CH═CH₂; R₂, R₇ and R₁₂ are each NH—SO₃H; R₄, R₅, R₉, R₁₀, R₁₄ and R₁₅ are each O—SO₃H; R₃, R₆, R₈, R₁₁, R₁₃ and R₁₆ are each O-benzyl; and R₁₇ is not O-para-methoxybenzyl.

and a pharmaceutically acceptable diluent or carrier.

In one aspect of the present invention, the pharmaceutical composition according to the present invention contains a cytokine, advantageously G-CSF, and/or other mobilising agents, advantageously AMD3100.

In one aspect of the invention, R₁ is selected from the group consisting of: O—C₁₋₁₀alkyl, O—C₂₋₁₀alkenyl and O—C₂₋₁₀alkynyl and OH. In a further aspect of the invention, R₁ is O—C₂₋₁₀alkynyl.

In another aspect of the invention, R₂, R₇ and R₁₂ are each independently selected from the group consisting of: NH—C₁₋₁₀acyl, NH—C₁₋₁₀acylC₃₋₁₀aryl, NH—SO₃H and NH₂. In a further aspect of the invention, R₂, R₇ and R₁₂ are each independently selected from the group consisting of: NH—C₁₋₁₀acyl and NH—C₁₋₁₀acylC₃₋₁₀aryl. In a further aspect of the invention, R₂, R₇ and R₁₂ are each NH—SO₃H.

In another aspect of the invention, R₃, R₄, R₅, R₆, R₈, R₉, R₁₀, R₁₁, R₁₃, R₁₄, R₁₅, R₁₆ and R₁₇ are each independently selected from the group consisting of: O—C₁₋₁₀alkyl, O—C₁₋₁₀alkylC₃₋₁₀aryl, O—C₁₋₁₀acyl, O—C₁₋₁₀acylC₃₋₁₀aryl, O—SO₃H and OH. In a further aspect of the invention, alkylaryl is selected from the group consisting of optionally substituted benzyl and phenylpropyl, wherein the optional substituent is a halo group. In a further aspect of the invention, alkyl is methyl.

In another aspect of the invention, R₃, R₆, R₈, R₁₁, R₁₃ and R₁₆ are each independently selected from the group consisting of: O—C₁₋₁₀alkylC₃₋₁₀aryl, O—C₁₋₁₀alkyl, O—SO₃H and OH. In a further aspect of the invention, R₃, R₆, R₈, R₁₁, R₁₃ and R₁₆ are each independently selected from O—C₁₋₁₀alkylC₃₋₁₀aryl and O—C₁₋₁₀alkyl. In a further aspect of the invention, R₃, R₆, R₈, R₁₁, R₁₃ and R₁₆ are each independently selected from the group consisting of: O—C₁₋₁₀alkylC₃₋₁₀aryl and OH. In a further aspect of the invention, R₃, R₆, R₈, R₁₁, R₁₃ and R₁₆ are each independently O—C₁₋₁₀alkylC₃₋₁₀aryl. In a further aspect of the invention, alkylaryl is benzyl. In a further aspect of the invention, alkyl is methyl.

In a further aspect of the invention, R₃, R₆, R₈, R₁₁, R₁₃ and R₁₆ are each independently selected from the group consisting of: O-benzyl, O-methyl, O—SO₃H and OH. In a further aspect of the invention, R₃, R₆, R₈, R₁₁, R₁₃ and R₁₆ are each independently selected from the group consisting of: O-benzyl and OH. In a further aspect of the invention, R₃, R₆, R₈, R₁₁, R₁₃, R₁₆ and R₁₇ are each O-benzyl. In a further aspect of the invention, R₃, R₆, R₈, R₁₁, R₁₃, R₁₆ and R₁₇ are each OH.

In another aspect of the invention, R₄, R₅, R₉, R₁₀, R₁₄ and R₁₅ are each independently selected from the group consisting of: OH and O—SO₃H. In a further aspect of the invention, R₄, R₅, R₉, R₁₀, R₁₄ and R₁₅ are each O—SO₃H.

In another aspect of the invention, R₄, R₉ and R₁₄ are each independently selected from the group consisting of: O—C₁₋₁₀alkyl, OH and O—SO₃H, advantageously from OH and O—SO₃H. In a further aspect of the invention, R₄, R₉, and R₁₄ are each O—SO₃H.

In another aspect of the invention, R₅, R₁₀ and R₁₅ are each O—SO₃H.

In another aspect of the invention, R₁₇ is selected from the group consisting of: O—C₁₋₁₀alkylC₃₋₁₀aryl, O—C₁₋₁₀alkyl and OH. In a further aspect of the invention, alkyl is methyl. In a further aspect of the invention, R₁₇ is selected from the group consisting of: O—C₁₋₁₀alkylC₃₋₁₀aryl and OH. In a further aspect of the invention, R₁₇ is O—C₁₋₁₀alkylC₃₋₁₀aryl. In a further aspect of the invention, alkylaryl is benzyl. In a further aspect of the invention, R₁₇ is selected from the group consisting of: O-benzyl, O-methyl and OH. In a further aspect of the invention, R₁₇ is selected from the group consisting of: O-benzyl and OH. In a further aspect of the invention, R₁₇ is selected from the group consisting of: O-methyl.

In another aspect of the invention, n is an integer selected from 0 to 4 and it can be 0, 1, 2, 3 and 4. In another aspect of the invention, n is 1. In a further aspect of the invention, n is 2. In a further aspect of the invention, n is an integer selected from 1 to 2.

In another aspect of the invention, 1 is 1. In another aspect of the invention, m is 1. In another aspect of the invention, p is 1.

In another aspect of the invention, R₂, R₇ and R₁₂ are each independently selected from the group consisting of: NH—C₁₋₁₀acyl and NH—C₁₋₁₀acylC₃₋₁₀aryl; R₃, R₆, R₈, R₁₁, R₁₃ and R₁₆ are each independently selected from the group consisting of: O—C₁₋₁₀alkylC₃₋₁₀aryl and OH; R₁₇ is selected from the group consisting of: O—C₁₋₁₀alkylC₃₋₁₀aryl and OH; n is an integer selected from 1 and 2; and l, m and p are each 1; wherein any of R₂, R₇ and R₁₂ are independently optionally substituted with one or more groups independently selected from C₁₋₁₀alkyl, C₃₋₁₀aryl, C₃₋₁₀arylCOOH, C₃₋₁₀arylSO₃H, C₁₋₁₀alkylCOOH.

In another aspect of the invention, R₂, R₇ and R₁₂ are each independently selected from the group consisting of: NH—C₁₋₁₀acyl and NH—C₁₋₁₀acylC₃₋₁₀aryl; R₃, R₆, R₈, R₁₁, R₁₃ and R₁₆ are each independently selected from the group consisting of: O-benzyl, O-methyl and OH; R₁₇ is selected from the group consisting of: O-benzyl, O-methyl and OH; n is an integer selected from 1 and 2; and l, m and p are each 1; wherein any NH₂ of R₂, R₇ and R₁₂ are independently optionally substituted with one or more groups independently selected from (CO)methyl, (CO)phenyl, (CO)phenylCOOH, (CO)phenylSO₃H, (CO)propanoylic acid.

In another aspect of the invention, R₅, R₁₀, and R₁₅ are each O—SO₃H; R₂, R₇ and R₁₂ are each NH—SO₃H; and R₄, R₉ and R₁₄ are each independently selected from the group consisting of: OH and O—SO₃H;

In another aspect of the invention, R₃, R₆, R₈, R₁₁, R₁₃ and R₁₆ are each independently selected from the group consisting of: O-benzyl, O-methyl and OH; R₁₇ is selected from the group consisting of: O-benzyl, O-methyl and OH; n is an integer selected from 1 and 2; and l, m and p are each 1.

In another aspect of the invention, R₂, R₇ and R₁₂ are each NH—SO₃H; n is an integer selected from 1 and 2; and l, m and p are each 1.

In another aspect of the invention, R₁ is O—C₂₋₁₀alkynyl; R₃, R₆, R₈, R₁₁, R₁₃ and R₁₆ are each O—C₁₋₁₀alkylC₃₋₁₀aryl; and R₁₇ is O—C₁₋₁₀alkylC₃₋₁₀aryl.

In another aspect of the invention, R₂, R₇ and R₁₂ are each NH—SO₃H; R₃, R₆, R₈, R₁₁, R₁₃, R₁₆ and R₁₇ are each OH; R₄, R₅, R₉, R₁₀, R₁₄ and R₁₅ are each O—SO₃H; and l, m and p are each 1.

In one aspect of the invention, any one of the groups R₁₋₁₇ is independently optionally substituted with one or more groups, in a further aspect 0, 1 or 2 groups, in yet a further aspect 0 or 1 groups, independently selected from C₁₋₁₀alkyl, C₂₋₁₀alkenyl, O—C₁₋₁₀alkyl, O—C₂₋₁₀alkenyl, O—C₁₋₁₀alkylC₃₋₁₀aryl, C₂₋₁₀alkynyl, C₃₋₁₀aryl, C₃₋₁₀arylSO₃H, C₃₋₁₀arylC₁₋₁₀alkyl, C₁₋₁₀alkylC₃₋₁₀aryl, COOH, C₁₋₁₀alkylCOOH, C₃₋₁₀arylCOOH, COOC₁₋₁₀alkyl, SH, S—C₁₋₁₀alkyl, SO₂H, SO₂ C₁₋₁₀alkyl, SO₂C₃₋₁₀aryl, SO₂ C₁₋₁₀alkyl C₃₋₁₀aryl, O—SO₃H, O—P(O)(OH)₂, halo, C₁₋₁₀alkylhalo, perhaloC₁₋₁₀alkyl, OH, ═O, NH₂, ═NH, NH—C₁₋₁₀alkyl, N(C₁₋₁₀alkyl)₂, ═NC₁₋₁₀alkyl, NH—C(O)C₁₋₁₀alkyl, C(O)NH₂, C(O)NHC₁₋₁₀alkyl, C(O)N(C₁₋₁₀alkyl)₂, C(O)NHC₃₋₁₀aryl, NO₂, ONO₂, CN, SO₂, SO₂NH₂, C(O)H, and C(O)C₁₋₁₀alkyl.

When targeting angiogenic proteins, it is preferable to use longer chain oligosaccharides. For example, octasaccharides show a greater interaction with growth factors than hexasaccharides and hexasaccharides show a greater interaction with growth factors than tetrasaccharides.

In another aspect of the invention, substitution at R₃, R₈ and R₁₃ and R₁₇ R₆, R₁₁ and R₁₆ can be rationalised as follows wherein substitutions are shown in order of preference with most preferred occurring first: R₃, R₈ and R₁₃ and R₁₇ R₆, R₁₁ and R₁₆ are O-benzyl; R₃, R₈ and R₁₃ is O-Me and R₁₇ R₆, R₁₁ and R₁₆ are both O-benzyl; R₃, R₈ and R₁₃ is O-benzyl and R₁₇ R₆, R₁₁ and R₁₆ is O-Me; R₃, R₈ and R₁₃ is O-Me and R₁₇ R₆, R₁₁ and R₁₆ is OH; R₃, R₈ and R₁₃ is OH and R₁₇ R₆, R₁₁ and R₁₆ is O-Me; R₃, R₈ and R₁₃ and R₁₇ R₆, R₁₁ and R₁₆ are both O-Me. Thus, preferably the substituents at both the R₃, R₈ and R₁₃ and R₁₇ R₆, R₁₁ and R₁₆ positions are O-benzyl. Preferably, the oligosaccharide used in this aspect of the invention is a hexasaccharide. More preferably, the hexasaccharide is formed when n, l, m and p are each 1.

In the present specification, the groups COOH, O—SO₃H and NH—SO₃H are represented in their acid form. It will be understood the representation in their acid form also extends to their salt form. In a one embodiment these groups are in their salt form, typically in their sodium or potassium salt form. Preferably, the groups are in their sodium salt form.

It will be appreciated that the oligosaccharides of the present invention are shown in a defined stereochemical configuration, which will be apparent to one skilled in the art. Positions of variable stereochemistry are indicated with wavy lines. Except where specifically indicated, the present invention extends to all such stereochemical forms. Accordingly, in one aspect of the invention, the group at the R₁ position in an alpha conformation. In an alternative aspect of the invention, the group at the R₁ position is in a beta conformation.

In a further aspect of the invention, the compound, salt, solvate or prodrug of the formula (I) according to the present invention is chosen from the group consisting of formula 215-216, 236-241, 244-263, 266-268, 274-283, 288 and 294, whose formula are indicated in the examples below.

The present invention also provides a method of making a pharmaceutical composition, comprising mixing the oligosaccharide of the present invention with a pharmaceutically acceptable diluent or carrier and eventually with a cytokine, advantageously G-CSF, and/or other mobilising agents, advantageously AMD3100.

In one aspect of the present invention, there is provided an oligosaccharide, as described in the present invention, for use in therapy.

In another aspect of the invention, there is provided an oligosaccharide, as defined in the present invention, for use in the treatment of cancer, in particular bone marrow and/or blood cancers, and/or for use in the treatment of pathological angiogenesis and/or for use in interfering with the interaction of one or more heparan sulphate binding protein with heparan sulphate and/or for use in promoting the mobilisation of stem cells, in particular hematopoietic stem cells, and/or for use in the treatment of diseases and conditions that are typically associated with patients suffering from blood and/or bone marrow cancers and/or solid tumours and/or for use in the treatment of acquired or congenital diseases mediated by hematological disorders.

The present invention also concerns a product containing an oligosaccharide, as defined in the present invention, and at least a cytokine, in particular G-CSF, and/or other mobilising agents, advantageously AMD3100, as a combined preparation for simultaneous, separate or sequential use in promoting the mobilisation of stem cells, in particular hematopoietic stem cells, and/or for use in the treatment of diseases and conditions that are typically associated with patients suffering from blood and/or bone marrow cancers and/or solid tumours and/or for use in the treatment of blood and bone marrow cancers and/or for use in the treatment of acquired or congenital diseases mediated by hematological disorders.

In another aspect of the invention, there is provided the use of an oligosaccharide, as defined in the present invention, in the manufacture of a medicament for the treatment of cancer, in particular blood marrow and/or blood cancers.

In another aspect of the invention, there is provided the use of an oligosaccharide, as defined in the present invention, in the manufacture of a medicament for the treatment of pathological angiogenesis.

In another aspect of the invention, there is provided the use of an oligosaccharide, as defined in the present invention, in the manufacture of a medicament for interfering with the interaction of one or more heparin sulphate binding protein with heparan sulphate.

In one aspect of the present invention, there is provided the use of an oligosaccharide, as defined in the present invention, in the manufacture of a medicament for mobilising stem cells, in particular hematopoietic stem cells.

In one aspect of the present invention, there is provided the use of an oligosaccharide, as defined in the present invention, in the manufacture of a medicament for the treatment of diseases and conditions that are typically associated with patients suffering from blood and/or bone marrow cancers and/or solid tumours.

In one aspect of the present invention, there is provided the use of an oligosaccharide, as defined in the present invention, in the manufacture of a medicament for the treatment of acquired or congenital diseases mediated by hematological disorders.

The present invention also provides a method of treating cancer, in particular bone marrow and/or blood cancers, in a patient comprising administering an effective amount of an oligosaccharide, as defined in the present invention.

The present invention also provides a method of treating pathological angiogenesis in a patient comprising administering an effective amount of an oligosaccharide, as defined in the present invention.

The present invention also provides a method of interfering with the interaction of one or more heparin sulphate binding protein with heparan sulphate in a patient comprising administering an effective amount of an oligosaccharide, as defined in the present invention.

In one aspect of the present invention, there is provided a method of mobilising stem cells, in particular hematopoietic stem cells, comprising the step of administering an effective amount of an oligosaccharide, as defined in the present invention.

In one aspect of the present invention, there is provided a method of treatment of diseases and conditions that are typically associated with patients suffering from blood and/or bone marrow cancers and/or solid tumours in a patient comprising administering an effective amount of an oligosaccharide, as defined in the present invention.

In one aspect of the present invention, there is provided a method of treating acquired or congenital diseases mediated by hematological disorders in a patient comprising administering an effective amount of an oligosaccharide, as defined in the present invention.

In another aspect of the present invention, the heparin sulphate binding protein is a growth factor, enzyme or chemokine.

In another aspect of the present invention, the growth factor of the present invention is selected from: VEGF-A, FGF-1, FGF-2, and PDGF-β.

In another aspect of the present invention, the enzyme of the present invention is heparanase.

In another aspect of the present invention, the chemokine of the present invention is SDF-1. In a further aspect of the present invention, the chemokine is SDF-1α.

In another aspect of the present invention, the cancer treated by the oligosaccharide defined in the present invention is selected from: breast, prostate, bladder, rhabdomyosarcoma, epidermoid, melanoma, liver, colon, blood, bone marrow and lung cancer.

In one aspect of the present invention, the acquired disease is a malignancy. In one aspect of the invention, the malignancy is for example independently selected from hematological malignancies, which include: leukaemias such as acute lymphoblastic leukaemia (ALL), acute myelogenous leukaemia (AML), chronic lymphocytic leukaemia (CLL) and chronic myelogenous leukaemia (CML); lymphomas such as Hodgkin's disease and non-Hodgkin's lymphoma; and myelomas such as multiple myeloma (Kahler's disease); and solid tumour cancers, which include neuroblastoma; dermoplastic small round cell tumour; Ewing's sarcoma; and choriocarcinoma.

In one aspect of the present invention, the acquired disease is a hematological disorder. In one aspect of the invention, the hematological disorder are independently selected from phagocyte disorders, which include myelodysplasia; anaemias, which include haemolytic anaemia (paroxysmal nocturnal haemoglobinuria); and aplastic anaemia such as acquired pure red cell aplasia; myeloproliferative disorders such as polycythemia vera and essential thrombocytosis; metabolic disorders, which include: amyloidoses such as amyloid light chain amyloidosis; and environmentally induced diseases such as radiation poisoning.

In one aspect of the present invention, the congenital disease is a lysosomal storage disorder. In one aspect of the invention, the lysosomal storage disorder is independently selected from lipidoses, which include: neuronal ceroid lipofuscinoses such as infantile neuronal ceroid lipofuscinosis (Santavuori disease) and Jansky-Bielschowsky disease (late infantile neuronal ceroid lipofuscinosis); sphingolipidoses such as Niemann-Pick disease and Gaucher disease; leukodystrophies such as adrenoleukodystrophy, 30 metachromatic leukodystrophy and krabbe disease (globoid cell leukodystrophy).

In one aspect of the present invention, the congenital disease is independently selected from mucopolysaccharidoses such as Hurler syndrome (MPS I H, a-L-iduronidase deficiency), Scheie syndrome (MPS I S), Hurler-Scheie syndrome (MPS I H-S), Hunter syndrome (MPS II, iduronidase sulfate deficiency), Sanfilippo syndrome (MPS III), Morquio syndrome (MPS IV), Maroteaux-Lamy syndrome (MPS VI) and Sly syndrome (MPS VII).

In one aspect of the present invention, the congenital disease is independently selected from glycoproteinoses such as Mucolipidosis II (I-cell disease), fucosidosis, aspartylglucosaminuria and alpha-mannosidosis.

In one aspect of the present invention, the congenital disease is wolman disease (acid lipase deficiency).

In one aspect of the present invention, the congenital disease is an immunodeficiency. In one aspect of the present invention, the immunodeficiency is independently selected from T-cell deficiencies, which include ataxia telangiectasia; and DiGeorge syndrome; combined T- and B-cell deficiencies, which include severe combined immunodeficiency (SCID), all types; well-defined syndromes, which include Wiskott-Aldrich syndrome; phagocyte disorders, which include Kostmann syndrome and Shwachman-Diamond syndrome; immune dysregulation diseases, which include Griscelli syndrome, type II; and innate immune deficiencies, which include NF-Kappa-B Essential Modulator (NEMO) deficiency (Inhibitor of Kappa Light Polypeptide Gene Enhancer in B Cells Gamma Kinase deficiency).

In one aspect of the present invention, the congenital disease is a hematological disease.

In one aspect of the invention, the hematological disease is independently selected from hemoglobinopathies, such as sickle cell disease and P-thalassemia major (Cooley's anaemia); anaemias, which include aplastic anaemia such as Diamond-Blackfan anaemia and Fanconi's anaemia; Cytopenias, which include amegakaryocytic thrombocytopenia; hemophagocytic syndromes such as hemophagocytic lymphohistiocytosis (HLH); and malignancies, which include solid tumour cancers such as neuroblastoma.

For the avoidance of doubt, the present invention extends to any combination of the aforementioned aspects.

### Definitions

### BRIEF DESCRIPTION OF THE DRAWINGS

FIG. 1 shows white blood cell mobilisation as a function of time expressed as a fold increase relative to the control group (PBS) in three C57BL/6 mice and the average for compounds 239 and 240 at a dose of 15 mg/kg body weight when administered intraperitoneally. The control group (PBS) is also shown in this figure.

FIG. 2 shows the white blood cell (WBC) concentration in peripheral blood as a function of time in C57BL/6 mice for compounds 239 and 240 at a dose of 15 mg/kg body weight when administered intravenously. The control group (PBS) is also shown in this figure.

FIG. 3 shows the white blood cell concentration in peripheral blood as a function of time in C57BL/6 mice for compounds 239 and 240 at a dose of 30 mg/kg body weight when administered intravenously. The control group (PBS) is also shown in this figure.

FIG. 4 shows the white blood cell concentration in peripheral blood and in femurs from the same C57BL/6 mice group following administration of compound 240. The control group (CTL) is also shown in this figure.

FIG. 5 shows the white blood cells (WBC) count as a function of time (FIG. 5b), blood LSK cell mobilisation in % as a function of time (FIG. 5a) and the absolute blood LSK number cell count as a function of time (FIG. 5c), with or without i.v. administration of compound 240 of the present invention at doses of 15 mg/kg, 30 minutes after AMD3100 s.c. administration at 5 mg/kg and 1 h30 after 2.5 μg G-CSF s.c. injection in two months aged-C57Bl/6 mice treated for two days with 2.5 μg G-CSF alone by s.c. injection. The control group (PBS) is also shown in this figure.

FIGS. 6 and 7 show the inhibition of FGF-2-induced normal human dermal fibroblast (NHDF) proliferation by a compound of the present invention.

FIGS. 8-12 show the inhibition of PDGF-β-induced NHDF proliferation by a compound of the present invention.

FIGS. 13-15 show the inhibition of control and VEGF-A-stimulated angiogenesis by 30 μM of compounds according to the present invention using anti-CD31 enzyme linked immunosorbent assay (ELISA; FIGS. 13a and 15a) and the AngioSys image analysis software (TCS CellWorks, England; FIGS. 14a, 14b, 14c and 14d)).

FIGS. 13b and 15b show photographs of stained endothelial tubules on the side of the anti-CD31 ELISA.

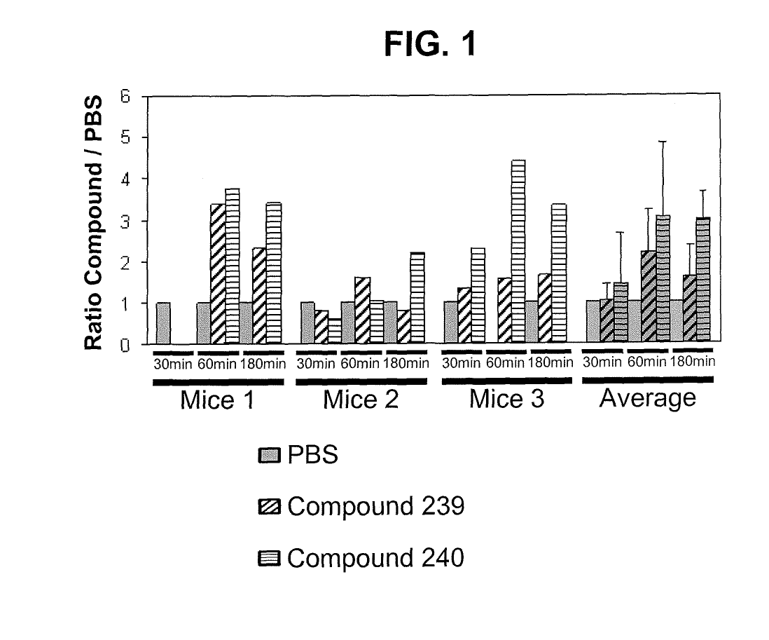

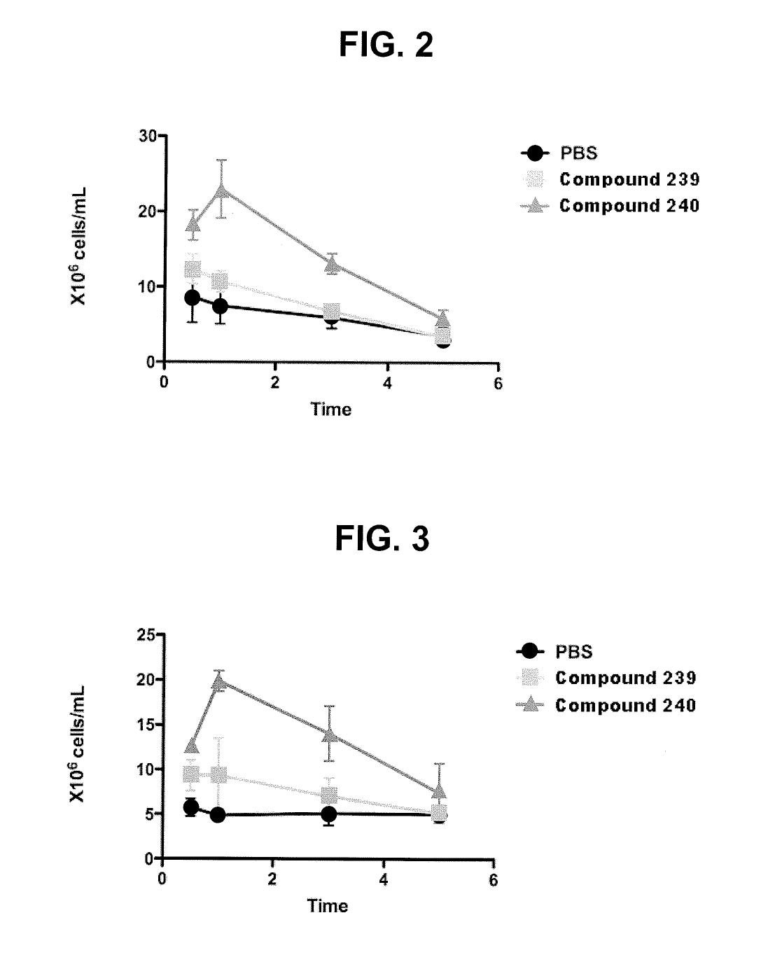

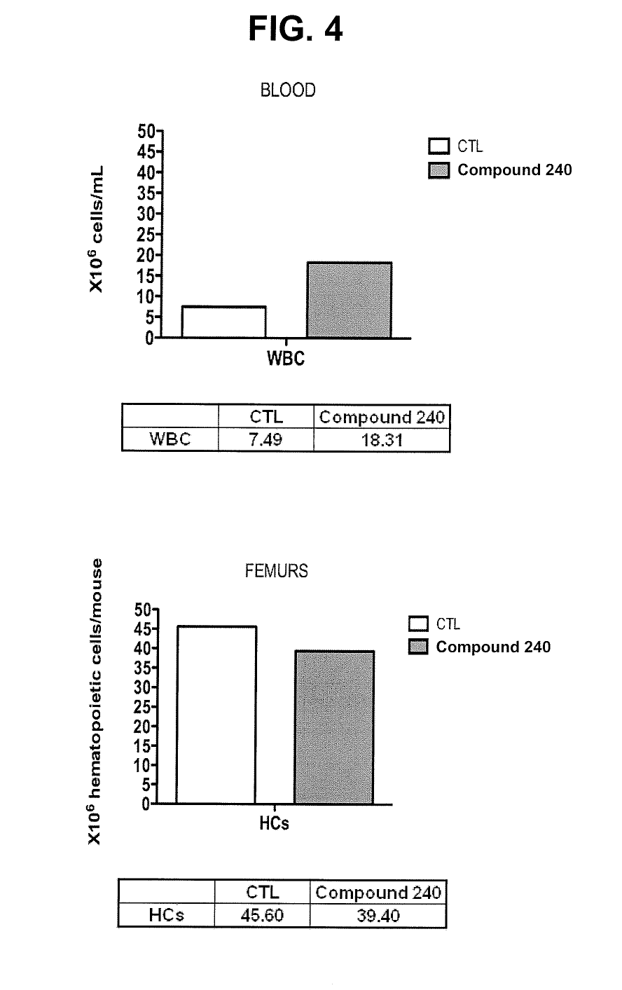

Pharmaceutical compositions The compounds of the present invention may also be present in the form of pharmaceutically acceptable salts. For use in medicine, the salts of the compounds of this invention refer to non-toxic “pharmaceutically acceptable salts.” FDA approved pharmaceutically acceptable salt forms (Gould, P. L. International J. Pharm., 1986, 33, 201-217; Berge, S. M. et al. J. Pharm. Sci., 1977, 66(1), 1-19) include pharmaceutically acceptable acidic/anionic or basic/cationic salts.

Pharmaceutically acceptable salts of the acidic or basic compounds of the invention can of course be made by conventional procedures, such as by reacting the free base or acid with at least a stoichiometric amount of the desired salt-forming acid or base.

Pharmaceutically acceptable salts of the acidic compounds of the invention include salts with inorganic cations such as sodium, potassium, calcium, magnesium, zinc, and ammonium, and salts with organic bases. Suitable organic bases include N-methyl-D-glucamine, arginine, benzathine, diolamine, olamine, procaine and tromethamine.

Pharmaceutically acceptable salts of the basic compounds of the invention include salts derived from organic or inorganic acids. Suitable anions include acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulphate, napsylate, nitrate, oleate, pamoate, phosphate, polygalacturonate, stearate, succinate, sulphate, sulphosalicylate, tannate, tartrate, terephthalate, tosylate and triethiodide. Hydrochloride salts are particularly preferred.

The invention also comprehends derivative compounds (“prodrugs”) which are degraded in vivo to yield the species of Formula (I). Prodrugs are usually (but not always) of lower potency at the target receptor than the species to which they are degraded. Prodrugs are particularly useful when the desired species has chemical or physical properties, which make its administration difficult or inefficient. For example, the desired species may be only poorly soluble, it may be poorly transported across the mucosal epithelium, or it may have an undesirably short plasma half-life. Further discussion of prodrugs may be found in Stella, V. J. et al. “Prodrugs”, Drug Delivery Systems, 1985, 112-176, Drugs, 1985, 29, 455-473 and “Design of Prodrugs”, ed. H. Bundgaard, Elsevier, 1985. Prodrug forms of the pharmacologically active compounds of the invention will generally be compounds according akin to those described in the claims.

Compounds of formula (I) having an amino group may be derivatised with a ketone or an aldehyde such as formaldehyde to form a Mannich base. This will hydrolyse with first order kinetics in aqueous solution.

Thus, in the methods of treatment of the present invention, the term “administering” shall encompass the treatment of the various disorders described with the compound specifically disclosed or with a compound which may not be specifically disclosed, but which converts to the specified compound in vivo after administration to the subject.

Pharmaceutically acceptable ester derivatives in which one or more free hydroxy groups are esterified in the form of a pharmaceutically acceptable ester are particularly prodrug esters that may be convertible by solvolysis under physiological conditions to the compounds of the present invention having free hydroxy groups.

It is anticipated that the compounds of the invention can be administered by oral or parenteral routes, including intravenous, intramuscular, intraperitoneal, subcutaneous, rectal and topical administration, and inhalation.

For oral administration, the compounds of the invention will generally be provided in the form of tablets or capsules or as an aqueous solution or suspension.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate and lactose. Corn starch and alginic acid are suitable disintegrating agents. Binding agents may include starch and gelatine. The lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatine capsules in which the active ingredient is mixed with a solid diluent and soft gelatine capsules wherein the active ingredient is mixed with water or an oil such as peanut oil, liquid paraffin or olive oil.

For intramuscular, intraperitoneal, subcutaneous and intravenous use, the compounds of the invention will generally be provided in sterile aqueous solutions or suspensions, buffered to an appropriate pH and isotonicity. Suitable aqueous vehicles include Ringer's solution and isotonic sodium chloride. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxy benzoate.

The pharmaceutical compositions of the present invention may, in particular, comprise more than one agent (multiple) of the present invention, e.g., two or more agents. The invention also provides a pharmaceutical preparation or system, comprising (a) a first agent, which is an agent of the invention; and (b) a second pharmaceutical agent. Said multiple agents of the invention or said first and second agents are formulated either in admixture or as separate compositions, e.g. for simultaneous though separate, or for sequential administration (see below).

Modes of Administration The compositions of the present invention can be delivered directly or in pharmaceutical compositions containing excipients (see above), as is well known in the art. The present methods of treatment involve administration of a therapeutically effective amount of an agent of the present invention to a subject.

The term “therapeutically effective amount” as used herein refers to an amount of an agent according to the present invention needed to treat, ameliorate, or prevent the targeted disease condition, or to exhibit a detectable therapeutic or preventative effect. In general, the therapeutically effective dose can be estimated initially either in cell culture assays or in animal models, for example, in non-human primates, mice, rabbits, dogs, or pigs. The animal model may also be used to determine the appropriate concentration range and route of administration. Such information can then be used to determine useful doses and routes for administration in humans.

Effective doses of the compounds of the present invention may be ascertained by conventional methods. The specific dosage level required for any particular patient will depend on a number of factors, including severity of the condition being treated, the route of administration, the general health of the patient (i.e. age, weight and diet), the gender of the patient, the time and frequency of administration, and tolerance/response to therapy. In general, however, the daily dose (whether administered as a single dose or as divided doses) will be in the range 0.001 to 5000 mg per day, more usually from 1 to 1000 mg per day, and most usually from 10 to 200 mg per day. Alternatively, dosages can be administered per unit body weight and in this instance a typical dose will be between 0.01 μg/kg and 50 mg/kg, especially between 10 μg/kg and 10 mg/kg, between 100 μg/kg and 2 mg/kg.

An advantage of the compounds of the present invention is that they permit administration to be limited to one, two, three or four times weekly or monthly.

An effective and convenient route of administration and an appropriate formulation of the agents of the invention in pharmaceutical compositions (see above) may also be readily determined by routine experimentation. Various formulations and drug delivery systems are available in the art (see, e.g., Gennaro A R (ed.). Remington: The Science and Practice of Pharmacy. Lippincott Williams & Wilkins. 21st edition. Jul. 3, 2005 and Hardman J G, Limbird L E, Alfred G. Gilman A G. Goodman & Gilman's The Pharmacological Basis of Therapeutics. McGraw-Hill; 10th edition. Aug. 13, 2001).

Suitable routes of administration may, for example, include vaginal, rectal, intestinal, oral, nasal (intranasal), pulmonary or other mucosal, topical, transdermal, ocular, aural, and parenteral administration.

Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

For compositions useful for the present methods of treatment, a therapeutically effective dose can be estimated initially using a variety of techniques well known in the art. Initial doses used in animal studies may be based on effective concentrations established in cell culture assays. Dosage ranges appropriate for human patients can be determined, for example, using data obtained from animal studies and cell culture assays.

A therapeutically effective dose or amount of an agent, agent, or drug of the present invention refers to an amount or dose of the agent, agent, or drug that results in amelioration of symptoms or a prolongation of survival in a patient. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. Agents that exhibit high therapeutic indices are preferred.

The effective amount or therapeutically effective amount is the amount of the agent or pharmaceutical composition that will elicit the biological or medical response of a tissue, system, animal, or human that is being sought by the researcher, veterinarian, medical doctor, or other clinician, e.g., regulation of glucose metabolism, decrease in elevated or increased blood glucose levels, treatment or prevention of a disorder associated with altered glucose metabolism, e.g., diabetes, etc.

Dosages preferably fall within a range of circulating concentrations that includes the ED₅₀ with little or no toxicity. Dosages may vary within this range depending upon the dosage form employed and/or the route of administration utilised. The exact formulation, route of administration, dosage, and dosage interval should be chosen according to methods known in the art, in view of the specifics of a patient's condition.

Dosage amount and interval may be adjusted individually to provide plasma levels of the active moiety that are sufficient to achieve the desired effects, i.e., minimal effective concentration (MEC). The MEC will vary for each agent but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the patient being treated, the severity of the affliction, the manner of administration, and the judgement of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising an agent of the invention formulated in a compatible pharmaceutical carrier may also be prepared, placed in an appropriate container, and labelled for treatment of an indicated condition.

Chemical Definitions Formulaic representation of apparent orientation of a functional group is not necessarily intended to represent actual orientation. Thus, for example, a divalent amide group represented as C(O)NH is also intended to cover NHC(O). In the interests of simplicity, terms which are normally used to refer to monovalent groups (such as “alkyl” or “alkynyl”) are also used herein to refer to divalent, trivalent or tetravalent bridging groups which are formed from the corresponding monovalent group by the loss of one or more hydrogen atom(s). Whether such a term refers to a monovalent group or to a polyvalent group will be clear from the context. Where a polyvalent bridging group is formed from a cyclic moiety, the linking bonds may be on any suitable ring atom, subject to the normal rules of valency.

The terms “comprising” and “comprises” means “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

“Optional” or “optionally” means that the subsequently described event or circumstances may or may not occur, and that the description includes instances where said event or circumstance occurs and instances in which it does not.

“May” means that the subsequently described event of circumstances may or may not occur, and that the description includes instances where said event or circumstance occurs and instances in which it does not.

Where the compounds according to this invention have at least one chiral centre, they may accordingly exist as enantiomers. Where the compounds possess two or more chiral centres, they may additionally exist as diastereomers. Where the processes for the preparation of the compounds according to the invention give rise to mixture of stereoisomers, these isomers may be separated by conventional techniques such as preparative chromatography. The compounds may be prepared in racemic form or individual enantiomers may be prepared by standard techniques known to those skilled in the art, for example, by enantiospecific synthesis or resolution, formation of diastereomeric pairs by salt formation with an optically active acid, followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column. It is to be understood that all such isomers and mixtures thereof are encompassed within the scope of the present invention.

As used herein, the term “substituted” is contemplated to include all permissible substituents of organic compounds. In a broad aspect, the permissible substituents 10 include acyclic and cyclic, branched and unbranched, carbocyclic and heterocyclic, aromatic and nonaromatic substituents of organic compounds. The permissible substituents can be one or more and the same or different for appropriate organic compounds. For purposes of this invention, heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

Where any particular moiety is substituted, for example a phenyl group comprising a substituent on the aryl ring, unless specified otherwise, the term “substituted” contemplates all possible isomeric forms. For example, substituted phenyl includes all of the following ortho-, meta- and para-permutations:

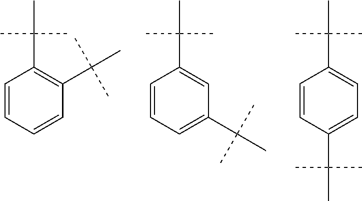

As used herein, when referring to a substitution, it means that the hydrocarbon chain is interrupted by one or more of the groups indicated. Where more than one substitution occurs, it may be adjacent to another or remote, i.e., separated by 0, 1, 2, 3, 4, 5, 6, 7, 8, 9, 10 or more carbon atoms.

Furthermore, the term “substituted” comprehends a substitution that may be adjacent or remote to the point of attachment of the group being substituted to the rest of the molecule. It also comprehends the group being the point of attachment to the rest of the molecule. Where a group comprises two or more moieties defined by a single carbon atom number, for example, C₂₋₁₀-alkoxyalkyl, the carbon atom number indicates the total number of carbon atoms in the group.

As used herein, the term “heteroatom” includes N, O, S, P, Si and halogen (including F, Cl, Br and I).

The term “halogen” or “halo” is used herein to refer to any of fluorine, chlorine, bromine and iodine. Most usually, however, halogen substituents in the compounds of the invention are chlorine, bromine and fluorine substituents. Groups such as halo(alkyl) includes mono-, di- or tri-halo substituted alkyl groups. Moreover, the halo substitution may be at any position in the alkyl chain. “Perhalo” means completely halogenated, e.g., trihalomethyl and pentachloroethyl.

As used herein, the term “alkyl” refers to a straight or branched saturated monovalent hydrocarbon radical, having the number of carbon atoms as indicated. For example, the term “C₁₋₁₀-alkyl” includes C₁, C₂, C₃, C₄, C₅, C₆, C₇, C₈, C₉ and C₁₀ alkyl groups. By way of non-limiting example, suitable alkyl groups include methyl, ethyl, propyl, iso-propyl, butyl, iso-butyl, tert-butyl, pentyl, hexyl, heptyl, octyl, nonyl, cyclopropyl, cyclobutyl, cyclopentyl, cyclohexyl, methylcyclohexyl, dimethylcyclohexyl, trimethylcyclohexyl, cycloheptyl, cyclooctyl, cyclononyl, and adamantyl, cyclopropylmethyl, cyclopropylethyl, cyclobutylmethyl, cyclobutylethyl, cyclopentylmethyl, cyclopentylethyl, cyclopentylpropyl, cyclohexylmethyl, cyclohexylethyl, cyclohexylpropyl, cyclohexylbutyl, methylcyclohexylmethyl, dimethylcyclohexylmethyl, trimethylcyclohexylmethyl, cycloheptylmethyl, cycloheptylethyl and cycloheptylpropyl. In one aspect of the present invention ranges of alkyl groups are: C₁₋₁₀-alkyl, C₁₋₉-alkyl, C₁₋₈-alkyl, C₁₋₇-alkyl, C₁₋₆-alkyl, C₁₋₅-alkyl, C₁₋₄-alkyl, C₁₋₃-alkyl and C₁₋₂-alkyl.

As used herein, the term “cycloalkyl” refers to cyclic saturated monovalent hydrocarbon radical, having the number of carbon atoms as indicated. For example, the term “C₃₋₁₀cycloalkyl” includes C₃, C₄, C₅, C₆, C₇, C₈, C₉ and C₁₀ cycloalkyl groups. By way of non limiting examples suitable cycloalkyl group include cyclopropyl, cyclobutyl, cyclopentyl, cyclohexyl, methylcyclohexyl, dimethylcyclohexyl, trimethylcyclohexyl, cycloheptyl, cyclooctyl, cyclononyl, and adamantyl, cyclopropylmethyl, cyclopropylethyl, cyclobutylmethyl, cyclobutylethyl, cyclopentylmethyl, cyclopentylethyl, cyclopentylpropyl, cyclohexylmethyl, cyclohexylethyl, cyclohexylpropyl, cyclohexylbutyl, methylcyclohexylmethyl, dimethylcyclohexylmethyl, trimethylcyclohexylmethyl, cycloheptylmethyl, cycloheptylethyl and cycloheptylpropyl.

As used herein, the term “alkenyl” refers to a straight or branched unsaturated monovalent hydrocarbon radical, having the number of carbon atoms as indicated, and the distinguishing feature of a carbon-carbon double bond. For example, the term “C₂₋₁₀-alkenyl” includes C₂, C₃, C₄, C₅, C₆, C₇, C₈, C₉ and C₁₀ alkenyl groups. By way of non-limiting example, suitable alkenyl groups include ethenyl, propenyl, butenyl, pentenyl, hexenyl, heptenyl, octenyl and nonenyl, wherein the double bond may be located anywhere in the carbon chain. In one aspect of the present invention ranges of alkenyl groups are: C₂₋₁₀-alkenyl, C₂₋₉-alkenyl, C₂₋₈-alkenyl, C₂₋₇-alkenyl, C₂₋₆-alkenyl, C₂₋₅-alkenyl, C₂₋₄-alkenyl and C₂₋₃-alkenyl.

As used herein, the term “cycloalkenyl” refers to cyclic unsaturated monovalent hydrocarbon radical, having the number of carbon atoms as indicated, and the distinguishing feature of a carbon-carbon double bond. For examples, the term “C₃-C₁₀ cycloalkenyl group” includes C₃, C₄, C₅, C₆, C₇, C₈, C₉ and C₁₀ cycloalkenyl group.

As used herein, the term “alkynyl” refers to a straight or branched unsaturated monovalent hydrocarbon radical, having the number of carbon atoms as indicated, and the distinguishing feature of a carbon-carbon triple bond. For example, the term “C₂₋₁₀ alkynyl” includes C₂, C₃, C₄, C₅, C₆, C₇, C₈, C₉ and C₁₀ alkynyl groups. By way of non-limiting example, suitable alkynyl groups include ethynyl, propynyl, butynyl, pentynyl, hexynyl, heptynyl, octynyl and nonynyl, wherein the triple bond may be located anywhere in the carbon chain. In one aspect of the present invention ranges of alkynyl groups are: C₂₋₁₀-alkynyl, C₂₋₉-alkynyl, C₂₋₈-alkynyl, C₂₋₇-alkynyl, C₂₋₆-alkynyl, C₂₋₅-alkynyl, C₂₋₄-alkynyl and C₂₋₃-alkynyl.

C₁₋₁₀acyl refers to the groups “C(O)—C₁₋₁₀alkyl” where alkyl is as defined above. By way of non limiting example, suitable acyl group includes acetyl, ethylcarbonyl, tert-butylcarbonyl or isopropylcarbonyl.

As used herein, the term “aryl” refers to monovalent unsaturated aromatic carbocyclic radical having one, two, or three rings, which may be fused or bicyclic. In one aspect of the present invention, the term “aryl” refers to an aromatic monocyclic ring containing 5 or 6 carbon atoms, which may be substituted on the ring with 1, 2, 3, 4 or 5 substituents as defined herein; an aromatic bicyclic or fused ring system containing 7, 8, 9 or 10 carbon atoms, which may be substituted on the ring with 1, 2, 3, 4, 5, 6, 7, 8 or 9 substituents as defined herein; or an aromatic tricyclic ring system containing 10 carbon atoms, which may be substituted on the ring with 1, 2, 3, 4, 5, 6, 7, 8, 9, 10, 11, 12 or 13 substituents as defined herein. By way of non-limiting example, suitable aryl groups include phenyl, biphenyl, indanyl, azulenyl, tetrahydronaphthyl, tolyl, chlorophenyl, dichlorophenyl, trichlorophenyl, methoxyphenyl, dimethoxyphenyl, trimethoxyphenyl, fluorophenyl, difluorophenyl, trifluorophenyl, nitrophenyl, dinitrophenyl, trinitrophenyl, aminophenyl, diaminophenyl, triaminophenyl, cyanophenyl, chloromethylphenyl, tolylphenyl, chloroethylphenyl, trichloromethylphenyl, dihydroindenyl, benzocycloheptyl and trifluoromethylphenyl. In one aspect of the present invention ranges of aryl groups are: C₃₋₁₀-aryl, C₄₋₉-aryl, C₅₋₈-aryl and C₆₋₇-aryl. The term “C₃₋₁₀cycloalkyl” refers to a saturated carbocyclic ring having 3 to 10 carbon atoms. By way of non limiting example, suitable C₃₋₁₀cycloalkyl includes cyclopropyl, cyclobutyl, cyclopentyl, cyclohexyl, cyclohepryl, cyclooctyl or cyclononyl.

The term “C₃₋₁₀heteroaryl” refers to monovalent unsaturated aromatic heterocyclic radicals containing 3 to 10 membres having one, two, three rings containing at least one hetereoatom, in particular O, N or S, advantageously two heteroatoms, in particular 3 heteroatoms, which may be fused or bicyclic. Suitably, the term “heteroaryl” encompasses heteroaryl moieties that are aromatic monocyclic ring systems containing five members of which at least one member is a N, O or S atom and which optionally contains one, two or three additional N atoms, an aromatic monocyclic ring having six members of which one, two or three members are a N atom, aromatic bicyclic or fused rings having nine members of which at least one member is a N, O or S atom and which optionally contains one, two or three additional N atoms or aromatic bicyclic rings having ten members of which one, two or three members are a N atom. By way of non-limiting example, suitable heteroaryl groups include furanyl, pyridyl, phthalimido, thiophenyl, pyrrolyl, imidazolyl, pyrazolyl, thiazolyl, isothiazolyl, oxazolyl, oxadiazolyl, pyronyl, pyrazinyl, tetrazolyl, thionaphthyl, benzo furanyl, indolyl, oxyindolyl, isoindolyl, indazolyl, indolinyl, azaindolyl, benzopyranyl, coumarinyl, isocoumarinyl, quinolyl, isoquinolyl, cinnolinyl, quinazolinyl, benzoxazinyl, chromenyl, chromanyl, isochromanyl, thiazolyl, isoxazolyl, isoxazolonyl, isothiazolyl, triazolyl, oxadiazolyl, thiadiazolyl, triazyl and pyridazyl, advantageously triazyl. In one aspect of the present invention ranges of heteroaryl groups are: C₄₋₉-heteroaryl, C₅₋₈-heteroaryl and C₆₋₇-heteroaryl.

As used herein, the term “arylalkyl” refers to an aryl group with an alkyl substituent. Binding is through the aryl group. Such groups have the number of carbon atoms as indicated. The alkyl and aryl moieties of such a group may be substituted as defined herein, with regard to the definitions of alkyl and aryl. The alkyl moiety may be straight or branched. Typical examples of alkaryl include tolyl, xylyl, butylphenyl, mesityl, ethyltolyl, methylindanyl, methylnaphthyl, methyltetrahydronaphthyl, ethylnaphthyl, dimethylnaphthyl, propylnaphthyl and butylnaphthyl.

As used herein, the term “alkylaryl” refers to an alkyl group with an aryl substituent. Binding is through the alkyl group. Such groups have the number of carbon atoms as indicated. The aryl and alkyl moieties of such a group may be substituted as defined herein, with regard to the definitions of aryl and alkyl. The alkyl moiety may be straight or branched. Typical examples of arylalkyl include benzyl, methylbenzyl, ethylbenzyl, dimethylbenzyl, diethylbenzyl, methylethylbenzyl, methoxybenzyl, chlorobenzyl, dichlorobenzyl, trichlorobenzyl, phenethyl, phenylpropyl, phenylbutyl, fluorobenzyl, difluorobenzyl, trifluorobenzyl, trifluoromethylbenzyl, bis(trifluoromethyl)benzyl, propylbenzyl, tolylmethyl, fluorophenethyl, fluorenylmethyl, methoxyphenethyl, dimethoxybenzyl, dichlorophenethyl, phenylethylbenzyl, isopropylbenzyl, diphenylmethyl, propylbenzyl, butylbenzyl, dimethylethylbenzyl, phenylpentyl, tetramethylbenzyl, phenylhexyl, dipropylbenzyl, triethylbenzyl, cyclohexylbenzyl, naphthylmethyl, diphenylethyl, triphenylmethyl and hexamethylbenzyl.

As used herein, the term “acylaryl” refer to an acyl group with an aryl substituent. Binding is through the acyl group. Such groups have the number of carbon atoms as indicated. The aryl and acyl moieties of such a group may be substituted as defined above. The acyl moiety may be straight or branched. Typical examples of acylaryl include, for example, ketobenzyl.

With regard to one or more substituents which are referred to as being on the carbon backbone of a group with a compound definition, for example, “alkaryl”, the substituent may be on either or both of the component moieties, e.g., on the alkyl and/or aryl moieties. Reference to cyclic systems, e.g., aryl, heteroaryl, cycloalkyl, etc., contemplates monocyclic and polycyclic systems. Such systems comprise fused, non-fused and spiro conformations, such as bicyclooctyl, adamantyl, and benzofuran.

The term “monosaccharide” means a sugar molecule having a chain of 3-10 carbon atoms in the form of an aldehyde (aldose) or ketone (ketose). Suitable monosaccharides for use in the invention include both naturally occurring and synthetic monosaccharides. Such monosaccharides include trioses, such as glycerone and dihydroxyacetone; textroses, such as erythanose and erythrulose; pentoses, such as xylose, arabinose, ribose, xylulose and ribulose; methyl pentoses (6-deoxyhexoses), such as rhamnose and fructose; hexoses, such as glucose, mannose, galactose, fructose and sorbose; heptoses, such as glucoheptose, galamannoheptose, sedoheptulose and mannoheptulose. Suitably the monosaccharides are hexoses.

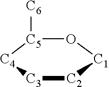

The monosaccharides may be attached to another monosaccharide group at the C₁, C₂, C₃, C₄, C₅ and C₆ position (shown above) to form a glycosyl bond and an oligosaccharide. In the present invention, a monosaccharide is attached to the C4 position through an oxygen atom attached to the C₁ carbon of another monosaccharide, which forms a glycosidic linkage and an oligosaccharide. Oligosaccharides that can be used in the present invention include: disaccharides, trisaccharides, tetrasaccharides, pentasaccharides, hexasaccharides, heptasaccharides, octasaccharides, nonasaccharides, decasaccharides, undecasaccharides and dodecasaccharides.

The group at the C1 position is also known as the anomeric or hemiacetal carbon. Stereoisomers of a saccharide in the cyclic form can differ only in the configuration at this position. For example, if the saccharide in question is glucose and the group at the C1 position is in the axial position, the saccharide is an alpha anomer. If, however, the same group at the C1 position is at the equatorial position, the saccharide is a beta anomer. By way of example, α-D-glucopyranose and β-D-glucopyranose, the two cyclic forms of glucose are shown below. For L-saccharides the alpha and beta anomers are contrariwise.

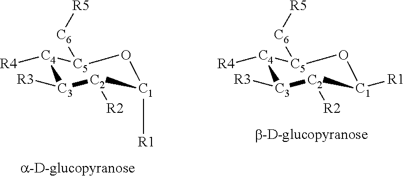

It will be appreciated that ionisable groups may exist in the neutral form shown in formulae herein, or may exist in charged form e.g. depending on pH. Thus, a carboxylate group may be shown as COOH, which is merely representative of the neutral carboxylate group. The present invention also encompasses other charged forms (i.e. COO⁻).

Similarly, references herein to cationic and anionic groups should be taken to refer to the charge that is present on that group under physiological conditions e.g. where a sulphate group O—SO₃H is deprotonated to give the anionic O—SO₃⁻ group, this deprotonation is one that occurs at physiological pH. In addition where a carboxyl group COOH is deprotonated to give the anionic COO⁻ group, this deprotonation is one that can occur at physiological pH. Moreover, charged salts of the molecules of the invention are encompassed. Saccharide rings can exist in an open and closed form, while closed forms are shown herein, open forms are also encompassed by the invention. Similarly, isomeric forms of the molecules of the invention are also encompassed, including tautomers, conformers, enantiomers and diastereoisomers, for example.

The counter-ions, which compensate the charged forms of the compounds of the present invention, are pharmaceutically acceptable counter-ions such as hydrogen, or typically alkali or alkali-earth metals ions, which include sodium, calcium, magnesium and potassium.

Other ‘compound’ group definitions will be readily understandable by the skilled person based on the previous definitions and the usual conventions of nomenclature.

Certain compounds of the invention exist in various regioisomeric, enantiomeric, tautomeric and diastereomeric forms. It will be understood that the invention comprehends the different regioisomers, enantiomers, tautomers and diastereomers in isolation from each other as well as mixtures.

The term “angiogenic protein” relates to a heparan sulphate binding protein that interacts with heparan sulphate that is involved in angiogenesis. Specifically, this term is meant to encompass growth factors, enzymes and chemokines, which are defined below.

The term “growth factor” relates to a naturally occurring protein capable of stimulating cellular proliferation and/or migration and/or cellular differentiation. Growth factors are important for regulating a variety of cellular processes. Growth factors typically act as signalling molecules between cells. Typical examples of growth factors include: transforming growth factor beta (TGF-β); granulocyte-colony stimulating factor (G-CSF); granulocyte-macrophage colony stimulating factor (GM-CSF); nerve growth factor (NGF); neurotrophins; platelet-derived growth factor (PDGF); erythropoietin (EPO); thrombopoietin (TPO); myostatin (GDF-8); growth differentiation factor-9 (GDF9); acidic fibroblast growth factor (aFGF or FGF-1); basic fibroblast growth factor (bFGF or FGF-2); epidermal growth factor (EGF); vascular endothelial growth factor (VEGF); placental growth factor (PlGF) and hepatocyte growth factor (HGF). In one aspect of the present invention suitable growth factors include: members of the VEGF family, such as VEGF-A; members of the FGF family, such as FGF-1; FGF-2; placental growth factor (PlGF); and PDGF-β.

The term “enzyme” refers to an enzyme that is involved in angiogenesis and/or metastasis. In particular, this term encompasses a moiety that interacts with heparan sulphate in angiogenesis and/or metastasis. An example of an enzyme of the present invention is heparanase.

The term “chemokine” refers to a chemokine that is involved in angiogenesis and/or metastasis. In particular, this term encompasses a moiety that is involved in angiogenesis and/or metastasis. An example of a chemokine of the present invention is SDF-1, such as SDF-1α, SDF-1β and SDF-1γ.

It will be appreciated that any optional feature that has been described above in relation to any one aspect of the invention may also be applicable to any other aspect of the invention.

### EXPERIMENTAL

Abbreviations used:
• DMF: N,N-Dimethylformamide; • Et₃N: triethylamine; • TEMPO: 2,2,6,6-Tetramethylpiperidin-1-oxyl; • DMAP: 4-dimethylaminopyridine; • EDAC: N-(3-dimethylaminopropyl)-N′-ethylcarbodiimide hydrochloride; • TBDPS: tert-butyldiphenylsilyl; • TBDMS: tert-butyldimethylsilyl; • Bn: benzyl; • Ph: phenyl; • Bz: benzoyl; • PMB: p-methoxybenzyl; • Me: methyl; • Ac: acetate; • Lev: levulinoyl

### I. Section 1

### Preparation

### A. General Methods

### Method A: General Method for O-Glycosylation

In a dry round-bottom flask, the saccharide donor (1.1 eq.) and the saccharide acceptor (1 eq.) were azeotropically dried with toluene and dissolved in anhydrous dichloromethane (0.07 M) under a nitrogen atmosphere containing 4 Å molecular sieves (1 weight eq.) previously activated at 400° C. After stirring for 30 min at room temperature, the reaction mixture was cooled down to 0° C. or 20° C. and N-iodosuccinimide (2.0 eq.) followed by triflic acid (0.12 eq. vs donor) were added. After stirring for 30 min at 0° C. or 20° C., the reaction mixture was filtered through a pad of Celite®, washed with dichloromethane and successively washed with a 1 M aqueous solution of Na₂S₂O₃ (to quench the excess of iodine), a saturated solution of NaHCO₃ and water. The organic layer was dried over MgSO₄, filtered, the solvent was evaporated under reduced pressure and the residue was purified by chromatography on silica gel column to afford the glycosylated compound.

### Method B: General Method for O-Glycosylation

In a dry round-bottom flask, the saccharide donor (1.3 eq.) and the saccharide acceptor (1 eq.) were dissolved in anhydrous toluene (0.2 to 0.4M/acceptor) under a nitrogen atmosphere containing 4 Å molecular sieves (1 weight eq.) previously activated at 400° C. After stirring for 30 min at room temperature, the solution was cooled down to 20° C. and a 0.1 M solution of tert-butyldimethylsilyl trifluoromethanesulfonate in toluene freshly prepared (0.2 eq. vs donor) was added dropwise. The reaction was warmed from 20° C. to 0° C. over 30 min and the reaction mixture was stirred at this temperature for 1 h. The reaction mixture was neutralized with Et₃N until pH 7, filtered through a pad of Celite® and concentrated to dryness under reduced pressure. The residue was purified by chromatography on silica gel column to afford the glycosylated compound.

### Method C: General Method for Isopropylidene Cleavage

The saccharide was dissolved in a 1/1 mixture of tetrahydrofurane/acetic acid 60% in water (0.16 M) at room temperature. The reaction mixture was stirred at 80° C. until complet conversion. The reaction mixture was concentrated under reduced pressure and coevaporated with toluene. The residue was dissolved in dichloromethane and successively washed with a saturated aqueous solution of NaHCO₃ and a brine solution. The organic layer was dried over MgSO₄, filtered and concentrated under reduced pressure to afford the crude compound.

### Method D: General Method for Oxidation

To a solution of 0.015 M of a saccharide in a mixture of acetonitrile/NaHCO_{3aq}. (50/50) at room temperature were added TEMPO (0.1 eq) and 1,3-dibromo-5,5-dimethylhydantoin (2 eq.). The reaction mixture was stirred for 2 h at room temperature after which a 1 M aqueous solution of Na₂S₂O₃ (to neutralize the 1,3-dibromo-5,5-dimethylhydantoin reagent) and ethyl acetate were added. The reaction mixture was cooled to 0° C. and an aqueous solution of 1 M H₂SO₄ was added. The organic layer was separated and the aqueous layer was extracted with ethyl acetate. The organic layers were combined, dried over MgSO₄, filtered and concentrated under reduced pressure to give the intermediate carboxylic acid which was directly used in the next step without any further purification.

### Method E: General Method for Esterification

To a solution of carboxylic acid in anhydrous DMF (0.1 M) under a nitrogen atmosphere were added iodomethane (10 eq.) followed by solid NaHCO₃ (10 eq.). The reaction mixture was stirred overnight at room temperature. The reaction mixture was diluted with ethyl acetate and successively washed with aqueous solution of Na₂S₂O₃ (1 M), a brine solution and water. The organic layer was dried over MgSO₄, filtered, concentrated under reduced pressure and the residue was purified by chromatography on silica gel column to give the desired compound.

### Method F: General Method for Acetolysis

In a dry round-bottom flask, the saccharide was dissolved in a mixture of acetic anhydride (100 eq.) and trifluoroacetic acid (11 eq.). The reaction mixture was stirred overnight at room temperature and solvents were removed under reduced pressure followed by co-evaporation with toluene. The residue was purified by flash chromatography on silica gel column to give the desired compound or directly used in the next step without any further purification after washing with a saturated aqueous solution of NaHCO₃.

### Method G: General Method for Selective Anomer Deacetylation

In a dry round-bottom flask, a solution of 0.1 M of a saccharide in a mixture of tetrahydrofurane/methanol (7/3) was introduced and cooled down to 0° C. After stirring for 15 min, the solution was bubbled with a gentle flow of ammonia for 30 min until complet conversion. The reaction mixture was then purged with nitrogen for 15 min and concentrated to dryness under reduced pressure. The crude product was purified by flash chromatography on silica gel column to give the desired compound or directly used in the next step without any further purification.

### Method H: General Method for Trichloroacetimidate Formation

To a solution of 0.1 M of a saccharide in anhydrous dichloromethane in a dry round-bottom flask under a nitrogen atmosphere, was added trichloroacetonitrile (6 eq.). The reaction mixture was cooled to 0° C. and cesium carbonate (1.8 eq.), previously activated at 400° C., was added. After stirring at room temperature for 2 h, the reaction mixture was filtered through a pad of Celite®, washed with dichloromethane and the filtrate was washed with water, dried over MgSO₄, filtered and concentrated to dryness. The residue was purified by flash chromatography on silica gel column to give the desired trichloroacetimidate or directly used in the next step without any further purification.

### B. Monosaccharides Preparations

### 1-Preparation 1: Synthesis of Elongating Monosaccharides 4, 5 and 8 (Scheme 1)

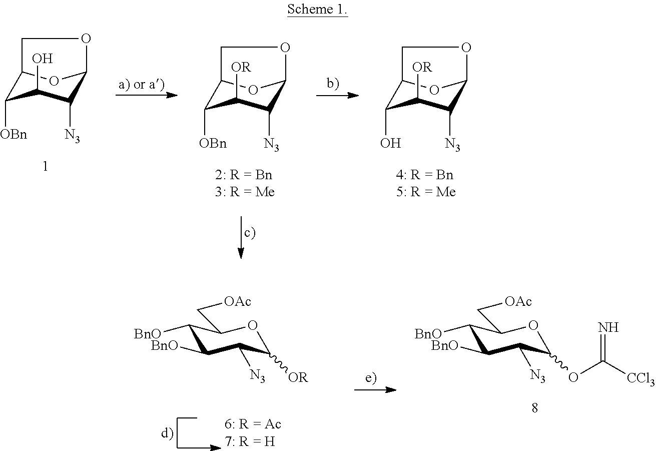

Step 1.a: Synthesis of compound 2: In a dry round-bottom flask, compound 1 (25 g, 90.2 mmol), which was prepared as described in Bull. Chem. Soc. Jpn., 1999, 72, 1857-1867, was dissolved in anhydrous DMF (300 mL) under a nitrogen atmosphere. Benzyl bromide (12.9 mL, 108.2 mmol, 1.2 eq.) was added and the reaction mixture was cooled down to 0° C. NaH (60% dispersion in oil, 5.41 g, 135.2 mmol, 1.5 eq.) was then added by portions over 5 min. After 2 h, the reaction was cooled to 0° C. and the excess of NaH was neutralized with methanol (100 mL). The reaction mixture was concentrated to ½ of the total volume, then diluted with ethyl acetate (1 L) and washed with a saturated aqueous solution of NaCl (3×400 mL) and water (400 mL). The organic layer was dried over MgSO₄, filtered and concentrated under reduced pressure to afford crude compound 2 as a clear yellow oil which was directly used in the next step without any further purification.

Step 1.a′: Synthesis of compound 3: In a dry round-bottom flask, compound 1 (35 g, 126.2 mmol) was dissolved in anhydrous DMF (350 mL) under a nitrogen atmosphere. Iodomethane (18.2 mL, 164.1 mmol, 1.3 eq.) was added and the reaction mixture was cooled down to 0° C. NaH (60% dispersion in oil, 6.06 g, 151.4 mmol, 1.2 eq.) was then added by portions over 5 min. After 1 h 30, the reaction was cooled down to 0° C. and the excess of NaH was neutralized with methanol (150 mL). The reaction mixture was concentrated to ½ of the total volume, then diluted with ethyl acetate (1 L) and washed with a saturated aqueous solution of NaCl (2×500 mL) and water (500 mL). The organic layer was dried over MgSO₄, filtered and concentrated under reduced pressure to afford crude compound 3 as a yellow oil which was directly used in the next step without any further purification.

Step 1.b: Synthesis of compound 4: In a dry round-bottom flask, compound 2 (90.2 mmol) was dissolved in dry dichloromethane (300 mL) under a nitrogen atmosphere. The reaction mixture was cooled down to 0° C. and titanium chloride (10.9 mL, 99.1 mmol, 1.1 eq.) was added dropwise. After 8 h stirring at room temperature, the reaction mixture was filtered through a pad of Celite®, concentrated under reduced pressure and directly purified by chromatography on silica gel (heptane/ethyl acetate: 8/2 to 5/5) to afford compound 4 (20.6 g, 91% over 2 steps) as a pale yellow solid. ¹H NMR (400 MHz, CDCl₃, ppm): δ=7.55-7.30 (m, 5H, arom.), 5.50 (br. s, 1H, H-1), 4.75-4.60 (q, 2H, J=11.5 Hz, CH₂-Ph), 2.60 (br. s, 1H, OH). MALDI-MS, positive mode, m/z: 299.95 [M+Na⁺], 315.89 [M+K⁺]. [α]_{D}²¹=2.5 (c=1.39, CHCl₃).

Preparation of monosaccharide 5 was carried out as described for 1,6-anhydro-2-azido-2-deoxy-3-O-benzyl-β-D-glucopyranose 4.

Compound 5: ¹H NMR (400 MHz, CDCl₃, ppm): δ=5.47 (br. s, 1H, H-1), 3.46 (s, 3H, OMe). MALDI-MS, positive mode, m/z: 223.85 [M+Na⁺], 239.87 [M+K⁺]. [α]_{D}²¹=29.2 (c=0.91, CHCl₃).

Step 1.c: Synthesis of compound 6: Acetolysis of crude compound 2 (7.21 mmol) was performed according to the general method F to afford crude compound 6 (α/β: 83/17) as a brown solid which was directly used in the next step without any further purification. MALDI-MS, positive mode, m/z: 492.10 [M+Na⁺].

Step 1.d: Synthesis of compound 7: Selective anomeric acetate hydrolysis of compound 6 (7.21 mmol) was performed according to the general method G. Compound 7 was obtained as a viscous brown solid which was directly used in the next step without any further purification. MALDI-MS, positive mode, m/z: 450.03 [M+Na⁺], 465.98 [M+K⁺].

Step 1.e: Synthesis of compound 8: Trichloroacetimidate formation of compound 7 (7.21 mmol) was performed according to the general method H. Purification was effected by chromatography on silica gel column (heptane/ethyl acetate: 9/1 to 7/3 with 1% Et₃N) to give compound 8 (2.00 g, 49% over 4 steps, α/β: 85/15) as a white amorphous compound. ¹H NMR (400 MHz, CDCl₃, ppm): δ=8.73 (s, 1H, NH), 7.47-7.20 (m, 10H, arom.), 6.41 (d, 1H, J=3.5 Hz, H-1+), 5.62 (d, 0.18H, J=8.3 Hz, H-1β), 4.94 (s, 2H, CH₂-Ph), 4.88, 4.60 (2d, 2H, J=10.7 Hz, CH₂-Ph), 4.34-4.19 (m, 2H, H-6a, H-6b), 4.11-4.00 (m, 2H, H-3, H-4), 3.72-3.62 (m, 2H, H-2, H-5), 2.04 (s, 3H, CH₃—OAc). MALDI-MS, positive mode, m/z: 449.91 [M+Na⁺—C(NH)CCl₃].

### 1-Preparation 2: Synthesis of Reducing Monosaccharides 19, 20, 21, 22, 23, 24, 25 and 26 (Scheme 2)

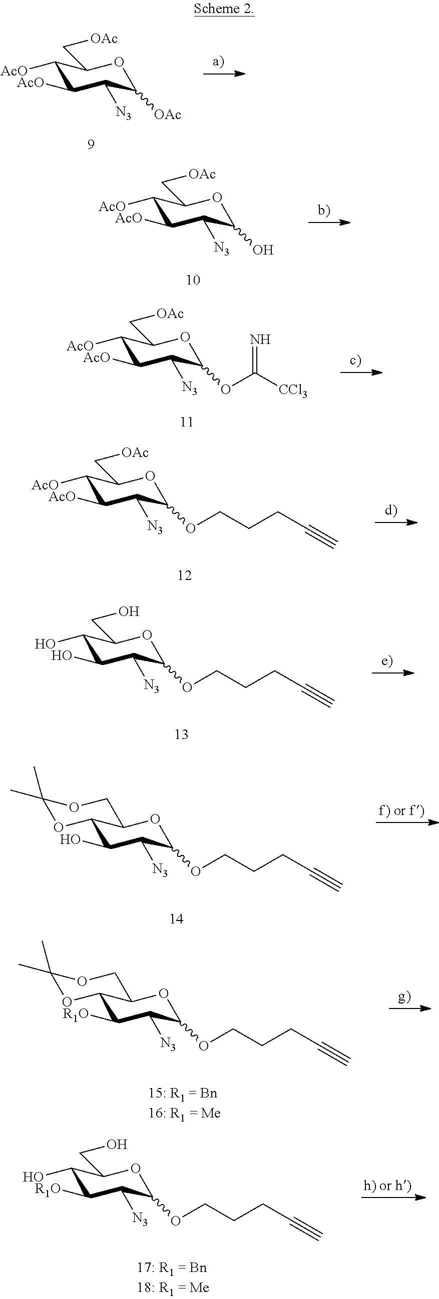

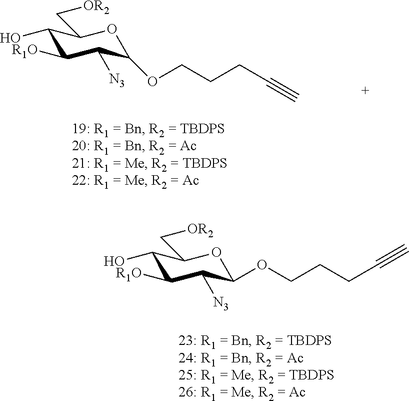

Step 2.a: Synthesis of compound 10: Selective anomeric acetate hydrolysis of 1,3,4,6-tetra-O-acetyl-2-azido-2-deoxy-α,β-D-glucopyranoside 9 (8.06 g, 21.6 mmol), which was prepared as described in Org. Lett. 2007, 9, 3797-3800, was performed according to the general method G. Compound 10 was obtained as a brown oil which was directly used in the next step without any further purification. MALDI-MS, positive mode, m/z: 353.96 [M+Na⁺], 369.93 [M+K⁺].

Step 2.b: Synthesis of compound 11: Trichloroacetimidate formation of compound 10 (21.6 mmol) was performed according to the general method H. Purification was effected by chromatography on silica gel column (heptane/ethyl acetate: 7/3 with 1% Et₃N) to give compound 11 (7.73 g, 75% over 2 steps, α/β: 93/7) as a pale yellow solid. ¹H NMR (400 MHz, CDCl₃, ppm): δ=8.83 (s, 1H, NHα), 8.80 (s, 0.07H, NHβ), 6.48 (d, 1H, J=3.6 Hz, H-1α), 5.71 (d, 0.07H, J=8.6 Hz, H-1β), 5.52 (t, 1H, J=9.7 Hz, H-3), 5.15 (t, 1H, J=9.7 Hz, H-4), 4.27 (dd, 1H, J=4.6 Hz, J=12.6 Hz, H-6a), 4.23-4.18 (m, 1H, H-5), 4.09 (dd, 1H, J=2.2 Hz, J=12.6 Hz, H-6b), 3.77 (dd, 1H, J=3.6 Hz, J=9.7 Hz, H-2), 2.10, 2.05, 2.04 (3s, 9H, CH₃—OAc). MALDI-MS, positive mode, m/z: 498.02 [M+Na⁺].

Step 2.c: Synthesis of compound 12: Coupling reaction of monosaccharide donor 11 (8.62 g, 18.12 mmol, 1 eq.) and 4-pentyn-1-ol (3.35 mL, 36.24 mmol, 2 eq.) was performed in anhydrous dichloromethane (C=0.06 M vs donor) with the activator trimethylsilyl trifluoromethanesulfonate (0.15 eq. vs donor) according to the general method B. The crude compound 12 was directly used in the next step without any further purification. MALDI-MS, positive mode, m/z: 420.07 [M+Na⁺], 436.05 [M+K⁺].

Step 2.d: Synthesis of compound 13: Crude compound 12 (18.12 mmol) was dissolved under a nitrogen atmosphere in a 1/1 mixture of dry tetrahydrofurane and methanol (120 mL) and the solution was cooled to 0° C. A 0.5 M solution of MeONa in methanol (54.4 mL, 27.18 mmol, 1.5 eq.) was slowly added and the resulting solution was stirred 3 h at room temperature. The reaction mixture was neutralized with Amberlite® IRA120 until acidic pH then filtered. The resin was washed several times with methanol. The filtrate was concentrated under reduced pressure to give compound 13 as a yellow oil which was directly engaged in the next step without any further purification. MALDI-MS, positive mode, m/z: 294.23 [M+Na⁺], 310.19 [M+K⁺].

Step 2.e: Synthesis of compound 14: Compound 13 (18.12 mmol) was dissolved under a nitrogen atmosphere in anhydrous DMF (70 mL) at room temperature. Camphor sulfonic acid (421 mg, 1.81 mmol, 0.1 eq.) followed by dimethoxypropane (45 mL, 0.362 mol, 20 eq.) were added. The reacting mixture was stirred overnight at room temperature and neutralized with a saturated aqueous solution of NaHCO₃ (80 mL). The reaction mixture was diluted with ethyl acetate (500 mL) and the organic layer was successively washed with a saturated solution of NaCl (2×100 mL) and water (100 mL). The organic layer was dried over MgSO₄, filtered, concentrated under reduced pressure and filtered though a pad of silica gel (heptane/ethyl acetate: 7/3+1% Et₃N) to give compound 14 as a white solid (4.96 g, 88% over 3 steps). MALDI-MS, positive mode, m/z: 334.26 [M+Na⁺].

Step 2.f: Synthesis of compound 15: In a dry round-bottom flask, compound 14 (6.9 g, 22.16 mmol) was dissolved in anhydrous DMF (100 mL) under a nitrogen atmosphere. Benzyl bromide (3.2 mL, 26.60 mmol, 1.2 eq.) was added and the reaction mixture was cooled to 0° C. NaH (60% dispersion in oil, 1.33 g, 33.24 mmol, 1.5 eq.) was then added by portions over 5 min. After 12 h at room temperature, the reaction was cooled down to 0° C. and the excess of NaH was neutralized with methanol (100 mL). The reaction mixture was diluted with ethyl acetate (600 mL) and washed with a saturated aqueous solution of NaCl (3×200 mL). The organic layer was dried over MgSO₄, filtered and concentrated under reduced pressure. The crude product was purified by flash chromatography on silica gel column (heptane/ethyl acetate: 9/1 to 8/2 with 1% Et₃N) to afford compound 15 (7.1 g, 80%) as a colourless oil. MALDI-MS, positive mode, m/z: 424.30 [M+Na⁺], 440.17 [M+K⁺].

Step 2.f′: Synthesis of compound 16: Monosaccharide 16 was prepared in a similar manner as described for 15, except the iodomethane reagent was used instead of benzyl bromide. Compound 16 was directly used in the next step without any further purification.

Step 2.g: Synthesis of compound 17: Isopropylidene cleavage of compound 15 (6.31 g, 15.72 mmol) was performed according to the general method C. Compound 17 was obtained as a colourless oil and directly used in the next step without any further purification. MALDI-MS, positive mode, m/z: 384.21 [M+Na⁺], 400.14 [M+K⁺].

Step 2.g: Synthesis of compound 18: Compound 18 was prepared in a similar manner as described for 17. Compound 18 was directly used in the next step without any further purification.

Step 2.h: Synthesis of compounds 19 and 23: In a dry round-bottom flask, compound 17 (15.72 mmol) was dissolved in dry dichloromethane (110 mL) under a nitrogen atmosphere. Tert-butylchlorodiphenylsilane (20.4 mL, 78.6 mmol, 5 eq.), Et₃N (10.9 mL, 78.6 mmol, 5 eq.) and DMAP (959 mg, 7.86 mmol, 0.5 eq.) were successively added and the reaction mixture was stirred overnight at room temperature. The solution was dissolved in dichloromethane (100 mL) and the organic layer was washed with a 5% aqueous solution of H₂SO₄ (20 mL), dried over MgSO₄, filtered and concentrated under reduced pressure. The crude residue was purified twice by chromatography on silica gel 6-35 μm column (petroleum ether/ether: 9/1 to 8/2) to give pure compound 19 (3.52 g, anomer α) and pure compound 23 (3.05 g, anomer β) as colourless oils with a global yield of 72% over 2 steps.

Compound 19: ¹H NMR (400 MHz, CDCl₃, ppm): δ=7.74-7.67 (m, 5H, arom.), 7.50-7.37 (m, 10H, arom.), 4.96-4.85 (m, 3H, H-1, CH₂-Ph), 3.94-3.69 (m, 6H, CH₍ₐ₎-pent-4-ynyl, H-4, H-3, H-5, H-6a, H-6b), 3.59-3.52 (m, 1H, CH_{(a′)}-pent-4-ynyl), 3.28 (dd, 1H, J=3.6 Hz, J=10.3 Hz, H-2), 2.56 (s, 1H, OH), 2.39-2.31 (m, 2H, CH_{2(c)}-pent-4-ynyl), 1.93 (t, 1H, J=2.6 Hz, CH_{(d)}-alkyne), 1.91-1.79 (m, 2H, CH_{2(b)}-pent-4-ynyl), 1.09 (s, 9H, C(CH₃)₃). MALDI-MS, positive mode, m/z: 622.11 [M+Na⁺], 638.03 [M+K⁺]. [α]_{D}²¹=+50.7 (c=1.79, CHCl₃).

Compound 23: ¹H NMR (400 MHz, CDCl₃, ppm): δ=7.74-7.67 (m, 5H, arom.), 7.50-7.37 (m, 10H, arom.), 4.94, 4.82 (2d, 2H, J=11.4 Hz, CH₂-Ph), 4.31 (d, 1H, J=7.9 Hz, H-1), 4.01-3.94 (m, 1H, CH₍ₐ₎-pent-4-ynyl), 3.93-3.87 (m, 2H, H-6a, H-6b), 3.76-3.70 (m, 1H, H-5), 3.69-3.62 (m, 1H, CH_{(a′)}-pent-4-ynyl), 3.42-3.33 (m, 2H, H-4, H-3), 3.32-3.25 (m, 1H, H-2) 2.72 (s, 1H, J=2.2 Hz, OH), 2.39-2.31 (m, 2H, CH_{2(c)}-pent-4-ynyl), 1.93 (t, J=2.6 Hz, 1H, CH_{(d)}-alkyne), 1.91-1.79 (m, 2H, CH_{2(b)}-pent-4-ynyl), 1.09 (s, 9H, C(CH₃)₃). MALDI-MS, positive mode, m/z: 622.16 [M+Na⁺], 638.13 [M+K⁺]. [α]_{D}²¹=18.1 (c=0.77, CHCl₃).

Step 2.h′: Synthesis of compounds 20 and 24: In a dry round-bottom flask, compound 17 (10.24 mmol) was dissolved in dry dichloromethane (100 mL) under a nitrogen atmosphere followed by addition of pyridine (830 μL, 10.24 mmol, 1 eq.) and DMAP (62 mg, 0.51 mmol, 0.05 eq.). The solution was cooled down to 0° C. and acetyl chloride (730 μL, 10.24 mmol, 1 eq.) was added dropwise. The reaction mixture was stirred overnight at 4° C. The solution was diluted in dichloromethane (100 mL), washed successively with a saturated aqueous solution of NaHCO₃ (50 mL) and a 5% aqueous solution of H₂SO₄ (50 mL), dried over MgSO₄, filtered and concentrated under reduced pressure. The crude residue was purified twice by chromatography on silica gel 6-35 μm column (petroleum ether/ether: 7/3 to 6/4) to give pure compound 20 (1.4 g, anomer α) and pure compound 24 (1.97 g, anomer β) as colourless oils with a global yield of 85% over 2 steps.

Compound 20: ¹H NMR (400 MHz, CDCl₃, ppm): δ=7.43-7.32 (m, 5H, arom.), 4.85, 4.74 (2d, 2H, J=11.5 Hz, CH₂-Ph), 4.84 (br. s, 1H, H-1), 3.78, 3.50 (m, 2H, CH₂₍ₐ₎-pent-4-ynyl), 2.64 (br. s, 1H, OH), 2.31-2.26 (m, 3H, CH_{2(c)}-pent-4-ynyl, CH_{(d)}-alkyne), 2.02 (s, 3H, CH₃—OAc), 1.86-1.70 (m, 2H, CH_{2(c)}-pent-4-ynyl). MALDI-MS, positive mode, m/z: 426.12 [M+Na⁺], 442.09 [M+K⁺]. [α]_{D}²¹=+60.6 (c=0.63, CHCl₃).

Compound 24: ¹H NMR (400 MHz, CDCl₃, ppm): δ=7.33-7.20 (m, 5H, arom.), 4.84, 4.64 (2d, 2H, J=11.3 Hz, CH₂-Ph), 4.36 (dd, 1H, J=4.1 Hz, J=12.2 Hz, H-6b), 4.21 (d, 1H, J=7.9 Hz, H-1), 4.18 (dd, 1H, J=2.2 Hz, J=12.2 Hz, H-6b), 3.94, 3.60 (m, 2H, CH₂₍ₐ₎-pent-4-ynyl), 2.60 (d, 1H, J=3.1 Hz, OH), 2.30-2.23 (m, 2H, CH_{2(c)}-pent-4-ynyl), 1.84 (t, 1H, J=2.6 Hz, CH_{(d)}-alkyne), 1.84-1.71 (m, 2H, CH_{2(b)}-pent-4-ynyl). MALDI-MS, positive mode, m/z: 425.96 [M+Na⁺], 441.92 [M+K⁺]. [α]_{D}²¹=21.0 (c=0.49, CHCl₃).

Monosaccharides 21 and 25 were prepared from compound 18 in a similar manner as described for 19 and 23.

Compound 21: ¹H NMR (400 MHz, CDCl₃, ppm): δ=7.64-7.57 (m, 4H, arom.), 7.41-7.29 (m, 6H, arom.), 4.76 (d, 1H, J=3.5 Hz, H-1), 3.62 (s, 3H, OMe), 2.72 (d, 1H, J=2.1 Hz, OH), 2.28-2.22 (m, 2H, CH_{2(c)}-pent-4-ynyl), 1.84 (t, 1H, J=2.6 Hz, CH_{(d)}-alkyne), 1.82-1.68 (m, 2H, CH_{2(b)}-pent-4-ynyl), 1.02 (s, 9H, C(CH₃)₃). MALDI-MS, positive mode, m/z: 546.25 [M+Na⁺], 562.18 [M+K⁺]. [α]_{D}²¹=+56.1 (c=1.8, CHCl₃).

Compound 25: ¹H NMR (400 MHz, CDCl₃, ppm): δ=7.63-7.58 (m, 4H, arom.), 7.40-7.29 (m, 6H, arom.), 4.20 (d, 1H, J=8.1 Hz, H-1), 3.61 (s, 3H, OMe), 2.29-2.21 (m, 2H, CH_{2(c)}-pent-4-ynyl), 1.84 (t, 1H, J=2.6 Hz, CH_{(d)}-alkyne), 1.82-1.68 (m, 2H, CH_{2(b)}-pent-4-ynyl), 0.99 (s, 9H, C(CH₃)₃). MALDI-MS, positive mode, m/z: 546.20 [M+Na⁺], 562.20 [M+K⁺]. [α]_{D}²¹=9.3 (c=1.3, CHCl₃).

Monosaccharides 22 and 26 were prepared from compound 18 in a similar manner as described for 20 and 24.

Compound 22: ¹H NMR (400 MHz, CDCl₃, ppm): δ=4.87 (d, 1H, J=3.8 Hz, H-1), 4.51 (dd, 1H, J=12.4 Hz, J=4.3 Hz, H-6a), 4.19 (dd, 1H, J=12.4 Hz, J=2.2 Hz, H-6b), 3.88-3.76 (m, 2H, H-5, CH₍ₐ₎-pent-4-ynyl), 3.68 (s, 3H, OMe), 3.62-3.51 (m, 2H, H-3, CH_{(a′)}-pent-4-ynyl), 3.42 (dd, 1H, J=9.8 Hz, J=8.7 Hz, H-4), 3.15 (dd, 1H, J=9.8 Hz, J=3.8 Hz, H-2), 2.37-2.30 (m, 2H, CH_{2(c)}-pent-4-ynyl), 2.10 (s, 3H, CH₃—OAc), 1.95 (t, 1H, J=2.6 Hz, CH_{(d)}-alkyne), 1.93-1.75 (m, 2H, CH_{2(b)}-pent-4-ynyl). MALDI-MS, positive mode, m/z: 350.01 [M+Na⁺].

Compound 26: ¹H NMR (400 MHz, CDCl₃, ppm): δ=4.24 (dd, 1H, J=12.4 Hz, J=3.8 Hz, H-6a), 4.30-4.23 (m, 2H, J=8.1 Hz, H-6b, H-1), 4.03-3.95 (m, 1H, CH₍ₐ₎-pent-4-ynyl), 3.71-3.62 (m, 4H, CH_{(a′)}-pent-4-ynyl, OMe), 3.42-3.35 (m, 2H, H-4, H-5), 3.27 (dd, 1H, J=9.8 Hz, J=8.1 Hz, H-2), 2.99 (t, 1H, J=9.8 Hz, H-3), 2.85 (d, 1H, J=2.8 Hz, OH), 2.37-2.30 (m, 2H, CH_{2(c)}-pent-4-ynyl), 2.10 (s, 3H, CH₃—OAc), 1.95 (t, 1H, J=2.7 Hz, CH_{(d)}-alkyne), 1.91-1.78 (m, 2H, CH_{2(b)}-pent-4-ynyl). MALDI-MS, positive mode, m/z: 349.93 [M+Na⁺], 365.83 [M+K⁺]. [α]_{D}²¹=17.9 (c=1.0, CHCl₃).

### 1. Preparation 3: Synthesis of Reducing Monosaccharide 33 (Scheme 3)

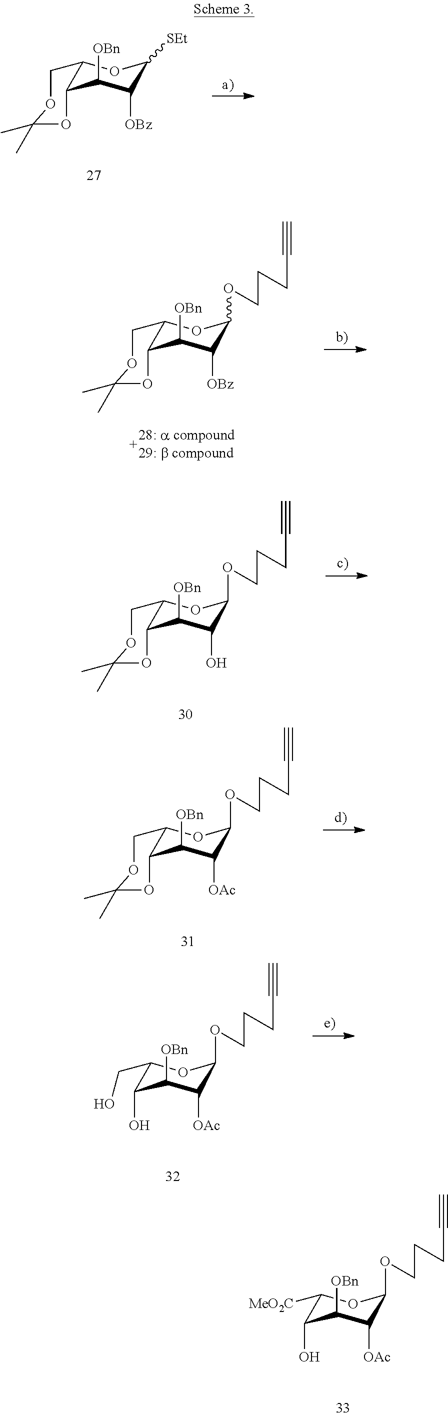

Step 3.a: Synthesis of compounds 28 and 29: In a dry round-bottom flask, the monosaccharide donor 27 (3 g, 6.54 mmol, 1 eq.), which was prepared as described in Carbohydr. Res. 1999, 317, 63-84 and prealably azeotropically dried with toluene, was dissolved in anhydrous dichloromethane (32 mL) under a nitrogen atmosphere containing 4 Å molecular sieves (3 g) previously activated at 400° C. After stirring for 30 min at room temperature, the reaction mixture was cooled down to 40° C. and N-iodosuccinimide (2.94 g, 13.08 mmol, 2.0 eq.), triflic acid (69 4, 0.78 mmol, 0.12 eq. vs donor) followed by 4-pentyn-1-ol (1.52 mL, 16.35 mmol, 2.5 eq.) were succcessively added. After stirring for 15 min at 40° C., the reaction mixture was filtered through a pad of Celite®, washed with dichloromethane, neutralized by Et₃N until pH 8 and successively washed with a 1 M aqueous solution of Na₂S₂O₃ (to quench the excess of iodine) and water. The organic layer was dried over MgSO₄, filtered, the solvent was evaporated under reduced pressure and the residue was purified by chromatography on silica gel column (heptane/ethyl acetate: 9/1 to 8/2+1% Et₃N) to give separately pure compound 28 (2.3 g, anomer α, 73%) and pure compound 29 (367 mg, anomer β, 12%) as colourless viscous oils with a global yield of 85%.

Compound 28: ¹H NMR (400 MHz, CDCl₃, ppm): δ=8.14-8.09 (m, 2H, arom.), 7.59-7.24 (m, 8H, arom.), 5.26 (dd, 1H, J=2.1 Hz, J=4.1 Hz, H-2), 5.03 (d, 1H, J=2.1 Hz, H-1), 4.83, 4.64 (2d, 2H, J=11.7 Hz, CH₂-Ph), 4.10 (dd, 1H, J=2.5 Hz, J=12.6 Hz, H-6a), 4.02 (t, 1H, J=2.5 Hz, H-4), 3.97-3.85 (m, 3H, H-6b, CH₍ₐ₎-pent-4-ynyl, H-5), 3.71 (m, 1H, H-3), 3.59-3.51 (m, 1H, CH_{(a′)}-pent-4-ynyl), 2.32-2.25 (m, 2H, CH_{2(c)}-pent-4-ynyl), 1.91 (t, 1H, J=2.7 Hz, CH_{(d)}-alkyne), 1.89-1.73 (m, 2H, CH_{2(b)}-pent-4-ynyl), 1.48, 1.45 (2s, 6H, C(CH₃)₂). MALDI-MS, positive mode, m/z: 503.17 [M+Na⁺], 519.12 [M+K⁺]. [α]_{D}²¹=23.6 (c=0.80, CHCl₃).

Compound 29: ¹H NMR (400 MHz, CDCl₃, ppm): δ=8.14-7.99 (m, 2H, arom.), 7.48-7.11 (m, 8H, arom.), 5.14-5.12 (m, 1H, H-2), 4.78 (d, 1H, J=1.4 Hz, H-1), 4.72, 4.57 (2d, 2H, J=11.7 Hz, CH₂-Ph), 4.01 (dd, 1H, J=2.4 Hz, J=12.9 Hz, H-6a), 3.97-3.88 (m, 2H, H-6b, CH₍ₐ₎-pent-4-ynyl), 3.77-3.72 (m, 2H, H-4, H-3), 3.56-3.53 (m, 1H, H-5), 3.52-3.45 (m, 1H, CH_{(a′)}-pent-4-ynyl), 2.15-2.08 (m, 2H, CH_{2(c)}-pent-4-ynyl), 1.78 (t, 1H, J=2.7 Hz, CH_{(d)}-alkyne), 1.76-1.58 (m, 2H, CH_{2(b)}-pent-4-ynyl), 1.31 (s, 6H, C(CH₃)₂). MALDI-MS, positive mode, m/z: 503.12 [M+Na⁺], 519.09 [M+K⁺]. [α]_{D}²¹=+57.3 (c=0.785, CHCl₃).

Step 3.b: Synthesis of compound 30: In a dry round-bottom flask, compound 28 (3.81 g, 7.92 mmol) was dissolved in a dry mixture of tetrahydrofurane/methanol (1/1, 80 mL) under a nitrogen atmosphere at 0° C. A solution of 0.5 M MeONa in methanol (7.9 mL, 3.96 mmol, 0.5 eq.) was added and the resulting solution was stirred overnight at room temperature. The reaction mixture was neutralized with Amberlite® IRA120 until pH 7-8, then filtered and concentrated to afford quantitatively compound 30 as a yellow viscous compound which was directly used in the next step without any further purification. MALDI-MS, positive mode, m/z: 400.08 [M+Na⁺].

Step 3.c: Synthesis of compound 31: In a dry round-bottom flask, compound 30 (7.92 mmol) was dissolved in dry dichloromethane (50 mL) at room temperature. The solution was cooled to 0° C. and acetic anhydride (3.7 mL, 39.61 mmol, 5 eq.), Et₃N (6.1 mL, 43.57 mmol, 5.5 eq.) and DMAP (483 mg, 3.96 mmol, 0.5 eq.) were successively added. The resulting mixture was stirred overnight at room temperature. The reaction mixture was then diluted with dichloromethane (150 mL) and the organic layer was successively washed with a 5% aqueous solution of H₂SO₄ (25 mL), a aqueous saturated solution of NaHCO₃ (25 mL) and a brine solution (25 mL), dried over MgSO₄, filtered and concentrated under reduced pressure to give crude compound 31 as a yellow viscous solid which was directly used in the next step without any further purification. MALDI-MS, positive mode, m/z: 440.94 [M+Na⁺].

Step 3.d: Synthesis of compound 32: Isopropylidene cleavage of compound 31 (7.92 mmol) was performed according to the general method C. Compound 32 was obtained as a viscous solid and directly used in the next step without any further purification. MALDI-MS, positive mode, m/z: 401.11 [M+Na⁺], 417.08 [M+K⁺].

Step 3.e: Synthesis of compound 33. Compound 33 was prepared by oxidation of primary alcohol 32 (7.92 mmol) according to the general method D followed by esterification of carboxylic acid according to the general method E. Purification was effected by chromatography on silica gel column (heptane/ethyl acetate: 7/3 to 5/5) to give compound 33 (1.76 g, 55% over 5 steps) as a colourless viscous compound. ¹H NMR (400 MHz, CDCl₃, ppm): δ=7.36-7.22 (m, 5H, arom.), 4.98 (sl, 1H, H-2), 4.93 (sl, 1H, H-1), 4.86 (sl, 1H, H-5), 4.74, 4.58 (2d, 2H, J=11.6 Hz, CH₂-Ph), 4.03 (sl, 1H, H-4), 3.94-3.85 (m, 1H, CH₍ₐ₎-pent-4-ynyl), 3.80 (s, 3H, CO₂Me), 3.69 (sl, 1H, H-3), 3.58-3.51 (m, 1H, CH_{(a′)}-pent-4-ynyl), 2.71 (sl, 1H, OH), 2.29-2.22 (m, 2H, CH_{2(c)}-pent-4-ynyl), 2.09 (s, 3H, CH₃—OAc), 1.90 (t, 1H, J=2.5 Hz, CH_{(d)}-alkyne), 1.87-1.70 (m, 2H, CH_{2(b)}-pent-4-ynyl). MALDI-MS, positive mode, m/z: 429.06 [M+Na⁺], 445.03 [M+K⁺]. [α]_{D}²¹=62.7 (c=1.14, CHCl₃).

### C. Disaccharides Preparations

### 1. Preparation 4: Synthesis of Reducing Disaccharides 54, 55, 56, 57 and 58 (Scheme 4)

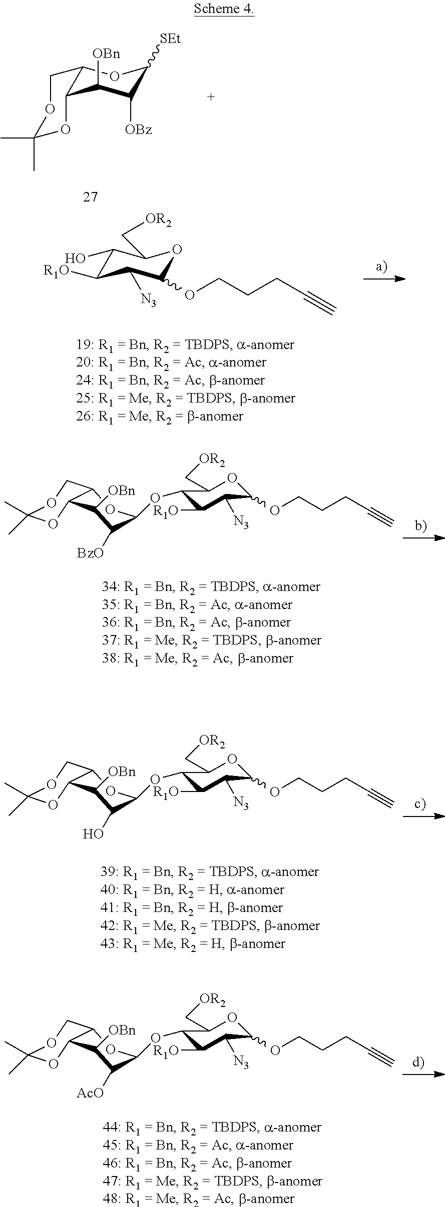

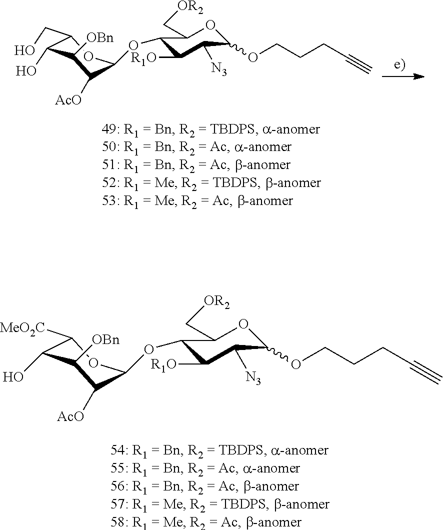

Step 4.a: Synthesis of compound 34: O-glycosylation reaction between monosaccharide donor 27 (460 mg, 1.0 mmol, 1 eq.) and monosaccharide acceptor 19 (602 mg, 1.0 mmol, 1 eq.) was performed according to the general method A. Purification was effected by chromatography on silica gel column (heptane/ethyl acetate: 8/2 with 1% Et₃N) to give compound 34 (932 mg, 93%) as a white solid. ¹H NMR (400 MHz, CDCl₃, ppm): δ=8.04-7.93 (m, 2H, arom.), 7.73-7.58 (m, 3H, arom.), 7.44-7.16 (m, 20H, arom.), 5.24 (s, 1H, H-1′), 5.22 (dd, 1H, J=2.4 Hz, J=4.4 Hz, H-2′), 4.88 (d, 1H, J=3.7 Hz, H-1), 4.79, 4.65 (d, 2H, J=11.5 Hz, CH₂-Ph), 4.77, 4.66 (d, 2H, J=11.5 Hz, CH₂-Ph), 4.02 (t, 1H, J=9.5 Hz, H-4), 3.88-3.62 (m, 8H, H-3′, H-4′, H-5′, H-3, CH₍ₐ₎-pent-4-ynyl, H-5, H-6a, H-6b), 3.53-3.47 (m, 1H, CH_{(a′)}-pent-4-ynyl), 3.43 (dd, 1H, J=2.5 Hz, J=12.9 Hz, H-6′ a), 3.32 (dd, 1H, J=3.7 Hz, J=10.2 Hz, H-2), 3.06 (dd, 1H, J=3.0 Hz, J=12.9 Hz, H-6′ b), 2.36-2.28 (m, 2H, CH_{2(c)}-pent-4-ynyl), 1.87-1.78 (m, 3H, CH_{(d)}-alkyne, CH_{2(b)}-pent-4-ynyl), 1.36, 1.26 (2s, 6H, C(CH₃)₂), 1.04 (s, 9H, C(CH₃)₃). MALDI-MS, positive mode, m/z: 1019.00 [M+Na⁺], 1034.93 [M+K⁺]. [α]_{D}²¹=+40.1 (c=0.76, CHCl₃).

Step 4.b: Synthesis of compound 39: In a dry round-bottom flask, compound 34 (288 mg, 0.289 mmol) was dissolved in a dry mixture of tetrahydrofurane/methanol (1/1, 1.5 mL) under a nitrogen atmosphere at 0° C. A solution of 0.5 M MeONa in methanol (0.58 mL, 0.289 mmol, 1 eq.) was added and the resulting solution was stirred overnight at room temperature. The reaction mixture was neutralized with Dowex 50WX8-200 until pH 7-8, then filtered and concentrated to afford quantitatively compound 39 as an orange syrup which was directly used in the next step without any further purification. MALDI-MS, positive mode, m/z: 914.53 [M+Na⁺], 930.49 [M+K⁺].

Step 4.c: Synthesis of compound 44: In a dry round-bottom flask, compound 39 (0.289 mmol) was dissolved in dry dichloromethane (1.40 mL) at room temperature. The solution was cooled to 0° C. and acetic anhydride (137 μL, 1.45 mmol, 5 eq.), Et₃N (220 μL, 1.59 mmol, 5.5 eq.) and DMAP (3.5 mg, 0.029 mmol, 0.1 eq.) were successively added. The resulting mixture was stirred for 2 h at room temperature. The reaction mixture was then diluted with ethyl acetate (30 mL) and the organic layer was successively washed with a 5% aqueous solution of H₂SO₄ (5 mL), a aqueous saturated solution of NaHCO₃ (5 mL) and a brine solution (5 mL), dried over MgSO₄, filtered and concentrated under reduced pressure to give crude compound 44 as a yellow oil which was directly used in the next step without any further purification. MALDI-MS, positive mode, m/z: 956.78 [M+Na⁺], 972.75 [M+K⁺].

Step 4.d: Synthesis of compound 49: Isopropylidene cleavage of compound 44 (0.289 mmol) was performed according to the general method C. Compound 49 was obtained as a yellow oil and directly used in the next step without any further purification. MALDI-MS, positive mode, m/z: 916.82 [M+Na⁺], 932.77 [M+K⁺].

Step 4.e: Synthesis of compound 54: Compound 54 was prepared by oxidation of primary alcohol 49 (0.289 mmol) according to the general method D followed by esterification of carboxylic acid according to the general method E. Purification was effected by chromatography on silica gel column (heptane/ethyl acetate: 7/3) to give compound 54 (183 mg, 69% over 5 steps) as a white amorphous solid. ¹H NMR (400 MHz, CDCl₃, ppm): δ=7.65-7.56 (m, 5H, arom.), 7.36-7.13 (m, 15H, arom.), 5.14 (s, 1H, H-1′), 4.89 (t, 1H, J=1.3 Hz, H-2′), 4.86 (d, 1H, J=1.7 Hz, H-5′), 4.78 (d, 1H, J=3.8, Hz H-1), 4.68-4.48 (m, 4H, 2×CH₂-Ph), 3.96 (t, 1H, J=9.5 Hz, H-4), 3.87-3.77 (m, 3H, H-6a, H-6b, H-4′), 3.74-3.59 (m, 4H, H-3, H-3′, H-5, CH₍ₐ₎-pent-4-ynyl), 3.46-3.39 (m, 1H, CH_{(a′)}-pent-4-ynyl), 3.34 (s, 3H, CO₂Me), 3.22 (dd, 1H, J=3.8 Hz, J=10.3 Hz, H-2), 2.50 (d, 1H, J=11.8 Hz, OH), 2.28-2.20 (m, 2H, CH_{2(c)}-pent-4-ynyl), 1.86 (s, 3H, CH₃—OAc), 1.82 (t, 1H, J=2.6 Hz, CH_{(d)}-alkyne), 1.80-1.69 (m, 2H, CH_{2(b)} pent-4-ynyl), 0.99 (s, 9H, C(CH₃)₃). MALDI-MS, positive mode, m/z: 944.61 [M+Na⁺], 960.50 [M+K⁺]. [60 ]_{D}²¹=+40.4 (c=0.71, CHCl₃).

Synthesis of disaccharides 55, 56, 57 and 58 were carried out as described for compound 54.

Compound 55: ¹H NMR (400 MHz, CDCl₃, ppm): δ=7.38-7.07 (m, 10H, arom.), 4.99 (s, 1H, H-1′), 4.89-4.80 (m, 3H, H-2′, H-1, H-5′), 4.72-4.53 (m, 4H, CH₂-Ph), 4.34 (d, 1H, J=12.3 Hz, H-6a), 4.15 (d, 1H, J=12.3 Hz, H-6b), 3.88 (d, 1H, J=10.5 Hz, H-4′), 3.85-3.71 (m, 4H, H-3, H-4, H-5, CH₍ₐ₎-pent-4-ynyl), 3.65 (br, 1H, H-3′), 3.54-3.46 (m, 1H, CH_{(a′)}-pent-4-ynyl), 3.40 (s, 3H, CO₂Me), 3.26 (dd, 1H, J=3.4 Hz, J=10.0 Hz, H-2), 2.52 (d, 1H, J=10.5 Hz, OH), 2.29 (m, 2H, CH_{2(c)-pent-}4-ynyl), 2.02, 2.00 (2s, 6H, CH₃—OAc), 1.89 (br, 1H, CH_{(d)}-alkyne), 1.86-1.73 (m, 2H, CH_{2(b)}-pent-4-ynyl). MALDI-MS, positive mode, m/z: 748.48 [M+Na⁺], 764.45 [M+K⁺]. [α]_{D}²¹=+33.4 (c=0.82, CHCl₃).

Compound 56: ¹H NMR (400 MHz, CDCl₃, ppm): δ=7.30-7.16 (m, 10H, arom.), 4.98 (s, 1H, H-1′), 4.87 (d, 1H, J=1.8 Hz, H-5′), 4.83 (br, 1H, H-2′), 4.69-4.53 (m, 4H, CH₂-Ph), 4.39 (dd, 1H, J=2.3 Hz, J=12.4 Hz, H-6a), 4.20 (d, 1H, J=8.0 Hz, H-1), 4.11 (dd, 1H, J=4.1 Hz, J=12.4 Hz, H-6b), 3.94-3.87 (m, 2H, H-4′, CH₍ₐ₎-pent-4-ynyl), 3.78 (t, 1H, J=9.1 Hz, H-4), 3.63 (br, 1H, H-3′), 3.56-3.53 (m, 1H, CH_{(a′)}-pent-4-ynyl), 3.43 (s, 3H, CO₂Me), 3.39-3.29 (m, 2H, H-5, H-2), 3.19 (t, 1H, J=9.4 Hz, H-3), 2.52 (d, 1H, J=11.2 Hz, OH), 2.29-2.23 (m, 2H, CH_{2(c)}-pent-4-ynyl), 2.02, 1.99 (2s, 6H, CH₃—OAc), 1.88 (t, J=2.6 Hz, 1H, CH_{(d)}-alkyne), 1.84-1.76 (m, 2H, CH_{2(b)}-pent-4-ynyl). MALDI-MS, positive mode, m/z: 748.67 [M+Na⁺]. [α]_{D}²¹=27.3 (c=0.27, CHCl₃).

Compound 57: ¹H NMR (400 MHz, CDCl₃, ppm): δ=7.77-7.60 (m, 4H, arom.), 7.47-7.24 (m, 11H, arom.), 4.92 (s, 1H, H-1′), 4.21 (d, 1H, J=7.6 Hz, H-1), 3.82 (s, 3H, CO₂Me), 3.45 (s, 3H, OMe), 3.91, 3.58 (m, 2H, CH₂₍ₐ₎-pent-4-ynyl), 2.68 (d, 1H, J=11.7 Hz, OH), 2.37-2.29 (m, 2H, CH_{2(c)}-pent-4-ynyl), 1.99 (s, 3H, CH₃—OAc), 1.95 (t, 1H, J=2.6 Hz, CH_{(d)}-alkyne), 1.91-1.79 (m, 2H, CH_{2(b)}-pent-4-ynyl), 1.06 (s, 9H, C(CH₃)₃). MALDI-MS, positive mode, m/z: 868.27 [M+Na⁺], 884.13 [M+K⁺]. [α]_{D}²¹=32.3 (c=1.60, CHCl₃).

Compound 58: ¹H NMR (400 MHz, CDCl₃, ppm): δ=7.40-7.20 (m, 5H, arom.), 4.98 (s, 1H, H-1′), 4.70, 4.61 (2d, 2H, J=11.8 Hz, CH₂-Ph), 4.23 (d, 1H, J=8.1 Hz, H-1), 3.96, 3.65 (m, 2H, CH₂₍ₐ₎-pent-4-ynyl), 3.82 (s, 3H, CO₂Me), 3.48 (s, 3H, OMe), 2.35-2.29 (m, 2H, CH_{2(c)}-pent-4-ynyl), 2.08, 2.07 (2s, 6H, CH₃—OAc), 1.94 (t, 1H, J=2.6 Hz, CH_{(d)}-alkyne), 1.88-1.78 (m, 2H, CH_{2(b)}-pent-4-ynyl). MALDI-MS, positive mode, m/z: 672.20 [M+Na⁺], 688.14 [M+K⁺]. [α]_{D}²¹=26.5 (c=1.50, CHCl₃).

### 1. Preparation 5: Synthesis of Reducing Disaccharides 80, 81, 82 and 83 (Scheme 5)

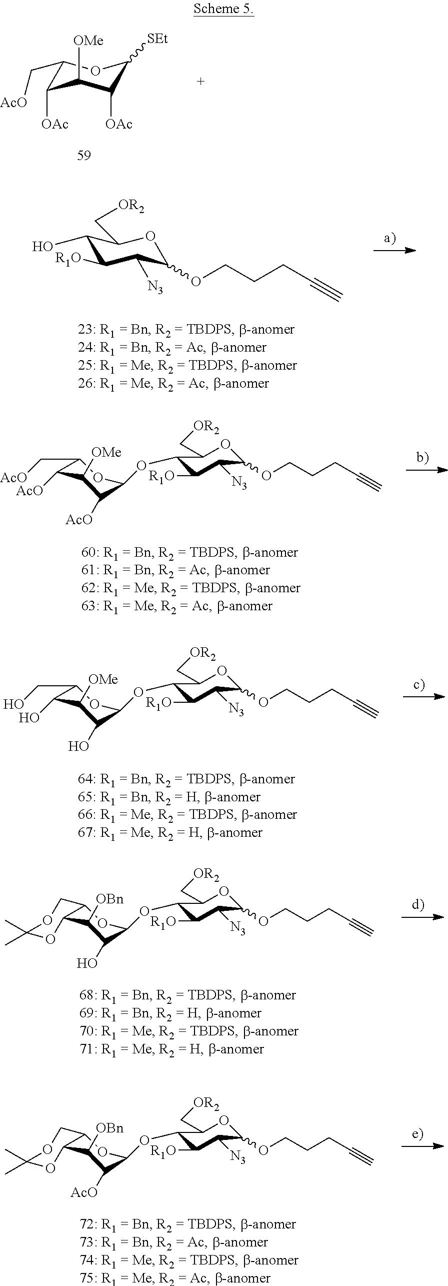

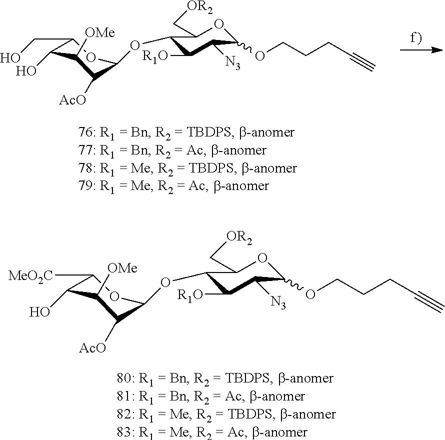

Step 5.a: Synthesis of compound 60:O-glycosylation reaction between monosaccharide donor 59 (270 mg, 0.78 mmol, 1 eq.), which was prepared as described in J. Carbohydr. Chem., 1985, 4, 293-321, and monosaccharide acceptor 23 (467 mg, 0.78 mmol, 1 eq.) was performed according to the general method A. Purification was effected by chromatography on silica gel column (heptane/ethyl acetate: 7/3) to give compound 60 (531 mg, 76%) as a white viscous solid. ¹H NMR (400 MHz, CDCl₃, ppm): δ=7.73-7.63 (m, 4H, arom.), 7.45-7.21 (m, 11H, arom.), 5.20 (s, 1H, H-1′), 4.86 (m, 2H, CH₂-Ph), 4.20 (d, 1H, J=7.3 Hz, H-1), 3.48 (s, 3H, OMe), 2.37-2.29 (m, 2H, CH_{2(c)}-pent-4-ynyl), 2.05, 1.94, 1.89 (3s, 9H, CH₃—OAc), 1.84 (t, 1H, J=2.6 Hz, CH_{(d)}-alkyne), 1.82-1.68 (m, 2H, CH_{2(b)}-pent-4-ynyl), 1.06 (s, 9H, C(CH₃)₃). MALDI-MS, positive mode, m/z: 924.31 [M+Na⁺], 940.23 [M+K⁺]. [α]_{D}²⁴=10.6 (c=0.44, CHCl₃).

Step 5.b: Synthesis of compound 64: Compound 60 (846 mg, 0.94 mmol) was dissolved in a dry mixture of tetrahydrofurane/methanol (1/1, 5.9 mL) under a nitrogen atmosphere at 0° C. A solution of 0.5 M MeONa in methanol (2.8 mL, 1.41 mmol, 1.5 eq.) was slowly added and the resulting solution was stirred overnight at room temperature. The reaction mixture was neutralized with Amberlite® IRA120 until acidic pH and filtered. The resin was washed several times with methanol and dichloromethane. The filtrate was concentrated under reduced pressure to give compound 64 as a yellow solid and was directly engaged in the next step without any further purification. MALDI-MS, positive mode, m/z: 798.56 [M+Na⁺], 814.53 [M+K⁺].

Step 5.c: Synthesis of compound 68: Compound 64 (0.94 mmol) was dissolved under a nitrogen atmosphere in dry DMF (4.7 mL) at room temperature. Dimethoxypropane (2.3 mL, 18.76 mmol, 20 eq.) and camphor sulfonic acid (22 mg, 0.094 mmol, 0.1 eq.) were successively added and the reacting mixture was stirred overnight at room temperature. The reaction mixture was neutralized with a saturated aqueous solution of NaHCO₃ (1 mL). The mixture was diluted with ethyl acetate (100 mL) and the organic layer was successively washed with a brine solution (2×10 mL) and water (10 mL). The organic layer was dried over MgSO₄, filtered and concentrated under reduced pressure to give crude compound 68 as a yellow oil which was directly used in the next step without any further purification. MALDI-MS, positive mode, m/z: 838.46 [M+Na⁺], 854.37 [M+K⁺].

Step 5.d: Synthesis of compound 72: Disaccharide 72 was prepared in a similar manner as described for compound 44. Compound 72 was directly used in the next step without any further purification. MALDI-MS, positive mode, m/z: 880.51 [M+Na⁺], 896.46 [M+K⁺].

Step 5.e: Synthesis of compound 76: Isopropylidene cleavage of compound 72 (0.94 mmol) was performed according to the general method C. Compound 76 was obtained as a yellow oil and was directly used in the next step without any further purification. MALDI-MS, positive mode, m/z: 840.54 [M+Na⁺], 856.41 [M+K⁺].

Step 5.f: Synthesis of compound 80: Compound 80 was prepared by oxidation of primary alcohol 76 (0.94 mmol) according to the general method D followed by esterification of carboxylic acid according to the general method E. Purification was effected by chromatography on silica gel column (heptane/ethyl acetate: 7/3 to 6/4) to give compound 80 (432 mg, 54% over 6 steps) as a white amorphous solid. ¹H NMR (400 MHz, CDCl₃, ppm): δ=7.75-7.64 (m, 4H, arom.), 7.45-7.21 (m, 11H, arom.), 4.87 (s, 1H, H-1′), 4.85-4.79 (2d, 2H, J=11.0 Hz, CH₂-Ph), 4.25 (d, 1H, J=7.9 Hz, H-1), 3.49 (s, 3H, CO₂Me), 3.48 (s, 3H, OMe), 2.62 (d, 1H, J=11.6 Hz, OH), 2.37-2.30 (m, 2H, CH_{2(c)}-pent-4-ynyl), 1.97 (s, 3H, CH₃—OAc), 1.93 (t, 1H, J=2.6 Hz, CH_{(d)}-alkyne), 1.91-1.78 (m, 2H, CH_{2(b)}-pent-4-ynyl), 1.07 (s, 9H, C(CH₃)₃). MALDI-MS, positive mode, m/z: 869.50 [M+Na⁺], 885.38 [M+K⁺]. [α]_{D}²¹=+1.4 (c=0.78, CHCl₃).

Synthesis of disaccharides 81, 82 and 83 were carried out as described for compound 80.

Compound 81: ¹H NMR (400 MHz, CDCl₃, ppm): δ=7.41-7.20 (m, 5H, arom.), 5.03 (s, 1H, H-1′), 4.87-4.77 (m, 2H, CH₂-Ph), 4.26 (d, 1H, J=8.1 Hz, H-1), 3.95, 3.64 (m, 2H, CH₂₍ₐ₎-pent-4-ynyl), 3.52 (s, 3H, CO₂Me), 3.48 (s, 3H, OMe), 2.66 (d, 1H, J=10.5 Hz, OH), 2.34-2.28 (m, 2H, CH_{2(c)}-pent-4-ynyl), 2.08, 2.06 (2s, 6H, CH₃—OAc), 1.94 (t, 1H, J=2.6 Hz, CH_{(d)}-alkyne), 1.88-1.79 (m, 2H, CH_{2(b)}-pent-4-ynyl). MALDI-MS, positive mode, m/z: 672.22 [M+Na⁺], 688.18 [M+K⁺]. [α]_{D}²¹=11.0 (c=1.13, CHCl₃).

Compound 82: ¹H NMR (400 MHz, CDCl₃, ppm): δ=7.64-7.54 (m, 4H, arom.), 7.34-7.24 (m, 6H, arom.), 5.09 (s, 1H, H-1′), 4.14 (d, 1H, J=8.1 Hz, H-1), 3.75 (s, 3H, CO₂Me), 3.46, 3.35 (2s, 6H, OMe), 2.26-2.21 (m, 2H, CH_{2(c)}-pent-4-ynyl), 1.90 (s, 3H, CH₃—OAc), 1.86 (t, J=2.6 Hz, 1H, CH_{(d)}-alkyne), 1.83-1.71 (m, 2H, CH_{2(b)}-pent-4-ynyl), 0.97 (s, 9H, C(CH₃)₃). MALDI-MS, positive mode, m/z: 792.48 [M+Na⁺], 808.40 [M+K⁺]. [α]_{D}²¹=21.3 (c=1.11, CHCl₃).

Compound 83: ¹H NMR (400 MHz, CDCl₃, ppm); MALDI-MS, positive mode.

### 1. Preparation 6: Synthesis of Elongating Disaccharides 100 and 101 (Scheme 6)

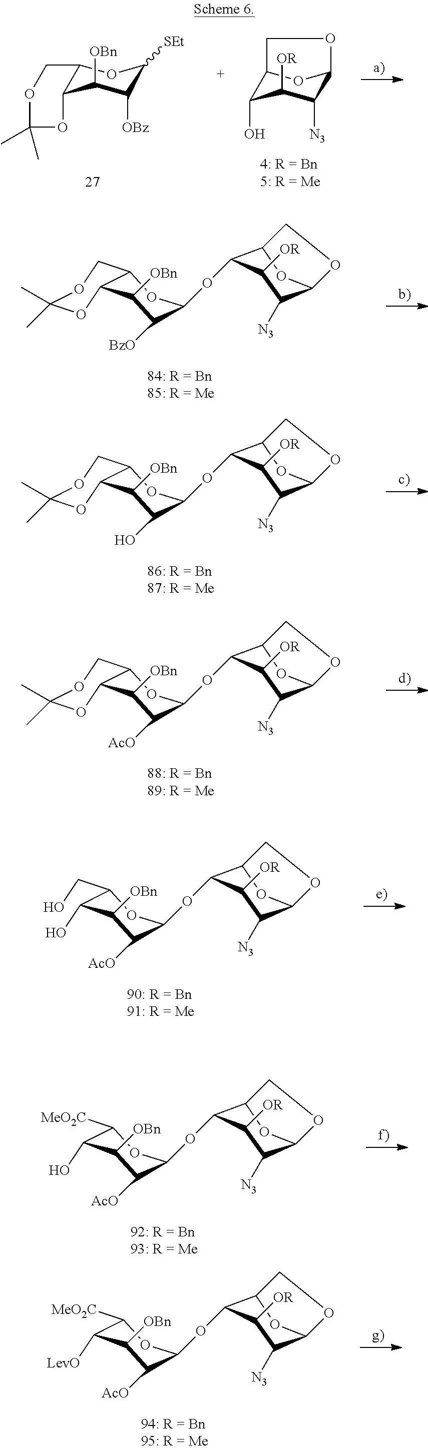

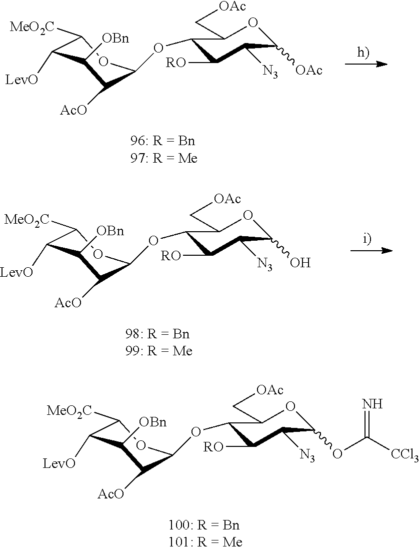

Step 6.a: Synthesis of compound 84: Compound 56 was prepared by conjugation of monosaccharide donor 27 (16.84 g, 36.72 mmol, 1.1 eq.) with monosaccharide acceptor 4 (9.26 g, 33.39 mmol, 1 eq.) according to the general method A. Purification was effected by chromatography on silica gel column (heptane/ethyl acetate: 9/1 to 7/3 with 1% Et₃N) to give compound 84 (18.85 g, 84%) as a white amorphous solid. ¹H NMR (400 MHz, CDCl₃, ppm): δ=8.13-8.07 (m, 2H, arom.), 7.45-7.20 (m, 13H, arom.), 5.46 (s, 1H, H-1), 5.32 (m, 1H, H-2′), 5.25 (s, 1H, H-1′), 4.87, 4.69 (d, 2H, J=11.5 Hz, CH₂-Ph), 4.69 (d, 1H, J=5.5 Hz, H-5), 4.62-4.51 (2d, 2H, J=11.4 Hz, CH₂-Ph), 3.99-3.94 (m, 2H, H-6a, H-3′), 3.85 (dd, 1H, J=12.5 Hz, J=2.0 Hz, H-6′ a), 3.82-3.78 (m, 2H, H-6′ b, H-5′), 3.76-3.71 (m, 3H, H-6b, H-4, H-4′), 3.65 (t, 1H, J=3.6 Hz, H-3), 3.26 (d, 1H, J=3.6 Hz, H-2), 1.46, 1.41 (2s, 6H, C(CH₃)₂). MALDI-MS, positive mode, m/z: 696.28 [M+Na⁺], 712.25 [M+K⁺]. [α]_{D}²¹=41.6 (c=0.87, CHCl₃).

Step 6.b: Synthesis of compound 86: In a dry round-bottom flask, compound 84 (12.60 g, 18.7 mmol) was dissolved in dry methanol (188 mL) under a nitrogen atmosphere. A solution of 0.5 M MeONa in methanol (37.4 mL, 18.7 mmol, 1 eq.) was added and the resulting solution was stirred overnight at room temperature. The reaction mixture was neutralized with Dowex 50WX8-200 until pH 7-8, then filtered and concentrated to afford compound 86 as a yellow oil which was directly used in the next step without any further purification.

Step 6.c: Synthesis of compound 88: In a dry round-bottom flask, compound 86 (18.7 mmol) was dissolved in dry pyridine (125 mL) at room temperature. The solution was cooled to 0° C. and acetic anhydride (7 mL, 74.80 mol, 4 eq.) and DMAP (228 mg, 1.87 mmol, 0.1 eq.) were successively added. The resulting mixture was stirred for 3 h at room temperature then concentrated to dryness, diluted with dichloromethane (500 mL) and washed with water (100 mL). The organic layer was dried over MgSO₄, filtered and concentrated under reduced pressure. The crude material was filtered through a pad of silica gel (heptane/ethyl acetate: 5/5 with 1% Et₃N) to give quantitatively compound 88 (11.40 g) as a colourless oil. ¹H NMR (400 MHz, CDCl₃, ppm): δ=7.32-7.10 (m, 10H, arom.), 5.40 (s, 1H, H-1), 5.04 (d, 1H, J=2.5 Hz, H-1′), 4.98 (dd, 1H, J=4.5 Hz, J=2.5 Hz, H-2′), 4.70-4.56 (m, 2H, CH₂-Ph), 4.59 (d, 1H, J=5.6 Hz, H-5), 4.50 (m, 2H, CH₂-Ph), 3.92 (d, 1H, J=7.4 Hz, H-6a), 3.84 (m, 1H, H-4′), 3.76 (dd, 1H, J=12.6 Hz, J=2.7 Hz, H-6′ a), 3.70-3.61 (m, 4H, H-5′, H-4, H-6b, H-6′ b), 3.58-3.53 (m, 2H, H-3, H-3′), 3.15 (d, 1H, J=3.5 Hz, H-2), 1.99 (s, 3H, CH₃—OAc), 1.34, 1.32 (2s, 6H, C(CH₃)₂). MALDI-MS, positive mode, m/z: 634.45 [M+Na⁺], 650.43 [M+K⁺]. [α]_{D}²¹=108.5 (c=1.77, CHCl₃).

Step 6.d: Synthesis of compound 90: Isopropylidene cleavage of compound 88 (11.40 g, 18.64 mmol) was performed according to the general method C. Compound 90 was obtained as a yellow oil and directly used in the next step without any further purification.

### Step 6.e: Synthesis of Compound 92

Preparation of compound 92 was carried out as described for 54. Purification was effected by chromatography on silica gel column (heptane/ethyl acetate: 1/9) to give compound 92 (9.34 g, 84%) as a white solid. ¹H NMR (400 MHz, CDCl₃, ppm): δ=7.41-7.22 (m, 10H, arom.), 5.51 (s, 1H, H-1), 5.20 (s, 1H, H-1′), 5.10-5.08 (m, 1H, H-2′), 4.81, 4.61 (2d, 3H, J=11.8 Hz, CH₂-Ph, H-5′), 4.59 (d, 1H, J=5.2 Hz, H-5), 4.56 (s, 2H, CH₂-Ph), 4.10-4.02 (m, 2H, H-4′, H-6a), 3.80-3.74 (m, 6H, H-4, H-6b, H-3′, CO₂Me), 3.65-3.62 (m, 1H, H-3), 3.25 (d, 1H, J=3.5 Hz, H-2), 2.69 (d, 1H, J=11.5 Hz, OH), 2.12 (s, 3H, CH₃—OAc). MALDI-MS, positive mode, m/z: 622.35 [M+Na⁺], 638.29 [M+K⁺]. [α]_{D}²¹=55.9 (c=0.48, CHCl₃).

Step 6.f: Synthesis of compound 94: In a dry round-bottom flask, compound 92 (5.09 g, 8.49 mmol) was dissolved in dry dichloromethane (60 mL) under a nitrogen atmosphere. Levulinic acid (1.74 mL, 16.97 mmol, 2 eq.) followed by DMAP (207 mg, 1.70 mmol, 0.2 eq.) were added to the solution, which was stirred at room temperature under nitrogen. After 5 min, EDAC (3.2 g, 16.97 mmol, 2 eq.) was added and the reaction mixture was stirred overnight at room temperature. The organic layer was diluted with dichloromethane (200 mL), washed with water (40 mL), dried over MgSO₄, filtered and concentrated under reduced pressure to give crude compound 94 which was directly used in the next step without any further purification. MALDI-MS, positive mode, m/z: 720.30 [M+Na⁺], 736.18 [M+K⁺].

Step 6.g: Synthesis of compound 96: Acetolysis of compound 94 (8.49 mmol) was performed according to the general method F. Purification was effected by chromatography on silica gel column (heptane/ethyl acetate: 5/5 to 4/6) to give compound 96 (5.71 g, 85% over 2 steps, α/β: 74/26) as a white amorphous solid. ¹H NMR (400 MHz, CDCl₃, ppm): δ=7.37-7.14 (m, 15H, arom.), 6.15 (d, 1H, J=3.5 Hz, H-1α), 5.38 (d, 0.35H, J=8.4 Hz, H-1β), 5.07 (d, 1H, J=2.5 Hz, H-1′), 5.02 (t, 1H, J=3.2 Hz, H-4′), 4.84-4.80 (m, 2H, H-5′, H-2′), 4.75-4.57 (m, 4H, CH₂-Ph), 4.36 (dd, 0.35H, J=2.4 Hz, J=12.7 Hz, H-6aβ), 4.18-4.10 (m, 1.35H, H-6aβ, H-6aα), 4.31 (dd, 1H, J=2.2 Hz, J=12.7 Hz, H-6bα), 3.96-3.79 (m, 2.35H, H-4α, H-5α, H-4β), 3.76-3.63 (m, 2H, H-3α, H-3′), 3.54 (dd, 1H, J=3.5 Hz, J=10.2 Hz, H-2α), 3.53-3.45 (m, 0.7H, H-5β, H-2β), 3.43 (s, 3H, CO₂Meα), 3.41 (s, 1.05H, CO₂Meβ), 3.28 (t, 0.35H, J=10.2 Hz, H-3β), 2.74-2.33 (m, 5.4H, CH₂-Lev), 2.12, 2.09, 2.02, 2.00, 1.99 (5s, 16.2H, CH₃-Lev, CH₃—OAc). MALDI-MS, positive mode, m/z: 822.25 [M+Na⁺], 838.23 [M+K⁺].

Step 6.h: Synthesis of compound 98: Selective hydrolysis of compound 96 (3.66 g, 4.58 mmol) was performed according to the general method G. Compound 98 was directly used in the next step without any further purification. MALDI-MS, positive mode, m/z: 779.95 [M+Na⁺], 795.91 [M+K⁺].

Step 6.i: Synthesis of compound 100: Trichloroacetimidate formation of compound 98 (4.58 mmol) was performed according to the general method H. Purification was effected by chromatography on silica gel column (heptane/ethyl acetate: 3/7 with 1% Et₃N) to give compound 100 (3.1 g, 75% over 2 steps, α/β: 76/24) as a white amorphous solid. ¹H NMR (400 MHz, CDCl₃, ppm), δ: 8.68 (s, 1H, NHα), 8.65 (s, 0.31H, NHβ), 7.36-7.11 (m, 10H, arom.), 6.34 (d, 1H, J=3.6 Hz, H-1α), 5.55 (d, 0.31H, J=8.5 Hz, H-1β), 5.05 (s, 1H, H-1′), 3.41 (s, 3H CO₂Me), 2.74-2.34 (m, 4H, CH₂-Lev), 2.09 (s, 3H, CH₃-Lev), 2.01, 2.00 (2s, 6H, CH₃—OAc). MALDI-MS, positive mode, m/z: 780.42 [M+Na⁺—C(NH)CCl₃], 796.38 [M+K⁺—C(NH)CCl₃].

Synthesis of disaccharide 101 was carried out of described for compound 100.

Compound 101: ¹H NMR (400 MHz, CDCl₃, ppm): δ=8.77 (s, 1H, NHα), 8.73 (s, 0.12H, NHβ), 7.39-7.30 (m, 5H, arom.), 6.38 (d, 1H, J=2.6 Hz, H-1α), 5.71 (d, 0.12H, J=8.7 Hz, H-1β), 5.10 (br. s, 1H, H-1′), 3.82 (s, 3H, CO₂Me), 3.51 (s, 3H, OMe), 2.87-2.48 (m, 4H, CH₂-Lev), 2.20 (s, 3H, CH₃-Lev), 2.11, 2.08 (2s, 6H, CH₃—OAc).

### 1. Preparation 7: Synthesis of Capping-End and Elongating Disaccharides 126, 127, 128 and 129 (Scheme 7)

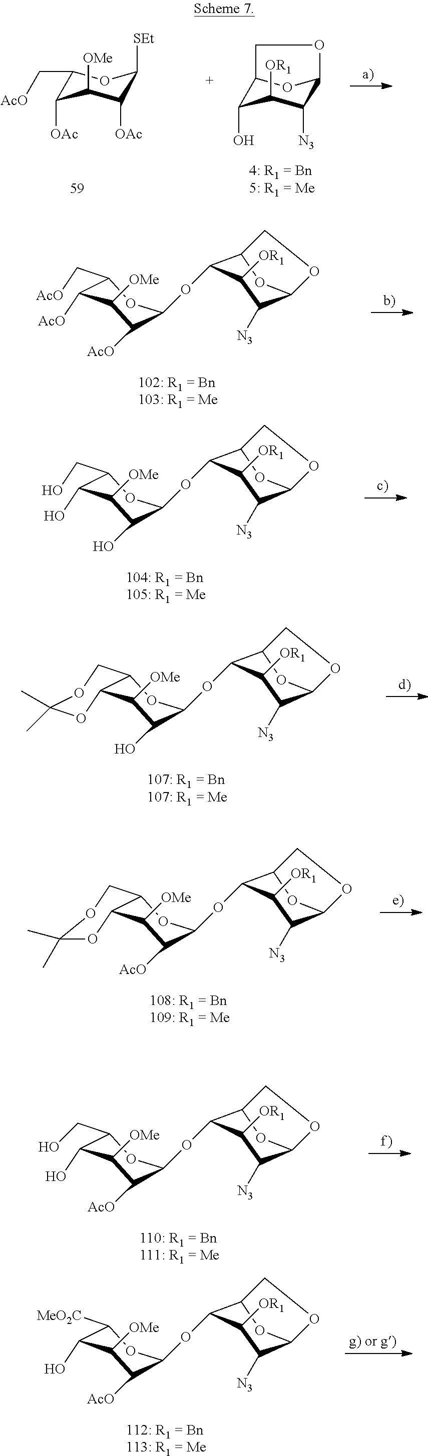

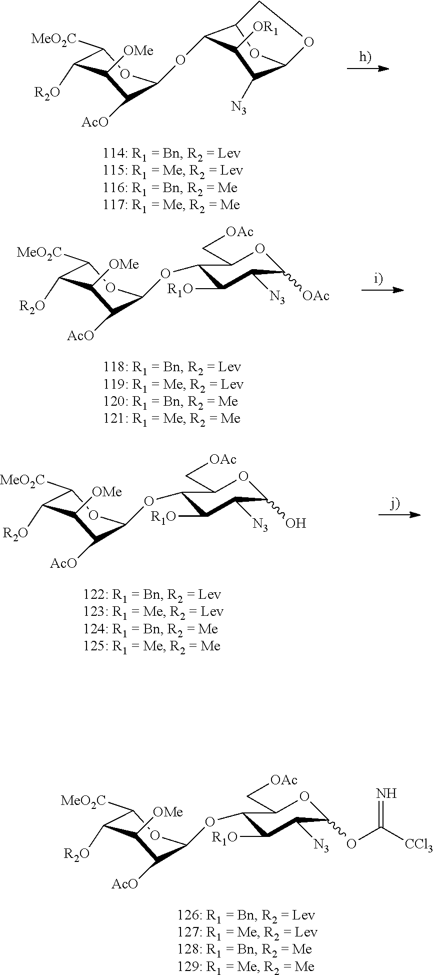

Step 7.a: Synthesis of compound 102: Compound 102 was prepared by conjugation of monosaccharide donor 59 (16.64 g, 45.66 mmol, 1 eq.) with monosaccharide acceptor 4 (12.66 g, 45.66 mmol, 1 eq.) according to the general method A. Purification was effected by chromatography on silica gel column (heptane/ethyl acetate: 6/4 to 5/5) to give compound 102 (20.75 g, 78%) as a white amorphous solid. ¹H NMR (400 MHz, CDCl₃, ppm): δ=7.39-7.28 (m, 5H, arom.), 5.50 (s, 1H, H-1), 5.09 (s, 1H, H-1′), 4.72-4.64 (2d, 2H, J=11.4 Hz, CH₂-Ph), 3.49 (s, 3H, OMe), 2.12, 2.09, 1.95 (3s, 9H, CH₃—OAc). ESI-MS, positive mode, m/z: 602.13 [M+Na⁺]. [α]_{D}²¹=18.9 (c=0.74, CHCl₃).

Step 7.b: Synthesis of compound 104: Compound 102 (20.75 g, 35.8 mmol) was dissolved in a dry mixture of tetrahydrofurane/methanol (1/1, 224 mL) under a nitrogen atmosphere and the solution was cooled to 0° C. A 0.5 M solution of MeONa in methanol (107 mL, 53.7 mmol, 1.5 eq.) was slowly added and the resulting solution was stirred overnight at room temperature. The reaction mixture was neutralized with Amberlite® IRA120 until acidic pH then filtered. The resin was washed several times with methanol and dichloromethane. The filtrate was concentrated under reduced pressure to give compound 104 as a yellow oil which was directly engaged in the next step without any further purification. ESI-MS, positive mode, m/z: 476.11 [M+Na⁺].

Step 7.c: Synthesis of compound 106: Compound 104 (35.8 mmol) was dissolved under a nitrogen atmosphere in anhydrous DMF (180 mL) at room temperature. Camphor sulfonic acid (831.6 mg, 3.58 mmol, 0.1 eq.) followed by dimethoxypropane (74.6 g, 0.72 mol, 20 eq.) were added. The mixture was stirred overnight at room temperature, then neutralized with a saturated aqueous solution of NaHCO₃ (30 mL). The reaction mixture was diluted with ethyl acetate (1 L) and the organic layer was successively washed with a saturated solution of NaCl (2×200 mL) and water (300 mL). The organic layer was dried over MgSO₄, filtered and concentrated under reduced pressure to give crude compound 106 as a yellow oil which was directly used in the next step without any further purification. MALDI-MS, positive mode, m/z: 516.28 [M+Na⁺], 532.24 [M+K⁺].

Step 7.d: Synthesis of compound 108: Crude compound 106 (22.37 mmol) was dissolved in dry dichloromethane (250 mL) at room temperature. Et₃N (32 mL, 0.228 mol, 10.2 eq.) followed by acetic anhydride (21 mL, 0.224 mol, 10 eq.) and DMAP (1.4 g, 11.19 mmol, 0.5 eq.) were added. The resulting mixture was stirred for 3 h at room temperature after which dichloromethane (250 mL) was added. The organic layer was successively washed with a 5% aqueous solution of H₂SO₄ (200 mL), water (200 mL), an aqueous saturated solution of NaHCO₃ (200 mL) and water. The organic layers were combined, dried over MgSO₄, filtered and concentrated under reduced pressure to give crude compound 108 as a yellow oil which was used in the next step without any further purification. MALDI-MS, positive mode, m/z: 558.34 [M+Na⁺], 574.32 [M+K⁺].

Step 7.e: Synthesis of compound 110: Isopropylidene cleavage of compound 108 (22.37 mmol) was performed according to the general method C. The crude material was filtered through a pad of silica gel (heptane/ethyl acetate: 1/9 to 0/10) to give compound 110 (10.27 g, 93% over 4 steps) as a colourless oil. MALDI-MS, positive mode, m/z: 518.29 [M+Na⁺], 534.26 [M+K⁺].

Step 7.f: Synthesis of compound 112: Preparation of compound 112 was carried out as described for 54. Purification was effected by chromatography on silica gel column (heptane/ethyl acetate: 4/6) to give compound 112 (533 mg, 61%) as a viscous solid. ¹H NMR (400 MHz, CDCl₃, ppm): δ=7.41-7.25 (m, 5H, arom.), 5.48 (s, 1H, H-1), 5.14 (s, 1H, H-1′), 4.64 (s, 2H, CH₂-Ph), 3.78 (s, 3H, CO₂Me), 3.50 (s, 3H, OMe), 2.75 (d, 1H, J=11.5 Hz, OH), 2.12 (s, 3H, CH₃—OAc). MALDI-MS, positive mode, m/z: 546.01 [M+Na⁺], 561.97 [M+K⁺]. [α]_{D}²¹=17.4 (c=0.70, CHCl₃).

Step 7.g: Synthesis of compound 114: In a dry round-bottom flask, compound 112 (2.72 g, 5.20 mmol) was dissolved in dry dichloromethane (37 mL) under a nitrogen atmosphere. Levulinic acid (1.07 mL, 10.4 mmol, 2 eq.) followed by DMAP (127 mg, 1.04 mmol, 0.2 eq.) were added to the solution, which was stirred at room temperature under nitrogen. After 5 min, EDAC (1.99 g, 10.4 mmol, 2 eq.) was added and the reaction mixture was stirred overnight at room temperature. The organic layer was diluted with dichloromethane (150 mL), washed with water (50 mL), dried over MgSO₄, filtered and concentrated under reduced pressure to give crude compound 114 which was directly used in the next step without any further purification.

Step 7.g′: Synthesis of compound 116: In a dry round-bottom flask, compound 113 (10.4 g, 19.87 mmol) was dissolved in dry dichloromethane (200 mL) under a nitrogen atmosphere. After cooling the solution to 0° C., proton sponge (25 g, 298 mmol, 15 eq.) followed by trimethyloxonium tetrafluoroborate (11.75 g, 79.46 mmol, 4 eq.) were added to the solution, which was continued to stir at room temperature under nitrogen atmosphere. Trimethyloxonium tetrafluoroborate was added dropwise two equivalents by two equivalents until ten over 20 h until complet conversion of starting material. The organic layer was filtered on Whatman paper, washed with a 1M H₂SO₄ aqueous solution (200 mL), dried over MgSO₄, filtered and concentrated under reduced pressure. The crude was purified by chromatography on silica gel column (heptane/ethyl acetate: 8/2 to 4/6) to give compound 116 (9.07 g, 85%) as a clear yellow viscous compound.

¹H NMR (400 MHz, CDCl₃, ppm): δ=7.40-7.19 (m, 5H, arom.), 5.48 (s, 1H, H-1), 5.23 (d, 1H, J=2.7 Hz, H-1′), 4.93 (t, 1H, J=2.7 Hz, H-2′), 4.74-4.57 (m, 4H, H-5, H-5′, CH₂-Ph), 4.07 (d, 1H, J=7.4 Hz, H-6a), 3.84 (sl, 1H, H-4), 3.79-3.67 (m, 6H, CO₂Me, H-6b, H-3, H-4′), 3.66-3.62 (m, 1H, H-3′), 3.52, 3.41 (2s, 6H, 2×OMe), 3.21 (sl, 1H, H-2), 2.10 (s, 3H, CH₃—OAc). MALDI-MS, positive mode, m/z: 560.28 [M+Na⁺], 576.22 [M+K⁺]. [α]_{D}²¹=23.2 (c=1.35, CHCl₃).

Step 7.h: Synthesis of compound 118: Acetolysis of compound 114 (5.20 mmol) was performed according to the general method F. Purification was effected by chromatography on silica gel column (heptane/ethyl acetate: 5/5 to 3/7) to give compound 118 (2.94 g, 78% over 2 steps, α/β: 78/22) as a white amorphous solid. ¹H NMR (400 MHz, CDCl₃, ppm): δ=7.50-7.24 (m, 5H, arom.), 6.24 (d, 1H, J=3.7 Hz, H-1α), 5.47 (d, 0.28H, J=8.3 Hz, H-1β), 5.13 (d, 1H, J=2.0 Hz, H-1′), 3.57 (s, 3H, CO₂Me), 3.51 (s, 3H, OMe), 2.84-2.42 (m, 4H, CH₂-Lev), 2.19 (s, 3H, CH₃-Lev), 2.18, 2.11 (3s, 9H, CH₃—OAc). MALDI-MS, positive mode, m/z: 746.32 [M+Na⁺], 762.30 [M+K⁺].

Step 7.i: Synthesis of compound 122: Selective hydrolysis of compound 118 (550 mg, 0.76 mmol) was performed according to the general method G. Compound 122 was directly used in the next step without any further purification. MALDI-MS, positive mode, m/z: 704.48 [M+Na⁺], 720.44 [M+K⁺].

Step 7.j: Synthesis of compound 126: Trichloroacetimidate formation of compound 122 (0.76 mmol) was performed according to the general method H. Purification was effected by chromatography on silica gel column (heptane/ethyl acetate: 5/5 with 1% Et₃N) to give compound 126 (421 mg, 67% over 2 steps, α/β: 86/14) as a white amorphous solid. ¹H NMR (400 MHz, CDCl₃, ppm): δ=7.42-7.25 (m, 5H, arom.), 8.74 (s, 1H, NHα), 8.73 (s, 0.16H, NHβ), 6.42 (d, 1H, J=3.6 Hz, H-1α), 5.71 (d, 0.16H, J=8.3 Hz, H-1β), 5.12 (br s, 1H, H-1′) 4.97, 4.87 (2d, 2H, J=11.2 Hz, CH₂-Ph), 3.57 (s, 3H, CO₂Me), 3.53 (s, 3H, OMe), 2.83-2.42 (m, 4H, CH₂-Lev), 2.17 (s, 3H, CH₃-Lev), 2.10, 2.08 (2s, 6H, CH₃—OAc). MALDI-MS, positive mode, m/z: 704.26 [M+Na⁺—C(NH)CCl₃], 720.24 [M+K⁺—C(NH)CCl₃].

Synthesis of disaccharide 127 was carried out as described for compound 126.

Compound 127: ¹H NMR (400 MHz, CDCl₃, ppm): δ=8.74 (s, 1H, NHα), 8.73 (s, 0.17H, NHβ), 6.38 (d, 1H, J=3.3 Hz, H-1α), 5.71 (d, 0.17H, J=8.3 Hz, H-1β), 5.16 (s, 1H, H-1′), 3.81 (s, 3H, CO₂Me), 3.63, 3.53 (2s, 6H, 2×OMe), 2.87-2.46 (m, 4H, CH₂-Lev), 2.18 (s, 3H, CH₃-Lev), 2.10, 2.08 (2s, 6H, CH₃—OAc).

Synthesis of disaccharides 128 and 129 were carried from respectively disaccharides 116 and 117 out as described for compound 126.

Compound 128: ¹H NMR (400 MHz, CDCl₃, ppm): δ=8.73 (s, 1H, NHα), 8.73 (s, 0.16H, NHβ), 7.48-7.23 (m, 5H, arom.), 6.38 (d, 1H, J=3.8 Hz, H-1α), 5.71 (d, 0.16H, J=8.4 Hz, H-1β), 5.21 (d, 1H, J=4.0 Hz, H-1′), 3.62 (s, 3H, CO₂Me), 3.52, 3.40 (2s, 6H, OMe), 2.11, 2.10 (2s, 6H, CH₃—OAc). MALDI-MS, positive mode, m/z: 620.19 [M+Na⁺—C(NH)CCl₃], 636.16 [M+K⁺—C(NH)CCl₃].

Compound 129: ¹H NMR (400 MHz, CDCl₃, ppm): δ=8.77 (s, 1H, NHα), 8.73 (s, 0.17H, NHβ), 6.38 (d, 1H, J=2.6 Hz, H-1α), 5.71 (d, 0.17H, J=8.7 Hz, H-1β), 5.13 (d, 1H, J=3.3 Hz, 1H, H-1′), 3.85 (s, 3H, CO₂Me), 3.68, 3.54, 3.46 (3s, 9H, OMe), 2.14, 2.11 (2s, 6H, CH₃—OAc).

### 1. Preparation 8: Synthesis of Capping-End Disaccharides 148, 149 and 150 (Scheme 8)

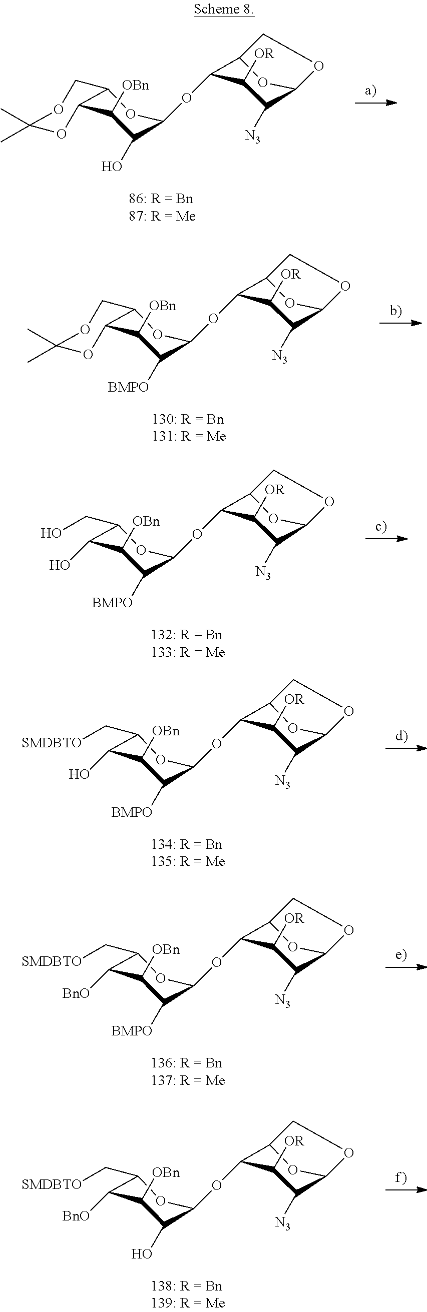

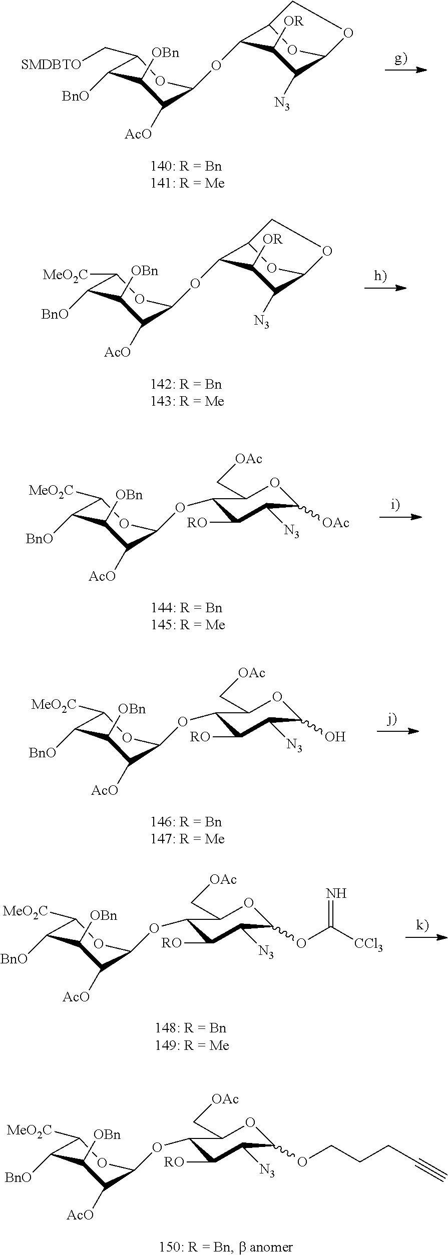

Step 8.a: Synthesis of compound 130: n a dry round-bottom flask, the crude compound 86 (10.61 mmol) was dissolved in anhydrous DMF (130 mL) under a nitrogen atmosphere. After cooling the solution to 0° C., NaH (60% dispersion in mineral oil, 721 mg, 18 mmol, 1.7 eq.) was added. The reaction mixture was stirred for 20 min at this temperature and para-methoxybenzylchloride (2.45 mL, 18 mmol, 1.7 eq.) was added. The solution was allowed to warm to room temperature and was stirred for 1 h. Methanol was then added dropwise to neutralize the excess of NaH and all solvents were evaporated under reduced pressure. The residue was then diluted with dichloromethane (300 mL) and the organic layer was washed with water (100 mL), dried over MgSO₄, filtered and concentrated under vacuum. The residue was purified by chromatography on silica gel column (heptane/ethyl acetate: 10/0 to 8/2 with 1% Et₃N) to afford compound 130 (6.92 g, 95% over 2 steps) as a colourless oil. ¹H NMR (400 MHz, CDCl₃, ppm): δ=7.41-7.26 (m, 12H, arom.), 6.89-6.85 (m, 2H, arom.), 5.55 (s, 1H, H-1), 4.97 (d, 1H, J=4.7 Hz, H-1′), 4.81-4.70 (m, 4H, 2×CH₂-Ph), 4.63 (s, 2H, CH₂—PMB), 4.60 (d, 1H, J=5.5 Hz, H-5), 4.10 (d, 1H, J=7.6 Hz, H-6a), 4.06-4.03 (m, 1H, H-4′), 3.96-3.91 (m, 1H, H-5′), 3.85 (dd, 1H, J=12.5 Hz, J=2.0 Hz, H-6′ a), 3.81 (s, 3H, OMe-PMB), 3.78-3.74 (m, 2H, H-6b, H-4), 3.73 (s, 1H, H-3), 3.70-3.68 (m, 2H, H-2′, H-3′), 3.63 (dd, 1H, J=12.2 Hz, J=4.7 Hz, H-6′ b), 3.23 (s, 1H, H-2), 1.43 (s, 6H, C(CH₃)₂). MALDI-MS, positive mode, m/z: 712.21 [M+Na⁺], 728.18 [M+K⁺]. [α]_{D}²¹=15.8 (c=1.45, CHCl₃).

Step 8.b: Synthesis of compound 132: sopropylidene cleavage of compound 130 (4.51 g, 6.54 mmol) was performed according to the general method C. The crude product was filtered through a pad of silica gel (heptane/ethyl acetate: 4/6) to give quantitatively compound 132.

Step 8.c: Synthesis of compound 134: a dry round-bottom flask, compound 132 (6.54 mmol) was dissolved in anhydrous dichloromethane (130 mL) under a nitrogen atmosphere. Tert-butyldimethylsilylchloride (1.38 g, 9.16 mmol, 1.4 eq.), Et₃N (1.09 mL, 7.85 mmol, 1.2 eq.) and a catalytic amount of DMAP (80 mg, 0.65 mmol, 0.1 eq.) were successively added to this solution and the resulting mixture was stirred for an additional for 17 h at room temperature. The solution was washed with a saturated aqueous solution of NaHCO₃ (40 mL), dried over MgSO₄, the solvent was evaporated and the residue was chromatographied on silica gel column (heptane/ethyl acetate: 8/2) to give compound 134 (4.25 g, 85% over 2 steps) as a white solid. ¹H NMR (400 MHz, CDCl₃, ppm): δ=7.35-7.20 (m, 12H, arom.), 6.90-6.85 (m, 2H, arom.), 5.56 (s, 1H, H-1), 5.21 (s, 1H, H-1′), 4.80 (d, 1H, J=5.4 Hz, H-5), 4.68-4.42 (m, 6H, CH₂—PMB, 2×CH₂-Ph), 4.16 (d, 1H, J=7.13 Hz, H-6a), 4.14-4.10 (m, 1H, H-5′), 3.92 (s, 1H, H-4), 3.84-3.81 (m, 5H, H-6′a, H-6′ b, CH₃—PMB), 3.81-3.69 (m, 4H, H-6b, H-2′, H-3′, H-4′), 3.68-3.67 (m, 1H, H-3), 3.17 (d, 1H, J=10.0 Hz, OH), 3.10 (s, 1H, H-2), 0.89 (s, 9H, C(CH₃)₃), 0.02 (2s, 6H, Si(CH₃)₂). MALDI-MS, positive mode, m/z: 786.41 [M+Na⁺], 802.37 [M+K⁺]. [α]_{D}²¹=3.4 (c=0.99, CHCl₃).

Step 8.d: Synthesis of compound 136: To a cooled (0° C.) solution of compound 134 (6.78 g, 8.78 mmol) in anhydrous DMF (178 mL) under a nitrogen atmosphere, NaH (60% dispersion in mineral oil, 391 mg, 9.76 mmol, 1.1 eq.) was added. The mixture was stirred for 30 min at 0° C. and benzyl bromide (1.37 mL, 11.54 mmol, 1.3 eq.) was added. Stirring was continued at room temperature for 1 h 30 and then methanol was added. The resulting mixture was concentrated under reduced pressure, then diluted in dichloromethane (200 mL) and the organic layer was washed with water (100 mL), dried over MgSO₄, filtered and concentrated under vacuum. The crude residue was purified by chromatography on silica gel column (heptane/ethyl acetate: 5/5) to afford compound 136 (7.53 g, 99%) as a colourless oil. ¹H NMR (400 MHz, CDCl₃, ppm): δ=7.38-7.24 (m, 17H, arom.), 6.89-6.82 (m, 2H, arom.), 5.55 (s, 1H, H-1), 5.13 (d, 1H, J=5.1 Hz, H-1′), 4.84-4.54 (m, 9H, CH₂—PMB, 3×CH₂-Ph, H-5), 4.10 (d, 1H, J=7.3 Hz, H-6a), 4.09-4.02 (m, 1H, H-5′), 3.92-3.86 (m, 1H, H-6′ a), 3.85-3.75 (m, 7H, CH₃—PMB, H-4, H-6b, H-4′, H-6′ b), 3.81 (s, 1H, H-3), 3.69 (dd, 1H, J=5.1 Hz, J=4.1 Hz, H-3′), 3.66 (dd, 1H, J=7.2 Hz, J=5.1 Hz, H-2′), 3.17 (s, 1H, H-2), 0.91 (s, 9H, C(CH₃)₃), 0.02 (2s, 6H, Si(CH₃)₂). MALDI-MS, positive mode, m/z: 876.18 [M+Na⁺], 892.14 [M+K⁺]. [α]_{D}²¹=+20.6 (c=2.22, CHCl₃).

Step 8.e: Synthesis of compound 138: To a solution of compound 136 (7.49 g, 8.77 mmol) in dichloromethane (150 mL) and water (6 mL) at 0° C. was added 2,3-dichloro-5,6-dicyano-1,4-benzoquinone (2.19 g, 9.65 mmol, 1.1 eq.) in portions over 5 minutes. The resulting mixture was stirred at room temperature for 3 h and a saturated aqueous solution of NaHCO₃ (100 mL) was added. The organic layer was separated and the aqueous layer was extracted with dichloromethane (3×150 mL). The combined organic layers were dried over MgSO₄, filtered and concentrated in vacuo. The residual oil was purified by chromatography on silica gel column (heptane/ethyl acetate: 8/2 to 5/5) to give alcohol disaccharide 138 (5.77 g, 90%) as a yellow oil. ¹H NMR (400 MHz, CDCl₃, ppm) δ: 7.45-7.25 (m, 15H, arom.), 5.53 (s, 1H, H-1), 5.18 (br s, 1H, H-1′), 4.79 (d, 1H, J=5.7 Hz, H-5), 4.75-4.53 (m, 2H, CH₂-Ph), 4.67-4.54 (m, 4H, 2×CH₂-Ph), 4.21 (m, 1H, H-5′), 4.12 (d, 1H, J=7.2 Hz H-6a), 3.92 (br. d, 1H, H-2′), 3.89-3.85 (m, 2H, H-3′, H-4), 3.83-3.77 (m, 2H, H-6b, H-6′ a), 3.71-3.64 (m, 3H, H-3, H-4′, H-6′ b), 3.52 (d, 1H, J=10.2 Hz, OH), 3.15 (s, 1H, H-2), 0.90 (s, 9H, C(CH₃)₃), 0.02 (2s, 6H, Si(CH₃)₂). MALDI-MS, positive mode, m/z: 756.15 [M+Na⁺], 772.09 [M+K⁺]. [α]_{D}²¹=27.9 (c=1.02, CHCl₃).

Step 8.f: Synthesis of compound 140: In a dry round-bottom flask, compound 138 (5.77 g, 7.86 mmol) was dissolved in anhydrous pyridine (52 mL) under a nitrogen atmosphere. After cooling the solution to 0° C., acetic anhydride (2.97 mL, 31.45 mmol, 4 eq.) and DMAP (96 mg, 0.79 mmol, 0.1 eq.) were successively added and the resulting solution was stirred for 2 h at room temperature. The reaction mixture was concentrated, diluted with dichloromethane (300 mL) and washed with water (50 mL). The organic layer was dried over MgSO₄, filtered, concentrated and purified by flash chromatography on silica gel column (heptane/ethyl acetate: 8/2) to give compound 140 (5.85 g, 96%) as a white amorphous solid. ¹H NMR (400 MHz, CDCl₃, ppm), δ: 7.38-7.23 (m, 15H, arom.), 5.51 (s, 1H, H-1), 5.24 (br s, 1H, H-1′), 5.06 (m, 1H, H-2′), 4.81-4.76 (m, 2H, H-5, CH-Ph), 4.68 (d, 1H, J=11.7 Hz, CH-Ph), 4.62-4.52 (m, 3H, CH₂-Ph, CH-Ph), 4.43 (d, 1H, J=11.7 Hz, CH-Ph), 4.21-4.16 (m, 1H, H-5′), 4.15-4.10 (m, 1H, H-6a), 3.90-3.84 (m, 2H, H-6′a, H-4), 3.83-3.80 (m, 1H, H-3′), 3.78 (m, 1H, H-6b), 3.73 (d, 1H, J=5.4 Hz, H-6′ b), 3.69 (s, 1H, H-3), 3.53 (m, 1H, H-4′), 3.15 (s, 1H, H-2), 2.05 (s, 3H, CH₃—OAc), 0.88 (s, 9H, C(CH₃)₃), 0.02 (2s, 6H, Si(CH₃)₂). MALDI-MS, positive mode, m/z: 798.69 [M+Na⁺], 814.65 [M+K⁺]. [α]_{D}²¹=34.8 (c=1.24, CHCl₃).

Step 8.g: Synthesis of compound 142: Chromium trioxide (1.95 g, 19.49 mmol, 2.6 eq.) in a 3.5 M aqueous solution of H₂SO₄ (8.15 mL) was added slowly to a solution of compound 140 (5.82 g, 7.50 mmol) in acetone (55 mL) at 0° C. After stirring for 2 h 30 at room temperature, the reaction mixture was diluted with dichloromethane (150 mL) and washed with water (2×100 mL). The organic layer was dried over MgSO₄, filtered and concentrated. The resulting carboxylic acid was converted to methyl ester according to the general method F. Purification was effected by chromatography on silica gel column (heptane/ethyl acetate: 6/4) to give compound 142 (3.86 g, 75%) as a colourless oil. ¹H NMR (400 MHz, CDCl₃, ppm): δ=7.41-7.16 (m, 15H, arom.), 5.51 (s, 1H, H-1), 5.32 (d, 1H, J=3.1 Hz, H-1′), 4.99 (t, 1H, J=3.1 Hz, H-2′), 4.76 (d, 1H, J=3.1 Hz, H-5′), 4.81, 4.61 (2d, 2H, J=11.8 Hz, CH₂-Ph), 4.73 (d, 1H, J=5.2 Hz, H-5), 4.65-4.40 (m, 4H, 2×CH₂-Ph), 4.09 (d, 1H, J=7.6 Hz, H-6a), 3.89 (t, 1H, J=3.1 Hz, H-4′), 3.82-3.79 (m, 1H, H-3′), 3.86 (s, 1H, H-4), 3.78 (d, 1H, J=7.6 Hz, H-6b), 3.70 (s, 1H, H-3), 3.69 (s, 3H, CO₂Me), 3.22 (s, 1H, H-2), 2.03 (s, 3H, CH₃—Ac). MALDI-MS, positive mode, m/z: 712.35 [M+Na⁺], 728.31 [M+K⁺]. [α]_{D}²¹=51.8 (c=1.10, CHCl₃).

Step 8.h: Synthesis of compound 144: Acetolysis of compound 142 (3.86 g, 5.60 mmol) was performed according to the general method F. Purification was effected by chromatography on silica gel column (heptane/ethyl acetate: 5/5) to give compound 144 (4.48 g, quant., α/β: 81/19) as a white solid. ¹H NMR (400 MHz, CDCl₃, ppm): δ=7.40-7.20 (m, 15H, arom.), 6.21 (d, 1H, J=3.6 Hz, H-1α), 5.44 (d, 0.23H, J=8.5 Hz, H-1β), 5.30 (d, 1H, J=5.1 Hz, H-1′), 4.99-4.86 (m, 2H, H-2′, CH-Ph), 4.56-4.44 (m, 2H, CH₂-Ph), 4.76-4.66 (m, 4H, CH-Ph, CH₂-Ph, H-5′), 4.36 (dd, 1H, J=2.0 Hz, J=12.6 Hz, H-6a), 4.20 (dd, 1H, J=3.5 Hz, J=12.4 Hz, H-6b), 3.94-3.76 (m, 5H, H-3, H-4, H-5, H-4′, H-3′), 3.58 (s, 3H, CO₂Me), 3.56 (dd, 1H, J=10.0 Hz, J=3.5 Hz, H-2), 2.19, 2.06, 2.05 (s, 9H, CH₃—Ac). MALDI-MS, positive mode, m/z: 814.49 [M+Na⁺], 830.41 [M+K⁺].

Step 8.i: Synthesis of compound 146: Selective hydrolysis of compound 144 (1.09 g, 1.38 mmol) was performed according to the general method G. Compound 146 was obtained as a yellow oil and used in the next step without any further purification. MALDI-MS, positive mode, m/z: 772.57 [M+Na⁺], 788.49 [M+K⁺].

Step 8.j: Synthesis of compound 148: Trichloroacetimidate formation of compound 146 (1.32 mmol) was performed according to the general method H. Purification was effected by chromatography on silica gel column (heptane/ethyl acetate: 6/4 with 1% Et₃N) to give compound 148 (1.02 g, 83% over 2 steps, α/β: 43/57) as a white amorphous solid. ¹H NMR (400 MHz, CDCl₃, ppm), δ: 8.74 (s, 0.75H, NHα), 8.72 (s, 1H, NHβ), 7.40-7.19 (m, 15H, arom.), 6.38 (d, 0.75H, J=3.6 Hz, H-1α), 5.61 (d, 1H, J=8.5 Hz, H-1β), 5.27 (d, 1H, J=4.2 Hz, H-1′), 4.96-4.86 (m, 3H, H-2′, CH₂-Ph), 4.77-4.68 (m, 5H, H-5′, 2×CH₂-Ph), 4.46-4.37 (m, 2H, H-6aα, H-6aβ), 4.26-4.19 (m, 2H, H-6bα, H-6bβ), 4.16-4.05 (m, 2H, H-4α, H-4β), 4.04-4.00 (m, 1H, H-5α), 3.91 (t, 1H, J=9.8 Hz, H-3α), 3.87-3.82 (m, 1H, H-4′), 3.82-3.77 (m, 1H, H-3′), 3.74-3.68 (m, 2H, H-2α, H-2β), 3.66-3.61 (m, 1H H-5β), 3.57, 3.56 (2s, 6H, CO₂Meα,β), 3.47 (t, 1H, J=9.6 Hz, H-3β), 2.07-2.03 (4s, 12H, CH₃—Ac). MALDI-MS, positive mode, m/z: 772.03 [M+Na⁺—C(NH)CCl₃], 787.98 [M+K⁺—C(NH)CCl₃].

Synthesis of disaccharide 149 was carried out as described for compound 148.

Compound 149: NMR (400 MHz, CDCl₃, ppm): δ=8.72 (s, 1H, NHα), 8.71 (s, 0.15H, NHβ), 7.48-7.23 (m, 10H, arom.), 6.42 (d, 1H, J=3.3 Hz, H-1α), 5.65 (d, 0.15H, J=8.2 Hz, H-1β), 5.22 (d, 1H, J=3.5 Hz, H-1′), 4.77, 4.66 (2d, 2H, J=11.5 Hz, CH₂-Ph), 4.60, 4.52 (2d, 2H, J=11.5 Hz, CH₂-Ph), 3.81 (s, 3H, CO₂Me), 3.62 (s, 3H, OMe), 1.99, 1.97 (2s, 6H, CH₃—OAc). MALDI-MS, positive mode, m/z: 696.27 [M+Na⁺—C(NH)CCl₃].

Step 8.k: Synthesis of compound 150: Coupling reaction of disaccharide donor 148 (200 mg, 0.224 mmol, 1 eq.) and 4-pentyn-1-ol (31 μL, 0.336 mmol, 1.5 eq.) was performed in anhydrous dichloromethane (C=0.3 M vs donor) with the activator tert-butyldimethylsilyl trifluoromethanesulfonate (0.15 eq. vs donor) according to the general method B. Purification was effected by chromatography on silica gel column (heptane/ethyl acetate: 7/3) to give compound 150 (91 mg, β anomer, 50%) and a mixture of α/β compounds (63 mg, 35%) as white amorphous solid and a global yield of 85%. ¹H NMR (400 MHz, CDCl₃, ppm): δ=7.42-7.13 (m, 15H, arom.), 5.23 (d, 1H, J=4.4 Hz, H-1′), 4.86 (t, 1H, J=4.4 Hz, H-2′), 4.80 (d, 1H, J=10.8 Hz, CH-Ph), 4.75-4.66 (m, 3H, H-5′, CH₂-Ph), 4.62 (d, 1H, J=10.8 Hz, CH-Ph), 4.47 (2d, 2H, J=11.5 Hz, CH₂-Ph), 4.42 (dd, 1H, J=12.2 Hz, J=2.2 Hz, H-6a), 4.23 (d, 1H, J=8.0 Hz, H-1), 4.17 (dd, 1H, J=12.2 Hz, J=4.3 Hz, H-6b), 4.0-3.89 (m, 2H, H-4, CH₍ₐ₎-pent-4-ynyl), 3.81 (t, 1H, J=4.4 Hz, H-4′), 3.76 (t, 1H, J=4.4 Hz, H-3′), 3.68-3.63 (m, 1H, CH_{(a′)}-pent-4-ynyl), 3.52 (s, 3H, CO₂Me), 3.45-3.27 (m, 3H, H-5, H-3, H-2), 2.37-2.29 (m, 2H, CH_{2(c)}-pent-4-ynyl), 2.05-2.00 (2s, 6H, CH₃—OAc), 1.94 (t, 1H, J=2.6 Hz, CH_{(d)}-alkyne), 1.89-1.75 (m, 2H, CH_{2(b)}-pent-4-ynyl). MALDI-MS, positive mode, m/z: 838.26 [M+Na⁺], 854.17 [M+K⁺]. [α]_{D}²¹=65.7 (c=0.3, CHCl₃).

### 1. Preparation 9: Synthesis of Capping-End and Elongating Disaccharides 157 and 158 (Scheme 9)

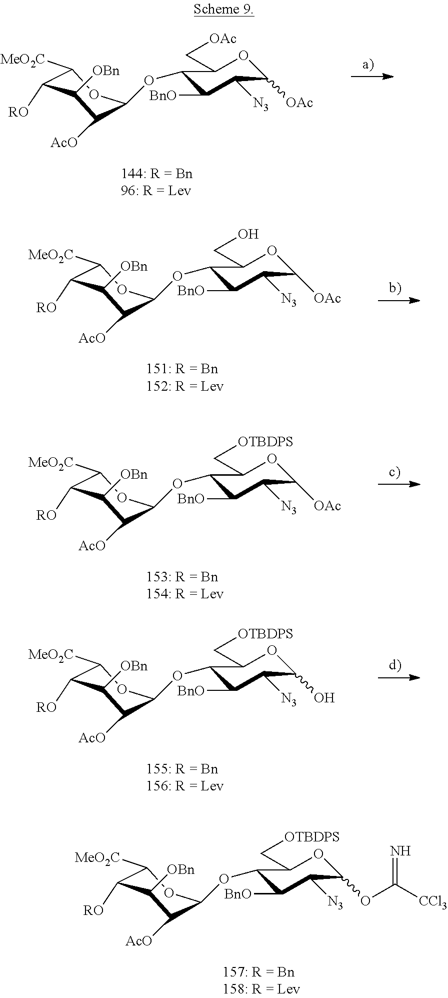

Step 9.a: Synthesis of compound 151: In a dry round-bottom flask, compound 144 (2.05 g, 2.59 mmol) was dissolved in a dry mixture of tetrahydrofurane/methanol (1/1, 52 mL) and the catalyst [tBu₂SnOH(Cl)]₂ (593 mg, 1.04 mmol, 0.4 eq.), which was prepared as described in J. Chem. Soc, 1971, 360 and J. Organomet. Chem. 1985, 287, 163-178, was added. The reaction mixture was stirred for 4 h at 45° C. and solvents were removed under reduced pressure. The crude residue was purified by chromatography on silica gel column (heptane/ethyl acetate: 4/6 to 1/9) to afford compound 151 (1.26 g, 65%) as a white solid. ¹H NMR (400 MHz, CDCl₃, ppm): δ=7.33-7.18 (m, 15H, arom.), 6.13 (d, 1H, J=3.8 Hz, H-1), 5.30 (d, 1H, J=4.2 Hz, H-1′), 4.88-4.83 (m, 2H, H-2′, CH-Ph), 4.67-4.57 (m, 4H, CH₂-Ph, CH-Ph, H-5′), 4.49-4.39 (m, 2H, CH₂-Ph), 3.97 (t, 1H, J=9.4 Hz, H-4), 3.81-3.64 (m, 6H, H-3, H-4′, H-5, H-3′, H-6a,b), 3.52 (dd, 1H, J=10.3 Hz, J=3.8 Hz, H-2), 3.47 (s, 3H, CO₂Me), 2.12, 1.95 (2s, 6H, CH₃—Ac). MALDI-MS, positive mode, m/z: 772.50 [M+Na⁺], 788.47 [M+K⁺]. [α]_{D}²¹ 1.8 (c=1.03, CHCl₃).

Step 9.b: Synthesis of compound 153: In a dry round bottom flask, compound 151 (1.24 g, 1.65 mmol) was dissolved in anhydrous dichloromethane (5.5 mL). Tert-butylchlorodiphenylsilane (2.15 mL, 8.27 mmol, 5 eq.), Et₃N (1.15 mL, 8.27 mmol, 5 eq.) and DMAP (101 mg, 0.83 mmol, 0.5 eq.) were successively added and the reaction mixture was stirred overnight at room temperature. The reaction mixture was diluted in dichloromethane (50 mL) and the organic layer was washed with water (5 mL), dried over MgSO₄, filtered and concentrated under reduced pressure. The crude residue was chromatographied by silica gel column (heptane/ethyl acetate: 8/2) to give compound 153 (1.38 g, 84%) as a white solid. ¹H NMR (400 MHz, CDCl₃, ppm): δ=7.69-7.63 (m, 4H, arom.), 7.36-7.21 (m, 21H, arom.), 6.18 (d, 1H, J=3.8 Hz, H-1), 5.44 (d, 1H, J=4.8 Hz, H-1′), 4.88-4.83 (m, 2H, H-2′, CH-Ph), 4.67-4.57 (m, 3H, CH₂-Ph, CH-Ph), 4.59 (d, 1H, J=4.8 Hz, H-5′), 4.46 (s, 2H, CH₂-Ph), 4.08 (t, 1H, J=9.4 Hz, H-4), 3.87-3.82 (m, 2H, H-6a, H-3′), 3.80-3.72 (m, 3H, H-3, H-6b, H-4′), 3.63-3.58 (m, 1H, H-5), 3.50 (dd, 1H, J=10.3 Hz, J=3.8 Hz, H-2), 3.45 (s, 3H, CO₂Me), 2.08, 1.75 (2s, 6H, CH₃—Ac), 0.99 (s, 9H, C(CH₃)₃). MALDI-MS, positive mode, m/z: 1010.19 [M+Na⁺], 1026.15 [M+K⁺]. [α]_{D}²¹=+15.8 (c=0.98, CHCl₃).

Step 9.c: Synthesis of compound 155: Selective hydrolysis of compound 153 (1.38 g, 1.40 mmol) was performed according to the general method G. Compound 155 was directly used in the next step without any further purification. MALDI-MS, positive mode, m/z: 968.21 [M+Na⁺], 984.16 [M+K⁺].

Step 9.d: Synthesis of compound 157: Trichloroacetimidate formation of compound 155 (1.40 mmol) was performed according to the general method H. Purification was effected by chromatography on silica gel column (heptane/ethyl acetate: 5/5 with 1% Et₃N) to give compound 157 (1.42 g, 93% over 2 steps, α/β: 52/48) as a white solid. ¹H NMR (400 MHz, CDCl₃, ppm): δ=8.64 (s, 1H, NHα), 8.62 (s, 0.92H, NHβ) 7.68-7.60 (m, 4H, arom.), 7.38-7.21 (m, 21H, arom.), 6.36 (d, 1H, J=3.6 Hz, H-1α), 5.55 (d, 0.92H, J=8.5 Hz, H-1β), 5.35 (d, 1H, J=3.8 Hz, H-1′), 3.41, 3.42 (2s, 5.76H, CO₂Me α, β), 1.75, 1.78 (2s, 5.76H, CH₃—OAc), 0.99 (2s, 17.28H, C(CH₃)₃).

Synthesis of disaccharide 158 was carried out as described for compound 157.

Compound 158: ¹H NMR (400 MHz, CDCl₃, ppm): δ=8.69 (s, 0.64H, NHβ), 8.68 (s, 1H, NHα), 7.73-7.64 (m, 4H, arom.), 7.46-7.15 (m, 16H, arom.), 6.42 (d, 1H, J=3.5 Hz, H-1α), 5.63 (d, 0.64H, J=8.5 Hz, H-1β), 5.29 (br. s, 1H, H-1′), 3.45 (s, 1.92H, CO₂Meβ), 3.44 (s, 3H, CO₂Meα), 2.83-2.44 (m, 6.56H, CH₂Lev), 2.17-2.16 (2s, 4.92H, CH₃-Lev,), 1.96, 195 (2s, 4.92H, CH₃—OAc), 1.08 (2s, 14.76H, C(CH₃)₃).

### D. Trisaccharides Preparations

1. Preparation 10: synthesis of Acceptor Trisaccharide 160 (Scheme 10)

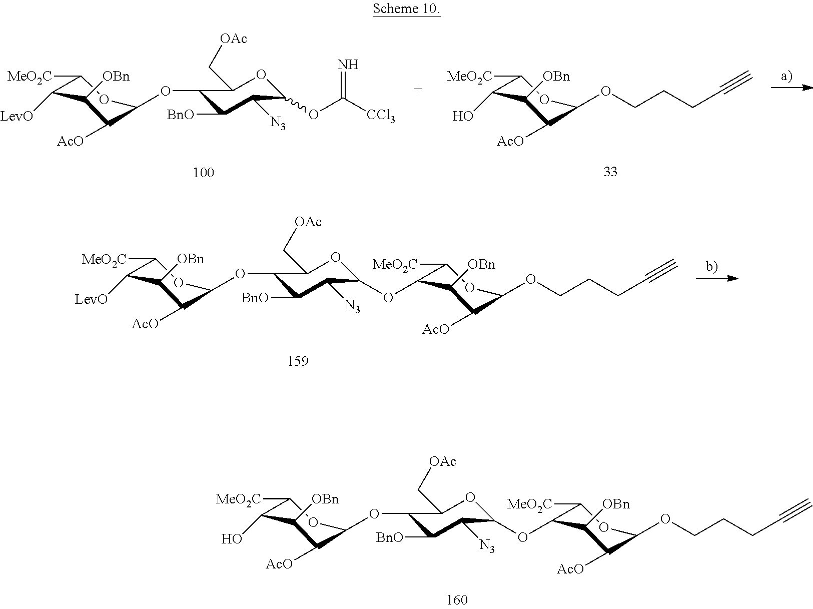

Step 10.a: Synthesis of compound 159: O-glycosylation reaction between disaccharide donor 100 (900 mg, 1 mmol, 1.2 eq.) and monosaccharide acceptor 33 (338 mg, 0.83 mmol, 1 eq.) was performed according to the general method B. Purification was effected by a Sephadex LH20 gel column (dichloromethane/ethanol: 7/3) to give trisaccharide 159 (648 mg, 68%) as a white amorphous solid. ¹H NMR (400 MHz, CDCl₃, ppm): δ=7.47-7.16 (m, 15H, arom.), 5.14 (d, 1H, J=3.7 Hz, H-1 IdoUA^{III}), 5.07 (t, 1H, J=3.7 Hz, H-4 IdoUA^{III}), 5.0 (sl, 1H, H-1 IdoUA^{I}), 4.92 (sl, 1H, H-2 IdoUA^{I}), 4.88 (d, 1H, J=3.7 Hz, H-1 Glc^{II}), 4.87-4.81 (m, 3H, H-2 IdoUA^{III}, H-5 IdoUA^{III}, H-5 IdoUA^{I}), 4.78, 4.63 (2d, 2H, J=11.5 Hz, CH₂-Ph), 4.77, 4.61 (2d, 2H, J=10.5 Hz, CH₂-Ph), 4.72-4.67 (m, 2H, CH₂-Ph), 4.46 (dd, 1H, J=12.6 Hz, J=1.7 Hz, H-6a Glc^{II}), 4.22 (dd, 1H, J=12.6 Hz, J=3.1 Hz, H-6b Glc^{II}), 4.07 (t, 1H, J=3.1 Hz, H-4 IdoUA^{I}), 3.95-3.82 (m, 4H, H-3 IdoUA^{I}, H-4 Glc^{II}, H-5 Glc^{II}, CH_{(a′)}-pent-4-ynyl), 3.78 (t, 1H, J=3.7 Hz, H-3 IdoUA^{III}), 3.74-3.71 (s, 4H, CO₂Me, H-3 Glc^{II}), 3.60-3.51 (m, 1H, CH_{(a′)}-pent-4-ynyl), 3.48 (s, 3H, CO₂Me), 3.31 (dd, 1H, J=10.3 Hz, J=3.7 Hz, H-2 Glc^{II}), 2.80-2.39 (m, 4H, CH₂-Lev), 2.29-2.20 (m, 2H, CH_{2(c)}-pent-4-ynyl), 2.16 (s, 3H, CH₃-Lev), 2.09, 2.06, 2.05 (3s, 9H, CH₃—OAc) 1.90 (t, 1H, J=2.7 Hz, CH_{(d)}-alkyne), 1.86-1.70 (m, 2H, CH_{2(b)}-pent-4-ynyl). MALDI-MS, positive mode, m/z: 1168.25 [M+Na⁺], 1184.16 [M+K⁺]. [α]_{D}²¹=18.0 (c=0.69, CHCl₃).

Step 10.b: Synthesis of compound 160: In a dry round-bottom flask, compound 159 (196 mg, 0.171 mmol) was dissolved in a mixture of pyridine/acetic acid 3/1 (1.7 mL) at 0° C. followed by the addition of hydrazine acetate (31 mg, 0.34 mmol, 2 eq.). The reaction was stirred at room temperature for 2 h after which it was diluted with dichloromethane (30 mL) and successively washed with a 5% aqueous solution of H₂SO₄ (5 mL), a saturated aqueous solution of NaHCO₃ (5 mL) and water (5 mL). The organic layer was dried over MgSO₄, filtered, the solvent was evaporated under reduced pressure and the residue was purified by flash chromatography on silica gel column (heptane/ethyl acetate: 8/2 to 4/6) to give trisaccharide acceptor 160 (161 mg, 90%) as a viscous compound. ¹H NMR (400 MHz, CDCl₃, ppm): δ=7.42-7.20 (m, 15H, arom.), 5.06 (sl, 1H, H-1 IdoUA^{III}), 5.00 (sl, 1H, H-1 IdoUA^{I}), 4.92 (sl, 1H, H-2 IdoUA^{I}), 4.88 (d, 2H, H-1 Glc^{II}, H-2 IdoUA^{III}), 4.85-4.70 (m, 5H, H-5 IdoUA^{I}, H-5 IdoUA^{III}, 3×CH-Ph), 4.66-4.59 (m, 3H, 3×CH-Ph), 4.46 (d, 1H, J=12.6 Hz, H-6a Glc^{II}), 4.21 (d, 1H, J=12.6 Hz, H-6b Glc^{II}), 4.07 (t, 1H, J=2.5 Hz, H-4 IdoUA^{I}), 3.98-3.92 (m, 1H, H-4 IdoUA^{III}), 3.92-3.81 (m, 4H, H-3 IdoUA^{I}, H-4 Glc^{II}, H-5 Glc^{II}, CH₍ₐ₎-pent-4-ynyl), 3.76-3.67 (m, 5H, H-3 IdoUA^{III}, H-3 Glc^{II}, CO₂Me), 3.60-3.52 (m, 1H, CH_{(a′)}-pent-4-ynyl), 3.49 (s, 3H, CO₂Me), 3.31 (dd, 1H, J=10.3 Hz, J=3.6 Hz, H-2 Glc^{II}), 2.54 (d, 1H, J=10.8 Hz, OH), 2.29-2.20 (m, 2H, CH_{2(c)}-pent-4-ynyl), 2.09, 2.06, 2.05 (3s, 9H, CH₃—OAc), 1.90 (t, 1H, J=2.7 Hz, CH_{(d)}-alkyne), 1.88-1.72 (m, 2H, CH_{2(b)}-pent-4-ynyl). MALDI-MS, positive mode, m/z: 1070.11 [M+Na⁺], 1086.04 [M+K⁺]. [α]_{D}²¹=6.3 (c=1.05, CHCl₃).

### E. Tetrasaccharides Preparations

### 1. Preparation 11: Synthesis of Donor Tetrasaccharide 164 (Scheme 11)

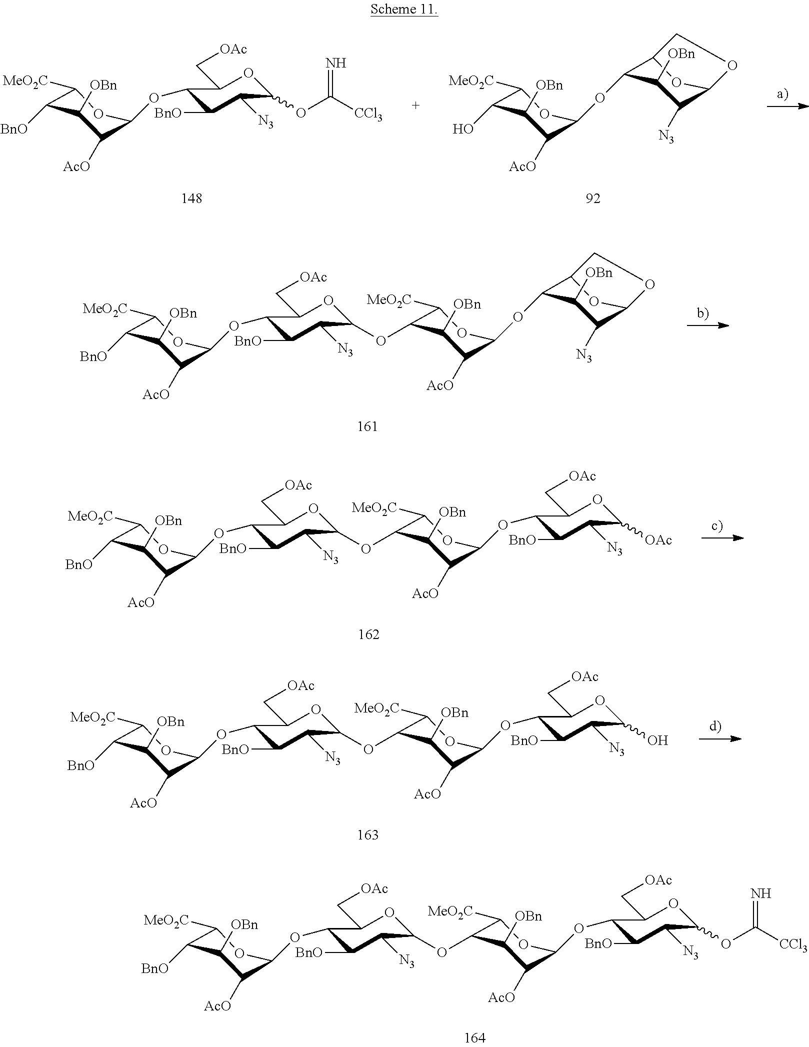

Step 11.a: Synthesis of compound 161: O-glycosylation of disaccharide donor 148 (850 mg, 0.95 mmol, 1.3 eq.) with disaccharide acceptor 92 (438 mg, 0.73 mmol, 1 eq.) was performed according to the general method B. Purification was effected by chromatography on silica gel column (heptane/ethyl acetate: 7/3 to 6/4) to give tetrasaccharide 161 (711 mg, 73%) as a white amorphous solid. ¹H NMR (400 MHz, CDCl₃, ppm): δ=7.40-7.20 (m, 25H, arom.), 5.50 (s, 1H, H-1 Glc^{I}), 5.32-5.28 (m, 2H, H-1 IdoUA^{II}, H-1 IdoUA^{IV}), 5.02 (t, 1H, J=2.1 Hz, H-2 IdoUA^{IV}), 4.96-4.79 (m, 5H, H-1 Glc^{III}, CH₂-Ph, H-2 IdoUA^{II}, H-5 IdoUA^{IV}), 4.74-4.47 (m, 10H, 4×CH₂-Ph, H-5 IdoUA^{II}, H-5 Glc^{I}), 4.44 (dd, 1H, J=12.7 Hz, J=1.9 Hz, H-6a Glc^{II}), 4.21 (dd, 1H, J=12.7 Hz, J=3.0 Hz, H-6b Glc^{II}), 4.17-3.94 (m, 4H, H-3 IdoUA^{IV}, H-4 IdoUA^{IV}, H-4 Glc^{III}, H-6a Glc^{I}), 3.87-3.64 (m, 10H, H-3 IdoUA^{II}, H-4 IdoUA^{II}, H-4 Glc^{I}, H-6b Glc^{I}, H-3 Glc^{I}, H-3 Glc^{III}, H-5 Glc^{III}, CO₂Me), 3.57 (s, 3H, CO₂Me), 3.33 (dd, 1H, J=10.3 Hz, J=3.5 Hz, H-2 Glc^{III}), 3.24 (d, 1H, J=2.5 Hz, H-2 Glc^{I}), 2.11, 2.07, 2.04 (3s, 9H, CH₃—OAc). MALDI-MS, positive mode, m/z: 1353.55 [M+Na⁺], 1369.46 [M+K⁺]. [α]_{D}²¹=19.7 (c=0.87, CHCl₃).

Step 11.b: Synthesis of compound 162: Acetolysis of compound 161 (711 mg, 0.53 mmol) was performed according to the general method F at 50° C. Compound 162 (α/β: 68/32) was obtained as a yellow oil and directly used in the next step without any further purification. ¹H NMR (400 MHz, CDCl₃, ppm): δ=7.33-7.10 (m, 25H, arom.), 6.13 (d, 1H, J=3.6 Hz, H-1 Glc^{I}α), 5.36 (d, J=8.4 Hz, 0.47H, H-1 Glc^{I}β), 5.23-5.19 (m, 2H, H-1 IdoUA^{II}, H-1 IdoUA^{IV}), 4.88-4.77 (m, 5H, H-1 Glc^{III}, H-2 IdoUA^{IV}, H-2 IdoUA^{II}, CH₂-Ph), 4.73-4.62 (m, 4H, 2×CH₂-Ph), 4.60-4.53 (m, 6H, 2×CH₂-Ph, H-5 IdoUA^{II}, H-5 IdoUA^{IV}), 4.35-4.26 (m, 3H, H-6a Glc^{I}β, H-6a Glc^{I}α, H-6a Glc^{III}), 4.18-4.05 (m, 3H, H-6b Glc^{I}β, H-6b Glc^{I}α, H-6b Glc^{III}), 3.95-3.68 (m, 10H, H-3 Glc^{I}α, H-4 Glc^{I}α, H-5 Glc^{I}α, H-4 Glc^{I}β, H-3 IdoUA^{II}, H-3 IdoUA^{IV}, H-4 IdoUA^{II}, H-4 IdoUA^{IV}, H-4 Glc^{III}, H-5 Glc^{III}), 3.62-3.42 (m, 10H, 2×CO₂Me, H-2 Glc^{I}α, H-2 Glc^{I}β, H-5 Glc^{I}β, H-3 Glc^{III}), 3.33 (t, J=9.3 Hz, 1H, H-3 Glc^{I}β), 3.24 (dd, J=3.4 Hz, J=10.4 Hz, 1H, H-2 Glc^{III}), 2.11, 2.04, 1.97, 1.95, 1.94 (5s, 15H, CH₃—OAc). MALDI-MS, positive mode, m/z: 1455 [M+Na⁺], 1471.46 [M+K⁺].

Step 11.c: Synthesis of compound 163: Selective hydrolysis of compound 162 (0.53 mmol) was performed according to the general method G. Purification was effected by chromatography on silica gel column (heptane/ethyl acetate: 5/5) to give compound 163 (670 mg, 90% over 2 steps) as a white solid. MALDI-MS, positive mode, m/z: 1413.31 [M+Na⁺].

Step 11.d: Synthesis of compound 164: Trichloroacetimidate formation of compound 163 (670 mg, 0.48 mmol) was performed according to the general method H. Compound 164 (706 mg, 96%, α/β: 20/80) was obtained as a yellow amorphous solid and directly used in the next step without any further purification. ¹H NMR (400 MHz, CDCl₃, ppm): δ=8.7 (s, 0.25H, NHα), 8.65 (s, 1H, NHβ), 7.32-7.10 (m, 25H, arom.), 6.32 (d, 0.25H, J=3.5 Hz, H-1 Glc^{I}α), 5.53 (d, 1H, J=8.4 Hz, H-1 Glc^{I}β), 5.23-5.19 (m, 2H, H-1 IdoUA^{II}, H-1 IdoUA^{IV}), 4.90-4.52 (m, 13H, H-1 Glc^{III}, H-2 IdoUA^{IV}, H-2 IdoUA^{II}, 4×CH₂-Ph, H-5 IdoUA^{II}, H-5 IdoUA^{IV}), 4.49-4.24 (m, 4H, H-6a Glc^{I}, H-6a Glc^{III}, CH₂-Ph), 4.20-4.05 (m, 2H, H-6b Glc^{I}, H-6b Glc^{III}), 3.97-3.68 (m, 6H, H-4 Glc^{I}, H-5 Glc^{I}, H-3 IdoUA^{II}, H-3 IdoUA^{IV}, H-4 IdoUA^{II}, H-4 IdoUA^{IV}), 3.65-3.52 (m, 3H, H-4 Glc^{III}, H-5 Glc^{III}, H-2 Glc^{I}), 3.48, 3.44 (2s, 6H, 2×CO₂Me), 3.33 (t, 1H, J=9.3 Hz, H-3 Glc^{I}), 3.23 (dd, 1H, J=3.4 Hz, J=10.2 Hz, H-2 Glc^{III}), 1.99-1.93 (4s, 12H, CH₃—OAc). MALDI-MS, positive mode, m/z: 1414.22 [M+Na⁺—C(NH)CCl₃], 1558.17 [M+Na⁺].

### 1. Preparation 12: Synthesis of Acceptor Tetrasaccharides 166, 167, 168, 169, 170, 171, 172, 173, 174, 175, 176 and 177 (Scheme 12)

Below is reported the general formula of all the acceptor tetrasaccharides synthesized.

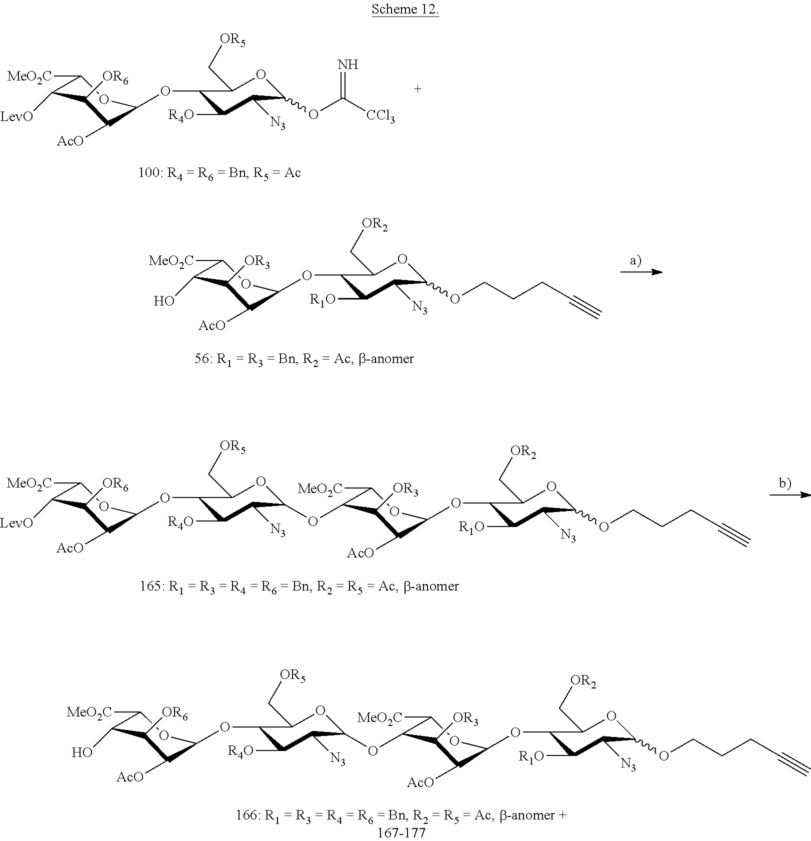

| Compound | R₁ | R₃ | R₄ | R₆ | R₂ | R₅ | anomer |
|---|---|---|---|---|---|---|---|
| 166 | Bn | Bn | Bn | Bn | Ac | Ac | β |
| 167 | Bn | Bn | Bn | Bn | Ac | Ac | α |
| 168 | Bn | Bn | Bn | Bn | TBDPS | TBDPS | α |
| 169 | Me | Bn | Me | Bn | TBDPS | Ac | β |
| 170 | Bn | Me | Bn | Me | TBDPS | Ac | β |
| 171 | Me | Me | Me | Me | TBDPS | Ac | β |
| 172 | Me | Bn | Bn | Bn | TBDPS | Ac | β |
| 173 | Me | Me | Bn | Bn | TBDPS | Ac | β |
| 174 | Bn | Bn | Me | Me | Ac | Ac | β |
| 175 | Bn | Me | Me | Me | TBDPS | Ac | β |
| 176 | Bn | Bn | Bn | Me | Ac | Ac | β |
| 177 | Me | Me | Me | Bn | TBDPS | Ac | β |

Step 12.a: Synthesis of compound 165:O-glycosylation reaction between disaccharide donor 100 (184 mg, 0.204 mmol, 1.3 eq.) and disaccharide acceptor 56 (114 mg, 0.157 mmol, 1 eq.) was performed according to the general method B. Purification was effected by chromatography on silica gel column (heptane/ethyl acetate: 6/4 to 4/6) to give tetrasaccharide 165 (172.8 mg, 70%) as a white amorphous solid. ¹H NMR (400 MHz, CDCl₃, ppm): δ=7.33-7.13 (m, 20H, arom.), 5.12 (d, 1H, J=3.2 Hz, H-1 IdoUA^{II}), 5.06 (d, 1H, J=2.9 Hz, H-1 IdoUA^{IV}), 5.00 (t, 1H, J=3.6 Hz, H-4 IdoUA^{IV}), 4.85-4.80 (m, 2H, H-2 IdoUA^{II}, H-1 Glc^{III}), 4.79-4.75 (m, 2H, H-2 IdoUA^{IV}, H-5 IdoUA^{IV}), 4.71 (d, 1H, J=3.6 Hz, H-5 IdoUA^{II}), 4.70-4.50 (m, 8H, 4×CH₂-Ph), 4.40-4.31 (m, 2H, H-6a Glc^{III}, H-6a Glc^{I}), 4.18 (d, 1H, J=7.9 Hz, H-1 Glc^{I}), 4.16-4.08 (m, 2H, H-6b Glc^{III}, H-6b Glc^{I}), 3.95-3.69 (m, 7H, H-3 IdoUA^{II}, H-4 IdoUA^{II}, H-3 IdoUA^{IV}, H-4 Glc^{I}, H-4 Glc^{III}, H-5 Glc^{III}, CH₍ₐ₎-pent-4-ynyl), 3.62-3.53 (m, 2H, H-3 Glc^{III}, CH_{(a′)}-pent-4-ynyl), 3.44-3.28 (m, 8H, H-2 Glc^{I}, H-5 Glc^{I}, 2×CO₂Me), 3.27-3.19 (m, 2H, H-3 Glc^{I}, H-2 Glc^{III}), 2.72-2.35 (m, 4H, CH₂-Lev), 2.33-2.24 (m, 2H, CH_{2(c)}-pent-4-ynyl), 2.09 (s, 3H, CH₃-Lev), 2.06-1.95 (m, 12H, CH₃—OAc), 1.89 (t, 1H, J=2.6 Hz, CH_{(d)}-alkyne), 1.87-1.74 (m, 2H, CH_{2(b)}-pent-4-ynyl). MALDI-MS, positive mode, m/z: 1487.68 [M+Na⁺]. [α]_{D}²¹=14.0 (c=0.48, CHCl₃).

Step 12.b: Synthesis of compound 166: In a dry round-bottom flask, compound 165 (271 mg, 0.185 mmol) was dissolved in a mixture of pyridine/acetic acid 3/1 (1.5 mL) at 0° C. followed by the addition of hydrazine acetate (34.1 mg, 0.37 mmol, 2 eq.). The reaction was stirred at room temperature for 2 h after which it was diluted with dichloromethane (100 mL) and successively washed with a 5% aqueous solution of H₂SO₄ (10 mL), a saturated aqueous solution of NaHCO₃ (10 mL) and water (10 mL). The organic layer was dried over MgSO₄, filtered, the solvent was evaporated under reduced pressure and the residue was purified by flash chromatography on silica gel column (heptane/ethyl acetate: 5/5 to 4/6) to give tetrasaccharide acceptor 166 (215 mg, 85%) as a white amorphous solid. ¹H NMR (400 MHz, CDCl₃, ppm): δ=7.35-7.12 (m, 20H, arom.), 5.11 (d, 1H, J=3.2 Hz, H-1 IdoUA^{II}), 4.98 (s, 1H, H-1 IdoUA^{IV}), 4.89-4.51 (m, 13H, H-1 Glc^{III}, H-2 IdoUA^{II}, H-5 IdoUA^{II}, H-2 IdoUA^{IV}, H-5 Glc^{III}, 4×CH₂-Ph), 4.38-4.29 (m, 2H, H-6a Glc^{I}, H-6a Glc^{II}), 4.18 (d, 1H, J=7.8 Hz, H-1 Glc^{I}), 4.16-4.08 (m, 2H, H-6b Glc^{III}, H-6b Glc^{I}), 3.97-3.46 (m, 12H, H-3 IdoUA^{II}, H-4 IdoUA^{II}, H-3 Glc^{III}, H-4 Glc^{III}, H-3 Glc^{I}, H-4 Glc^{I}, H-5 Glc^{I}, H-3 IdoUA^{IV}, H-4 IdoUA^{IV}, H-5 IdoUA^{IV}, CH₂₍ₐ₎-pent-4-ynyl), 3.42, 3.36 (2s, 6H, CO₂Me), 3.26 (dd, 1H, J=8.3 Hz, J=10.1 Hz, H-2 Glc^{I}), 3.20 (dd, 1H, J=3.3 Hz, J=10.5 Hz, H-2 Glc^{II}), 2.46 (d, 1H, J=10.7 Hz, OH IdoUA^{IV}), 2.33-2.24 (m, 2H, CH_{2(c)}-pent-4-ynyl), 1.89 (t, 1H, J=2.6 Hz, CH_{(d)}-alkyne), 2.06-1.95 (m, 12H, CH₃—OAc), 1.87-1.74 (m, 2H, CH_{2(b)}-pent-4-ynyl). MALDI-MS, positive mode, m/z: 1389.61 [M+Na⁺], 1405.68 [M+K⁺]. [α]_{D}²¹=5.9 (c=0.307, CHCl₃).

Preparation of all tetrasaccharides 167, 168, 169, 170, 171, 172, 173, 174, 175, 176, 177 were carried out as described for compound 166.

Compound 167: ¹H NMR (400 MHz, CDCl₃, ppm): δ=7.37-7.10 (m, 20H, arom.), 5.15 (d, 1H, J=3.2 Hz, H-1 IdoUA^{II}), 4.99 (s, 1H, H-1 IdoUA^{IV}), 4.89-4.51 (m, 14H, H-2 IdoUA^{II}, H-5 IdoUA^{II}, H-2 IdoUA^{IV}, H-1 Glc^{I}, H-5 Glc^{III}, H-1 Glc^{III}, 4×CH₂-Ph), 4.38-4.29 (m, 2H, H-6a Glc^{I}, H-6a Glc^{III}), 4.18 (d, 1H, J=12.8 Hz, H-6b Glc^{I}), 4.10 (d, 1H, J=11.8 Hz, H-6b Glc^{III}), 3.97-3.46 (m, 12H, H-3 IdoUA^{II}, H-4 IdoUA^{II}, H-3 Glc^{III}, H-4 Glc^{III}, H-3 Glc^{I}, H-4 Glc^{I}, H-5 Glc^{I}, H-3 IdoUA^{IV}, H-4 IdoUA^{IV}, H-5 IdoUA^{IV}, CH_{2(b)}-pent-4-ynyl), 3.42, 3.36 (2s, 6H, CO₂Me), 3.26 (dd, 1H, J=3.5 Hz, J=9.4 Hz, H-2 Glc^{I}), 3.20 (dd, 1H, J=3.3 Hz, J=10.5 Hz, H-2 Glc^{III}), 2.50 (d, 1H, J=10.5 Hz, OH IdoUA^{IV}), 2.33-2.24 (m, 2H, CH_{2(c)}-pent-4-ynyl), 1.89 (t, J=2.6 Hz, 1H, CH_{(d)}-alkyne), 2.06-1.95 (m, 12H, CH₃—OAc), 1.87-1.74 (m, 2H, CH_{2(b)}-pent-4-ynyl). MALDI-MS, positive mode, m/z: 1389.52 [M+Na⁺], 1405.48 [M+K⁺]. [α]_{D}²¹=+32.7 (c=0.35, CHCl₃).

Compound 168: ¹H NMR (400 MHz, CDCl₃, ppm): δ=7.42-7.13 (m, 40H, arom.), 5.32 (d, 1H, J=3.1 Hz, H-1 IdoUA^{IV}), 5.30 (s, 1H, H-1 IdoUA^{II}), 4.93-4.81 (m, 2H, H-1 Glc^{III}, H-1 Glc^{I}), 3.40, 3.21 (2s, 6H, CO₂Me), 2.32-2.26 (m, 2H, CH_{2(c)}-pent-4-ynyl), 1.90 (s, 3H, CH₃—OAc), 1.88-1.84 (m, 4H, CH_{(d)}-alkyne, CH₃—OAc), 1.82-1.74 (m, 2H, CH_{2(b)}-pent-4-ynyl), 1.06-1.00 (2s, 18H, C(CH₃)₃). MALDI-MS, positive mode, m/z: 1781.25 [M+Na⁺], 1797.17 [M+K⁺]. [α]_{D}²¹=+27.8 (c=0.54, CHCl₃).

Compound 169: ¹H NMR (400 MHz, CDCl₃, ppm): δ=7.73-7.64 (m, 4H, arom.), 7.43-7.22 (m, 16H, arom.), 5.23 (s, 1H, H-1 IdoUA^{II}), 5.01 (s, 1H, H-1 IdoUA^{IV}), 4.83 (br s, 1H, H-1 Glc^{III}), 4.20 (d, 1H, J=7.7 Hz, H-1 Glc^{I}), 3.91, 3.55 (m, 2H, CH₂₍ₐ₎-pent-4-ynyl), 3.83 (s, 3H, CO₂Me), 3.60, 3.49, 3.41 (3s, 9H, CO₂Me, 2×OMe), 2.35-2.29 (m, 2H, CH_{2(c)}-pent-4-ynyl), 2.08 (s, 6H, CH₃—OAc), 1.94 (s, 4H, CH₃—OAc, CH_{(d)}-alkyne), 1.91-1.78 (m, 2H, CH_{2(b)}-pent-4-ynyl), 1.07 (s, 9H, C(CH₃)₃). MALDI-MS, positive mode, m/z: 1433.37 [M+Na⁺], 1449.24 [M+K⁺]. [α]_{D}²¹=13.0 (c=0.96, CHCl₃).

Compound 170: ¹H NMR (400 MHz, CDCl₃, ppm): δ=7.73-7.64 (m, 4H, arom.), 7.43-7.22 (m, 16H, arom.), 5.36 (d, 1H, J=1.8 Hz, H-1 IdoUA^{II}), 5.06 (s, 1H, H-1 IdoUA^{IV}), 5.02 (d, 1H, J=3.5 Hz, H-1 Glc^{III}), 4.24 (d, 1H, J=7.9 Hz, H-1 Glc^{II}), 4.90, 4.73 (2d, 2H, J=11.5 Hz, CH₂-Ph), 4.82 (s, 2H, CH₂-Ph), 3.92, 3.57 (m, 2H, CH₂₍ₐ₎-pent-4-ynyl), 3.51, 3.52 (2s, 6H, CO₂Me), 3.49, 3.42 (2s, 6H, OMe), 2.35 (m, 3H, CH_{2(c)}-pent-4-ynyl, CH_{(d)}-alkyne), 2.59 (d, 1H, J=10.1 Hz, OH IdoUA^{IV}), 2.08, 2.07, 1.99 (3s, 9H, CH₃—OAc), 1.87-1.74 (m, 2H, CH_{2(b)}-pent-4-ynyl), 1.07 (s, 9H, C(CH₃)₃). MALDI-MS, positive mode, m/z: 1433.84 [M+Na⁺]. [α]_{D}²¹=+24.1 (c=0.68, CHCl₃).

Compound 171: ¹H NMR (400 MHz, CDCl₃, ppm): δ=7.74-7.63 (m, 4H, arom.), 7.44-7.31 (m, 6H, arom.), 5.22 (s, 1H, H-1 IdoUA^{II}), 5.02-4.94 (m, 2H, H-1 IdoUA^{IV}, H-1 Glc^{III}), 4.22-4.17 (m, 1H, H-1 Glc^{I}), 3.88, 3.55 (m, 2H, CH₂₍ₐ₎-pent-4-ynyl), 3.82, 3.78 (2s, 6H, CO₂Me), 3.53, 3.51, 3.45, 3.39 (4s, 12H, OMe), 2.68 (d, 1H, J=11.2 Hz, OH), 2.34-2.29 (m, 2H, CH_{2(c)}-pent-4-ynyl), 2.09, 2.07, 2.01 (3s, 9H, CH₃—OAc), 1.93 (t, 1H, J=2.6 Hz, CH_{(d)}-alkyne), 1.90-1.78 (m, 2H, CH_{2(b)}-pent-4-ynyl), 1.05 (s, 9H, C(CH₃)₃). MALDI-MS, positive mode, m/z: 1281.57 [M+Na⁺], 1297.44 [M+K⁺]. [α]_{D}²¹=+5.1 (c=0.37, CHCl₃).

Compound 172: ¹H NMR (400 MHz, CDCl₃, ppm): δ=7.75-7.63 (m, 4H, arom.), 7.47-7.19 (m, 21H, arom.), 5.22 (sl, 1H, H-1 IdoUA^{II}), 5.08-5.04 (sl, 1H, H-1 IdoUA^{IV}), 4.86-4.82 (m, 1H, H-1 Glc^{III}), 4.79, 4.61 (2d, 2H, J=11.0 Hz, CH₂-Ph), 4.69-4.58 (m, 4H, CH₂-Ph), 4.19 (d, 1H, J=7.9 Hz, H-1 Glc^{I}), 3.69, 3.49 (2s, 6H, CO₂Me), 3.42 (s, 3H, OMe), 3.89, 3.55 (m, 2H, CH₂₍ₐ₎-pent-4-ynyl), 2.56 (d, 1H, J=10.7 Hz, OH IdoUA^{IV}), 2.35-2.27 (m, 2H, CH_{2(c)}-pent-4-ynyl), 2.08, 2.05 (2s, 6H, CH₃—OAc), 1.93 (sl, 4H, CH_{(d)}-alkyne, CH₃—OAc), 1.89-1.78 (m, 2H, CH_{2(b)}-pent-4-ynyl), 1.05 (s, 9H, C(CH₃)₃). MALDI-MS, positive mode, m/z: 1509.10 [M+Na⁺]. [α]_{D}²¹=10.2 (c=0.43, CHCl₃).

Compound 173: ¹H NMR (400 MHz, CDCl₃, ppm): δ=7.76-7.61 (m, 4H, arom.), 7.47-7.19 (m, 16H, arom.), 5.23 (s, 1H, H-1 IdoUA^{II}), 5.08 (s, 1H, H-1 IdoUA^{IV}), 5.02 (sl, 1H, H-1 Glc^{III}), 4.83-4.57 (m, 4H, CH₂-Ph), 4.19 (d, 1H, J=7.9 Hz, H-1 Glc^{I}), 3.90, 3.55 (m, 2H, CH₂₍ₐ₎-pent-4-ynyl), 3.69, 3.52 (2s, 6H, CO₂Me), 3.50, 3.45 (2s, 6H, OMe), 2.56 (d, 1H, J=10.7 Hz, OH IdoUA^{IV}), 2.35-2.27 (m, 2H, CH_{2(c)}-pent-4-ynyl), 2.10, 2.06, 1.96 (3s, 9H, CH₃—OAc), 1.93 (t, 1H, J=2.8 Hz, CH_{(d)}-alkyne), 1.91-1.78 (m, 2H, CH_{2(b)}-pent-4-ynyl), 1.06 (s, 9H, C(CH₃)₃). MALDI-MS, positive mode, m/z: 1432.93 [M+Na⁺], 1448.86 [M+K⁺]. [α]_{D}²¹=1.0 (c=0.60, CHCl₃).

Compound 174: ¹H NMR (400 MHz, CDCl₃, ppm): δ=7.39-7.20 (m, 10H, arom.), 5.15 (d, 1H, J=2.8 Hz, H-1 IdoUA^{II}), 4.94 (m, 1H, H-1 IdoUA^{IV}), 4.86 (sl, 1H, H-1 Glc^{III}), 4.78-4.64 (m, 4H, CH₂-Ph), 4.25 (d, 1H, J=7.9 Hz, H-1 Glc^{I}), 3.94, 3.57 (m, 2H, CH₂₍ₐ₎-pent-4-ynyl), 3.82, 3.51 (2s, 6H, CO₂Me), 3.50, 3.43 (2s, 6H, OMe), 2.65 (d, 1H, J=11.8 Hz, OH IdoUA^{IV}), 2.36-2.28 (m, 2H, CH_{2(c)}-pent-4-ynyl), 2.10, 2.09, 2.06, 2.04 (4s, 12H, CH₃—OAc), 1.94 (t, 1H, J=2.8 Hz, CH_{(d)}-alkyne), 1.93-1.75 (m, 2H, CH_{2(b)}-pent-4-ynyl). MALDI-MS, positive mode, m/z: 1237.97 [M+Na⁺], 1253.93 [M+K⁺]. [α]_{D}²¹=4.4 (c=0.41, CHCl₃).

Compound 175: ¹H NMR (400 MHz, CDCl₃, ppm): δ=7.77-7.64 (m, 4H, arom.), 7.45-7.23 (m, 11H, arom.), 5.33 (sl, 1H, H-1 IdoUA^{II}), 4.96-4.94 (m, 2H, H-1 IdoUA^{IV}, H-1 Glc^{III}), 4.80 (sl, 2H, CH₂-Ph), 4.21 (d, 1H, J=7.9 Hz, H-1 Glc^{I}), 3.89, 3.54 (m, 2H, CH₂₍ₐ₎-pent-4-ynyl), 3.82, 3.50 (2s, 6H, CO₂Me), 3.49, 3.43, 3.36 (3s, 9H, OMe), 2.34-2.28 (m, 2H, CH_{2(c)}-pent-4-ynyl), 2.08, 2.04, 1.98 (3s, 9H, CH₃—OAc), 1.93 (t, 1H, CH_{(d)}-alkyne), 1.90-1.77 (m, 2H, CH_{2(b)}-pent-4-ynyl), 1.05 (s, 9H, C(CH₃)₃). MALDI-MS, positive mode, m/z: 1356.95 [M+Na⁺], 1372.90 [M+K⁺]. [α]_{D}²¹=+14.6 (c=0.63, CHCl₃).

Compound 176: ¹H NMR (400 MHz, CDCl₃, ppm): δ=7.45-7.23 (m, 15H, arom.), 5.19 (d, 1H, J=2.8 Hz, H-1 IdoUA^{II}), 5.06 (s, 1H, H-1 IdoUA^{IV}), 4.93 (m, 1H, H-1 Glc^{III}), 4.91-4.68 (m, 6H, CH₂-Ph), 4.28 (d, 1H, J=7.9 Hz, H-1 Glc^{I}), 4.00, 3.68 (m, 2H, CH₂₍ₐ₎-pent-4-ynyl), 3.55, 3.51 (2s, 6H, CO₂Me), 3.49 (s, 3H, OMe), 2.41-2.31 (m, 2H, CH_{2(c)}-pent-4-ynyl), 2.13, 2.10, 2.09, 2.08 (4s, 12H, CH₃—OAc), 1.97 (t, 1H, J=2.8 Hz, CH_{(d)}-alkyne), 1.94-1.78 (m, 2H, CH_{2(b)}-pent-4-ynyl). MALDI-MS, positive mode, m/z: 1313.18 [M+Na⁺]. [α]_{D}²¹=+2.6 (c=0.46, CHCl₃).

Compound 177: ¹H NMR (400 MHz, CDCl₃, ppm): δ=7.74-7.60 (m, 4H, arom.), 7.44-7.21 (m, 11H, arom.), 5.21 (sl, 1H, H-1 IdoUA^{II}), 5.00-4.95 (m, 2H, H-1 IdoUA^{IV}, H-1 Glc^{III}), 4.66, 4.57 (2d, 2H, J=11.8 Hz, CH₂-Ph), 4.19 (d, 1H, J=7.9 Hz, H-1 Glc^{I}), 3.88, 3.54 (m, 2H, CH₂₍ₐ₎-pent-4-ynyl), 3.82, 3.59 (2s, 6H, CO₂Me), 3.50, 3.49, 3.43 (3s, 9H, OMe), 2.34-2.28 (m, 2H, CH_{2(c)}-pent-4-ynyl), 2.08 (s, 6H, CH₃—OAc), 1.94 (sl, 4H, CH_{(d)}-alkyne, CH₃—OAc), 1.89-1.78 (m, 2H, CH_{2(b)}-pent-4-ynyl), 1.05 (s, 9H, C(CH₃)₃). MALDI-MS, positive mode, m/z: 1357.59 [M+Na⁺], 1373.38 [M+K⁺]. [α]_{D}²¹=1.4 (c=0.66, CHCl₃).

### 1. Preparation 13: Synthesis of Tetrasaccharide 178 and 179 (Scheme 13)

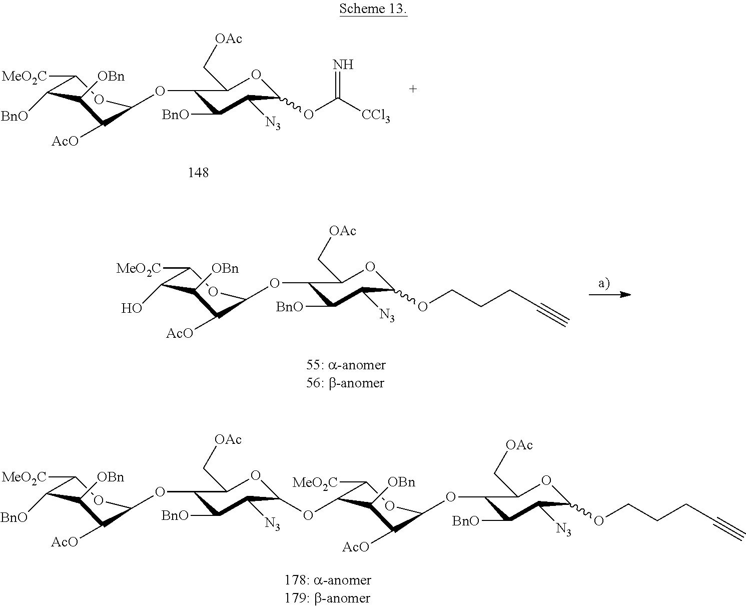

Step 13.a: Synthesis of compound 178: O-glycosylation reaction between disaccharide donor 148 (164.8 mg, 0.184 mmol, 1.3 eq.) with disaccharide acceptor 55 (102.9 mg, 0.142 mmol, 1 eq.) was performed according to the general method B. Purification was effected by chromatography on silica gel column (heptane/ethyl acetate: 7/3 to 6/4) to give tetrasaccharide 178 (157.6 mg, 76%) as a white solid. ¹H NMR (400 MHz, CDCl₃, ppm): δ=7.38-7.18 (m, 25H, arom.), 5.28 (d, J=5.4 Hz, 1H, H-1 IdoUA^{II}), 5.25 (d, J=3.7 Hz, 1H, H-1 IdoUA^{IV}), 4.95-4.80 (m, 6H, H-2 IdoUA^{IV}, H-2 IdoUA^{II}, H-1Glc^{I}, H-1 Glc^{III}, CH₂-Ph), 4.75-4.59 (m, 8H, H-5 IdoUA^{IV}, H-5 IdoUA^{II}, 3×CH₂-Ph), 4.53-4.48 (dd, 2H, J=11.6 Hz, CH₂-Ph), 4.40 (d, 1H, J=11.8 Hz, H-6a Glc^{III}), 4.35 (dd, 1H, J=12.6 Hz, J=1.8 Hz, H-6a Glc^{I}), 4.24 (d, 1H, J=11.8 Hz, H-6b Glc^{III}), 4.16 (dd, 1H, J=12.6 Hz, J=3.0 Hz, H-6b Glc^{I}), 4.01-3.76 (m, 10H, H-3 IdoUA^{IV}, H-4 IdoUA^{IV}, H-3 IdoUA^{II}, H-4 IdoUA^{II}, H-4 Glc^{I}, H-5 Glc^{I}, H-3 Glc^{IIIl}, H-4 Glc^{IIIl}, H-5 Glc^{IIIl}, CH₍ₐ₎-pent-4-ynyl), 3.65 (t, 1H, J=9.3 Hz, H-3 Glc^{I}), 3.57 (m, 1H, CH₍ₐ₎-pent-4-ynyl), 3.55, 3.50 (2s, 6H, CO₂Me), 3.36-3.26 (m, 2H, H-2 Glc^{III}, H-2 Glc^{I}), 2.39-2.33 (m, 2H, CH_{2(c)}-pent-4-ynyl), 2.12, 2.05, 2.02, 2.01 (4s, 12H, CH₃—OAc), 1.96 (t, J=2.6 Hz, 1H, CH_{(d)}-alkyne), 1.94-1.74 (m, 2H, CH_{2(b)}-pent-4-ynyl). MALDI-MS, positive mode, m/z: 1480.15 [M+Na⁺], 1496.15 [M+K⁺]. [α]_{D}²¹=+13.1 (c=0.53, CHCl₃).

Preparation of tetrasaccharide 179 was carried out as described for compound 178.

Compound 179: ¹H NMR (400 MHz, CDCl₃, ppm): δ=7.41-7.13 (m, 25H, arom.), 5.26 (d, 1H, J=5.4 Hz, H-1 IdoUA^{II}), 5.2 (d, 1H, J=3.3 Hz, H-1 IdoUA^{IV}), 4.91-4.59 (m, 8H, 4×CH₂-Ph), 4.88 (m, 1H, H-1 Glc^{III}), 4.49 (2d, 2H, J=11.8 Hz, CH₂-Ph), 4.24 (d, 1H, J=8.0 Hz, H-1 Glc^{I}), 3.94, 3.64 (m, 2H, CH₂₍ₐ₎-pent-4-ynyl), 3.53, 3.48 (2s, 6H, CO₂Me), 2.35-2.28 (m, 2H, CH_{2(c)}-pent-4-ynyl), 2.09, 2.03, 2.01 (4s, 12H, CH₃—OAc), 1.93 (t, 1H, J=2.8 Hz, CH_{(d)}-alkyne), 1.88-1.78 (m, 2H, CH_{2(b)}-pent-4-ynyl). MALDI-MS, positive mode, m/z: 1479.45 [M+Na⁺], 1495.34 [M+K⁺]. [α]_{D}²¹=5.5 (c=0.82, CHCl₃).

### 1. Preparation 14: Synthesis of Tetrasaccharide 180 (Scheme 14)

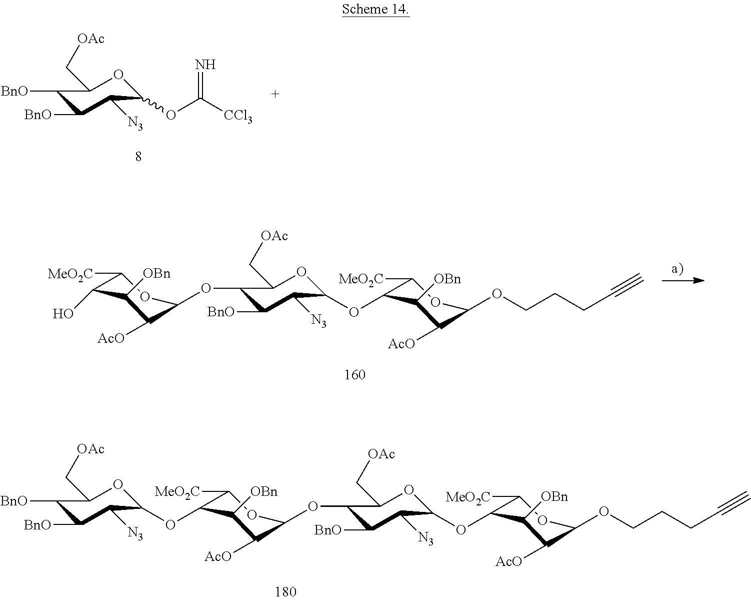

Step 14.a: Synthesis of compound 180: O-glycosylation reaction between monosaccharide donor 8 (142 mg, 0.25 mmol, 1.3 eq.) with trisaccharide acceptor 160 (200 mg, 0.19 mmol, 1 eq.) was performed according to the general method B. Purification was effected by a Sephadex LH20 gel column (dichloromethane/ethanol: 7/3) to give tetrasaccharide 180 (274 mg, 98%) as a white amorphous compound. ¹H NMR (400 MHz, CDCl₃, ppm): δ=7.40-7.20 (m, 25H, arom.), 5.28 (d, 1H, J=4.4 Hz, H-1 IdoUA^{III}), 5.02-4.99 (m, 2H, H-1 Glc^{IV}, H-1 IdoUA^{I}), 4.94-4.90 (m, 3H, H-2 IdoUA^{III}, H-2 IdoUA^{I}, CH-Ph), 4.89-4.84 (m, 3H, H-1 Glc^{II}, CH₂-Ph), 4.83-4.80 (sl, 2H, H-5 IdoUA^{III}, CH-Ph), 4.78 (d, 1H, J=11.6 Hz, CH-Ph), 4.73 (s, 2H, CH₂-Ph), 4.67-4.62 (m, 3H, 2×CH-Ph, H-5 IdoUA^{I}), 4.57 (d, 1H, J=11.0 Hz, CH-Ph), 4.42 (dd, 1H, J=12.3 Hz, J=1.5 Hz, H-6a Glc^{IV}), 4.29-4.20 (m, 2H, H-6b Glc^{IV}, H-6a Glc^{II}), 4.16 (dd, 1H, J=12.3 Hz, J=3.7 Hz, H-6b Glc^{II}), 4.08 (m, 1H, H-4 IdoUA^{III}), 4.03 (t, 1H, J=5.4 Hz, H-4 IdoUA^{I}), 3.94 (t, 1H, J=5.4 Hz, H-3 IdoUA^{I}), 3.92-3.84 (m, 5H, H-3 IdoUA^{III}, CH₍ₐ₎-pent-4-ynyl, H-5 Glc^{IV}, H-5 Glc^{II}, H-4 Glc^{II}), 3.83-3.68 (m, 5H, H-3 Glc^{IV}, H-3 Glc^{II}, CO₂Me), 3.61-3.48 (m, 5H, CO₂Me, H-4 Glc^{IV}, CH_{(a′)}-pent-4-ynyl), 3.30 (dd, 1H, J=10.2 Hz, J=3.6 Hz, H-2 Glc^{II}), 3.26 (dd, 1H, J=10.2 Hz, J=3.4 Hz, H-2 Glc^{IV}), 2.30-2.23 (m, 2H, CH_{2(c)}-pent-4-ynyl), 2.11, 2.09, 2.08, 1.96 (4s, 12H, CH₃—OAc), 1.93 (t, 1H, J=2.6 Hz, CH_{(d)}-alkyne), 1.87-1.72 (m, 2H, CH_{2(b)}-pent-4-ynyl). MALDI-MS, positive mode, m/z: 1479.50 [M+Na⁺]. [α]_{D}²¹=+8.7 (c=1.19, CHCl₃).

### F. Pentasaccharides Preparations

### 1. Preparation 15: Synthesis of Acceptor Pentasaccharide 182 (Scheme 15)

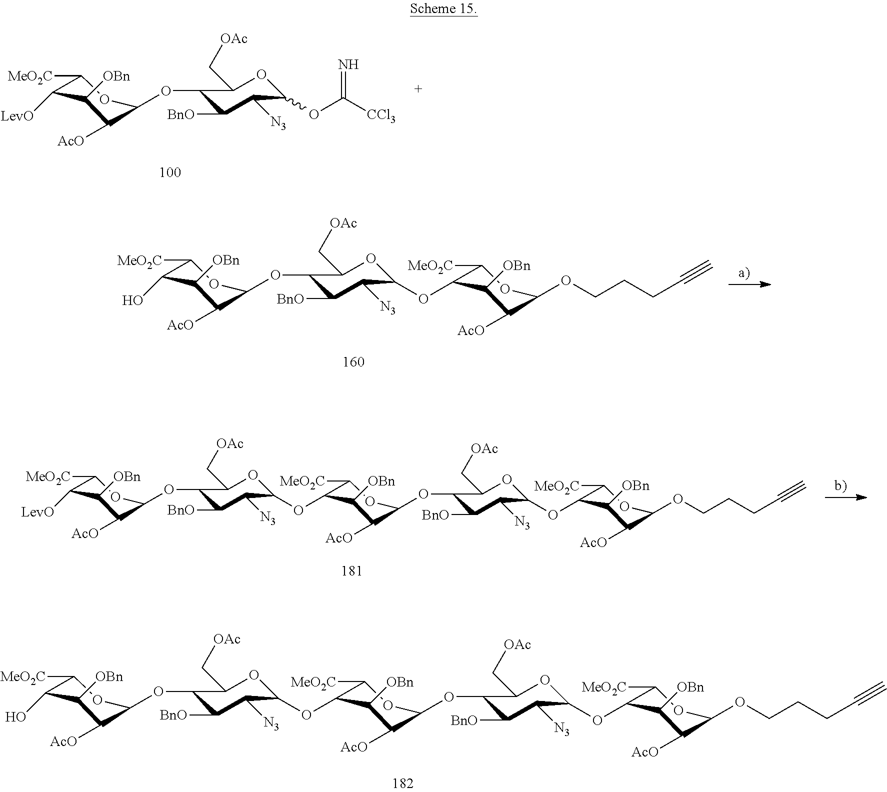

Step 15.a: Synthesis of compound 181: O-glycosylation reaction between disaccharide donor 100 (408 mg, 0.45 mmol, 1.3 eq.) and trisaccharide acceptor 160 (365 mg, 0.35 mmol, 1 eq.) was performed according to the general method B. Purification was effected by a Sephadex LH20 gel column (dichloromethane/ethanol: 7/3) to give pentasaccharide 181 (450 mg, 72%) as a white amorphous solid. ¹H NMR (400 MHz, CDCl₃, ppm): δ=7.43-7.16 (m, 25H, arom.), 5.27 (d, 1H, J=4.6 Hz, H-1 IdoUA^{III}), 5.13 (d, 1H, J=2.2 Hz, H-1 IdoUA^{V}), 5.07 (t, 1H, J=3.8 Hz, H-4 IdoUA^{V}), 4.99 (s, 1H, H-1 IdoUA^{I}), 4.96 (d, 1H, J=3.8 Hz, H-1 Glc^{IV}), 4.93-4.58 (m, 17H, H-2 IdoUA^{III}, H-5 IdoUA^{III}, H-2 IdoUA^{V}, H-5 IdoUA^{V}, H-2 IdoUA^{I}, H-5 IdoUA^{I}, 5×CH₂-Ph, H-1 Glc^{II}), 4.45-4.36 (m, 2H, H-6a Glc^{II}, H-6a Glc^{IV}), 4.22 (dd, 1H, J=12.8 Hz, J=2.7 Hz, H-6b Glc^{II}), 4.17 (dd, 1H, J=12.7 Hz, J=3.1 Hz, H-6b Glc^{IV}), 4.07 (sl, 1H, H-4 IdoUA^{I}), 3.98 (t, 1H, J=5.5 Hz, H-4 IdoUA^{III}), 3.94-3.51 (m, 14H, H-3 IdoUA^{III}, H-3 IdoUA^{V}, H-3 IdoUA^{I}, H-3 Glc^{IV}, H-4 Glc^{IV}, H-5 Glc^{IV}, H-3 Glc^{II}, H-4 Glc^{II}, H-5 Glc^{II}, CH₂₍ₐ₎-pent-4-ynyl, CO₂Me), 3.48, 3.47 (2s, 6H, 2×CO₂Me), 3.33-3.24 (m, 2H, H-2 Glc^{II}, H-2 Glc^{IV}), 2.79-2.39 (m, 4H, CH₂-Lev), 2.28-2.21 (m, 2H, CH₂₍ₐ₎-pent-4-ynyl), 2.15 (s, 3H, CH₃-Lev), 2.10, 2.08, 2.05, 2.04, 2.03 (5s, 15H, CH₃—OAc), 1.90 (t, 1H, J=2.6 Hz, CH_{(d)}-alkyne), 1.85-1.71 (m, 2H, CH_{2(b)}-pent-4-ynyl). MALDI-MS, positive mode, m/z: 1809.53 [M+Na⁺], 1825.32 [M+K⁺]. [α]_{D}²¹=12.5 (c=0.61, CHCl₃).

Step 15.b: Synthesis of compound 182: In a dry round-bottom flask, compound 181 (450 mg, 0.25 mmol) was dissolved in a mixture of pyridine/acetic acid 3/1 (2.5 mL) at 0° C. followed by the addition of hydrazine acetate (46 mg, 0.50 mmol, 2 eq.). The reaction was stirred at room temperature for 2 h after which it was diluted with dichloromethane (50 mL) and successively washed with a 5% aqueous solution of H₂SO₄ (10 mL), a saturated aqueous solution of NaHCO₃ (10 mL) and water (10 mL). The organic layer was dried over MgSO₄, filtered, the solvent was evaporated under reduced pressure and the residue was purified by flash chromatography on silica gel column (heptane/ethyl acetate: 8/2 to 4/6) to give pentasaccharide acceptor 182 (325 mg, 77%) as a viscous translucid compound. ¹H NMR (400 MHz, CDCl₃, ppm): δ=7.43-7.20 (m, 25H, arom.), 5.24 (d, 1H, J=4.4 Hz, H-1 IdoUA^{III}), 5.05 (s, 1H, H-1 IdoUA^{V}), 4.99 (s, 1H, H-1 IdoUA^{I}), 4.96 (d, 1H, J=3.6 Hz, H-1 Glc^{IV}), 4.93-4.58 (m, 17H, H-2 IdoUA^{III}, H-5 IdoUA^{III}, H-2 IdoUA^{V}, H-5 IdoUA^{V}, H-2 IdoUA^{I}, H-5 IdoUA^{I}, 5×CH₂-Ph, H-1 Glc^{II}), 4.45-4.36 (m, 2H, H-6a Glc^{II}, H-6a Glc^{IV}), 4.23 (dd, 1H, J=12.8 Hz, J=2.7 Hz, H-6b Glc^{II}), 4.17 (dd, 1H, J=12.7 Hz, J=3.1 Hz, H-6b Glc^{IV}), 4.09-4.04 (t, 1H, H-4 IdoUA^{I}), 4.01-3.67 (m, 14H, H-4 IdoUA^{III}, H-3 IdoUA^{III}, H-3 IdoUA^{V}, H-4 IdoUA^{V}, H-3 IdoUA^{I}, H-4 Glc^{IV}, H-5 Glc^{IV}, H-3 Glc^{II}, H-4 Glc^{II}, H-5 Glc^{II}, CH₍ₐ₎-pent-4-ynyl, CO₂Me), 3.65-3.43 (m, 8H, H-3 Glc^{IV}, 2×CO₂Me, CH₍ₐ₎-pent-4-ynyl), 3.33-3.22 (m, 2H, H-2 Glc^{II}, H-2 Glc^{IV}), 2.50 (d, 1H, OH IdoUA^{V}), 2.29-2.21 (m, 2H, CH_{2(c)}-pent-4-ynyl), 2.09, 2.08, 2.05, 2.04, 2.03 (5s, 15H, CH₃—OAc), 1.90 (t, 1H, J=2.6 Hz, CH_{(d)}-alkyne), 1.87-1.71 (m, 2H, CH_{2(b)}-pent-4-ynyl). MALDI-MS, positive mode, m/z: 1711.14 [M+Na⁺]. [α]_{D}²¹=3.0 (c=1.15, CHCl₃).

### G. Hexasaccharides Preparations

### 1. Preparation 16: Synthesis of Protected Hexasaccharides 183 to 201 (Scheme 16)

Below is reported the general formula of all the protected hexasaccharides synthesized.

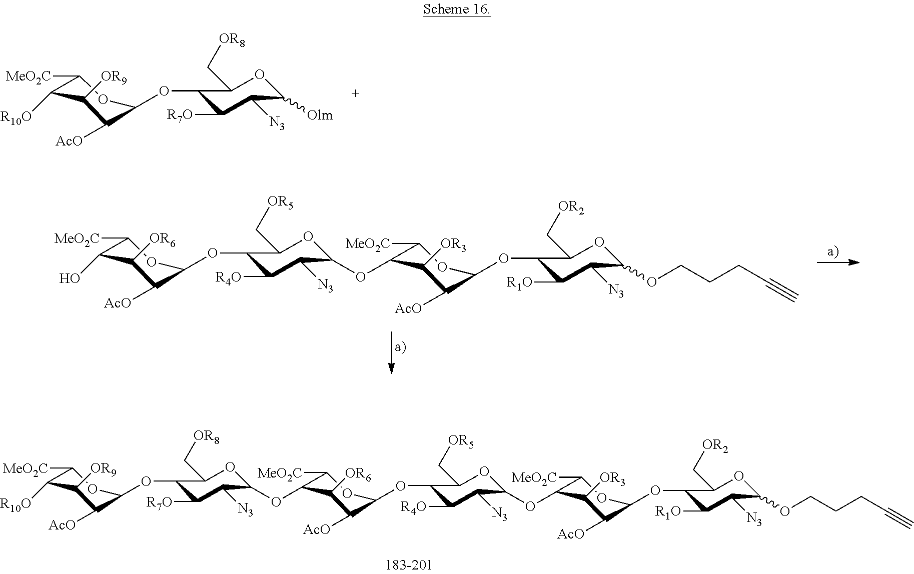

| Compound | R₁ | R₃ | R₄ | R₆ | R₇ | R₉/R₁₀ | R₂ | R₅ | R₈ | anomer |
|---|---|---|---|---|---|---|---|---|---|---|
| 183 | Bn | Bn | Bn | Bn | Bn | Bn | Ac | Ac | Ac | β |
| 184 | Bn | Bn | Bn | Bn | Bn | Bn | Ac | Ac | Ac | α |
| 185 | Bn | Bn | Bn | Bn | Bn | Bn | TBDPS | TBDPS | TBDPS | α |
| 186 | Me | Bn | Me | Bn | Me | Bn | TBDPS | Ac | Ac | β |
| 187 | Bn | Me | Bn | Me | Bn | Me | TBDPS | Ac | Ac | β |
| 188 | Me | Me | Me | Me | Me | Me | TBDPS | Ac | Ac | β |
| 189 | Me | Bn | Bn | Bn | Bn | Bn | TBDPS | Ac | Ac | β |
| 190 | Me | Me | Bn | Bn | Bn | Bn | TBDPS | Ac | Ac | β |
| 191 | Bn | Bn | Bn | Bn | Bn | Me | Ac | Ac | Ac | β |
| 192 | Bn | Bn | Me | Me | Bn | Bn | Ac | Ac | Ac | β |
| 193 | Bn | Bn | Bn | Bn | Me | Me | Ac | Ac | Ac | β |
| 194 | Me | Bn | Bn | Bn | Bn | Me | TBDPS | Ac | Ac | β |
| 195 | Me | Me | Me | Bn | Bn | Bn | TBDPS | Ac | Ac | β |
| 196 | Bn | Bn | Bn | Me | Me | Me | Ac | Ac | Ac | β |
| 197 | Me | Me | Bn | Bn | Bn | Me | TBDPS | Ac | Ac | β |
| 198 | Me | Bn | Bn | Bn | Me | Me | TBDPS | Ac | Ac | β |
| 199 | Bn | Me | Me | Me | Me | Bn | TBDPS | Ac | Ac | β |
| 200 | Me | Me | Bn | Bn | Me | Me | TBDPS | Ac | Ac | β |
| 201 | Bn | Bn | Bn | Bn | Bn | Bn/Lev | Ac | Ac | Ac | β |

Step 16.a: Synthesis of compound 183: O-glycosylation reaction between disaccharide donor 148 (114 mg, 0.127 mmol, 1.3 eq.) and tetrasaccharide acceptor 166 (134.1 mg, 0.098 mmol, 1 eq.) was performed according to the general method B. Purification was effected by chromatography on silica gel column (heptane/ethyl acetate: 7/3 to 6/4) to give hexasaccharide 183 (158.5 mg, 77%) as a viscous colourless compound. ¹H NMR (400 MHz, CDCl₃, ppm): δ=7.31-7.12 (m, 35H, arom.), 5.22 (d, 2H, J=5.3 Hz, H-1 IdoUA^{II}, H-1 IdoUA^{IV}), 5.14 (d, 1H, J=3.2 Hz, H-1 IdoUA^{VI}), 4.89 (d, 1H, J=3.7 Hz, H-1 Glc^{III}), 4.85-4.75 (m, 6H, H-2 IdoUA^{II}, H-2 IdoUA^{IV}, H-2 IdoUA^{VI}, H-1 Glc^{V}, CH₂-Ph), 4.71-4.53 (m, 12H, 5×CH₂-Ph, H-5 IdoUA^{VI}, H-5 IdoUA^{II}), 4.49 (d, 1H, J=4.5 Hz, H-5 IdoUA^{IV}), 4.46-4.42 (m, 2H, CH₂-Ph), 4.38 (dd, 1H, J=12.1 Hz, J=2.3 Hz, H-6a Glc^{I}), 4.33-4.24 (m, 2H, H-6a Glc^{V}, H-6a Glc^{III}), 4.18 (d, 1H, J=7.9 Hz, H-1 Glc^{I}), 4.15-4.05 (m, 3H, H-6b Glc^{V}, H-6b Glc^{III}, H-6b Glc^{I}), 3.96-3.68 (m, 11H, H-4 IdoUA^{VI}, H-4 IdoUA^{II}, H-4 Glc^{IIIl}, H-3 IdoUA^{IV}, H-3 IdoUA^{VI}, H-3 IdoUA^{II}, H-4 Glc^{I}, H-5 Glc^{I}, H-4 Glc^{V}, H-5 Glc^{IIIl}, CH₍ₐ₎-pent-4-ynyl), 3.63-3.52 (m, 3H, H-3 Glc^{V}, H-3 Glc^{III}, CH_{(a′)}-pent-4-ynyl), 3.49, 3.46, 3.42 (3s, 9H, CO₂Me), 3.39-3.28 (m, 2H, H-5 Glc^{V}, H-2 Glc^{I}), 3.26-3.18 (m, 3H, H-2 Glc^{V}, H-2 Glc^{III}, H-3 Glc^{I}), 2.29-2.23 (m, 2H, CH_{2(c)}-pent-4-ynyl), 2.04, 2.02, 1.97, 1.96-1.93 (6s, 18H, CH₃—OAc), 1.88 (t, 1H, J=2.6 Hz, CH_{(d)}-alkyne), 1.83-1.71 (m, 2H, CH_{2(b)}-pent-4-ynyl). MALDI-MS, positive mode, m/z: 2122.16 [M+Na⁺], 2138.05 [M+K⁺]. [α]_{D}²¹=11.0 (c=0.40, CHCl₃).

Preparation of all protected hexasaccharides 184 to 201 were carried out as described for compound 183.

Compound 184: ¹H NMR (400 MHz, CDCl₃, ppm): δ=7.31-7.08 (m, 35H, arom.), 5.22-5.19 (m, 2H, H-1 IdoUA^{II}, H-1 IdoUA^{IV}), 5.16 (d, 1H, J=3.2 Hz, H-1 IdoUA^{VI}), 4.87 (d, 1H, J=3.7 Hz, H-1 Glc^{III}), 4.87-4.72 (m, 7H, H-2 IdoUA^{II}, H-2 IdoUA^{IV}, H-2 IdoUA^{VI}, H-1 Glc^{V}, CH₂-Ph, H-1 Glc^{I}), 4.70-4.51 (m, 10H, 4×CH₂-Ph, H-5 IdoUA^{VI}, H-5 IdoUA^{II}), 4.48 (d, 1H, J=4.8 Hz, H-5 IdoUA^{IV}), 4.48-4.41 (m, 2H, CH₂-Ph), 4.35-4.23 (m, 3H, H-6a Glc^{V}, H-6a Glc^{III}, H-6a Glc^{I}), 4.20-4.05 (m, 3H, H-6b Glc^{V}, H-6b Glc^{III}, H-6b Glc^{I}), 3.94-3.68 (m, 12H, H-4 IdoUA^{VI}, H-4 IdoUA^{II}, H-4 Glc^{III}, H-3 IdoUA^{IV}, H-3 IdoUA^{VI}, H-4 Glc^{I}, H-5 Glc^{I}, H-4 Glc^{V}, H-5 Glc^{III}, CH₍ₐ₎-pent-4-ynyl, H-3 IdoUA^{II}, H-5 Glc^{V}), 3.61-3.48 (m, 3H, H-3 Glc^{V}, H-3 Glc^{III}, CH_{(a′)}-pent-4-ynyl), 3.47, 3.45, 3.42 (3s, 9H, CO₂Me), 3.31-3.29 (m, 1H, H-3 Glc^{I}), 3.28-3.16 (m, 3H, H-2 Glc^{V}, H-2 Glc^{III}, H-2 Glc^{I}), 2.29-2.23 (m, 2H, CH_{2(c)}-pent-4-ynyl), 2.04, 2.02, 1.97, 1.96-1.93 (6s, 18H, CH₃—OAc), 1.88 (t, 1H, J=2.6 Hz, CH_{(d)}-alkyne), 1.83-1.71 (m, 2H, CH_{2(b)}-pent-4-ynyl). MALDI-MS, positive mode, m/z: 2122.29 [M+Na⁺], 2138.21 [M+K⁺]. [α]_{D}²¹=+17.3 (c=0.51, CHCl₃).

Compound 185: ¹H NMR (400 MHz, CDCl₃, ppm): δ=7.79-7.68 (m, 5H, arom.), 7.43-7.15 (m, 60H, arom.), 5.54-5.51 (m, 2H, H-1 IdoUA^{II}, H-1 IdoUA^{IV}), 5.38 (d, 1H, J=4.1 Hz, H-1 IdoUA^{VI}), 4.99 (d, 1H, J=3.4 Hz, H-1 Glc^{III}), 4.94-4.50 (m, 20H, H-1 Glc^{V}, H-1 Glc^{I}, H-2 IdoUA^{II}, H-2 IdoUA^{IV}, H-2 IdoUA^{VI}, H-5 IdoUA^{VI}, 7×CH₂-Ph), 4.52 (d, 1H, J=4.6 Hz, H-5 IdoUA^{II}), 4.34 (d, 1H, J=5.2 Hz, H-5 IdoUA^{IV}), 4.11 (t, 1H, J=9.5 Hz, H-4 Glc^{III}), 4.06-3.69 (m, 24H, CO₂Me, H-6a/b Glc^{I}, H-6a/b Glc^{III}, H-6a/b Glc^{V}, H-4 IdoUA^{VI}, H-4 IdoUA^{II}, H-3 IdoUA^{IV}, H-3 IdoUA^{VI}, H-3 Glc^{I}, H-4 Glc^{I}, H-5 Glc^{I}, H-4 Glc^{V}, H-5 Glc^{III}, CH₂₍ₐ₎-pent-4-ynyl, H-3 IdoUA^{II}, H-5 Glc^{V}, H-3 Glc^{V}, H-3 Glc^{III}), 3.39, 3.33 (2s, 6H, CO₂Me), 3.30-3.24 (m, 2H, H-2 Glc^{III}, H-2 Glc^{I}), 3.22 (dd, 1H, J=3.4 Hz, J=10.3 Hz, H-2 Glc^{V}), 2.33-2.28 (m, 2H, CH_{2(c)}-pent-4-ynyl), 1.86-1.76 (3s+m, 12H, 3×CH₃—OAc, CH_{2(b)}-pent-4-ynyl, CH_{(d)}-alkyne), 1.07-1.01 (3s, 27H, C(CH₃)₃). MALDI-MS, positive mode, m/z: 2709.54 [M+Na⁺], 2725.46 [M+K⁺]. [α]_{D}²¹=+25.5 (c=0.52, CHCl₃).

Compound 186: ¹H NMR (400 MHz, CDCl₃, ppm): δ=7.73-7.64 (m, 4H, arom.), 7.42-7.24 (m, 26H, arom.), 5.23 (s, 1H, H-1 IdoUA^{II}), 5.13 (d, 1H, J=3.5 Hz, H-1 IdoUA^{IV}), 5.10 (d, 1H, J=2.7 Hz, H-1 IdoUA^{VI}), 4.86-4.79 (m, 2H, H-1 Glc^{III}, H-1 Glc^{V}), 4.22-4.15 (m, 1H, H-1 Glc^{I}), 3.90, 3.56 (m, 2H, CH₂₍ₐ₎-pent-4-ynyl), 3.70, 3.68, 3.65 (3s, 9H, CO₂Me), 3.50, 3.48, 3.42 (3s, 9H, OMe), 2.36-2.28 (m, 2H, CH_{2(c)}-pent-4-ynyl), 2.09, 2×2.04, 2.03, 1.93 (5s, 15H, CH₃—OAc), 1.93 (br. s, 1H, CH_{(d)}-alkyne), 1.90-1.79 (m, 2H, CH_{2(b)}-pent-4-ynyl), 1.05 (s, 9H, C(CH₃)₃). MALDI-MS, positive mode, m/z: 2090.32 [M+Na⁺], 2106.28 [M+K⁺]. [α]_{D}²¹=6.6 (c=0.77, CHCl₃).

Compound 187: ¹H NMR (400 MHz, CDCl₃, ppm): δ=7.73-7.69 (m, 4H, arom.), 7.45-7.25 (m, 21H, arom.), 5.34 (d, 1H, J=2.3 Hz, H-1 IdoUA^{II}), 5.26 (d, 1H, J=4.2 Hz, H-1 IdoUA^{IV}), 5.22 (d, 1H, J=4.4 Hz, H-1 IdoUA^{VI}), 5.04 (d, 1H, J=3.4 Hz, H-1 Glc^{III}), 4.99 (d, 1H, J=3.6 Hz, H-1 Glc^{V}), 4.25-4.21 (m, 1H, H-1 Glc^{I}), 3.91, 3.57 (m, 2H, CH₂₍ₐ₎-pent-4-ynyl), 3.60, 3.56, 3.50 (3s, 9H, CO₂Me), 3.50, 3.49, 3.48, 3.41 (4s, 12H, OMe), 2.37-2.30 (m, 2H, CH_{2(c)}-pent-4-ynyl), 2.11, 2.10, 2.09, 2.08, 1.98 (5s, 15H, CH₃—OAc), 1.95 (t, 1H, J=2.6 Hz, CH_{(d)}-alkyne), 1.91-1.80 (m, 2H, CH_{2(b)}-pent-4-ynyl), 1.08 (s, 9H, C(CH₃)₃). MALDI-MS, positive mode, m/z: 2013.04 [M+Na⁺], 2028.95 [M+K⁺]. [α]_{D}²¹=+23.3 (c=1.17, CHCl₃).

Compound 188: ¹H NMR (400 MHz, CDCl₃, ppm): δ=5.23 (s, 1H, H-1 IdoUA^{II}), 5.09-5.06 (m, 2H, H-1 IdoUA^{IV}, H-1 IdoUA^{VI}), 4.97 (d, 2H, J=3.5 Hz, H-1 Glc^{III}, H-1 Glc^{V}), 4.25-4.21 (m, 1H, H-1 Glc^{I}), 3.88, 3.55 (m, 2H, CH₂₍ₐ₎-pent-4-ynyl), 3.79, 3.78, 3.77 (3s, 9H, CO₂Me), 3.55, 3.52, 3.51, 3.48, 3.45, 3.43, 3.42 (7s, 21H, OMe), 2.35-2.29 (m, 2H, CH_{2(c)}-pent-4-ynyl), 2.12, 2.11, 2.10, 2.09, 2.00 (5s, 15H, CH₃—OAc), 1.94 (t, 1H, J=2.6 Hz, CH_{(d)}-alkyne), 1.91-1.78 (m, 2H, CH_{2(b)}-pent-4-ynyl), 1.08 (s, 9H, C(CH₃)₃). MALDI-MS, positive mode, m/z: 1785.84 [M+Na⁺], 1801.67 [M+K⁺]. [α]_{D}²¹=+10.0 (c=0.12, CH₂Cl₂).

Compound 189: ¹H NMR (400 MHz, CDCl₃, ppm): δ=7.75-7.64 (m, 4H, arom.), 7.45-7.15 (m, 36H, arom.), 5.31-5.26 (m, 2H, H-1 IdoUA^{II}, H-1 IdoUA^{IV}), 5.22 (s, 1H, H-1 IdoUA^{VI}), 4.93 (d, 1H, J=2.5 Hz, H-1 Glc^{III}), 4.87, 4.64 (m, 2H, CH₂-Ph), 4.76-4.60 (m, 8H, 4×CH₂-Ph), 4.84 (m, 1H, H-1 Glc^{V}), 4.52, 4.48 (2d, 2H, J=11.7 Hz, CH₂-Ph), 4.18 (d, 1H, J=8.0 Hz, H-1 Glc^{I}), 3.90, 3.56 (m, 2H, CH₂₍ₐ₎-pent-4-ynyl), 3.70, 3.55, 3.50 (3s, 9H, CO₂Me), 3.42 (s, 3H, OMe), 2.36-2.29 (m, 2H, CH_{2(c)}-pent-4-ynyl), 2.10, 2.02, 2.01, 2.00 (4s, 12H, CH₃—OAc), 1.95 (sl, 4H, CH_{(d)}-alkyne, CH₃—OAc), 1.89-1.79 (m, 2H, CH_{2(b)}-pent-4-ynyl), 1.05 (s, 9H, C(CH₃)₃). MALDI-MS, positive mode, m/z: 2241.63 [M+Na⁺]. [α]_{D}²¹=3.7 (c=0.91, CHCl₃).

Compound 190: ¹H NMR (400 MHz, CDCl₃, ppm): δ=7.71-7.54 (m, 4H, arom.), 7.38-7.04 (m, 31H, arom.), 5.26-5.19 (m, 2H, H-1 IdoUA^{II}, H-1 IdoUA^{IV}), 5.14 (s, 1H, H-1 IdoUA^{VI}), 4.91 (m, 1H, H-1 Glc^{III}), 4.87 (d, 1H, J=3.5 Hz, H-1 Glc^{V}), 4.81, 4.58 (m, 4H, 2×CH₂-Ph), 4.68-4.58 (m, 4H, 2×CH₂-Ph), 4.48-4.40 (m, 2H, CH₂-Ph), 4.14 (d, 1H, J=8.0 Hz, H-1 Glc^{I}), 3.82, 3.47 (m, 2H, CH₂₍ₐ₎-pent-4-ynyl), 3.65, 3.48, 3.45 (3s, 9H, CO₂Me), 3.44, 3.37 (2s, 6H, OMe), 2.30-2.21 (m, 2H, CH_{2(c)}-pent-4-ynyl), 2.05, 1.95, 1.94, 1.93, 1.90 (5s, 15H, CH₃—OAc), 1.86 (br, 1H, CH_{(d)}-alkyne), 1.83-1.71 (m, 2H, CH_{2(b)}-pent-4-ynyl), 0.98 (s, 9H, C(CH₃)₃). MALDI-MS, positive mode, m/z: 2165.42 [M+Na⁺]. [α]_{D}²¹=1.5 (c=1.69, CHCl₃).

Compound 191: ¹H NMR (400 MHz, CDCl₃, ppm): δ=7.45-7.15 (m, 25H, arom.), 5.28 (d, 1H, J=4.5 Hz, H-1 IdoUA^{II}), 5.23-5.19 (m, 2H, H-1 IdoUA^{IV}, H-1 IdoUA^{VI}), 4.96 (d, 1H, J=3.8 Hz, H-1 Glc^{III}), 4.94-4.62 (m, 10H, 5×CH₂-Ph), 4.89 (m, 1H, H-1 Glc^{V}), 4.24 (d, 1H, J=7.9 Hz, H-1 Glc^{I}), 3.96, 3.64 (m, 2H, CH₄₀-pent-4-ynyl), 3.59, 3.57, 3.48 (3s, 9H, CO₂Me), 3.49, 3.40 (2s, 6H, OMe), 2.35-2.29 (m, 2H, CH_{2(c)}-pent-4-ynyl), 2.12-2.02 (6s, 18H, CH₃—OAc), 1.94 (sl, 1H, CH_{(d)}-alkyne), 1.91-1.77 (m, 2H, CH_{2(b)}-pent-4-ynyl). MALDI-MS, positive mode, m/z: 1969.05 [M+Na⁺]. [α]_{D}²¹=5.5 (c=1.75, CHCl₃).

Compound 192: ¹H NMR (400 MHz, CDCl₃, ppm): δ=7.36-7.10 (m, 25H, arom.), 5.25 (d, 1H, J=5.5 Hz, H-1 IdoUA^{II}), 5.13 (d, 1H, J=3.2 Hz, H-1 IdoUA^{IV}), 5.04 (d, 1H, J=2.9 Hz, H-1 IdoUA^{VI}), 4.95 (d, 1H, J=3.6 Hz, H-1 Glc^{III}), 4.87, 4.60 (m, 2H, CH₂-Ph), 4.71-4.61 (m, 6H, 3×CH₂-Ph), 4.48, 4.43 (2d, 2H, J=11.5 Hz, CH₂-Ph), 4.84 (m, 1H, H-1 Glc^{V}), 4.24 (d, 1H, J=7.8 Hz, H-1 Glc^{I}), 3.92, 3.62 (m, 2H, CH₂₍ₐ₎-pent-4-ynyl), 3.69, 3.51, 3.47 (3s, 9H, CO₂Me), 3.46, 3.41 (2s, 6H, OMe), 2.31-2.21 (m, 2H, CH_{2(c)}-pent-4-ynyl), 2.07-1.98 (6s, 18H, CH₃—OAc), 1.90 (sl, 1H, CH_{(d)}-alkyne), 1.84-1.75 (m, 2H, CH_{2(b)}-pent-4-ynyl). MALDI-MS, positive mode, m/z: 1969.39 [M+Na⁺]. [α]_{D}²¹=0.4 (c=2.54, CHCl₃).

Compound 193: ¹H NMR (400 MHz, CDCl₃, ppm): δ=7.39-7.07 (m, 20H, arom.), 5.20 (d, 1H, J=4.4 Hz, H-1 IdoUA^{II}), 5.16 (d, 1H, J=2.9 Hz, H-1 IdoUA^{IV}), 5.01 (d, 1H, J=3.3 Hz, H-1 IdoUA^{VI}), 4.90-4.85 (m, 2H, H-1 Glc^{III}, H-1 Glc^{V}), 4.20 (d, 1H, J=7.7 Hz, H-1 Glc^{I}), 4.81-4.56 (m, 8H, 4×CH₂-Ph), 3.92, 3.61 (m, 2H, CH₂₍ₐ₎-pent-4-ynyl), 3.74, 3.51, 3.43 (3s, 9H, CO₂Me), 3.49, 3.42, 3.37 (3s, 9H, OMe), 2.30-2.25 (m, 2H, CH_{2(c)}-pent-4-ynyl), 2.08-1.98 (6s, 18H, CH₃—OAc), 1.91 (t, 1H, J=2.5 Hz, CH_{(d)}-alkyne), 1.87-1.77 (m, 2H, CH_{2(b)}-pent-4-ynyl). MALDI-MS, positive mode, m/z: 1893.66 [M+Na⁺], 1909.52 [M+K⁺]. [α]_{D}²¹=5.5 (c=1.39, CHCl₃).

Compound 194: ¹H NMR (400 MHz, CDCl₃, ppm): δ=7.70-7.51 (m, 4H, arom.), 7.40-7.13 (m, 26H, arom.), 5.25 (d, 1H, J=4.3 Hz, H-1 IdoUA^{II}), 5.19-5.13 (m, 2H, H-1 IdoUA^{IV}, H-1 IdoUA^{VI}), 4.90-4.79 (m, 2H, CH₂-Ph), 4.89 (m, 1H, H-1 Glc^{III}), 4.79 (m, 1H, H-1 Glc^{V}), 4.70-4.56 (m, 6H, 3×CH₂-Ph), 4.15 (d, 1H, J=7.9 Hz, H-1 Glc^{I}), 3.90, 3.50 (m, 2H, CH₂₍ₐ₎-pent-4-ynyl), 3.67, 3.54, 3.51 (3s, 9H, CO₂Me), 3.44, 3.38, 3.34 (3s, 9H, OMe), 2.30-2.24 (m, 2H, CH_{2(c)}-pent-4-ynyl), 2.07-2.01 (3s, 9H, CH₃—OAc), 1.99, 1.90 (2s, 6H, CH₃—OAc), 1.89 (m, 1H, CH_{(d)}-alkyne), 1.85-1.73 (m, 2H, CH_{2(b)}-pent-4-ynyl), 1.00 (s, 9H, C(CH₃)₃). MALDI-MS, positive mode, m/z: 2089.24 [M+Na⁺]. [α]_{D}²¹=2.3 (c=1.22, CHCl₃).

Compound 195: ¹H NMR (400 MHz, CDCl₃, ppm): δ=7.71-7.61 (m, 4H, arom.), 7.40-7.10 (m, 26H, arom.), 5.25 (d, 1H, J=5.3 Hz, H-1 IdoUA^{II}), 5.18 (sl, 1H, H-1 IdoUA^{IV}), 5.10 (d, 1H, J=2.9 Hz, H-1 IdoUA^{VI}), 4.93 (d, 1H, J=2.9 Hz, H-1 Glc^{III}), 4.87, 4.60 (m, 2H, CH₂-Ph), 4.83 (m, 1H, H-1 Glc^{V}), 4.68-4.59 (m, 4H, 2×CH₂-Ph), 4.51-4.42 (m, 2H, CH₂-Ph), 4.16 (d, 1H, J=7.8 Hz, H-1 Glc^{I}), 3.86, 3.51 (m, 2H, CH₂₍ₐ₎-pent-4-ynyl), 3.72, 3.62, 3.52 (3s, 9H, CO₂Me), 3.48, 3.47, 3.41 (3s, 9H, OMe), 2.33-2.25 (m, 2H, CH_{2(c)}-pent-4-ynyl), 2.07 (s, 3H, CH₃—OAc), 2.01 (s, 9H, CH₃—OAc), 1.94 (s, 3H, CH₃—OAc), 1.89 (t, 1H, J=2.7 Hz, CH_{(d)}-alkyne), 1.87-1.74 (m, 2H, CH_{2(b)}-pent-4-ynyl), 1.00 (s, 9H, C(CH₃)₃). MALDI-MS, positive mode, m/z: 2089.32 [M+Na⁺]. [α]_{D}²¹=1.6 (c=1.02, CHCl₃).

Compound 196: ¹H NMR (400 MHz, CDCl₃, ppm): δ=7.36-7.11 (m, 15H, arom.), 5.15 (d, 1H, J=4.5 Hz, H-1 IdoUA^{II}), 5.12 (d, 1H, J=3.2 Hz, H-1 IdoUA^{IV}), 4.98 (d, 1H, J=3.4 Hz, H-1 IdoUA^{VI}), 4.92 (d, 1H, J=3.7 Hz, H-1 Glc^{III}), 4.82 (m, 1H, H-1 Glc^{V}), 4.80-4.59 (m, 6H, 3×CH₂-Ph), 4.17 (d, 1H, J=7.9 Hz, H-1 Glc^{I}), 3.88, 3.57 (m, 2H, CH₂₍ₐ₎-pent-4-ynyl), 3.51, 3.47, 3.45 (3s, 9H, CO₂Me), 3.45, 3.42, 3.38, 3.32 (4s, 12H, OMe), 2.28-2.21 (m, 2H, CH_{2(c)}-pent-4-ynyl), 2.05-1.96 (6s, 18H, CH₃—OAc), 1.88 (t, 1H, J=2.7 Hz, CH_{(d)}-alkyne), 1.83-1.70 (m, 2H, CH_{2(b)}-pent-4-ynyl). MALDI-MS, positive mode, m/z: 1816.46 [M+Na⁺]. [α]_{D}²¹=+3.8 (c=1.55, CHCl₃).

Compound 197: ¹H NMR (400 MHz, CDCl₃, ppm): δ=7.77-7.61 (m, 4H, arom.), 7.45-7.19 (m, 21H, arom.), 5.29 (d, 1H, J=4.5 Hz, H-1 IdoUA^{II}), 5.23-5.19 (m, 2H, H-1 IdoUA^{IV}, H-1 IdoUA^{VI}), 4.99 (m, 1H, H-1 Glc^{III}), 4.95 (m, 1H, H-1 Glc^{V}), 4.94-4.84 (m, 2H, CH₂-Ph), 4.76-4.66 (m, 2H, CH₂-Ph), 4.71-4.64 (m, 2H, CH₂-Ph), 4.20 (d, 1H, J=7.9 Hz, H-1 Glc^{I}), 3.72, 3.59, 3.57 (3s, 9H, CO₂Me), 3.51, 3.49, 3.44, 3.40 (4s, 12H, OMe), 2.34-2.29 (m, 2H, CH_{2(c)}-pent-4-ynyl), 2.12, 2.09, 2.07, 2.04, 1.98 (5s, 15H, CH₃—OAc), 1.93 (t, 1H, J=2.7 Hz, CH_{(d)}-alkyne), 1.91-1.78 (m, 2H, CH_{2(b)}-pent-4-ynyl), 1.05 (s, 9H, C(CH₃)₃). MALDI-MS, positive mode, m/z: 2013.17 [M+Na⁺].

Compound 198: ¹H NMR (400 MHz, CDCl₃, ppm): δ=7.72-7.56 (m, 4H, arom.), 7.40-7.16 (21H, arom.), 5.19 (d, 1H, J=4.3 Hz, H-1 IdoUA^{II}), 5.17 (sl, 1H, H-1 IdoUA^{IV}), 4.99 (sl, 1H, H-1 IdoUA^{VI}), 4.85 (m, 1H, H-1 Glc^{III}), 4.79 (m, 1H, H-1 Glc^{V}), 4.15 (d, 1H, J=7.9 Hz, H-1 Glc^{I}), 4.80-4.58 (m, 2H, CH₂-Ph), 4.64-4.57 (m, 2H, CH₂-Ph), 4.64 (sl, 2H, CH₂-Ph), 3.84, 3.50 (m, 2H, CH₂₍ₐ₎-pent-4-ynyl), 3.73, 3.66, 3.50 (3s, 9H, CO₂Me), 3.47, 3.45, 3.41, 3.35 (4s, 12H, OMe), 2.29-2.24 (m, 2H, CH_{2(c)}-pent-4-ynyl), 2.08-1.97 (5s, 15H, CH₃—OAc), 1.89 (sl, 1H, CH_{(d)}-alkyne), 1.86-1.80 (m, 2H, CH_{2(b)}-pent-4-ynyl), 0.99 (s, 9H, C(CH₃)₃). MALDI-MS, positive mode, m/z: 2013.57 [M+Na⁺]. [α]_{D}²¹=8.1 (c=1.60, CHCl₃).

Compound 199: ¹H NMR (400 MHz, CDCl₃, ppm): δ=7.74-7.65 (m, 4H, arom.), 7.43-7.16 (m, 21H, arom.), 5.32 (d, 1H, J=1.9 Hz, H-1 IdoUA^{II}), 5.13 (d, 1H, J=3.5 Hz, H-1 IdoUA^{IV}), 5.05 (d, 1H, J=2.6 Hz, H-1 IdoUA^{VI}), 4.94 (2d, 2H, J=3.3 Hz, H-1 Glc^{III}, H-1 Glc^{V}), 4.20 (d, 1H, J=7.6 Hz, H-1 Glc^{I}), 4.78 (sl, 2H, CH₂-Ph), 4.64, 4.58 (2d, 2H, J=12.0 Hz, CH₂-Ph), 4.51, 4.43 (2d, 2H, J=11.7 Hz, CH₂-Ph), 3.88, 3.53 (m, 2H, CH₂₍ₐ₎-pent-4-ynyl), 3.69, 3.65, 3.46 (3s, 9H, CO₂Me), 3.50, 3.48, 3.38 (4s, 12H, OMe), 2.36-2.26 (m, 2H, CH_{2(c)}-pent-4-ynyl), 2.06-2.02 (4s, 12H, CH₃—OAc), 1.96 (s, 3H, CH₃—OAc), 1.92 (t, 1H, J=2.7 Hz, CH_{(d)}-alkyne), 1.90-1.76 (m, 2H, CH_{2(b)}-pent-4-ynyl), 0.99 (s, 9H, C(CH₃)₃). MALDI-MS, positive mode, m/z: 2013.23 [M+Na⁺]. [α]_{D}²¹=+13.3 (c=1.39, CHCl₃).

Compound 200: ¹H NMR (400 MHz, CDCl₃, ppm): δ=7.76-7.60 (m, 4H, arom.), 7.40-7.16 (16H, arom.), 5.26 (d, 1H, J=4.2 Hz, H-1 IdoUA^{II}), 5.21 (sl, 1H, H-1 IdoUA^{IV}), 5.05 (d, 1H, J=3.3 Hz, H-1 IdoUA^{VI}), 4.98 (m, 1H, H-1 Glc^{III}), 4.90 (m, 1H, H-1 Glc^{V}), 4.18 (d, 1H, J=7.6 Hz, H-1 Glc^{I}), 4.85, 4.64 (m, 2H, CH₂-Ph), 4.69 (sl 2H, CH₂-Ph), 3.88, 3.54 (m, 2H, CH₂₍ₐ₎-pent-4-ynyl), 3.77, 3.71, 3.55 (3s, 9H, CO₂Me), 3.53-3.50 (m, 6H, OMe), 3.45, 3.44, 3.40 (3s, 9H, OMe), 2.35-2.27 (m, 3H, CH_{2(c)}-pent-4-ynyl, CH_{(d)}-alkyne), 2.11, 2.08, 2.04, 2.03, 1.97 (5s, 15H, CH₃—OAc), 1.97 (t, 1H, J=2.7 Hz, CH_{(d)}-alkyne), 1.88-1.77 (m, 2H, CH_{2(b)}-pent-4-ynyl), 1.04 (s, 9H, C(CH₃)₃). MALDI-MS, positive mode, m/z: 1937.77 [M+Na⁺]. [α]_{D}²¹=+4.4 (c=2.50, CHCl₃).

Compound 201: ¹H NMR (400 MHz, CDCl₃, ppm): δ=7.45-7.21 (m, 30H, arom.), 5.29 (d, 1H, J=4.4 Hz, H-1 IdoUA^{II}), 5.23 (d, 1H, J=3.3 Hz, H-1 IdoUA^{IV}), 5.17 (d, 1H, J=3.1 Hz, H-1 IdoUA^{VI}), 5.00 (d, 1H, J=3.6 Hz, H-1 Glc^{III}), 4.93 (m, 1H, H-1 Glc^{V}), 4.27 (d, 1H, J=7.9 Hz, H-1 Glc^{I}), 4.90-4.62 (m, 12H, 6×CH₂-Ph), 4.00, 3.68 (m, 2H, CH₂₍ₐ₎-pent-4-ynyl), 3.51 (s, 3H, CO₂Me), 3.50 (sl, 6H, CO₂Me), 2.82-2.43 (m, 4H, CH₂-Lev), 2.38-2.32 (m, 2H, CH_{2(c)}-pent-4-ynyl), 2.18 (s, 3H, CH₃-Lev), 2.13, 2.11, 2.09, 2.08, 2.07, 2.04 (6s, 18H, CH₃—OAc), 1.98 (t, 1H, J=2.7 Hz, CH_{(d)}-alkyne), 1.94-1.81 (m, 2H, CH_{2(b)}-pent-4-ynyl). MALDI-MS, positive mode, m/z: 2128.84 [M+Na⁺], 2144.76 [M+K⁺]. [α]_{D}²¹=6.6 (c=1.03, CHCl₃).

### 1. Preparation 17: Synthesis of Protected Hexasaccharide 202 (Scheme 17)

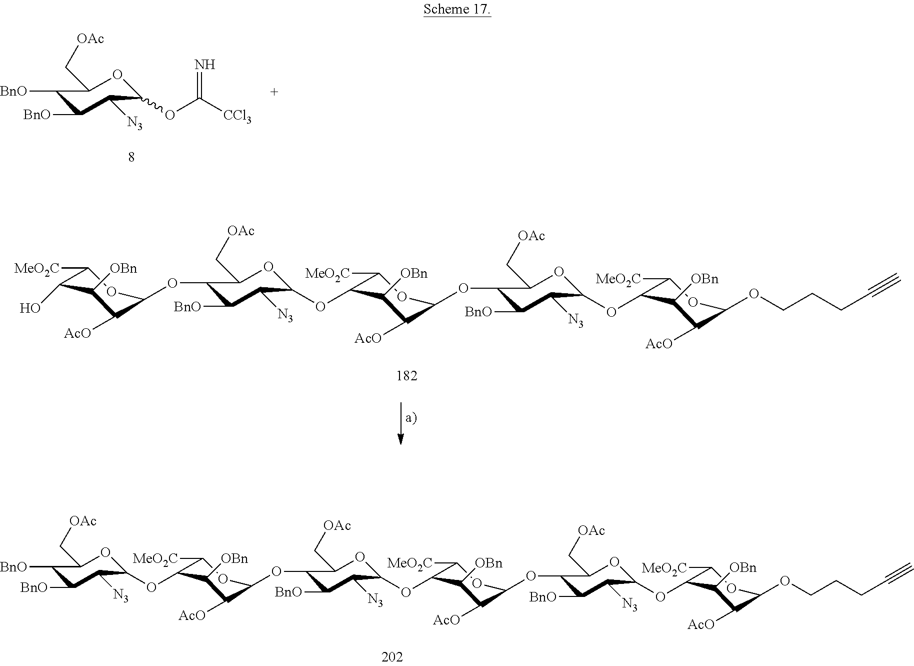

Step 17.a: Synthesis of compound 202: O-glycosylation reaction between monosaccharide donor 8 (44 mg, 0.077 mmol, 1.3 eq.) with pentasaccharide acceptor 182 (100 mg, 0.059 mmol, 1 eq.) was performed according to the general method B. Purification was effected by chromatography on silica gel column (heptane/ethyl acetate: 9/1 to 5/5) to give hexasaccharide 202 (92 mg, 74%) as a viscous colourless compound. ¹H NMR (400 MHz, CDCl₃, ppm): δ=7.45-7.18 (m, 35H, arom.), 5.33-5.25 (m, 2H, H-1 IdoUA^{III}, H-1 IdoUA^{V}), 5.03 (d, 1H, J=3.6 Hz, H-1 Glc^{VI}), 5.01 (s, 1H, H-1 IdoUA^{I}), 4.97 (d, 1H, J=3.5 Hz, H-1 Glc^{IV}), 4.95-4.55 (m, 21H, H-5 IdoUA^{V}, H-5 IdoUA^{III}, H-2 IdoUA^{V}, H-2 IdoUA^{III}, H-5 IdoUA^{I}, H-2 IdoUA^{I}, H-1 Glc^{II}, 7×CH₂-Ph), 4.43 (d, 1H, J=12.8 Hz, H-6a Glc^{VI}), 4.36 (d, 1H, J=12.4 Hz, H-6a Glc^{IV}), 4.29-4.13 (m, 4H, H-6b Glc^{VI}, H-6b Glc^{IV}, H-6a/b Glc^{II}), 4.08 (sl, 1H, H-4 IdoUA^{I}), 4.06-3.70 (m, 16H, H-3 IdoUA^{V}, H-3 IdoUA^{III}, H-4 IdoUA^{V}, H-4 IdoUA^{III}, H-3 Glc^{VI}, H-5 Glc^{VI}, H-3 IdoUA^{I}, H-4 Glc^{IV}, H-5 Glc^{IV}, H-3 Glc^{II}, H-4 Glc^{II}, H-5 Glc^{II}, CO₂Me, CH₍ₐ₎-pent-4-ynyl), 3.64 (t, 1H, J=9.8 Hz, H-3 Glc^{IV}), 3.61-3.49 (m, 8H, 2×CO₂Me, H-4 Glc^{VI}, CH₍ₐ₎-pent-4-ynyl), 3.34-3.23 (m, 3H, H-2 Glc^{VI}, H-2 Glc^{IV}, H-2 Glc^{II}), 2.31-2.22 (m, 2H, CH_{2(c)}-pent-4-ynyl), 2.12, 2.09, 2.08, 2.06, 2.04, 1.93 (6s, 18H, CH₃—OAc), 1.90 (t, 1H, J=2.6 Hz, CH_{(d)}-alkyne), 1.85-1.71 (m, 2H, CH_{2(b)}-pent-4-ynyl). MALDI-MS, positive mode, m/z: 2121.49 [M+Na⁺], 2137.38 [M+K⁺]. [c]_{D}²¹=+9.3 (c=0.70, CHCl₃).

### H. Octasaccharides Preparations

### 1. Preparation 18: Synthesis of Protected Octasaccharides 203, 204, 205 and 206 (Scheme 18)

Below is reported the general formula of all the protected octasaccharides synthesized.

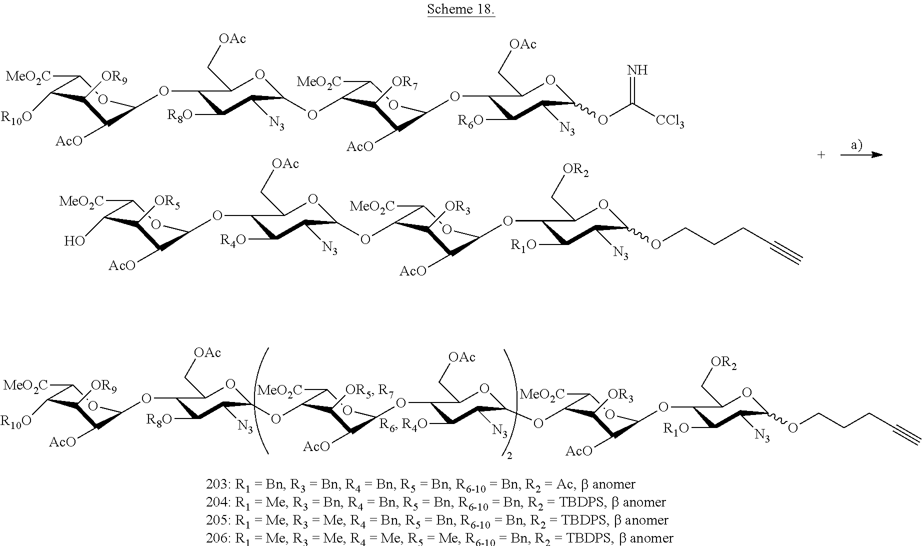

Step 18.a: Synthesis of compound 203:O-glycosylation reaction between tetrasaccharide donor 164 (173.7 mg, 0.113 mmol, 1.3 eq.) and tetrasaccharide acceptor 166 (119 mg, 0.087 mmol, 1 eq.) was performed according to the general method B. Purification was effected by chromatography on silica gel column (heptane/ethyl acetate: 7/3 to 5/5) to give octasaccharide 203 (169 mg, 71%) as a white amorphous solid. ¹H NMR (400 MHz, CDCl₃, ppm): δ=7.32-7.16 (m, 45H, arom.), 5.25-5.20 (m, 3H, H-1 IdoUA^{IV}, H-1 IdoUA^{II}, H-1 IdoUA^{VI}), 5.14 (d, 1H, J=3.2 Hz, H-1 IdoUA^{VIII}), 4.90 (d, 2H, J=3.4 Hz, H-1 Glc^{II}, H-1 Glc^{VII}), 4.86-4.75 (m, 9H, H-1 Glc^{V}, 2×CH₂-Ph, H-2 IdoUAV^{III}, H-2 IdoUA^{IV}, H-2 IdoUA^{II}, H-2 IdoUA^{VI}), 4.74-4.52 (m, 16H, 7×CH₂-Ph, H-5 IdoUA^{VIII}, H-5 IdoUA^{II}), 4.50, 4.48 (2d, 2H, J=4.5 Hz, H-5 IdoUA^{IV}, H-5 IdoUA^{VI}), 4.38 (d, 1H, J=12.2 Hz, H-6a Glc^{I}), 4.33-4.24 (m, 3H, H-6a Glc^{V}, H-6a Glc^{II}, H-6a Glc^{VII}), 4.18 (d, 1H, J=7.8 Hz, H-1 Glc^{I}), 4.15-4.05 (m, 4H, H-6b Glc^{V}, H-6b Glc^{II}, H-6b Glc^{VIII}, H-6b Glc^{I}), 3.96-3.69 (m, 17H, H-4 IdoUA^{IV}, H-4 IdoUA^{VI}, H-4 IdoUA^{VIII}, H-4 Glc^{III}, H-4 Glc^{V}, H-3 IdoUA^{IV}, H-3 IdoUA^{VI} H-3 IdoUA^{VIII}, H-4 Glc^{I}, H-5 Glc^{I}, H-4 IdoUA^{II}, H-3 IdoUA^{II}, H-5 Glc^{V}, H-5 Glc^{IIIl}, H-5 Glc^{VIII}, H-4 Glc^{VIII}, CH₍ₐ₎-pent-4-ynyl), 3.63-3.52 (m, 4H, H-3 Glc^{VIII}, H-3 Glc^{V}, H-3 Glc^{IIIl}, CH₍ₐ₎-pent-4-ynyl), 3.49, 3.47, 3.46, 3.42 (4s, 12H, CO₂Me), 3.38-3.27 (m, 1H, H-2 Glc^{I}), 3.26-3.17 (m, 4H, H-2 Glc^{IIIl}, H-2 Glc^{V}, H-3 Glc^{I}, H-2 Glc^{VII}), 2.29-2.23 (m, 2H, CH_{2(c)}-pent-4-ynyl), 2.03, 2.02, 2.01, 1.97, 1.95, 1.94, 2×1.93 (8s, 24H, CH₃—OAc), 1.89 (t, 1H, J=2.6 Hz, CH_{(d)}-alkyne), 1.83-1.71 (m, 2H, CH_{2(b)}-pent-4-ynyl). MALDI-MS, positive mode, m/z: 2763.50 [M+Na⁺], 2779.41 [M+K⁺]. [α]_{D}²¹=3.9 (c=0.46, CHCl₃).

Preparation of protected octasaccharides 204, 205 and 206 were carried out as described for octasaccharide 203.

Compound 204: ¹H NMR (400 MHz, CDCl₃, ppm); MALDI-MS, positive mode, m/z: 2883.34 [M+Na⁺]. [α]_{D}²¹=2.6 (c=1.82, CHCl₃).

Compound 205: ¹H NMR (400 MHz, CDCl₃, ppm); MALDI-MS, positive mode, m/z: 2806.84 [M+Na⁺]. [α]_{D}²¹=+2.1 (c=0.48, CHCl₃).

Compound 206: ¹H NMR (400 MHz, CDCl₃, ppm); MALDI-MS, positive mode, m/z: 2654.36 [M+Na⁺]. [α]_{D}²¹=+6.4 (c=2.00, CHCl₃).

### I. Decasaccharides Preparations

### 1. Preparation 19: Synthesis of Nrotected Decasaccharide 208 (Scheme 19)

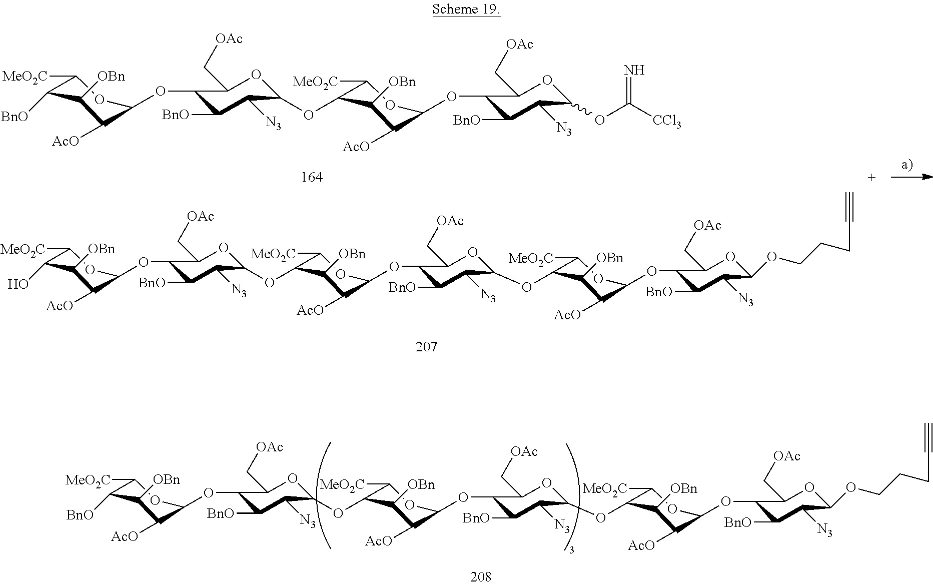

Hexasaccharide acceptor 207 was prepared from 201 by levuniloyl cleavage.

O-glycosylation reaction between tetrasaccharide donor 164 (54.7 mg, 0.036 mmol, 1.3 eq.) and hexasaccharide acceptor 207 (55 mg, 0.027 mmol, 1 eq.) was performed according to the general method B. Purification was effected by chromatography on silica gel column (heptane/ethyl acetate: 7/3) to give decasaccharide 208 (51.7 mg, 56%) as a white amorphous compound. ¹H NMR (400 MHz, CDCl₃, ppm); MALDI-MS, positive mode, m/z: 3402.92 [M+Na⁺]. [α]_{D}²¹=+32.4 (c=1.25, CH₂Cl₂).

### II. Section 2

### Examples

### A. General Methods:

### Method I: General Method for Deacetylation

The compound was stirred for 1 h with dry potassium carbonate (0.5 eq.) in anhydrous methanol (0.02 M) under a nitrogen atmosphere. The reaction mixture was neutralized with Dowex 50WX8-200 resin until pH 7, filtered, concentrated and dried under vacuum to give the deacetylated compound which was directly used in the next step without any further purification.

### Method J: General Method for Desilylation

The saccharide was stirred overnight with hydrogen fluoride pyridine (100 eq./TBDPS function) in anhydrous pyridine (0.04 M) under a nitrogen atmosphere. The reaction mixture was neutralized with methoxytrimethylsilane (1.1 eq./eq. HF.Py), stirred for 1 h at room temperature, filtered through a pad of Celite®, concentrated in vacuo and the residue was purified by Sephadex LH-20 gel column or by chromatography on silica gel column to give the desilylated compound.

### Method K: General Method for Desilylation

To a solution of saccharide in anhydrous methanol (0.02 M) was added tetraammonium fluoride (20 eq./TBDPS function). After stirring at 50° C. overnight, the reaction mixture was neutralized with an aqueous saturated solution of NaHCO₃ until pH 7-8 and directly poured onto Sephadex LH20 gel column to give the desilylated compound.

### Method L: General Method for Selective Azide Reduction

The aza compound was dissolved in anhydrous methanol (0.02 M) under a nitrogen atmosphere. 1,3-propanedithiol (10 eq./N₃ function) and Et₃N (10 eq./N₃ function) were successively added. The reaction mixture was protected from light and stirred 2 days at room temperature or 40° C. The reaction mixture was concentrated to dryness under reduced pressure and the residue was purified by chromatography on silica gel column or by a Sephadex LH20 gel column to afford the desired product.

### Method M: General Method for O,N-Sulfation

Sulfur trioxide pyridine complex (5 eq./OH or NH₂ function) was added to a solution of the saccharide, previously coevaporated with pyridine, in anhydrous pyridine (0.02 M) under a nitrogen atmosphere. The reaction mixture was protected from light and stirred overnight at 55° C. After cooling the reaction mixture to 0° C., methanol (16 eq./eq. Py.SO₃) and triethylamine (1.8 eq./eq. Py.SO₃) were then added to quench the reaction. The reaction mixture was stirred for 1 h at room temperature and directly poured onto Sephadex LH20 gel column to give O, N-sulfated compound.

### Method N: General Method for Saponification

Lithium hydroxide (25 eq./CO₂Me function) was added dropwise to a solution of the O, N-sulfated compound dissolved in water (0.03 M) at 0° C. The reaction mixture was stirred for 2 days at room temperature and directly poured onto Sephadex G25F column (0.2 M NaCl). The combined fractions were concentrated and desalted on Sephadex G25F column (water). The combined fractions were concentrated and lyophilized to give the corresponding saponified compound.

### Method O: General Method for Hydrogenolysis

A solution of benzylated compound in a mixture of tent-butanol/water (1/1, 0.1 mL/mg of the compound) was degassed and stirred under hydrogen (1 bar) in the presence of Pd(OH)₂ catalyst (1× weight of the compound) for 48 h at room temperature. The reaction mixture was filtered through a pad of coton and Celite® and concentrated. The product was diluted in water and filtered through a syringe driven filter unit (0.22 μm×13 mm) and lyophilized to give the corresponding debenzylated compound.

### Method P: General Method for Hydrogenolysis

A solution of benzylated compound in a 100 mM phosphate buffer pH 7.0 (0.1 mL/mg of the compound) was degassed and stirred under hydrogen (1 bar) in the presence of Pd(OH)₂ catalyst (2× weight of the compound) for 72 h at room temperature. The reaction mixture was filtered through a pad of coton and Celite® and concentrated in vacuo. The residue was diluted in water and poured onto Sephadex G25F column (water) for desalting. The combined fractions were concentrated and lyophilized to give the corresponding debenzylated compound.

### Method Q: General Method for N-Sulfation

Sodium bicarbonate (40 eq./NH₂ function) was added to a solution of saccharide in freshly prepared and degazed (with argon) aqueous solution of NaHCO₃ (0.01 M) at room temperature under an atmosphere (bubbling) of argon. The solution was cooled to 0° C. and sulfur trioxide pyridine complex (20 eq./NH₂ function) was added in seven portions at t=0; 30 min; 1 h; 1 h30; 2 h; 2 h30; 3 h. The reaction mixture was vigorously stirred for 24 h at 0-4° C. and directly poured onto Sephadex G25F (0.2 M NaCl). The combined fractions were concentrated and desalted on Sephadex G25F (water). The combined fractions were concentrated and lyophilized to give the corresponding N-sulfated compound.

### Method R: General Method for N-Acylation

Sodium bicarbonate (5 eq./NH₂ function) was added to a solution of saccharide in aqueous solution of NaHCO₃ (0.015 M) at room temperature. Then, the acyl anhydride (solubilized in acetonitrile 0.4 M if necessary) was added dropwise at 0° C. 2 eq. by 2 eq. every 30 min until starting material and intermediates disappeared by TLC (time 17 h to 2 days). The reaction mixture was directly poured onto Sephadex G25F (0.2 M NaCl). The combined fractions were concentrated and desalted on Sephadex G25F (water). The combined fractions were concentrated and lyophilized to give the corresponding N-acylated compound.

B. Examples from 2′-Ido and 2,6-Glc Sulfated Oligosaccharides Family:

1. Preparation of Fully Benzylated Compounds

a) Preparation of Examples 215 and 216 (Scheme 20)

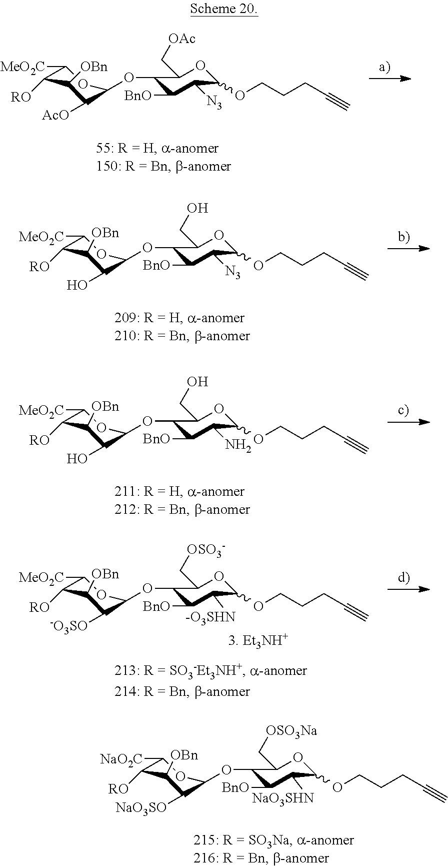

Step 20.a: Synthesis of compound 209: Deacetylation of compound 55 (100 mg, 0.138 mmol) was performed according to the general method I. Compound 209 was obtained as a white solid and directly used in the next step without any further purification. ¹H NMR (400 MHz, CDCl₃, ppm): δ=7.47-7.19 (m, 10H, arom.), 5.21 (s, 1H, H-1′), 4.92 (d, 1H, J=3.4 Hz, H-1), 4.80 (d, 1H, J=1.9 Hz, H-5′), 4.75-4.57 (m, 4H, CH₂-Ph), 4.02-3.94 (m, 3H, H-2′, H-4′, H-6a), 3.89-3.73 (m, 6H, CH₍ₐ₎-pent-4-ynyl, H-3′, H-6b, H-3, H-4, H-5), 3.58 (m, 1H, CH₍ₐ₎-pent-4-ynyl), 3.44 (s, 3H, CO₂Me), 3.35 (dd, 1H, J=3.4 Hz, J=10.1 Hz, H-2), 3.03 (br, 1H, OH), 2.42-2.35 (m, 2H, CH_{2(c)}-pent-4-ynyl), 2.08 (t, 1H, J=2.6 Hz, CH_{(d)}-alkyne), 1.97-1.82 (m, 2H, CH_{2(b)}-pent-4-ynyl). MALDI-MS, positive mode, m/z: 664.05 [M+Na⁺], 680.09 [M+K⁺].

Step 20.b: Synthesis of compound 211: Selective azide reduction of compound 209 (0.138 mmol) was performed according to the general method L. Purification was effected by chromatography on silica gel column (dichloromethane/methanol: 100/0 to 90/10 with 1% Et₃N) to give compound 211 (72 mg, 85% over 2 steps) as a white solid. ¹H NMR (400 MHz, CDCl₃, ppm): δ=7.52-7.26 (m, 10H, arom.), 5.32 (s, 1H, H-1′), 4.99-4.92 (m, 2H, CH-Ph, H-5′), 4.90 (d, 1H, J=3.4 Hz, H-1), 4.79 (q, 2H, J=11.5 Hz, CH₂-Ph), 4.64 (d, 1H, J=11.6 Hz, CH-Ph), 4.10 (br, 1H, H-4′), 4.02 (br, 1H, H-2′), 4.00-3.85 (m, 5H, H-3′, H-4, H-6a, H-6b, CH₍ₐ₎-pent-4-ynyl), 3.84-3.78 (m, 1H, H-5), 3.63 (s, 3H, CO₂Me), 3.61-3.49 (m, 2H, H-3, CH_{(a′)}-pent-4-ynyl), 2.88 (dd, 1H, J=3.4 Hz, J=9.8 Hz, H-2), 2.44-2.37 (m, 2H, CH_{2(c)}-pent-4-ynyl), 2.08 (t, 1H, J=2.6 Hz, CH_{(d)}-alkyne), 1.99-1.88 (m, 2H, CH_{2(b)}-pent-4-ynyl). MALDI-MS, positive mode, m/z: 638.48 [M+Na⁺], 654.46 [M+K⁺].

Step 20.c: Synthesis of compound 213: O,N-Sulfation of compound 211 (56.9 mg, 0.09 mmol) was performed according to the general method M. Purification was effected by size exclusion (Sephadex LH20 dichloromethane/ethanol: 1/1) to give quantitatively compound 213 as a yellow oil.

Step 20.d: Synthesis of compound 215: Saponification of compound 213 (0.09 mmol) was performed according to the general method N. Purification was effected by size exclusion (Sephadex G25 NaCl 0.2M, then G25 water) to give the saponified compound 215 (79 mg, 83% over 2 steps) as a white hygroscopic solid. ¹H NMR (400 MHz, D₂O, ppm): δ=7.57-7.35 (m, 10H, arom.), 5.30 (s, 1H, H-1′), 5.12 (d, 1H, J=3.4 Hz, H-1), 4.87 (d, 1H, J=1.9 Hz, H-5′), 4.86-4.81 (m, 4H, CH₂-Ph, CH-Ph, H-4′), 4.64 (d, 1H, J=11.0 Hz, CH-Ph), 4.51 (s, 1H, H-2′), 4.47 (s, 1H, H-3′), 4.38 (dd, 1H, J=2.2 Hz, J=11.6 Hz, H-6a), 4.32 (dd, 1H, J=4.2 Hz, J=11.6 Hz, H-6b), 4.10-4.05 (m, 1H, H-5), 3.97-3.86 (m, 2H, H-4, CH₍ₐ₎-pent-4-ynyl), 3.73 (t, 1H, J=10.2 Hz, H-3), 3.63-3.56 (m, 1H, CH_{(a′)}-pent-4-ynyl), 3.38 (dd, 1H, J=3.4 Hz, J=10.2 Hz, H-2), 2.42-2.36 (m, 3H, CH_{(d)}-alkyne, CH_{2(c)}-pent-4-ynyl), 1.99-1.78 (m, 2H, CH_{2(b)}-pent-4-ynyl). ESI-MS, negative mode, m/z: 1178.38 [M+2 DBA-3H]⁻, 1049.23 [M+1 DBA-2H]⁻, 920.07 [M-H].

Preparation of example 216 was carried out as described for example 215.

Compound 216: ¹H NMR (400 MHz, D₂O, ppm): δ=7.57-7.20 (m, 15H, arom.), 5.18 (s, 1H, H-1′), 4.82 (d, 1H, J=6.1 Hz, H-1), 4.78-4.58 (m, 4H, 2×CH₂-Ph), 4.50 (d, 1H, J=1.9 Hz, H-5′), 4.43 (sl, 2H, CH₂-Ph), 4.38-4.27 (m, 3H, H-2′, H-6a/b), 4.22-4.15 (m, 1H, H-5), 4.11-4.06 (m, 1H, H-4), 4.04-3.93 (m, 3H, H-3′, H-3, CH₍ₐ₎-pent-4-ynyl), 3.89-3.86 (sl, 1H, H-4′), 3.83-3.75 (m, 1H, CH_{(a′)}-pent-4-ynyl), 3.45 (dd, 1H, J=6.1 Hz, J=3.9 Hz, H-2), 2.49-2.28 (m, 3H, CH_{(d)}-alkyne, CH_{2(c)}-pent-4-ynyl), 1.91-1.81 (m, 2H, CH_{2(b)}-pent-4-ynyl). ESI-MS, negative mode, m/z: 464.52 [M-2H]²⁻, 309.33 [M-3H]³. [α]_{D}²¹=25.3 (c=1.50, H₂O).

b) Preparation of Examples 236, 237, 238, 239, 240 and 241 (Scheme 21)

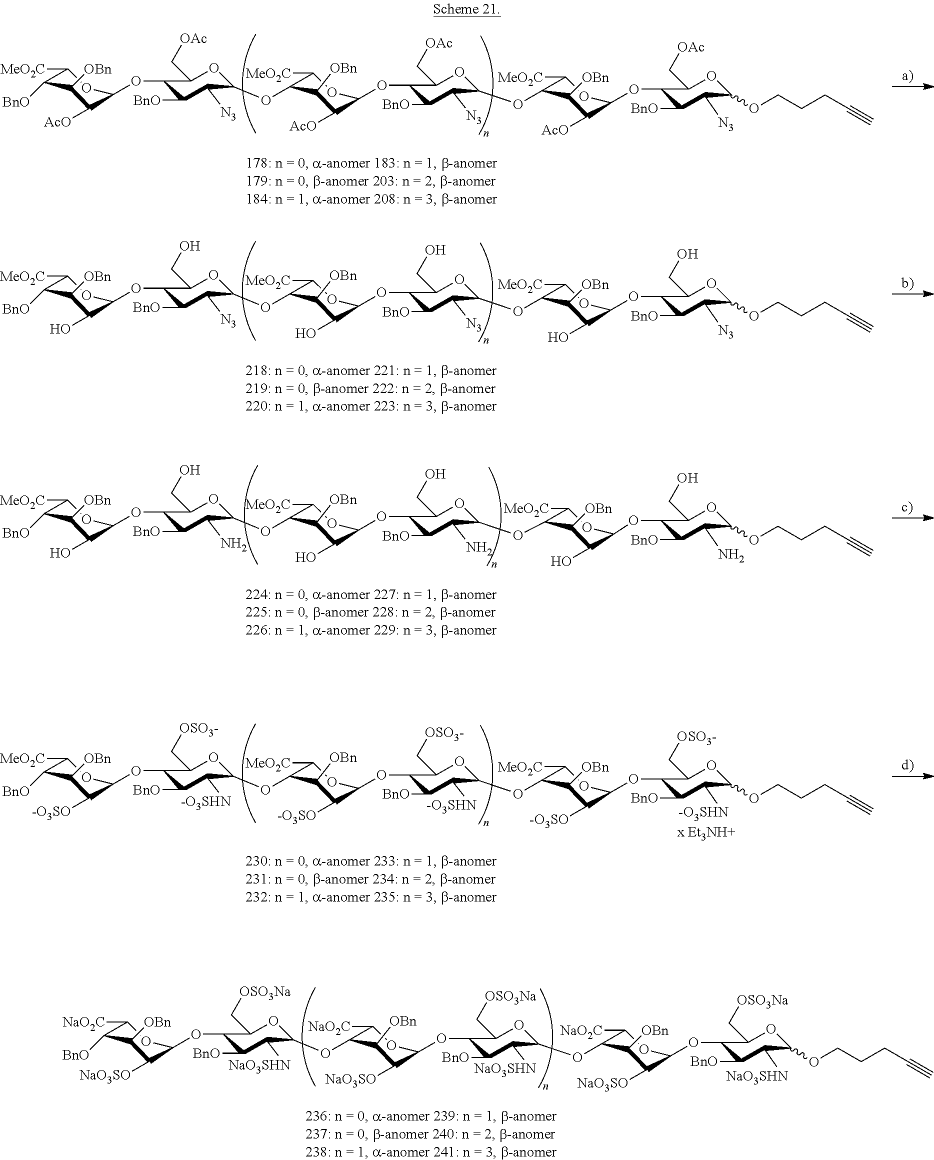

Step 21.a: Synthesis of compound 218: Deacetylation of compound 178 (117 mg, 0.080 mmol) was performed according to the general method I. Compound 218 was obtained as a white solid and directly used in the next step without any further purification. MALDI-MS, positive mode, m/z: 1311.24 [M+Na⁺], 1327.18 [M+K⁺].

Step 21.b: Synthesis of compound 224: Selective azide reduction of compound 218 (0.080 mmol) was performed according to the general method L. Purification was effected by chromatography on silica gel column (dichloromethane/methanol: 10/0 to 9/1 with 1% Et₃N) to give compound 224 (92.1 mg, 93% over 2 steps) as a white solid. ¹H NMR (400 MHz, CDCl₃, ppm): δ=7.45-7.13 (m, 25H, arom.), 5.31 (br., 1H, H-1 IdoUA^{II}), 5.26 (d, 1H, J=2.3 Hz, H-1 IdoUA^{IV}), 4.99, 4.87 (2d, 2H, J=11.3 Hz, CH₂-Ph), 4.93 (d, 1H, J=3.4 Hz, H-1 Glc^{III}), 4.91 (d, 1H, J=3.2 Hz, H-5 IdoUA^{II}), 4.83 (d, 1H, J=3.5 Hz, H-1 Glc^{I}), 4.78 (br, 1H, H-5 IdoUA^{IV}), 4.75, 4.59 (2d, 2H, J=11.6 Hz, CH₂-Ph), 4.66-4.38 (m, 6H, 3×CH₂-Ph), 4.23 (t, 1H, J=3.2 Hz, H-4 IdoUA^{II}), 4.03 (t, 1H, J=9.4 Hz, H-4 Glc^{I}), 3.96 (br., 1H, H-3 IdoUA^{II}), 3.90 (dd, 1H, J=3.2 Hz, J=12.2 Hz, H-6a Glc^{I}), 3.84-3.70 (m, 9H, H-6b Glc^{I}, H-6a Glc^{III}, H-6b Glc^{IIIl}, CH₍ₐ₎-pent-4-ynyl, H-2 IdoUA^{II}, H-3 IdoUA^{IV}, H-5 Glc^{I}, H-4 Glc^{III}, H-4 IdoUA^{IV}), 3.67 (br, 1H, H-2 IdoUA^{IV}), 3.53 (s, 3H, CO₂Me), 3.52-3.46 (m, 3H, H-3 Glc^{I}, H-5 Glc^{III}, CH_{(a′)}-pent-4-ynyl), 3.44 (s, 3H, CO₂Me), 3.37 (t, 1H, J=9.5 Hz, H-3 Glc^{III}), 2.86-2.78 (m, 2H, H-2 Glc^{I}, H-2 Glc^{III}), 2.34-2.27 (m, 2H, CH_{2(c)}-pent-4-ynyl), 1.95 (t, 1H, J=2.6 Hz, CH_{(d)}-alkyne), 1.88-1.77 (m, 2H, CH_{2(b)}-pent-4-ynyl). MALDI-MS, positive mode, m/z: 1259.23 [M+Na⁺], 1275.18 [M+K⁺]. [α]_{D}²¹=+44.9 (c=1.0, CH₂Cl₂).

Step 21.c: Synthesis of compound 230: O,N-Sulfation of compound 224 (34.4 mg, 0.0278 mmol) was performed according to the general method M. Purification was effected by size exclusion (Sephadex LH20 methanol/water: 100/1) to give compound 230 as a yellow solid. ESI-MS, negative mode, m/z: 986.42 [M+2 DBA-4H]²⁻, 921.84 [M+1 DBA-3H]²⁻, 857.25 [M-2H]²⁻, 571.16 [M-3H]³⁻.

Step 21.d: Synthesis of compound 236: Saponification of compound 230 (0.0278 mmol) was performed according to the general method N. Purification was effected by size exclusion (Sephadex G25 NaCl 0.2M, then G25 water) to give compound 236 (47.7 mg, 92% over 2 steps) as a white hygroscopic solid. ¹H NMR (400 MHz, D₂O, ppm): δ=7.49-7.20 (m, 25H, arom.), 5.41 (s, 1H, H-1 IdoUA^{II}), 5.27-5.18 (br s, 2H, H-1 IdoUA^{IV}, H-1 Glc^{III}), 5.03 (d, 1H, J=3.5 Hz, H-1 Glc^{I}), 4.84-4.63 (m, 2H, H-5 IdoUA^{IV}, H-5 IdoUA^{II}), 4.62-3.99 (m, 18H, 5×CH₂-Ph, H-2 IdoUA^{II}, H-3 IdoUA^{II}, H-4 IdoUA^{II}, H-6a Glc^{III}, H-6b Glc^{III}, H-6a Glc^{I}, H-6b Glc^{I}, H-5 Glc^{I}), 3.88-3.74 (m, 4H, H-5 Glc^{III}, H-4 Glc^{III}, CH₍ₐ₎-pent-4-ynyl, H-4 Glc^{I}), 3.71-3.58 (m, 2H, H-3 Glc^{III}, H-3 Glc^{I}), 3.55-3.47 (m, 1H, CH(a′)-pent-4-ynyl), 3.40-3.31 (m, 2H, H-2 Glc^{III}, H-2 Glc^{I}), 2.34-2.26 (m, 3H, CH_{2(c)}-pent-4-ynyl, CH_{(d)}-alkyne), 1.86-1.71 (m, 2H, CH_{2(b)}-pent-4-ynyl). ESI-MS, negative mode, m/z: 972.72 [M+2 DBA-4H]²⁻, 907.65 [M+1 DBA-3H]²⁻, 843.08 [M-2H]², 561.72 [M-3H]³⁻, 421.03 [M-4H]⁴⁻. [α]_{D}²¹=+12.7 (c=0.33, H₂O).

Preparation of example 237, 238, 239, 240 and 241 were carried out as described for example 236.

Compound 237: ¹H NMR (400 MHz, D₂O, ppm): δ=7.62-7.33 (m, 25H, arom.), 5.44 (sl, 1H, H-1 IdoUA^{II}), 5.38 (sl, 1H, H-1 IdoUA^{IV}), 5.24 (d, 1H, J=3.0 Hz, H-1 Glc^{III}), 4.82 (m, 1H, H-1 Glc^{I}), 4.89-4.53 (m, 10H, 5×CH₂-Ph), 4.06-3.84 (m, 2H, CH₂₍ₐ₎-pent-4-ynyl), 3.47 (dd, 1H, J=3.0 Hz, J=10.0 Hz, H-2 Glc^{III}), 3.42 (t, 1H, J=6.1 Hz, H-2 Glc^{I}), 2.46-2.36 (m, 3H, CH_{2(c)}-pent-4-ynyl, CH_{(d)}-alkyne), 1.93-1.83 (m, 2H, CH_{2(b)}-pent-4-ynyl). ESI-MS, negative mode, m/z: 972.81 [M+2 DBA-4H]²⁻, 907.72 [M+1 DBA-3H]²⁻, 843.14 [M-2H]²⁻, 561.75 [M-3H]³⁻. [α]_{D}²¹=9.6 (c=0.82, H₂O).

Compound 238: ¹H NMR (400 MHz, D₂O, ppm): δ=7.53-7.17 (m, 35H, arom.), 5.48-5.33 (m, 3H, H-1 IdoUA^{IV}, H-1 IdoUA^{II}, H-1 IdoUA^{VI}), 5.24 (d, 1H, J=3.1 Hz, H-1 Glc^{III}), 5.20 (d, 1H, J=2.5 Hz, H-1 Glc^{V}), 5.01 (d, 1H, J=3.4 Hz, H-1 Glc^{I}), 3.79, 3.51 (m, 2H, CH₂₍ₐ₎-pent-4-ynyl), 2.31-2.26 (m, 3H, CH_{2(c)}-pent-4-ynyl, CH_{(d)}-alkyne), 1.83-1.71 (m, 2H, CH_{2(b)}-pent-4-ynyl). ESI-MS, negative mode, m/z: 943.91 [M+3 DBA-6H]³⁻, 900.89 [M+2 DBA-5H]³⁻, 857.84 [M+1 DBA-4H]³⁻, 814.46 [M-3H]³⁻. [α]_{D}²¹=+18.7 (c=0.39, H₂O).

Compound 239: ¹H NMR (400 MHz, D₂O, ppm): δ=7.66-7.21 (m, 35H, arom.), 5.47-5.41 (m, 3H, H-1 IdoUA^{II}, H-1 IdoUA^{IV}, H-1 IdoUA^{VI}), 5.26 (br. s, 1H, H-1 Glc^{III}), 5.19 (br. s, 1H, H-1 Glc^{V}), 4.92-4.61 (m, 10H, H-5 IdoUA^{II}, H-5 IdoUA^{IV}, H-5 IdoUA^{VI}, H-1 Glc^{I}, 3×CH₂-Ph), 4.59-4.17 (m, 16H, H-6a/b Glc^{I}, H-2 IdoUA^{IV}, H-2 IdoUA^{VI}, H-2 IdoUA^{II}, H-3 IdoUA^{IV}, H-3 IdoUA^{VI}, H-3 IdoUA^{II}, 4×CH₂-Ph), 4.10 (m, 3H, H-4 IdoUA^{IV}, H-4 IdoUA^{VI}, H-4 IdoUA^{II}), 4.05-3.55 (m, 10H, H-5 Glc^{I}, H-4 Glc^{I}, H-3 Glc^{I}, H-5 Glc^{IIIl}, H-4 Glc^{IIIl}, H-3 Glc^{IIIl}, H-4 Glc^{V}, H-3 Glc^{V}, CH₂₍ₐ₎-pent-4-ynyl), 3.48-3.31 (m, 3H, H-2 Glc^{IIIl}, H-2 Glc^{V}, H-2 Glc^{I}), 2.38-2.31 (m, 3H, CH_{2(c)}-pent-4-ynyl, CH_{(d)}-alkyne), 1.88-1.78 (m, 2H, CH_{2(b)}-pent-4-ynyl). ESI-MS, negative mode, m/z: 1480.87 [M+4 DBA-6H]²⁻, 943.86 [M+3 DBA-6H]³⁻, 900.81 [M+2 DBA-5H]³⁻, 857.43 [M+1 DBA-4H]³⁻, 814.71 [M-3H]³⁻, 610.53 [M-4H]⁴⁻, 488.42 [M-5H]⁵⁻. [α]_{D}²¹=+3.0 (c=0.34, H₂O).

Compound 240: ¹H NMR (400 MHz, D₂O, ppm): δ=7.59-7.28 (m, 45H, arom.), 5.50 (br s, 1H, H-1 IdoUA^{II(IV,VI)}, 5.42 (br s, 2H, H-1 IdoUA^{IV(II,VI)}, H-1 IdoUA^{VI(IV,II)}), 5.38-5.26 (m, 4H, H-1 Glc^{VII}, H-1 Glc^{III}, H-1 Glc^{V}, H-1 IdoUA^{VIII}), 4.93-4.80 (m, 6H, 3×CH₂-Ph), 4.77-4.65 (m, 17H, H-5 IdoUA^{II}, H-5 IdoUA^{IV}, H-5 IdoUA^{VI}, H-5 IdoUA^{VIII}, 6×CH₂-Ph, H-1 Glc^{I}), 4.64-4.40 (m, 8H, H-2 IdoUA^{II}, H-2 IdoUA^{IV}, H-2 IdoUA^{VI}, H-2 IdoUA^{VIII}, H-3 IdoUA^{II}, H-3 IdoUA^{IV}, H-3 IdoUA^{VI}, H-3 IdoUA^{VIII}), 4.39-4.03 (m, 2H, H-5 Glc^{I}, H-4 Glc^{I}), 4.02-3.90 (m, 5H, H-4 Glc^{V}, H-4 Glc^{III}, H-4 Glc^{VII}, H-3 Glc^{I}, CH₍ₐ₎-pent-4-ynyl), 3.89-3.69 (m, 4H, H-3 Glc^{V}, H-3 Glc^{III}, H-3 Glc^{VII}, CH_{(a′)}-pent-4-ynyl), 3.51-3.33 (m, 4H, H-2 Glc^{V}, H-2 Glc^{III}, H-2 Glc^{VII}, H-2 Glc^{I}), 2.40-2.32 (m, 3H, CH_{2(c)}-pent-4-ynyl, CH_{(d)}-alkyne), 1.88-1.78 (m, 2H, CH_{2(b)}-pent-4-ynyl). ESI-MS, negative mode, m/z: 1368.87 [M+7 DBA-10H]³⁻, 1325.81 [M+6 DBA-9H]³⁻, 1282.77 [M+5 DBA-8H]³⁻, 1239.72 [M+4 DBA-7H]³⁻, 962.06 [M+5 DBA-9H]⁴⁻, 929.52 [M+4 DBA-8H]⁴⁻, 896.98 [M+3 DBA-7H]⁴⁻, 864.70 [M+2 DBA-6H]⁴⁻, 832.41 [M+DBA-4H]⁴⁻, 800.12 [M-4H]⁴⁻.

Compound 241: ¹H NMR (400 MHz, D₂O, ppm); ESI-MS, negative mode, m/z: 1183.56 [M+6 DBA-10H]⁴⁻, 1131.27 [M+5DBA-SO₃-9H]⁴⁻, 1098.72 [M+4 DBA-SO₃-8H]⁴⁻, 1066.67 [M+3 DBA-SO₃-7H]⁴⁻, 894.77 [M+4 DBA-9H]⁵⁻, 869.14 [M+3 DBA-8H]⁵⁻, 843.31 [M+2 DBA-7H]⁵⁻, 817.28 [M+1 DBA-6H]⁵⁻, 775.63 [M—SO₃-5H]⁵⁻, 659.36 [M-6H]⁶⁻.

b) Preparation of Examples 244 and 245 (Scheme 22)

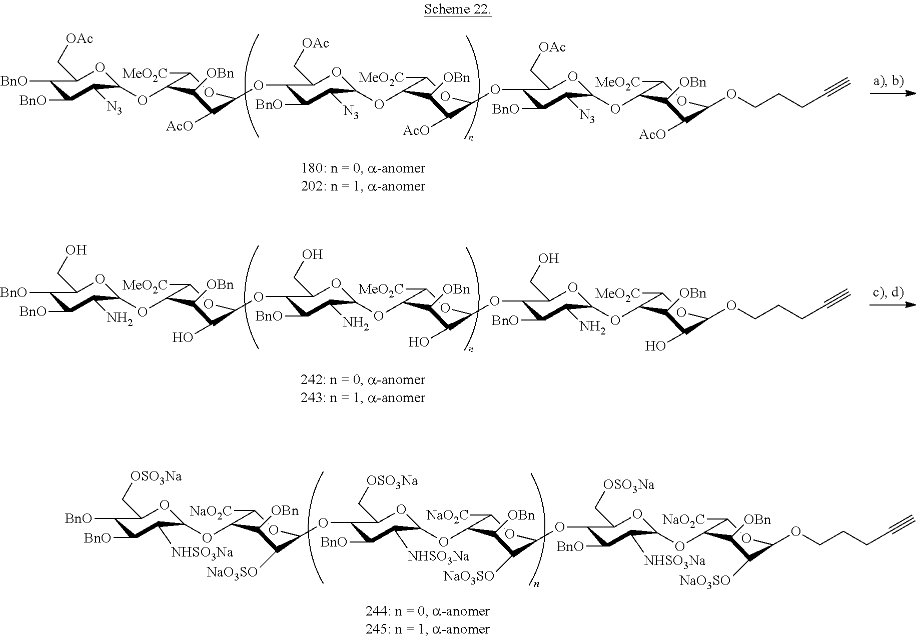

Examples 244 and 245 were prepared exactly following the same procedure used to get example 236.

Step 22.a+b: Synthesis of compound 242: Deacetylation of compound 180 (261 mg, 0.179 mmol) followed by selective azide reduction were successively performed according to the general method I and L. Purification was effected by chromatography on silica gel column (dichloromethane/methanol: 100/0 to 95/5 with 1% Et₃N) to give compound 242 (182 mg, 82% over 2 steps) as a white amorphous compound. ¹H NMR (400 MHz, CDCl₃, ppm): δ=7.43-7.14 (m, 25H, arom.), 5.28 (d, 1H, J=3.7 Hz, H-1 IdoUA^{III}), 5.04-4.85 (m, 7H, H-1 IdoUA^{I}, H-5 IdoUA^{III}, H-5 IdoUA^{I}, H-1 Glc^{IV}, H-1 Glc^{II}, CH₂-Ph), 4.81 (d, 1H, J=11.0 Hz, CH-Ph), 4.72 (dd, 2H, J=11.6 Hz, CH₂-Ph), 4.64 (dd, 2H, J=11.4 Hz, CH₂-Ph), 4.56 (t, 2H, J=11.6 Hz, CH₂-Ph), 4.42 (d, 1H, J=11.4 Hz, CH-Ph), 4.24-4.16 (m, 2H, H-4 IdoUA^{III}, H-4 IdoUA^{I}), 3.99 (t, 1H, J=9.6 Hz, H-4 Glc^{IV}), 3.96-3.86 (m, 3H, H-3 IdoUA^{I}, H-3 IdoUA^{III}, H-6a Glc^{IV}), 3.84-3.39 (m, 18H, H-2 IdoUA^{I}, H-2 IdoUA^{III}, H-6b Glc^{IV}, H-3 Glc^{IV}, H-5 Glc^{IV}, H-6a/b Glc^{II}, H-5 Glc^{II}, H-4 Glc^{II}, H-3 Glc^{II}, CH₂₍ₐ₎-pent-4-ynyl, 2×CO₂Me), 2.89 (dd, 1H, J=9.7 Hz, J=3.8 Hz, H-2 Glc^{IV}), 2.80 (m, 1H, H-2 Glc^{II}), 2.30-2.23 (m, 2H, CH_{2(c)}-pent-4-ynyl), 1.91 (t, 1H, J=2.5 Hz, CH_{(d)}-alkyne), 1.89-1.76 (m, 2H, CH_{2(b)}-pent-4-ynyl). MALDI-MS, positive mode, m/z: 1237.32 [M], 1259.30 [M+Na⁺]. [α]_{D}²¹=+39.2 (c=0.53, CHCl₃).

Step 22.c+d: Synthesis of compound 244: O,N-Sulfation of compound 242 (44 mg, 0.035 mmol) followed by saponification were successively performed according to the general method M and N. Purification was effected by size exclusion (Sephadex G25 NaCl 0.2M, then G25 water) to give compound 244 (49.1 mg, 74% over 2 steps) as a white hygroscopic solid. ¹H NMR (400 MHz, D₂O, ppm): δ=7.64-7.25 (m, 25H, arom.), 5.43 (s, 1H, H-1 IdoUA^{III}), 5.34 (s, 1H, H-1 IdoUA^{I}), 5.32-5.25 (m, 2H, H-1 Glc^{IV}, H-1Glc^{II}), 5.07 (d, 1H, J=10.6 Hz, CH-Ph), 4.92 (d, 1H, J=10.8 Hz, CH-Ph), 4.81-4.53 (m, 10H, H-5 IdoUA^{III}, H-2 IdoUA^{III}, H-5 IdoUA^{I}, 3×CH₂-Ph, CH-Ph), 4.48-4.05 (m, 10H, CH-Ph, H-3 IdoUA^{III}, H-4 IdoUA^{III}, H-2 IdoUA^{I}, H-3 IdoUA^{I}, H-4 IdoUA^{I}, H-6a/b Glc^{II}, H-6a/b Glc^{IV}), 3.98-3.79 (m, 5H, H-4 Glc^{IV}, H-5 Glc^{IV}, H-3 Glc^{II}, H-5 Glc^{II}, CH₍ₐ₎-pent-4-ynyl), 3.76-3.59 (m, 3H, H-4 Glc^{II}, H-3 Glc^{IV}, CH₍ₐ₎-pent-4-ynyl), 3.49-3.38 (m, 2H, H-2 Glc^{II}, H-2 Glc^{IV}), 2.36 (sl, 1H, CH_{(d)}-alkyne), 2.29-2.20 (m, 2H, CH_{2(c)}-pent-4-ynyl), 1.89-1.76 (m, 2H, CH_{2(b)}-pent-4-ynyl). ESI-MS, negative mode, m/z: 972.28 [M+2 DBA-4H]²⁻, 907.71 [M+1 DBA-3H]²⁻, 843.14 [M-2H]²⁻, 604.81 [M+1 DBA-4H]³⁻, 561.75 [M-3H]³⁻. [c]_{D}²¹=+12.0 (c=1.0, H₂O).

Preparation of example 245 was carried out as described for example 244.

Compound 245: ¹H NMR (400 MHz, D₂O, ppm): δ=7.73-7.22 (m, 35H, arom.), 5.54 (sl, 1H, H-1 IdoUA^{III}), 5.44 (sl, 1H, H-1 IdoUA^{V}), 5.36-5.24 (m, 4H, H-1 Glc^{VI}, H-1 IdoUA^{I}, H-1 Glc^{IV}, H-1 Glc^{II}), 5.08 (d, 1H, J=10.8 Hz, CH-Ph), 4.89 (d, 1H, J=10.8 Hz, CH-Ph), 4.85-4.51 (m, 17H, H-5 IdoUA^{V}, H-5 IdoUA^{III}, H-5 IdoUA^{I}, H-2 IdoUA^{V}, H-2 IdoUA^{III}, 5×CH₂-Ph, 2×CH-Ph), 4.50-4.13 (m, 13H, H-3 IdoUA^{III}, H-4 IdoUA^{III}, H-3 IdoUA^{V}, H-4 IdoUA^{V}, H-4 IdoUA^{I}, H-3 IdoUA^{I}, H-2 IdoUA^{I}, H-6a/b Glc^{VI}, H-6a/b Glc^{IV}, H-6a/b Glc^{II}), 4.59-3.59 (m, 11H, CH₂₍ₐ₎-pent-4-ynyl, H-3 Glc^{VI}, H-3 Glc^{II}, H-3 Glc^{IV}, H-4 Glc^{VI}, H-4 Glc^{II}, H-4 Glc^{IV}, H-5 Glc^{VI}, H-5 Glc^{II}, H-5 Glc^{IV}), 3.50-3.38 (m, 3H, H-2 Glc^{VI}, H-2 Glc^{IV}, H-2 Glc^{II}), 2.37 (t, 1H, J=2.2 Hz, CH_{(d)}-alkyne), 2.29-2.20 (m, 2H, CH_{2(c)}-pent-4-ynyl), 1.92-1.64 (m, 2H, CH_{2(b)}-pent-4-ynyl). ESI-MS, negative mode, m/z: 943.92 [M+3 DBA-6H]³⁻, 900.88 [M+2 DBA-5H]³⁻, 857.84 [M+1 DBA-4H]³⁻, 814.79 [M-3H]³⁻, 643.14 [M+1 DBA-5H]⁴⁻, 610.84 [M-4H]⁵⁻. [α]_{D}²¹=+29.7 (c=0.87, H₂O).

2. Preparation of Partially Benzylated and Methylated Compounds

a) Preparation of examples 246 to 260 (Scheme 23)

Below is reported the general formula of all the sulfated partially benzylated and methylated hexasaccharides synthesized.

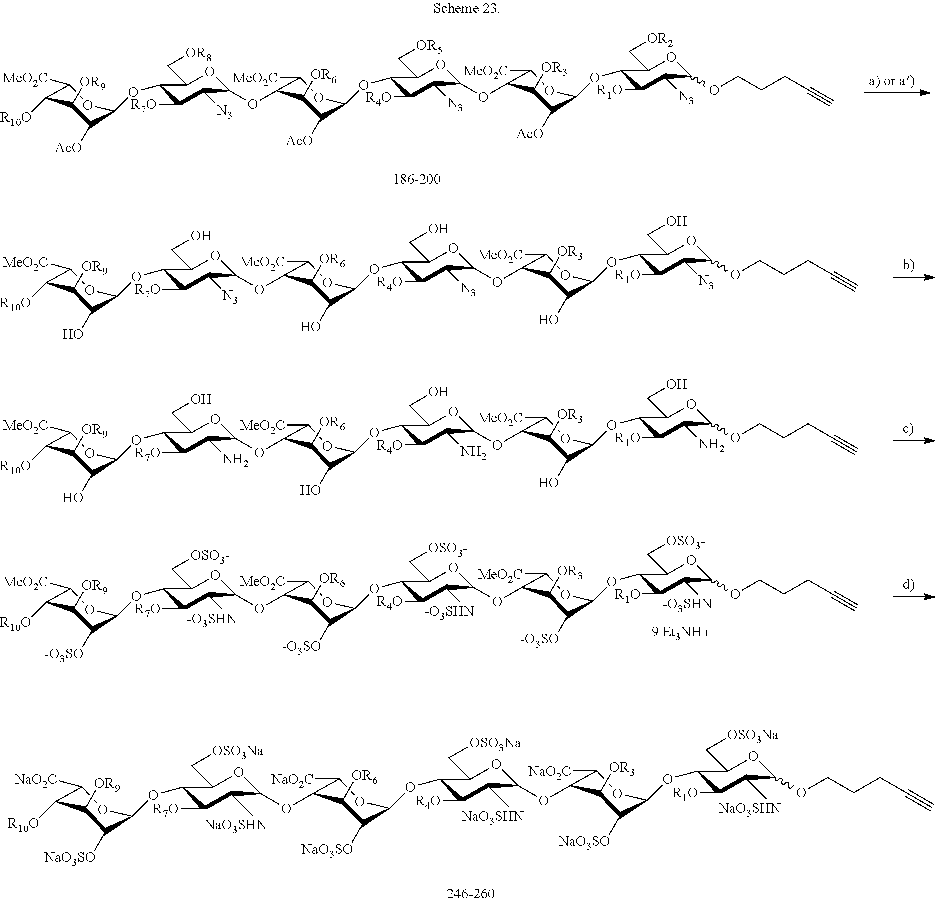

Examples 246 to 260 were prepared from protected hexasaccharides 186 to 200 in a similar manner as described for hexasaccharides 238 or 239 full benzylated, except for compounds 186 to 190, 194, 195, 197, 198, 199 and 200 for which step a′) was successively a desilylation reaction (method K) followed by a deacetylation reaction (method J).

| Compound | R₁ | R₃ | R₄ | R₆ | R₇ | R₉/R₁₀ | anomer |
|---|---|---|---|---|---|---|---|
| 246 | Me | Bn | Me | Bn | Me | Bn | β |
| 247 | Bn | Me | Bn | Me | Bn | Me | β |
| 248 | Me | Me | Me | Me | Me | Me | β |
| 249 | Me | Bn | Bn | Bn | Bn | Bn | β |
| 250 | Me | Me | Bn | Bn | Bn | Bn | β |
| 251 | Bn | Bn | Bn | Bn | Bn | Me | β |
| 252 | Bn | Bn | Me | Me | Bn | Bn | β |
| 253 | Bn | Bn | Bn | Bn | Me | Me | β |
| 254 | Me | Bn | Bn | Bn | Bn | Me | β |
| 255 | Me | Me | Me | Bn | Bn | Bn | β |
| 256 | Bn | Bn | Bn | Me | Me | Me | β |
| 257 | Me | Me | Bn | Bn | Bn | Me | β |
| 258 | Me | Bn | Bn | Bn | Me | Me | β |
| 259 | Bn | Me | Me | Me | Me | Bn | β |
| 260 | Me | Me | Bn | Bn | Me | Me | β |

Compound 246: ¹H NMR (400 MHz, D₂O, ppm): δ=7.52-7.13 (m, 20H, arom.), 5.24-5.17 (m, 2H, H-1 IdoUA^{IV}, H-1 IdoUA^{II}), 5.15-5.11 (m, 3H, H-1 Glc^{III}, H-1 Glc^{V}), 5.09 (br.s, 1H, H-1 IdoUA^{VI}), 4.49 (m, 1H, H-1 Glc^{I}), 3.89, 3.69 (m, 2H, CH₂₍ₐ₎-pent-4-ynyl), 3.44, 3.43, 3.39 (3s, 9H, OMe), 2.33-2.23 (m, 3H, CH_{2(c)}-pent-4-ynyl, CH_{(d)}-alkyne), 1.88-1.80 (m, 2H, CH_{2(b)}-pent-4-ynyl). EsI-ms, negative mode, m/z: 1367.42 [M+4 DBA-6H]²⁻, 1302.84 [M+3 DBA-5H]²⁻, 1238.26 [M+2 DBA-4H]²⁻, 1173.68 [M+DBA-3H]²⁻, 825.14 [M+2 DBA-5H]³⁻, 781.73 [M+DBA-4H]³⁻, 738.68 [M-3H]³⁻. [α]_{D}²¹=16.9 (c=0.63, H₂O).

Compound 247: ¹H NMR (400 MHz, D₂O, ppm): δ=7.55-7.22 (m, 15H, arom.), 5.33-5.20 (m, 5H, H-1 IdoUA^{IV}, H-1 IdoUA^{II}, H-1 Glc^{III}, H-1 Glc^{V}, H-1 IdoUA^{VI}), 4.75 (m, 1H, H-1 Glc^{I}), 3.92, 3.66 (m, 2H, CH₂₍ₐ₎-pent-4-ynyl), 3.51, 3.50, 3.47, 3.34 (4s, 12H, OMe), 2.33-2.20 (m, 3H, CH_{2(c)}-pent-4-ynyl, CH_{(d)}-alkyne), 1.81-1.71 (m, 2H, CH_{2(b)}-pent-4-ynyl). ESI-MS, negative mode, m/z: 1328.93 [M+4 DBA-6H]²⁻, 1264.35 [M+3 DBA-5H]²⁻, 1199.77 [M+2 DBA-4H]²⁻, 799.48 [M+2 DBA-5H]³⁻, 756.64 [M+DBA-4H]³⁻, 713.59 [M-3H]³⁻. [α]_{D}²¹=+17.5 (c=0.59, H₂O).

Compound 248: ¹H NMR (400 MHz, D₂O, ppm): δ=5.22-5.05 (m, 5H, H-1 IdoUA^{IV}, H-1 IdoUA^{II}, H-1 Glc^{III}, H-1 Glc^{V}, H-1 IdoUA^{VI}), 4.55 (m, 1H, H-1 Glc^{I}), 3.91, 3.68 (m, 2H, CH₂₍ₐ₎-pent-4-ynyl), 3.49 (6s, 18H, 6×OMe), 3.38 (s, 3H, OMe), 2.34-2.24 (m, 3H, CH_{2(c)}-pent-4-ynyl, CH_{(d)}-alkyne), 1.81-1.73 (m, 2H, CH_{2(b)}-pent-4-ynyl). ESI-MS, negative mode, m/z: 1215.31 [M+4 DBA-6H]²⁻, 1150.73 [M+3 DBA-5H]²⁻, 1086.15 [M+2 DBA-4H]²⁻, 723.74 [M+2 DBA-5H]³⁻, 680.33 [M+DBA-4H]³⁻, 637.26 [M-3H]³⁻. [α]_{D}²¹=+7.1 (c=0.51, H₂O).

Compound 249: ¹H NMR (400 MHz, D₂O, ppm): δ=7.65-7.26 (m, 30H, arom.), 5.59-5.24 (m, 5H, H-1 IdoUA^{II}, H-1 IdoUA^{IV}, H-1 IdoUA^{VI}, H-1 Glc^{III}, H-1 Glc^{V}), 4.62 (m, 1H, H-1 Glc^{I}), 3.54 (s, 3H, OMe), 4.00, 3.78 (m, 2H, CH₂₍ₐ₎-pent-4-ynyl), 2.34-2.14 (m, 3H, CH_{(d)}-alkyne, CH_{2(c)}-pent-4-ynyl), 1.90-1.80 (m, 2H, CH_{2(b)}-pent-4-ynyl). ESI-MS, negative mode, m/z: 874.85 [M+2 DBA-5H]³⁻, 831.80 [M+1 DBA-4H]³⁻, 591.30 [M-4H]⁴⁻. [α]_{D}²¹=+5.2 (c=1.84, H₂O).

Compound 250: ¹H NMR (400 MHz, D₂O, ppm): δ=7.67-7.15 (m, 25H, arom.), 5.59-5.24 (m, 5H, H-1 IdoUA^{II}, H-1 IdoUA^{IV}, H-1 IdoUA^{VI}, H-1 Glc^{III}, H-1 Glc^{V}), 4.63 (m, 1H, H-1 Glc^{I}), 3.57 (s, 6H, OMe), 3.96, 3.75 (m, 2H, CH₂₍ₐ₎-pent-4-ynyl), 2.42-2.33 (m, 3H, CH_{(d)}-alkyne, CH_{2(c)}-pent-4-ynyl), 1.90-1.81 (m, 2H, CH_{2(b)}-pent-4-ynyl). ESI-MS, negative mode, m/z: 892.56 [M+3 DBA-6H]³⁻, 849.51 [M+2 DBA-5H]³⁻, 806.45 [M+1 DBA-4H]³⁻, 736.75 [M-SO₃-3H]³⁻, 572.30 [M-4H]⁴⁻. [α]_{D}²¹=+0.7 (c=0.98, H₂O).

Compound 251: ¹H NMR (400 MHz, D₂O, ppm): δ=7.67-7.27 (m, 25H, arom.), 5.50 (sl, 1H, H-1 IdoUA^{II}), 5.42 (sl, 1H, H-1 IdoUA^{IV}), 5.30 (d, 1H, J=3.2 Hz, H-1 Glc^{III}), 5.27 (sl, 1H, H-1 IdoUA^{VI}), 5.21 (d, 1H, J=3.2 Hz, H-1 Glc^{V}), 4.76 (m, 1H, H-1 Glc^{I}), 4.98-4.73 (m, 8H, 4×CH₂-Ph), 4.67-4.55 (m, 2H, CH₂-Ph), 4.01, 3.79 (m, 2H, CH₂₍ₐ₎-pent-4-ynyl), 3.56, 3.44 (2s, 6H, OMe), 2.42-2.34 (m, 3H, CH_{2(c)}-pent-4-ynyl, CH_{(d)}-alkyne), 1.90-1.80 (m, 2H, CH_{2(b)}-pent-4-ynyl). ESI-MS, negative mode, m/z: 892.55 [M+3 DBA-6H]³⁻, 849.50 [M+2 DBA-5H]³⁻, 806.45 [M+1 DBA-4H]³⁻, 736.75 [M-SO₃-3H]³⁻, 572.29 [M-4H]⁴⁻. [α]_{D}²¹=+6.5 (c=1.90, H₂O).

Compound 252: ¹H NMR (400 MHz, D₂O, ppm): δ=7.58-7.26 (m, 25H, arom.), 5.34-5.24 (m, 4H, H-1 IdoUA^{II}, H-1 IdoUA^{IV}, H-1 IdoUA^{VI}, H-1 Glc^{III}), 5.13 (d, 1H, J=3.4 Hz, H-1 Glc^{V}), 4.73 (m, 1H, H-1 Glc^{I}), 4.82-4.47 (m, 10H, 5×CH₂-Ph), 3.98, 3.76 (m, 2H, CH₂₍ₐ₎-pent-4-ynyl), 3.55, 3.53 (2s, 6H, OMe), 2.38-2.30 (m, 3H, CH_{2(c)}-pent-4-ynyl, CH_{(d)}-alkyne), 1.87-1.77 (m, 2H, CH_{2(b)}-pent-4-ynyl). ESI-MS, negative mode, m/z: 849.50 [M+2 DBA-5H]³⁻, 806.78 [M+1 DBA-4H]³⁻, 572.29 [M-4H]⁴⁻. [α]_{D}²¹=+1.2 (c=2.80, H₂O).

Compound 253: ¹H NMR (400 MHz, D₂O, ppm): δ=7.61-7.26 (m, 20H, arom.), 5.45-5.28 (m, 2H, H-1 IdoUA^{II}, H-1 IdoUA^{IV}), 5.25 (d, 1H, J=3.2 Hz, H-1 Glc^{III}), 5.22 (d, 1H, J=3.1 Hz, H-1 Glc^{V}), 5.13 (s, 1H, H-1 IdoUA^{VI}), 4.76 (m, 1H, H-1 Glc^{I}), 4.95-4.73 (m, 6H, 3×CH₂-Ph), 4.63-4.53 (m, 2H, CH₂-Ph), 4.00, 3.78 (m, 2H, CH₂₍ₐ₎-pent-4-ynyl), 3.55, 3.51, 3.43 (3s, 9H, OMe), 2.41-2.33 (m, 3H, CH_{2(c)}-pent-4-ynyl, CH_{(d)}-alkyne), 1.87-1.77 (m, 2H, CH_{2(b)}-pent-4-ynyl). ESI-MS, negative mode, m/z: 824.50 [M+2 DBA-5H]³⁻, 781.46 [M+1 DBA-4H]³⁻, 713.75 [M-SO₃-3H]³⁻. [α]_{D}²¹=+8.6 (c=0.91, H₂O).

Compound 254: ¹H NMR (400 MHz, D₂O, ppm): δ=7.61-7.29 (m, 20H, arom.), 5.46 (sl, 1H, H-1 IdoUA^{II}), 5.35-5.23 (m, 4H, H-1 IdoUA^{IV}, H-1 IdoUA^{VI}, H-1 Glc^{III}, H-1 Glc^{V}), 4.96-4.73 (m, 6H, 3×CH₂-Ph), 4.66-4.52 (m, 2H, CH₂-Ph), 4.62 (m, 1H, H-1 Glc^{I}), 4.00, 3.77 (m, 2H, CH₂₍ₐ₎-pent-4-ynyl), 3.56, 3.53, 3.43 (3s, 9H, OMe), 2.41-2.31 (m, 3H, CH_{2(c)}-pent-4-ynyl, CH_{(d)}-alkyne), 1.90-1.78 (m, 2H, CH_{2(b)}-pent-4-ynyl). ESI-MS, negative mode, m/z: 1365.89 [M+4 DBA-6H]²⁻, 867.20 [M+3 DBA-6H]³⁻, 824.15 [M+2 DBA-5H]³⁻, 781.10 [M+1 DBA-4H]³⁻, 711.40 [M-SO₃-3H]³⁻, 553.28 [M-4H]⁴⁻. [α]_{D}²¹=+10.0 (c=1.53, H₂O).

Compound 255: ¹H NMR (400 MHz, D₂O, ppm): δ=7.64-7.15 (m, 20H, arom.), 5.42-5.20 (m, 5H, H-1 IdoUA^{II}, H-1 IdoUA^{IV}, H-1 Glc^{III}, H-1 IdoUA^{VI}, H-1 Glc^{V}), 4.66 (m, 1H, H-1 Glc^{I}), 4.95-4.63 (m, 8H, 4×CH₂-Ph), 4.01, 3.77 (m, 2H, CH₂₍ₐ₎-pent-4-ynyl), 3.58, 3.57, 3.49 (3s, 9H, OMe), 2.43-2.33 (m, 3H, CH_{2(c)}-pent-4-ynyl, CH_{(d)}-alkyne), 1.91-1.82 (m, 2H, CH_{2(b)}-pent-4-ynyl). ESI-MS, negative mode, m/z: 1365.91 [M+4 DBA-6H]²⁻, 867.21 [M+3 DBA-6H]³⁻, 824.16 [M+2 DBA-5H]³⁻, 781.12 [M+1 DBA-4H]³⁻, 711.40 [M-SO₃-3H]³⁻, 553.30 [M-4H]⁴⁻. [α]_{D}²¹=4.2 (c=1.0, H₂O).

Compound 256: ¹H NMR (400 MHz, D₂O, ppm): δ=7.65-7.33 (m, 15H, arom.), 5.36 (sl, 2H, H-1 IdoUA^{II}, H-1 IdoUA^{IV}), 5.27 (d, 1H, J=3.0 Hz, H-1 Glc^{III}), 5.23 (d, 1H, J=3.0 Hz, H-1 Glc^{V}), 5.13 (s, 1H, H-1 IdoUA^{VI}), 4.90-4.71 (m, 6H, 3×CH₂-Ph), 4.75 (m, 1H, H-1 Glc^{I}), 3.99, 3.76 (m, 2H, CH₂₍ₐ₎-pent-4-ynyl), 3.58, 3.56, 3.54, 3.45 (4s, 12H, OMe), 2.40-2.29 (m, 3H, CH_{2(c)}-pent-4-ynyl, CH_{(d)}-alkyne), 1.90-1.80 (m, 2H, CH_{2(b)}-pent-4-ynyl). ESI-MS, negative mode, m/z: 1327.90 [M+4 DBA-6H]²⁻, 798.82 [M+2 DBA-5H]³⁻, 755.77 [M+1 DBA-4H]³⁻, 686.07 [M-SO₃.3H]³⁻, 534.28 [M-4H]⁴⁻. [α]_{D}²¹=+12.2 (c=1.0, H₂O).

Compound 257: ¹H NMR (400 MHz, D₂O, ppm): δ=7.65-7.31 (m, 15H, arom.), 5.52 (sl, 1H, H-1 IdoUA^{II}), 5.37-5.25 (m, 4H, H-1 IdoUA^{IV}, H-1 Glc^{III}, H-1 IdoUA^{VI}, H-1 Glc^{V}), 4.97-4.76 (m, 6H, 3×CH₂-Ph), 4.68 (m, 1H, H-1 Glc^{I}), 4.02, 3.80 (m, 2H, CH₂₍ₐ₎-pent-4-ynyl), 3.65-3.56 (3s, 9H, OMe), 3.45 (s, 3H, OMe), 2.47-2.30 (m, 3H, CH_{2(c)}-pent-4-ynyl, CH_{(d)}-alkyne), 1.94-1.78 (m, 2H, CH_{2(b)}-pent-4-ynyl). ESI-MS, negative mode, m/z: 1327.91 [M+4 DBA-6H]²⁻, 841.88 [M+3 DBA-6H]³⁻, 798.83 [M+2 DBA-5H]³⁻, 755.78 [M+1 DBA-4H]³⁻, 712.72 [M-3H]³⁻, 534.29 [M-4H]⁴⁻.

Compound 258: ¹H NMR (400 MHz, D₂O, ppm): δ=7.60-7.29 (m, 15H, arom.), 5.41 (sl, 1H, H-1 IdoUA^{II}), 5.33-5.28 (m, 2H, H-1 IdoUA^{IV}, H-1 Glc^{III}), 5.26 (d, 1H, J=3.3 Hz, H-1 Glc^{V}), 5.12 (sl, 1H, H-1 IdoUA^{VI}), 4.93-4.72 (m, 4H, 2×CH₂-Ph), 4.62-4.58 (m, 2H, CH₂-Ph), 4.61 (m, 1H, H-1 Glc^{I}), 3.99, 3.77 (m, 2H, CH₂₍ₐ₎-pent-4-ynyl), 3.56, 3.53, 3.52, 3.45 (4s, 12H, OMe), 2.40-2.34 (m, 3H, CH_{2(c)}-pent-4-ynyl, CH_{(d)}-alkyne), 1.89-1.81 (m, 2H, CH_{2(b)}-pent-4-ynyl). ESI-MS, negative mode, m/z: 842.44 [M+3 DBA-6H]³⁻, 799.40 [M+2 DBA-5H]³⁻, 756.35 [M+1 DBA-4H]³⁻, 713.31 [M-3H]³⁻, 534.22 [M-4H]⁴⁻. [α]_{D}²¹=+3.5 (c=0.92, H₂O).

Compound 259: ¹H NMR (400 MHz, D₂O, ppm): δ=7.55-7.29 (m, 15H, arom.), 5.34 (sl, 1H, H-1 IdoUA^{II}), 5.32-5.23 (m, 3H, H-1 IdoUA^{IV}, H-1 Glc^{III}, H-1 Glc^{V}), 5.18 (sl, 1H, H-1 IdoUA^{VI}), 4.81-4.73 (m, 2H, CH₂-Ph), 4.71, 4.61 (2d, 2H, J=12.3 Hz, CH₂-Ph), 4.47-4.40 (m, 2H, CH₂-Ph), 4.78 (m, 1H, H-1 Glc^{I}), 4.00, 3.76 (m, 2H, CH₂₍ₐ₎-pent-4-ynyl), 3.58, 3.57, 3.56, 3.53 (4s, 12H, OMe), 2.43-2.29 (m, 3H, CH_{2(c)}-pent-4-ynyl, CH_{(d)}-alkyne), 1.90-1.80 (m, 2H, CH_{2(b)}-pent-4-ynyl). ESI-MS, negative mode, m/z: 755.86 [M+1 DBA-4H]³⁻, 534.35 [M-4H]⁴⁻.

Compound 260: ¹H NMR (400 MHz, D₂O, ppm): δ=7.58-7.29 (m, 10H, arom.), 5.47 (sl, 1H, H-1 IdoUA^{II}), 5.36-5.30 (m, 2H, H-1 IdoUA^{IV}, H-1 Glc^{III}), 5.25 (d, 1H, J=3.2 Hz, H-1 Glc^{V}), 5.14 (sl, 1H, H-1 IdoUA^{VI}), 4.89, 4.73 (2d, 2H, J=11.0 Hz, CH₂-Ph), 4.65 (m, 1H, H-1 Glc^{I}), 4.60 (sl, 2H, CH₂-Ph), 4.00, 3.78 (m, 2H, CH₂₍ₐ₎-pent-4-ynyl), 3.60-3.52 (m, 12H, OMe), 3.46 (s, 3H, OMe), 2.43-2.33 (m, 3H, CH_{2(c)}-pent-4-ynyl, CH_{(d)}-alkyne), 1.91-1.81 (m, 2H, CH_{2(b)}-pent-4-ynyl). ESI-MS, negative mode, m/z: 1289.88 [M+4 DBA-6H]²⁻, 1225.25 [M+3 DBA-5H]²⁻, 816.52 [M+3 DBA-6H]³⁻, 773.47 [M+2 DBA-5H]³⁻, 730.42 [M+1 DBA-4H]³⁻, 687.37 [M-3H]³⁻, 660.72 [M-SO₃-3H]³⁻, 515.28 [M-4H]⁴⁻. [α]_{D}²¹=+5.6 (c=1.27, H₂O).

b) In Preparation of Examples 261, 262 and 263 (Scheme 24)

Below is reported the general formula of all the sulfated partially benzylated and methylated octasaccharides synthesized.

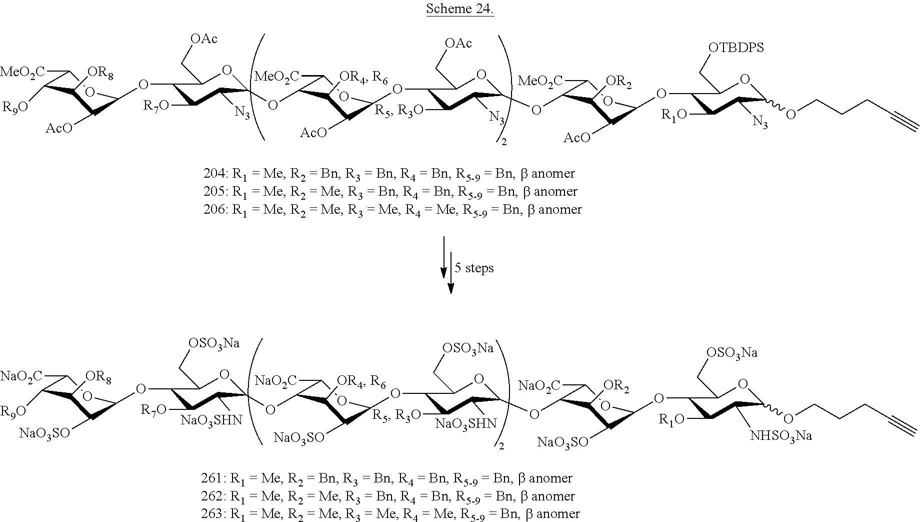

Examples 261, 262, 263 were prepared respectively from octasaccharides 204, 205 and 206 and were carried out as described for compound 240 in 5 steps (desilylation, deacetylation, selective azide reduction, O,N-sulfation and saponification reactions).

Compound 261: ¹H NMR (400 MHz, D₂O, ppm); ESI-MS, negative mode, m/z: 1300.25 [M+6 DBA-9H]³⁻, 1257.20 [M+5 DBA-8H]³⁻, 1214.14 [M+4 DBA-7H]³⁻, 910.35 [M+4 DBA-8H]⁴⁻, 878.06 [M+3 DBA-7H]⁴⁻, 845.77 [M+2 DBA-6H]⁴⁻, 813.47 [M+DBA-5H]⁴⁻, 624.74 [M-5H]⁵⁻. [α]_{D}²¹=+3.3 (c=0.66, H₂O).

Compound 262: ¹H NMR (400 MHz, D₂O, ppm); ESI-MS, negative mode, m/z: 1274.96 [M+5 DBA-8H]³⁻, 1231.91 [M+4 DBA-7H]³⁻, 1188.85 [M+3 DBA-6H]³⁻, 1145.80 [M+2 DBA-5H]³⁻, 891.38 [M+3 DBA-7H]⁴⁻, 859.09 [M+2 DBA-6H]⁴⁻, 826.79 [M+DBA-5H]⁴⁻, 794.50 [M-4H]⁴⁻. [α]_{D}²¹=+4.5 (c=1.5, H₂O).

Compound 263: ¹H NMR (400 MHz, D₂O, ppm); ESI-MS, negative mode, m/z: 1181.52 [M+5 DBA-8H]³⁻, 1138.14 [M+4 DBA-7H]³⁻, 821.07 [M+3 DBA-7H]⁴⁻, 788.79 [M+2 DBA-6H]⁴⁻, 755.76 [M+1 DBA-5H]⁴⁻, 703.98 [M-SO₃-4H]⁴⁻, 578.97 [M-5H]⁵⁻.

3. Preparation of Partially Hydroxylated and Methylated Compounds

a) Preparation of Examples 266, 267 and 268 (Scheme 25)

Below is reported the general formula of all the sulfated partially hydroxylated and methylated compounds synthesized.

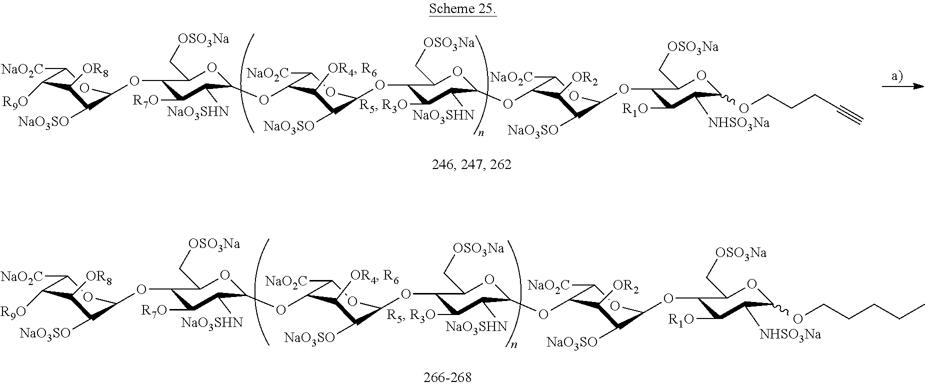

| Compound | n | R₁ | R₂ | R₃ | R₄ | R₅ | R₆ | R₇ | R_{8/}R₉ | anomer |
|---|---|---|---|---|---|---|---|---|---|---|
| 266 | 1 | Me | H | Me | H | / | / | Me | H/H | β |
| 267 | 1 | H | Me | H | Me | / | / | H | Me/Me | β |
| 268 | 2 | Me | Me | H | H | H | H | H | H/H | β |

Examples 266, 267 and 268 were prepared respectively from examples 246, 247 and 262 in one step by hydrogenolysis reaction.

Step 25.a: Synthesis of compound 266: Hydrogenolysis of compound 246 (22 mg, 11.8 mop was performed according to the general method P. Purification was effected by size exclusion (Sephadex G25 water) to give debenzylated compound 266 (14 mg, 84%) as a white hygroscopic solid. ¹H NMR (400 MHz, D₂O, ppm): δ=5.42-5.33 (m, 2H, H-1 Glc^{III}, H-1 Glc^{V}), 5.20-5.10 (m, 4H, H-1 Glc^{I}, H-1 IdoUA^{II}, H-1 IdoUA^{IV}, H-1 IdoUA^{VI}), 3.52, 3.51, 3.50 (3s, 9H, OMe), 1.64-1.54 (m, 2H, CH_{2(b)})pentyl), 1.34-1.25 (m, 4H, CH_{2(c,d)}-pentyl), 0.85 (t, 3H, J=6.9 Hz, CH₃-pentyl). ESI-MS, negative mode, m/z: 1188.35 [M+45 DBA-6H]²⁻, 1123.77 [M+3 DBA-5H]²⁻, 1059.19 [M+2 DBA-4H]²⁻, 705.75 [M+2 DBA-5H]³⁻, 662.69 [M+1 DBA-4H]³⁻, 619.64 [M-3H]³⁻.

Preparation of examples 267 and 268 were carried out as described for example 266.

Compound 267: ¹H NMR (400 MHz, D₂O, ppm): δ=5.28-5.10 (m, 5H, H-1 IdoUA^{IV}, H-1 IdoUA^{II}, H-1 Glc^{III}, H-1 Glc^{V}, H-1 IdoUA^{VI}), 4.49 (m, 1H, H-1 Glc^{I}), 3.80, 3.60 (m, 2H, CH₂₍ₐ₎-pentyl), 3.47 (3s, 9H, 3×OMe), 3.36 (s, 3H, OMe), 1.58-1.50 (m, 2H, CH_{2(b)}-pentyl), 1.30-1.19 (m, 4H, CH_{2(c,d)}-pentyl), 0.81 (t, 3H, J=6.8 Hz, CH₃-pentyl). ESI-MS, negative mode, m/z: 1260.34 [M+5 DBA-7H]²⁻, 1195.76 [M+4 DBA-6H]²⁻, 1131.18 [M+3 DBA-5H]²⁻, 1066.66 [M+2 DBA-4H]²⁻, 1002.02 [M+1 DBA-3H]²⁻, 710.37 [M+2 DBA-5H]³⁻, 667.32 [M+1 DBA-4H]³⁻, 624.25 [M-3H]³⁻.

Compound 268: ¹H NMR (400 MHz, D₂O, ppm); ESI-MS, negative mode, m/z: 1065.99 [M+6 DBA-9H]³⁻, 1022.93 [M+5 DBA-8H]³⁻, 979.87 [M+4 DBA-7H]³⁻, 734.66 [M+4 DBA-8H]⁴⁻, 702.37 [M+3 DBA-7H]⁴⁻, 670.08 [M+2 DBA-6H]⁴⁻, 637.78 [M+DBA-5H]⁴⁻, 483.81 [M-5H]⁵⁻. [α]_{D}²¹=+21.3 (c=1.02, H₂O).

C. Examples from N-Acylated Sulfated Oligosaccharides Family:

### 1. Preparation of Fully Benzylated Examples 274, 275, 276, 277 and 278 (Scheme 27)

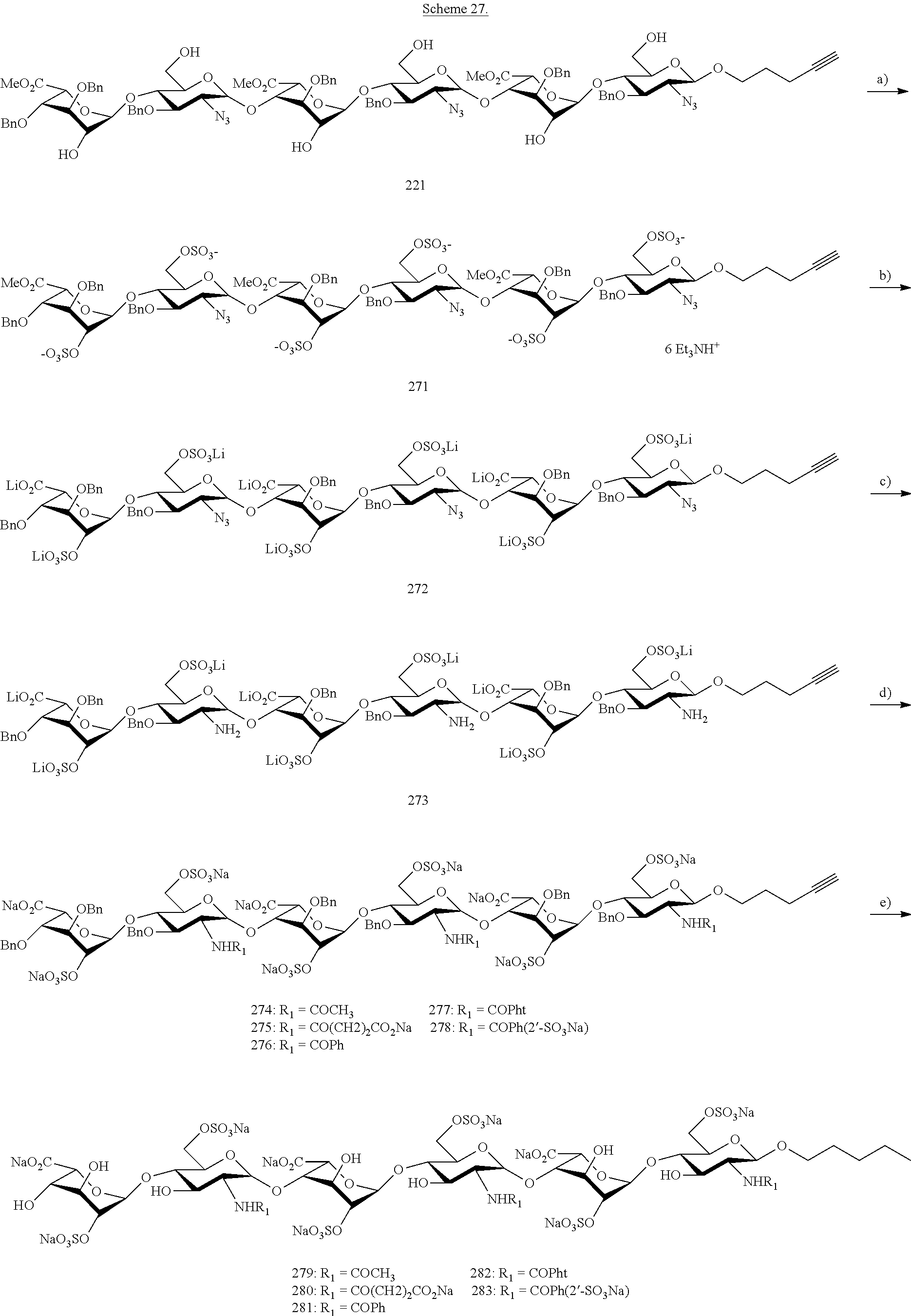

Step 27.a: Synthesis of compound 271: O-Sulfation of compound 221 (0.091 mmol) was performed according to the general method M. Purification was effected by size exclusion (Sephadex LH20 dichloromethane/ethanol: 1/1) to give compound 271 as a clear yellow oil. ESI-MS, negative mode, m/z: 1420.56 [M+4 DBA-6H]²⁻, 1355.97 [M+3 DBA-5H]²⁻, 1291.40 [M+2 DBA-4H]²⁻, 1226.82 [M+1 DBA 3H]²⁻, 774.50 [M-3H]³⁻.

Step 27.b: Synthesis of compound 272: Saponification of compound 271 (0.091 mmol) was performed according to the general method N in a 1/1 mixture of tetrahydrofurane/methanol (0.02 M). Purification was effected by size exclusion (Sephadex LH20 methanol/water: 100/1) to give the saponified compound 272 (196.1 mg, 92% over 3 steps) as a white solid. ¹H NMR (400 MHz, D₂O, ppm): δ=7.45-7.22 (m, 35H, arom.), 5.27 (s, 1H, H-1 IdoUA^{II}), 5.23-5.17 (m, 2H, H-1 IdoUA^{IV}, H-1 IdoUA^{VI}), 4.95 (d, 1H, J=3.5 Hz, H-1 Glc^{III}), 4.88-4.69 (m, 13H, H-1 Glc^{I}, H-5 IdoUA^{II}, H-5 IdoUA^{IV}, 5×CH₂-Ph), 4.65-4.48 (m, 5H, H-5 IdoUA^{VI}, 2×CH₂-Ph), 4.48-4.37 (m, 5H, H-1 Glc^{V}, H-2 IdoUA^{IV}, H-2 IdoUA^{VI}, H-2 IdoUA^{II}, H-3 IdoUA^{II}), 4.37-4.18 (m, 6H, H-6a/b Glc^{I}, H-6a/b Glc^{III}, H-6a/b Glc^{V}), 4.00-3.70 (m, 8H, CH₂₍ₐ₎-pent-4-ynyl, H-4 Glc^{V}, H-5 Glc^{V}, H-3 Glc^{I}, H-4 Glc^{I}, H-3 Glc^{III}, H-3 Glc^{V}) 3.47-3.39 (m, 2H, H-2 Glc^{I}, H-2 Glc^{III}), 3.32 (t, 1H, J=9.2 Hz, H-2 Glc^{V}), 2.29-2.22 (m, 2H, CH_{2(c)}-pent-4-ynyl), 1.80-1.70 (m, 2H, CH_{2(b)}-pent-4-ynyl, CH_{(d)}-alkyne). ESI-MS, negative mode, m/z: 1399.58 [M+3 DBA-5H]²⁻, 1335.00 [M+2 DBA-4H]²⁻, 1270.42 [M+1 DBA-3H]²⁻, 1205.83 [M-2H]²⁻, 760.84 [M-3H]³⁻.

Step 27.c: Synthesis of compound 273: Selective azide reduction of compound 272 (100 mg, 0.042 mmol) was performed according to the general method L at 40° C. Purification was effected by size exclusion (Sephadex LH20 methanol/water: 100/1) to give compound 273 (95 mg, 98%) as a white solid. ¹H NMR (400 MHz, D₂O, ppm): δ=7.48-7.17 (m, 35H, aromatic), 5.31 (s, 1H, H-1 IdoUA^{II}), 5.25 (s, 1H, H-1 IdoUA^{IV}), 5.23 (s, 1H, H-1 IdoUA^{VI}), 4.87-4.72 (m, 7H, H-1 Glc^{III}, H-1 Glc^{I}, H-5 IdoUA^{II}, H-5 IdoUA^{IV}, H-1 Glc^{V}, CH₂-Ph), 4.65-4.57 (m, 3H, H-5 IdoUA^{VI}, CH₂-Ph), 4.49-4.14 (m, 21H, H-2 IdoUA^{IV}, H-2 IdoUA^{VI}, H-2 IdoUA^{II}, H-4 IdoUA^{IV}, H-4 IdoUA^{VI}, 5×CH₂-Ph, H-6a/b Glc^{I}, H-6a/b Glc^{III}, H-6a/b Glc^{V}), 4.06-3.83 (m, 8H, CH₍ₐ₎-pent-4-ynyl, H-4 Glc^{I}, H-4 Glc^{III}, H-4 Glc^{V}, H-3 IdoUA^{IV}, H-3 IdoUA^{VI}, H-3 IdoUA^{II}, H-4 IdoUA^{II}), 3.70-3.61 (m, 3H, CH_{(a′)}-pent-4-ynyl, H-3 Glc^{III}, H-3 Glc^{I}), 3.40 (t, 1H, J=9.2 Hz, H-3 Glc^{V}), 2.71-2.61 (m, 3H, H-2 Glc^{III}, H-2 Glc^{I}, H-2 Glc^{V}), 2.28-2.20 (m, 2H, CH_{2(c)}-pent-4-ynyl), 1.79-1.69 (m, 2H, CH_{2(b)}-pent-4-ynyl). ESI-MS, negative mode, m/z: 1231.31 [M+2 DBA-4H]²⁻, 1166.74 [M+1 DBA-3H]²⁻, 1120.16 [M-2H]²⁻, 734.44 [M-3H]³⁻, 550.57 [M-4H]⁴⁻.

Step 27.d: Synthesis of compound 274: N-acylation of compound 273 (20.1 mg, 8.89 mop was performed according to the general method R with acetic anhydride reagent. Purification was effected by size exclusion (Sephadex G25 NaCl 0.2M, then G25 water) to give compound 274 (18.8 mg, 83%) as a white hygroscopic solid. ¹H NMR (400 MHz, D₂O, ppm): δ=7.56-7.17 (m, 35H, arom.), 5.39-5.25 (m, 3H, H-1 IdoUA^{II}, H-1 IdoUA^{IV}, H-1 IdoUA^{VI}), 4.98-4.75 (m, 5H, CH₂-Ph, H-5 IdoUA^{II}, H-5 IdoUA^{IV}, CH-Ph), 4.79-4.66 (m, 2H, CH-Ph, H-5 IdoUA^{VI}), 4.57-4.28 (m, 14H, H-2 IdoUA^{IV}, H-2 IdoUA^{VI}, H-2 IdoUA^{II}, 5×CH₂-Ph, H-1 Glc^{I}), 4.22-3.65 (m, 10H, H-3 IdoUA^{IV}, H-3 IdoUA^{VI}, H-3 IdoUA^{II}, H-4 IdoUA^{IV}, H-4 IdoUA^{VI}, H-4 IdoUA^{II}, CH₂₍ₐ₎-pent-4-ynyl, H-2 Glc^{I}, H-3 Glc^{I}), 2.35 (t, 1H, J=2.3 Hz, CH_{(d)}-alkyne), 2.27-2.20 (m, 2H, CH_{2(c)}-pent-4-ynyl), 1.77-1.68 (3s+m, 11H, CH_{2(b)}-pent-4-ynyl, 3×CH₃—NHAc). ESI-MS, negative mode, m/z: 1424.14 [M+4 DBA-6H]²⁻, 1359.06 [M+3 DBA-5H]²⁻, 1294.52 [M+2 DBA-4H]²⁻, 1254.49 [M+1 DBA-3H]²⁻, 905.72 [M+3 DBA-6H]³⁻, 863.00 [M+2 DBA-5H]³⁻, 819.59 [M+1 DBA-4H]³⁻, 776.55 [M-3H]³⁻. [α]_{D}²¹=+1.6 (c=0.58, H₂O).

Preparation of examples 275, 276, 277 and 278 were carried out as described for example 274.

Synthesis of compound 275: Compound 275 was prepared from 273 according to the general method R with succinic anhydride reagent (yield: 68%). ¹H NMR (400 MHz, D₂O, ppm): δ=7.55-7.18 (m, 35H, aromatic), 5.43-5.28 (m, 3H, H-1 IdoUA^{IV}, H-1 IdoUA^{II}, H-1 IdoUA^{VI}), 5.08 (br. s, 1H, H-1 Glc^{II}), 3.98, 3.70 (m, 2H, CH₂₍ₐ₎-pent-4-ynyl), 2.43-1.96 (m, 15H, 3×(CH₂)₂-succinate, CH_{2(c)}-pent-4-ynyl, CH_{(d)}-alkyne), 1.82-1.75 (m, 2H, CH_{2(b)}-pent-4-ynyl). ESI-MS, negative mode, m/z: 1511.41 [M+4 DBA-6H]²⁻, 1446.80 [M+3 DBA-5H]²⁻, 1382.25 [M+2 DBA-4H]²⁻, 964.21 [M+3 DBA-6H]³⁻, 921.16 [M+2 DBA-5H]³⁻, 877.76 [M+1 DBA-4H]³⁻, 834.69 [M-3H]³⁻. [α]_{D}²¹=+8.9 (c=0.58, H₂O).

Synthesis of compound 276: Compound 276 was prepared from 273 according to the general method R with benzoic anhydride reagent (yield: 75%). ¹H NMR (400 MHz, D₂O, ppm): δ=7.67-7.0 (m, 50H, arom.), 5.40 (s, 1H, H-1 Glc^{III(V)}), 5.36 (d, 1H, J=2.1 Hz, H-1 Glc^{V(III)}), 5.30 (br. s, 1H, H-1 IdoUA^{IV}), 5.08 (d, 1H, J=3.5 Hz, H-1 IdoUA^{II}), 4.99 (d, 1H, J=3.6 Hz, H-1 IdoUA^{VI}), 4.74-4.66 (m, 1H, H-1 Glc^{I}), 4.01-3.74 (m, 2H, CH₂₍ₐ₎-pent-4-ynyl), 2.15-2.08 (m, 2H, CH_{2(c)}-pent-4-ynyl), 1.80-1.65 (m, 2H, CH_{2(b)}-pent-4-ynyl). ESI-MS, negative mode, m/z: 1517.54 [M+4 DBA-6H]²⁻, 1452.44 [M+3 DBA-5H]²⁻, 1387.85 [M+2 DBA-4H]²⁻, 1323.26 [M+1 DBA-3H]²⁻, 968.24 [M+3 DBA-6H]³⁻, 925.17 [M+2 DBA-5H]³⁻, 881.78 [M+1 DBA-4H]³⁻, 838.72 [M-3H]³⁻. [α]_{D}²¹=+23.3 (c=0.58, H₂O).

Synthesis of compound 277: Compound 277 was prepared from 273 according to the general method R with phthalic anhydride reagent (yield: 84%). ¹H NMR (400 MHz, D₂O, ppm): δ=7.92-6.85 (m, 47H, arom.), 5.41 (br. s, 2H, H-1 IdoUA^{IV}, H-1 IdoUA^{II}), 5.10 (br. s, 3H, H-1 IdoUA^{VI}, H-1 Glc^{V}, H-1 Glc^{II}), 4.00-3.70 (m, 2H, CH₂₍ₐ₎-pent-4-ynyl), 2.34-2.27 (m, 3H, CH_{2(c)}-pent-4-ynyl, CH_{(d)}-alkyne), 1.87-1.78 (m, 2H, CH_{2(b)}-pent-4-ynyl). ESI-MS, negative mode, m/z: 1648.13 [M+5 DBA-7H]²⁻, 1583.55 [M+4 DBA-6H]²⁻, 1518.93 [M+3 DBA-5H]²⁻, 1454.33 [M+2 DBA-4H]²⁻, 1389.81 [M+1 DBA-3H]²⁻, 1012.30 [M+3 DBA-6H]³⁻, 969.23 [M+2 DBA-5H]³⁻, 926.14 [M+1 DBA-4H]³⁻, 883.07 [M-3H]³⁻. [α]_{D}²¹=+8.2 (c=0.58, H₂O).

Synthesis of compound 278: Compound 278 was prepared from 273 according to the general method R with 2-sulfobenzoic acid cyclic anhydride reagent (yield: 87%). ¹H NMR (400 MHz, D₂O, ppm): δ=7.95-7.84 (m, 2H, arom.), 7.64-7.12 (m, 43H, arom.), 6.86-6.69 (m, 2H, arom.), 5.41-5.30 (m, 3H, H-1 IdoUA^{IV}, H-1 IdoUA^{II}, H-1 Glc^{II}), 5.21-5.10 (m, 2H, H-1 IdoUA^{VI}, H-1 Glc^{V}), 3.99-3.82 (m, 2H, CH₂₍ₐ₎-pent-4-ynyl), 2.31-2.23 (m, 3H, CH_{(d)}-alkyne, CH_{2(c)}-pent-4-ynyl), 1.87-1.77 (m, 2H, CH_{2(b)}-pent-4-ynyl). ESI-MS, negative mode, m/z: 1091.64 [M+4 DBA-7H]³⁻, 1048.25 [M+3 DBA-6H]³⁻, 1005.18 [M+2 DBA-5H]³⁻, 962.12 [M+1 DBA-4H]³⁻, 919.04 [M-3H]³⁻, 689.02 [M-4H]⁴⁻. [α]_{D}²¹=+13.1 (c=0.58, H₂O).

### Preparation of Fully Hydroxylated Examples 279, 280, 281, 282 and 283 (Schema 27)

Step 27.e: Synthesis of compound 279: Hydrogenolysis of compound 274 (10.7 mg, 4.23 μmol) was performed according to the general method O and gave the debenzylated compound 279 (8.1 mg, quant.) as a white hygroscopic solid. ¹H NMR (400 MHz, D₂O, ppm): δ=5.23-5.14 (m, 5H, H-1 Glc^{II}, H-1 Glc^{V}, H-1 IdoUA^{II}, H-1 IdoUA^{IV}, H-1 IdoUA^{VI}), 4.96 (br. s, 1H, H-5 IdoUA^{II}), 4.86-4.82 (m, 2H, H-5 IdoUA^{IV}, H-5 IdoUA^{VI}), 4.55 (d, 1H, J=7.8 Hz, H-1 Glc^{I}), 4.44-4.25 (m, 9H, H-6a/b Glc^{II}, H-6a/b Glc^{I}, H-6a/b Glc^{V}, H-2 IdoUA^{IV}, H-2 IdoUA^{VI}, H-2 IdoUA^{II}), 4.16-3.98 (m, 9H, H-3 IdoUA^{IV}, H-3 IdoUA^{VI}, H-3 IdoUA^{II}, H-4 IdoUA^{IV}, H-4 IdoUA^{VI}, H-4 IdoUA^{II}, H-3 Glc^{I}, H-2 Glc^{III}, H-2 Glc^{V}), 3.95-3.87 (m, 1H, CH₍ₐ₎-pentyl), 3.85-3.68 (m, 5H, H-2 Glc^{I}, H-4 Glc^{I}, H-3 Glc^{III}, H-3 Glc^{V}, CH_{(a′)}-pentyl), 2.08 (s, 6H, CH₃—NHAc), 2.05 (s, 3H, CH₃—NHAc), 1.63-1.51 (m, 2H, CH_{2(b)}-pentyl), 1.36-1.28 (m, 4H, CH_{2(c,d)}-pentyl), 0.90 (t, 3H, J=6.4 Hz, CH₃-pentyl). ESI-MS, negative mode, m/z: 1109.84 [M+4 DBA-6H]²⁻, 1045.29 [M+3 DBA-5H]²⁻, 980.70 [M+2 DBA-4H]²⁻, 916.13 [M+1 DBA-3H]²⁻, 851.54 [M-2H]²⁻, 610.41 [M+1 DBA-4H]³⁻, 567.36 [M-3H]³⁻. [α]_{D}²¹=+15.1 (c=0.33, H₂O).

Preparation of examples 280, 281, 282 and 283 were carried out as described for example 279.

Synthesis of compound 280: Compound 280 was prepared from 275 according to the general method O (yield: 79%). ¹H NMR (400 MHz, D₂O, ppm): δ=5.26-5.13 (m, 5H, H-1 Glc^{IIIl}, H-1 Glc^{V}, H-1 IdoUA^{IV}, H-1 IdoUA^{II}, H-1 IdoUA^{VI}, 4.55 (d, 1H, J=7.3 Hz, H-1 Glc^{I}), 3.90, 3.64 (m, 2H, CH₂₍ₐ₎-pentyl), 2.67-2.45 (m, 12H, 3×(CH₂)₂-succinate), 1.62-1.53 (m, 2H, CH_{2(b)}-pentyl), 1.35-1.28 (m, 4H, CH_{2(c,d)}-pentyl), 0.90 (t, 3H, J=7.0 Hz, CH₃-pentyl). ESI-MS, negative mode, m/z: 1197.79 [M+4 DBA-6H]²⁻, 1132.71 [M+3 DBA-5H]²⁻, 1068.11 [M+2 DBA-4H]²⁻, 1003.53 [M+1 DBA-3H]²⁻, 938.95 [M-2H]²⁻, 711.68 [M+2 DBA-5H]³⁻, 668.63 [M+1 DBA-4H]³⁻, 625.54 [M-3H]³⁻. [α]_{D}²¹=+14.4 (c=0.34, H₂O).

Synthesis of compound 281: Compound 281 was prepared from 276 according to the general method O (yield: 80%). ¹H NMR (400 MHz, D₂O, ppm): δ=7.88-7.79 (m, 5H, arom.), 7.68-7.52 (m, 10H, arom.), 5.41-5.33 (m, 2H, H-1 Glc^{III}, H-1 Glc^{V}), 5.25-5.14 (m, 3H, H-1 IdoUA^{IV}, H-1 IdoUA^{II}, H-1 IdoUA^{VI}), 4.69 (d, 1H, J=8.0 Hz, H-1 Glc^{I}), 3.93, 3.63 (m, 2H, CH₂₍ₐ₎-pentyl), 1.57-1.47 (m, 2H, CH_{2(b)}-pentyl), 1.19-1.09 (m, 4H, CH_{2(c,d)}-pentyl), 0.63 (t, 3H, J=6.9 Hz, CH₃-pentyl). ESI-MS, negative mode, m/z: 1268.37 [M+5 DBA-7H]²⁻, 1203.79 [M+4 DBA-6H]²⁻, 1139.18 [M+3 DBA-5H]²⁻, 1074.09 [M+2 DBA-4H]²⁻, 1009.49 [M+1 DBA-3H]²⁻, 944.71 [M-2H]²⁻, 672.62 [M+1 DBA-4H]³⁻, 629.56 [M-3H]³⁻. [α]_{D}²¹=+30.8 (c=0.34, H₂O).

Synthesis of compound 282: Compound 282 was prepared from 277 according to the general method O (yield: 65%). ¹H NMR (400 MHz, D₂O, ppm): δ=7.75-7.67 (m, 2H, arom.), 7.66-7.55 (m, 10H, arom.), 5.42 (d, 2H, J=3.5 Hz, H-1 Glc^{III}, H-1 Glc^{V}), 5.24 (br. s, 1H, H-1 IdoUA^{IV,(II)}), 5.18-5.12 (m, 2H, H-1 IdoUA^{II,(IV)}, H-1 IdoUA^{VI}), 4.69 (d, 1H, J=8.0 Hz, H-1 Glc^{I}), 3.93, 3.68 (m, 2H, CH₂₍ₐ₎-pentyl), 1.64-1.56 (m, 2H, CH_{2(b)}-pentyl), 1.34-1.24 (m, 4H, CH_{2(c,d)}-pentyl), 0.80 (t, 3H, J=7.0 Hz, CH₃-pentyl). ESI-MS, negative mode, m/z: 1269.79 [M+4 DBA-6H]²⁻, 1205.18 [M+3 DBA-5H]²⁻, 1140.58 [M+2 DBA-4H]²⁻, 1076.02 [M+1 DBA-3H]²⁻, 1012.55 [M-2H]²⁻, 803.06 [M+3 DBA-6H]³⁻, 760.02 [M+2 DBA-5H]³⁻, 716.96 [M+1 DBA-4H]³⁻, 673.90 [M-3H]³⁻. [α]_{D}²¹=+20.8 (c=0.33, H₂O).

Synthesis of compound 283: Compound 283 was prepared from 278 according to the general method O (yield: 86%). ¹H NMR (400 MHz, D₂O, ppm): δ=7.98-7.90 (m, 2H, arom.), 7.71-7.55 (m, 10H, arom.), 5.42-5.34 (2d, 2H, J=3.5 Hz, H-1 Glc^{III}, H-1 Glc^{V}), 5.24 (br. s, 1H, H-1 IdoUA^{IV,(II)}), 5.13 (br. s, 2H, H-1 IdoUA^{II,(IV)}, H-1 IdoUA^{VI}), 4.66 (d, 1H, J=8.1 Hz, H-1 Glc^{I}), 3.93, 3.69 (m, 2H, CH₂₍ₐ₎-pentyl), 1.68-1.60 (m, 2H, CH_{2(b)}-pentyl), 1.37-1.26 (m, 4H, CH_{2(c,d)}-pentyl), 0.85 (t, 3H, J=6.9 Hz, CH₃-pentyl). ESI-MS, negative mode, m/z: 1518.18 [M+7 DBA-9H]²⁻, 1453.58 [M+6 DBA-8H]²⁻, 1388.97 [M+5 DBA-7H]²⁻, 1324.36 [M+4 DBA-6H]²⁻, 1259.24 [M+3 DBA-5H]²⁻, 1195.28 [M+2 DBA-4H]²⁻, 1130.08 [M+1 DBA-3H]²⁻, 968.32 [M+6 DBA-9H]³⁻, 925.59 [M+5 DBA-8H]³⁻, 882.52 [M+4 DBA-7H]³⁻, 839.09 [M+3 DBA-6H]³⁻, 796.08 [M+2 DBA-5H]³⁻, 753.35 [M+1 DBA-4H]³⁻, 710.02 [M-3H]³⁻. [α]_{D}²¹=+19.7 (c=0.30, H₂O).

D. Examples from 6-O-desulfated Oligosaccharides Family:

1. Preparation of Example 288 (Scheme 28)

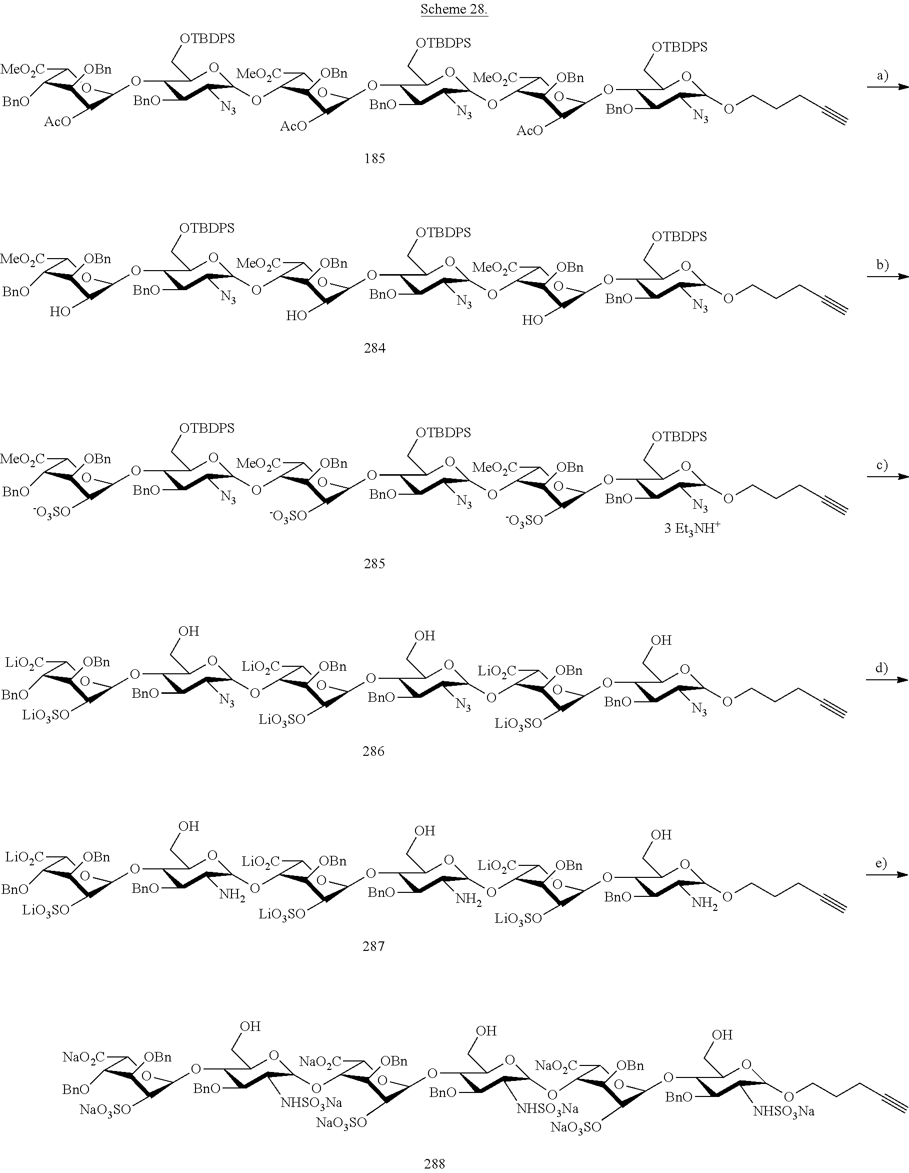

Step 28.a: Synthesis of compound 284: Deacetylation of compound 185 (117 mg, 0.043 mmol) was performed according to the general method I. Compound 284 was obtained as a white solid and directly used in the next step without any further purification. MALDI-MS, positive mode, m/z: 2585.60 [M+Na⁺], 2601.53 [M+K⁺].

Step 28.b: Synthesis of compound 285: O-Sulfation of compound 284 (0.043 mmol) was performed according to the general method M. Purification was effected by size exclusion (Sephadex LH20 dichloromethane/ethanol: 1/1) to give compound 285 as a clear yellow solid. ESI-MS, negative mode, m/z: 1465.04 [M+1 DBA-3H]²⁻, 1400.46 [M-2H]²⁻, 974.12 [M+1 DBA-4H]³⁻, 932.94 [M-3H]³⁻.

Step 28.c: Synthesis of compound 286: Desilylation of compound 285 (10.3 mop was performed according to the general method K. After purification by size exclusion (Sephadex LH20 methanol/water: 100/1) the desilylated compound was directly engaged in a saponification reaction according to the general method N in a 1/1 mixture of tetrahydrofurane/methanol (0.02 M). Purification was effected by size exclusion (Sephadex LH20 methanol/water: 100/1) to give compound 286 (16.3 mg, 76% over 3 steps) as a white solid. ¹H NMR (400 MHz, D₂O, ppm): δ=7.58-7.24 (m, 35H, arom.), 5.35-5.28 (m, 2H, H-1 IdoUA^{II}, H-1 IdoUA^{IV}), 5.25 (br. s, 1H, H-1 IdoUA^{VI}), 5.16 (d, 1H, J=3.7 Hz, H-1 Glc^{III}), 5.04-4.98 (m, 2H, H-1 Glc^{V}, H-1 Glc^{I}), 2.42 (t, 1H, J=2.7 Hz, CH_{(d)}-alkyne), 2.39-2.33 (m, 2H, CH_{2(c)}-pent-4-ynyl), 1.92-1.77 (m, 2H, CH_{2(b)}-pent-4-ynyl). ESI-MS, negative mode, m/z: 1085.82 [M+1 DBA-3H]²⁻, 1024.24 [M-2H]²⁻, 680.40 [M-3H]³⁻.

Step 28.d: Synthesis of compound 287: Selective azide reduction of compound 286 (16.3 mg, 7.83 mop was performed according to the general method L at 40° C. Purification was effected by size exclusion (Sephadex LH20 methanol/water: 100/1) to give compound 287 (15 mg, 95%) as a white solid. ¹H NMR (400 MHz, D₂O, ppm): δ=7.54-7.15 (m, 35H, arom.), 5.40 (br s, 1H, H-1 IdoUA^{II}), 5.37 (br s, 1H, H-1 IdoUA^{IV}), 5.31 (d, 1H, J=3.5 Hz, H-1 Glc^{III}), 5.28 (br s, 1H, H-1 IdoUA^{VI}), 5.20 (d, 1H, J=3.1 Hz, H-1 Glc^{V}), 4.97 (d, 1H, J=3.2 Hz, H-1 Glc^{I}), 5.04 (d, 2H, J=11.9 Hz, CH₂-Ph), 4.91 (d, 1H, J=11.4 Hz, CH—OBn), 4.79-4.73 (m, 3H, CH₂—OBn, H-5 IdoUA^{IV}), 4.69-4.59 (m, 4H, CH₂OBn, CH—OBn, H-5 IdoUA^{II}), 4.57-4.37 (m, 10H, H-5 IdoUA^{VI}, H-2 IdoUA^{II}, H-2 IdoUA^{IV}, H-2 IdoUA^{VI}, 3×CH₂-Ph), 4.33-3.81 (m, 11H, H-4 IdoUA^{II}, H-4 IdoUA^{IV}, H-4 IdoUA^{VI}, H-3 IdoUA^{II}, H-3 IdoUA^{IV}, H-3 IdoUA^{VI}, H-3 Glc^{III}, H-3 Glc^{V}, H-4 Glc^{V}, H-4 Glc^{I}, CH₍ₐ₎-pent-4-ynyl), 3.76 (t, 1H, J=3.1 Hz, H-3 Glc^{I}), 3.59-3.51 (m, 1H, CH_{(a′)}-pent-4-ynyl), 3.46-3.37 (m, 2H, H-2 Glc^{III}, H-2 Glc^{V}), 3.10 (br. dd, 1H, J=3.2 Hz, J=9.8 Hz, H-2 Glc^{I}), 2.40-2.32 (m, 3H, CH_{(d)}-alkyne, CH_{2(b)}-pent-4-ynyl), 1.89-1.78 (m, 2H, CH_{2(b)}-pent-4-ynyl). ESI-MS, negative mode, m/z: 983.15 [M-2H]²⁻, 655.11 [M-3H]³⁻.

Step 28.e: Synthesis of compound 288: N-sulfation of compound 287 (15 mg, 7.42 mol) was performed according to the general method Q. Purification was effected by size exclusion (Sephadex G25 NaCl 0.2M, then G25 water) to give compound 288 (13.8 mg, 78%) as a white hygroscopic solid. ¹H NMR (400 MHz, D₂O, ppm): δ=7.48-7.13 (m, 35H, arom.), 5.36 (br s, 2H, H-1 IdoUA^{II}, H-1 IdoUA^{IV}), 5.24 (d, 1H, J=3.5 Hz, H-1 Glc^{III}), 5.20 (d, 1H, J=3.5 Hz, H-1 Glc^{V}), 5.17 (br s, 1H, H-1 IdoUA^{VI}), 5.00 (d, 1H, J=3.5 Hz, H-1 Glc^{I}), 4.76 (d, 1H, J=10.5 Hz, CH-Ph), 4.67-4.30 (m, 19H, H-5 IdoUA^{II}, H-5 IdoUA^{IV}, H-5 IdoUA^{VI}, H-2 IdoUA^{II}, H-2 IdoUA^{IV}, H-2 IdoUA^{VI}, 6×CH₂-Ph, CH-Ph), 4.22 (br s, 1H, H-3 IdoUA^{II}), 4.14 (br s, 1H, H-4 IdoUA^{II}), 4.19 (br s, 1H, H-3 IdoUA^{IV}), 4.07 (br s, 1H, H-4 IdoUA^{IV}), 3.92-3.62 (m, 7H, H-4 Glc^{III}, H-3 Glc^{V}, H-4 Glc^{V}, H-3 IdoUA^{VI}, H-4 IdoUA^{VI}, H-4 Glc^{I}, CH₍ₐ₎-pent-4-ynyl), 3.60-3.51 (m, 2H, H-3 Glc^{III}, H-3 Glc^{I}), 3.47-3.39 (m, 1H, CH_{(a′)}-pent-4-ynyl), 3.33-3.17 (m, 3H, H-2 Glc^{III}, H-2 Glc^{V}, H-2 Glc^{I}), 2.28-2.18 (m, 3H, CH_{2(c)}-pent-4-ynyl, CH_{(d)}-alkyne), 1.79-1.64 (m, 2H, CH_{2(b)}-pent-4-ynyl). ESI-MS, negative mode, m/z: 1296.45 [M+3 DBA-5H]²⁻, 1231.85 [M+2 DBA-4H]²⁻, 1167.25 [M+1 DBA-3H]²⁻, 734.74 [M-3H]³⁻. [α]_{D}²¹=+23.5 (c=0.52, H₂O).

E. Examples from 2′-O-desulfated Oligosaccharides Family:

1. Preparation of Example 294 (Scheme 29)

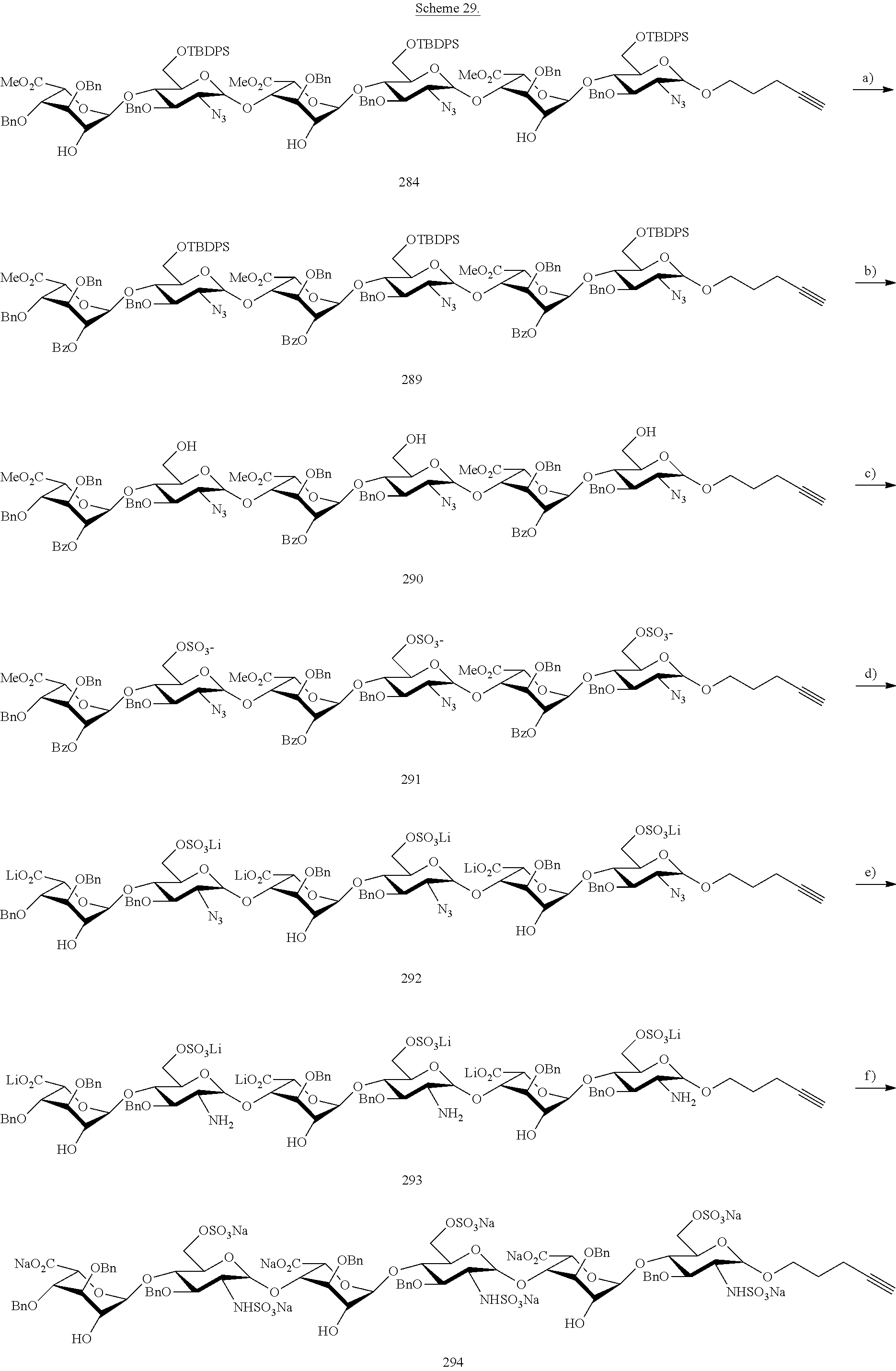

Step 29.a: Synthesis of compound 289: In a dry round-bottom flask, compound 284 (71.4 mg, 0.028 mmol) was dissolved in anhydrous pyridine (930 μL) under a nitrogen atmosphere. Benzoyl chloride (274 μL, 2.37 mmol, 85 eq.) and a catalytic amount of DMAP (1.7 mg, 0.014 mmol, 0.5 eq.) were successively added to this solution and the resulting mixture was stirred overnight at room temperature. The reaction mixture was directly poured on a sephadex LH-20 column (dichloromethane/ethanol: 1/1) to give after concentration compound 289 (76.3 mg, 95%) as a pale yellow solid. ¹H NMR (400 MHz, CDCl₃, ppm): δ=7.91-7.63 (m, 20H, arom.), 7.41-7.14 (m, 60H, arom.), 5.71-5.66 (m, 2H, H-1 IdoUA^{II}, H-1 IdoUA^{IV}), 5.50 (d, 1H, J=3.8 Hz, H-1 IdoUA^{VI}), 5.22 (t, 1H, J=5.3 Hz, H-2 IdoUA^{II}), 5.16 (t, 1H, J=5.4 Hz, H-2 IdoUA^{IV}), 5.10 (t, 1H, J=4.3 Hz, H-2 IdoUA^{VI}), 4.85-4.81 (m, 2H, H-1 Glc^{III}, H-1 Glc^{V}), 4.80-4.73 (m, 4H, 2×CH₂-Ph), 4.73-4.61 (m, 6H, 2×CH₂-Ph, H-1 Glc^{I}, H-5 IdoUA^{VI}), 4.65-4.42 (m, 7H, 3×CH₂-Ph, H-5 IdoUA^{II}), 4.27-4.12 (m, 4H, H-4 Glc^{I}, H-3 IdoUA^{IV}, H-3 IdoUA^{II}, H-5 IdoUA^{IV}), 4.08 (t, 1H, J=5.7 Hz, H-3 IdoUA^{VI}), 4.04-3.84 (m, 5H, H-4 IdoUA^{II}, H-4 IdoUA^{VI}, H-4 Glc^{I}, H-5 Glc^{V}, H-6a Glc^{III}), 3.83-3.53 (m, 10H, H-4 IdoUA^{IV}, H-6a/b Glc^{I}, H-6b Glc^{III}, H-6a/b Glc^{V}, H-4 Glc^{V}, H-3 Glc^{V}, H-3 Glc^{III}, CH₍ₐ₎-pent-4-ynyl), 3.48, 3.21, 3.08 (3s, 9H, CO₂Me), 3.51-3.38 (m, 4H, H-5 Glc^{I}, H-5 Glc^{III}, H-3 Glc^{I}, CH_{(a′)}-pent-4-ynyl), 3.27 (dd, 1H, J=3.5 Hz, J=10 Hz, H-2 Glc^{V}), 3.25-3.18 (m, 2H, H-2 Glc^{III}, H-2 Glc^{I}), 2.31-2.23 (m, 2H, CH_{2(c)}-pent-4-ynyl), 1.81-1.72 (m, 3H, CH_{(d)}-alkyne, CH_{2(b)} pent-4-ynyl), 1.07-1.01 (m, 27H, C(CH₃)₃). MALDI-MS, positive mode, m/z: 2896.32 [M+Na⁺].

Step 29.b: Synthesis of compound 290: Desilylation of compound 289 (64 mg, 0.022 mmol) was performed according to the general method J. Purification was effected by chromatography on silica gel column (heptane/ethyl acetate: 5/5 to 4/6) to give the desilylated compound 290 (41 mg, 85%) as a white solid. ¹H NMR (400 MHz, CDCl₃, ppm): δ=8.10-8.05 (m, 4H, arom.), 7.96-7.91 (m, 2H, arom.), 7.37-7.12 (m, 44H, aromatic), 5.49-5.42 (m, 3H, H-1 IdoUA^{II}, H-1 IdoUA^{IV}, H-1 IdoUA^{VI}), 5.21-5.16 (m, 3H, H-2 IdoUA^{II}, H-2 IdoUA^{IV}, H-2 IdoUA^{VI}), 4.88-4.83 (m, 3H, H-1 Glc^{III}, CH₂-Ph), 4.80-4.64 (m, 12H, H-1 Glc^{I}, H-1 Glc^{V}, H-5 IdoUA^{VI}, H-5 IdoUA^{II}, 4×CH₂-Ph), 4.62 (d, 1H, J=4.1 Hz, H-5 IdoUA^{IV}), 4.57, 4.25 (2d, 2H, J=10.5 Hz, CH₂-Ph), 4.48 (2d, 2H, J=11.6 Hz, CH₂-Ph), 4.15-4.06 (m, 2H, H-3 IdoUA^{IV}, H-3 IdoUA^{II}), 4.02-3.81 (m, 8H, H-3 IdoUA^{VI}, H-4 IdoUA^{II}, H-4 IdoUA^{VI}, H-4 IdoUA^{IV}, H-4 Glc^{V}, H-4 Glc^{I}, H-3 Glc^{III}, CH₍ₐ₎-pent-4-ynyl), 3.54, 3.42, 3.39 (3s, 9H, CO₂Me), 3.54-3.48 (m, 4H, CH_{(a′)}-pent-4-ynyl, H-3 Glc^{V}, H-3 Glc^{I}, H-4 Glc^{III}), 3.33-3.21 (m, 3H, H-2 Glc^{V}, H-2 Glc^{I}, H-2 Glc^{III}), 2.35-2.30 (m, 2H, CH_{2(c)}-pent-4-ynyl), 1.87 (t, 1H, J=2.5 Hz, CH_{(d)}-alkyne), 1.86-1.76 (m, 2H, CH_{2(b)}-pent-. MALDI-MS, positive mode, m/z: 2181.78 [M+Na⁺], 2197.75 [M+K⁺].

Step 29.c: Synthesis of compound 291: O-sulfation of compound 290 (31.6 mg, 14.6 mop was performed according to the general method M. Purification was effected by size exclusion (Sephadex LH20 dichloromethane/ethanol: 1/1) to give compound 291 as a clear yellow solid. ESI-MS, negative mode, m/z: 1263.37 [M+1 DBA-3H]²⁻, 798.84 [M-3H]³⁻.

Step 29.d: Synthesis of compound 292: Saponification of compound 291 (14.6 mop was performed according to the general method N. Purification was effected by size exclusion (Sephadex LH20 methanol/water: 100/1) to give compound 292 (30.2 mg, 99% over 2 steps) as a white solid. ¹H NMR (400 MHz, CD₃OD, ppm): δ=7.40-6.98 (m, 35H, arom.), 5.16-5.07 (m, 3H, H-1 IdoUA^{II}, H-1 IdoUA^{IV}, H-1 IdoUA^{VI}), 5.03 (d, 1H, J=3.5 Hz, H-1 Glc^{II}), 4.99 (d, 1H, J=3.3 Hz, H-1 Glc^{I}), 4.86-4.74 (m, 2H, H-1 Glc^{V}, CH-Ph), 4.71-4.31 (m, 16H, CH-Ph, H-5 IdoUA^{VI}, H-5 IdoUA^{II}, H-5 IdoUA^{IV}, 6×CH₂-Ph), 4.30-4.07 (m, 7H, H-6a/b Glc^{I}, H-6a/b Glc^{III}, H-6a/b Glc^{V}, H-4 IdoUA^{VI}), 4.07-4.02 (br, 1H, H-4 IdoUA^{IV}), 3.95-3.51 (m, 13H, CH₍ₐ₎-pent-4-ynyl, H-2 IdoUA^{II}, H-2 IdoUA^{IV}, H-2 IdoUA^{VI}, H-3 IdoUA^{II}, H-3 IdoUA^{IV}, H-3 IdoUA^{VI}, H-4 IdoUA^{II}, H-4 Glc^{I}, H-4 Glc^{III}, H-3 Glc^{I}, H-3 Glc^{III}, H-3 Glc^{V}), 3.45-3.33 (m, 2H, CH_{(a′)}-pent-4-ynyl, H-2 Glc^{II}), 3.28 (dd, 1H, J=3.4 Hz, J=9.6 Hz, H-2 Glc^{V}), 3.28 (dd, 1H, J=3.6 Hz, J=9.6 Hz, H-2 Glc^{I}), 2.23-2.17 (m, 2H, CH_{2(c)}-pent-4-ynyl), 2.13 (t, 1H, J=2.5 Hz, CH_{(d)}-alkyne), 1.76-1.64 (m, 2H, CH_{2(b)}-pent-4-ynyl). ESI-MS, negative mode, m/z: 1086.29 [M+1 DBA-3H]²⁻, 1021.21 [M-2H]²⁻, 680.44 [M-3H]³⁻, 510.10 [M-4H]⁴⁻.

Step 29.e: Synthesis of compound 293: Selective azide reduction of compound 292 (30.2 mg, 14.6 mop was performed according to the general method L at 40° C. Purification was effected by size exclusion (Sephadex LH20 methanol/water: 100/1) to give compound 293 (27.8 mg, 95%) as a white solid. ¹H NMR (400 MHz, CD₃OD, ppm): δ=7.43-7.02 (m, 35H, arom.), 5.31-4.96 (m, 8H, H-1 IdoUA^{II}, H-1 IdoUA^{IV}, H-1 IdoUA^{VI}, H-1 Glc^{III}, H-1 Glc^{V}, H-1 Glc^{I}, CH₂-Ph), 4.69-4.42 (m, 14H, H-5 IdoUA^{VI}, H-5 IdoUA^{II}, 6×CH₂-Ph), 4.39-3.62 (m, 11H, H-4 IdoUA^{IV}, H-4 IdoUA^{II}, H-3 IdoUA^{IV}, H-3 IdoUA^{II}, H-2 IdoUA^{IV}, H-2 IdoUA^{VI}, H-2 IdoUA^{II}, H-4 Glc^{III}, H-3 Glc^{III}, H-3 Glc^{I}, CH₍ₐ₎-pent-4-ynyl), 3.58-3.49 (m, 1H, CH_{(a′)}-pent-4-ynyl), 3.21-3.12 (m, 3H, H-2 Glc^{III}, H-2 Glc^{I}, H-2 Glc^{V}), 2.26-2.15 (m, 3H, CH_{2(c)}-pent-4-ynyl, CH_{(d)}-alkyne), 1.83-1.66 (m, 2H, CH_{2(b)}-pent-4-ynyl). ESI-MS, negative mode, m/z: 982.16 [M-2H]²⁻, 654.43 [M-3H]³⁻.

Step 29.f: Synthesis of compound 294: N-sulfation of compound 293 (10 mg, 4.99 mop was performed according to the general method Q. Purification was effected by size exclusion (Sephadex G25 NaCl 0.2M, then G25 water) to give compound 294 (8 mg, 88%) as a white hygroscopic solid. ¹H NMR (400 MHz, D₂O, ppm): δ=7.47-7.24 (m, 35H, arom.), 5.15 (d, 1H, J=3.5 Hz, H-1 Glc^{III}), 5.08 (d, 1H, J=3.5 Hz, H-1 Glc^{V}), 5.06-5.02 (m, 2H, H-1 Glc^{I}, H-1 IdoUA^{II}), 5.00 (s, 1H, H-1 IdoUA^{IV}), 4.97 (s, 1H, H-1 IdoUA^{VI}), 4.76-4.41 (m, 11H, H-5 IdoUA^{VI}, H-5 IdoUA^{II}, H-5 IdoUA^{IV}, 4×CH₂-Ph), 4.32-4.02 (m, 9H, H-4 IdoUA^{II}, H-4 IdoUA^{IV}, H-3 IdoUA^{IV}, 3×CH₂-Ph), 3.99-3.57 (m, 12H, H-4 IdoUA^{VI}, H-3 IdoUA^{VI}, H-3 IdoUA^{II}, H-2 IdoUA^{IV}, H-2 IdoUA^{VI}, H-2 IdoUA^{II}, CH₍ₐ₎-pent-4-ynyl, H-4 Glc^{III}, H-4 Glc^{V}, H-3 Glc^{III}, H-3 Glc^{V}, H-3 Glc^{I}), 3.54-3.45 (m, 1H, CH_{(a′)}-pent-4-ynyl), 3.36-3.25 (m, 3H, H-2 Glc^{III}, H-2 Glc^{I}, H-2 Glc^{V}), 2.30-2.23 (m, 3H, CH_{2(c)}-pent-4-ynyl, CH_{(d)}-alkyne), 1.81-1.63 (m, 2H, CH_{2(b)}-pent-4-ynyl). ESI-MS, negative mode, m/z: 1167.25 [M+1 DBA-3H]²⁻, 1113.65 [M-2H]²⁻, 734.78 [M-3H]³⁻. [α]_{D}²¹=+19.0 (c=0.40, H₂O).

### Biological Testing

It will be understood that a variety of assays are suitable for testing the biological activity of the compounds of the present invention. However, suitable methods for testing the biological activity of the compounds of the present invention are listed below.

### Proliferation Assay

### Assay: General Methods

The activities of the compounds of the present invention were tested using a proliferation assay, such as the one described by N. Ali et al., J Pharmacol Sci, 2005, 98, 130-141. A defined number of cells is seeded in each well of a culture plate. In order to stimulate cell proliferation, a growth factor is added in some wells, while others remain unstimulated (basal proliferation conditions). To monitor the effects of substances of interest (oligosaccharides compounds according to the present invention) on basal and growth factor-induced cell proliferation, substances of interest are added at different concentrations (i.e. 0.1, 0.3, 1, 3, 10 or 30 μM) in presence or absence of the growth factor, respectively. After an incubation period of 24 hours, the total number of cells is estimated in all samples (generally through an indirect method, such as incorporation of radioactivity into the newly synthesised DNA or colorimetric assays based on cellular enzyme activities or metabolite production). The total number of cells for the control sample in which no substance of interest nor growth factor have been added is set to 1. The total number of cells in all the other samples is compared to this value in order to obtain the relative proliferation index.

Typically, cells are seeded in 48 or 96-well plates. After 2 h (i.e. the time required for cells to adhere to the support) the normal culture media is replaced by a minimal essential culture media in which cells are grown for 24 h (starvation period). This step is used to reduce cell growth (cell metabolism is slowed down in order to better visualise the growth factor stimulation effect) before adding an angiogenic protein, such as a growth factor i.e. FGF-2 or PDGF-β. No growth factor is added in basal proliferation conditions (independently of the presence or absence of the oligosaccharide of the present invention). To evaluate the inhibition of the oligosaccharide compounds according to the present invention on cell proliferation, the angiogenic protein (growth factor) is added at a fixed concentration (from 5 ng/ml to 10 ng/ml) with increasing amounts of oligosaccharide compounds (0.1, 0.3, 1, 3, 10 or 30 μM), which allows the IC₅₀ value to be estimated (i.e. the oligosaccharide concentration at which the stimulatory effect of the growth factor is inhibited by 50%).

Following addition of the angiogenic protein (growth factor) over a 24 h stimulation period, a commercial reagent containing a substrate for a cellular enzyme is added and incubated for couple of hours. The degradation of the substrate by the enzyme leads to the production of a coloured product which is titrated by absorbance measurement. Absorbencies are converted into numbers of cells using a standard curve derived from the incubation of known numbers of cells with the reagent.

### Results

In the following experiments the effects of compounds 240 and 241 (concentrations from 0.1 to 30 μM) were evaluated on FGF-2-induced NHDF (Normal Human Dermal Fibroblast) cell proliferation. The experiment was performed at a concentration of FGF-2 of 5 ng/ml.

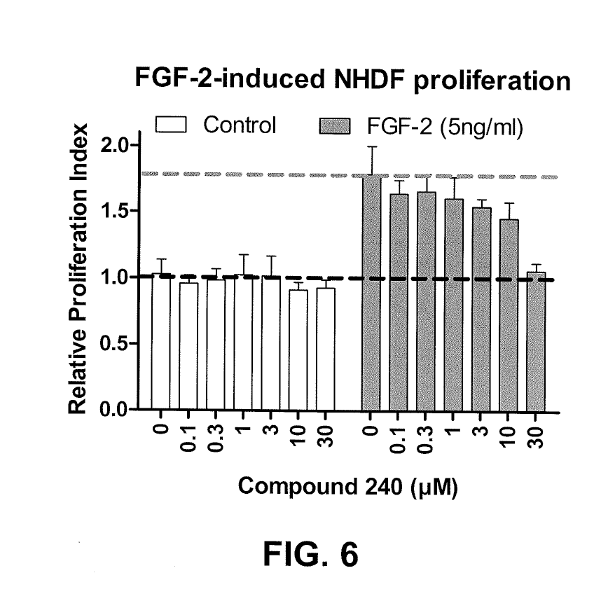

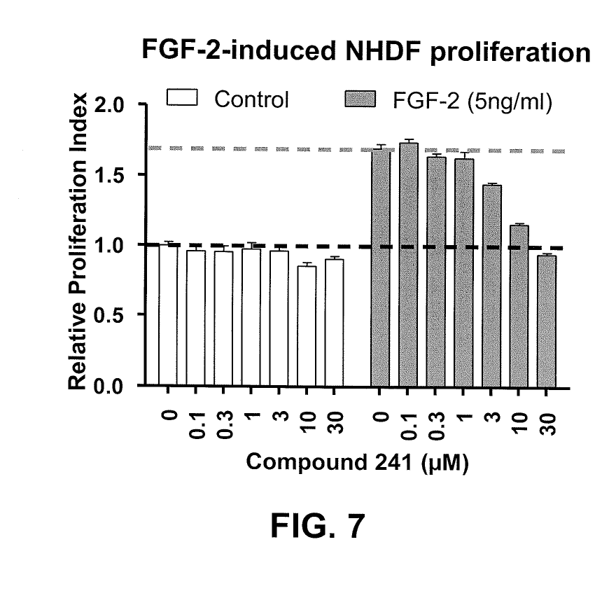

As can be seen from FIGS. 6-7, these compounds inhibit FGF-2-induced fibroblast proliferation (starting in the micromolar range) while basal NHDF proliferation did not seem to be affected by the oligosaccharide compounds (control conditions).

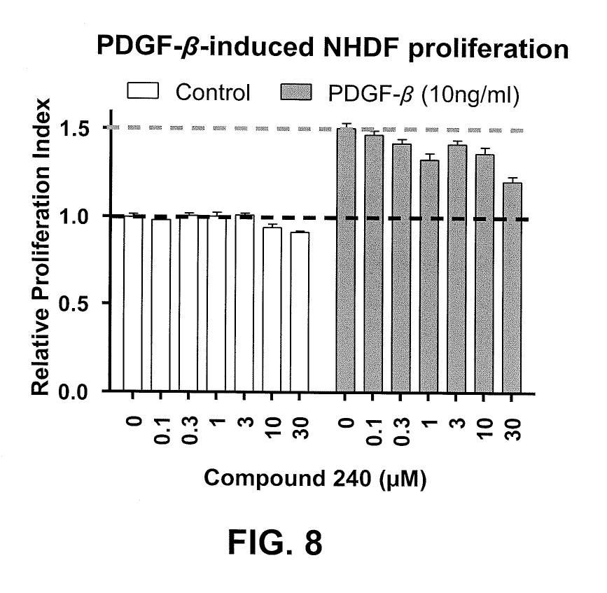

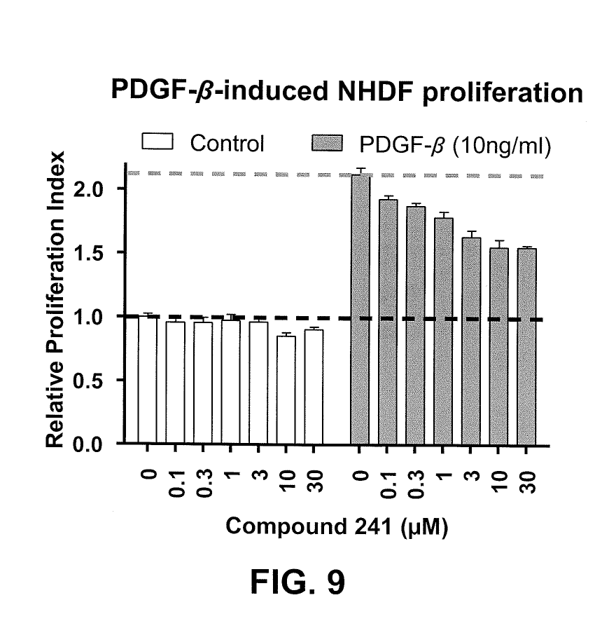

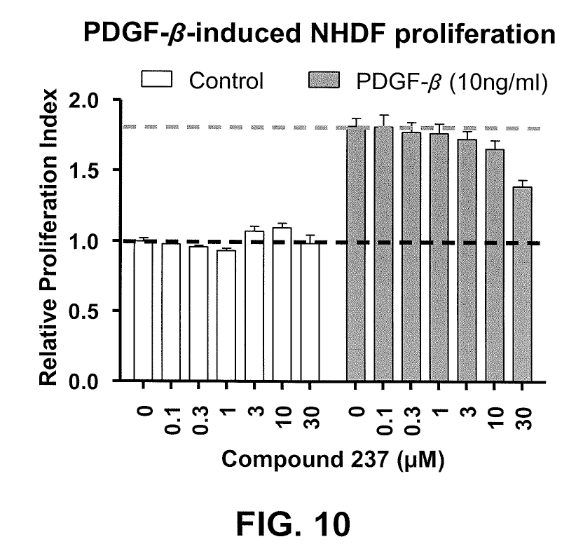

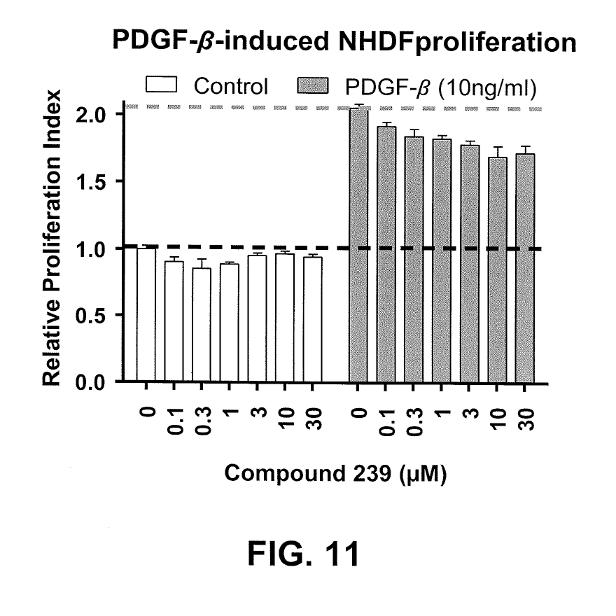

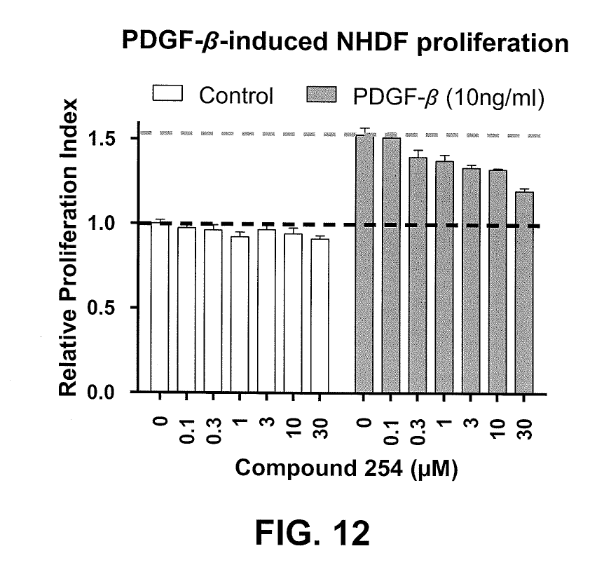

Similarly, the effects of compounds 237, 239, 240, 241 and 254 on PDGF-β were evaluated as shown in FIGS. 8-12, which show that these compounds inhibit PDGF-β proliferation (starting in the micromolar range). The experiments were performed at a concentration of PDGF-β of 10 ng/ml.

### In Vitro Angiogenesis Assays

### Introduction

These experiments are also known as endothelial tubule formation assays and they are used to evaluate the compound effects on in vitro angiogenesis (the neo-formation of blood vessels from pre-existing ones). This process is critical in tumour development as bloodstream brings all the necessary nutrients and oxygen to cancer cells. Not surprisingly, angiogenesis (with metastasis) remains a principal target for the development of anti-cancer drugs.

### Assay: General Methods

The assay uses a commercially available product (AngioKit) specially designed for screening experiments. The product consists of a 24 or 96-well culture plate containing endothelial and fibroblast cells. A similar assay has been developed “in-house” using 48-well culture plates.

Endothelial cells will, on a ten-day period, develop into a branched network of endothelial tubules or “primitive” blood vessels. This process requires a co-culture with fibroblasts, these latter cells secreting essential growth factors for endothelial cells. At the end of the incubation period (about ten days), angiogenesis is monitored both by ELISA and image analysis software quantifying different blood vessel parameters (number and average length of tubules, field area, number of junctions for example). All reagents and material (except the oligosaccharide compounds, the VEGF-A and the 48-well plate AngioKits) are bought from TCS CellWorks (http://www.tcscellworks.co.uk) which provides:
24 and 96-well plate AngioKits (including culture medium)
Anti-CD31 (a cell marker specifically expressed on endothelial cells) reagents for ELISA and tubule staining procedures.
AngioSys image analysis software

For home-made 48-well plate AngioKits, NHDF and HUVEC cells were independently purchased while TCS CellWorks culture medium and protocol were used.

The cell medium is replaced with fresh one (day 1) and cells are incubated at 37° C. with the compounds of interest (oligosaccharides and/or angiogenic proteins, such as growth factors). During the course of the experiment, the culture medium (including oligosaccharides and/or angiogenic proteins) is replaced at days 4 and 7.

At day 10, the assay is terminated by the labelling of endothelial cells by an ELISA procedure (indirect colorimetric titration of the endothelial cell number through dosage of the endothelial-specific CD31 marker) and the staining of endothelial tubules. Photographs of stained tubules are taken and analysed with the AngioSys image analysis software.

### Results

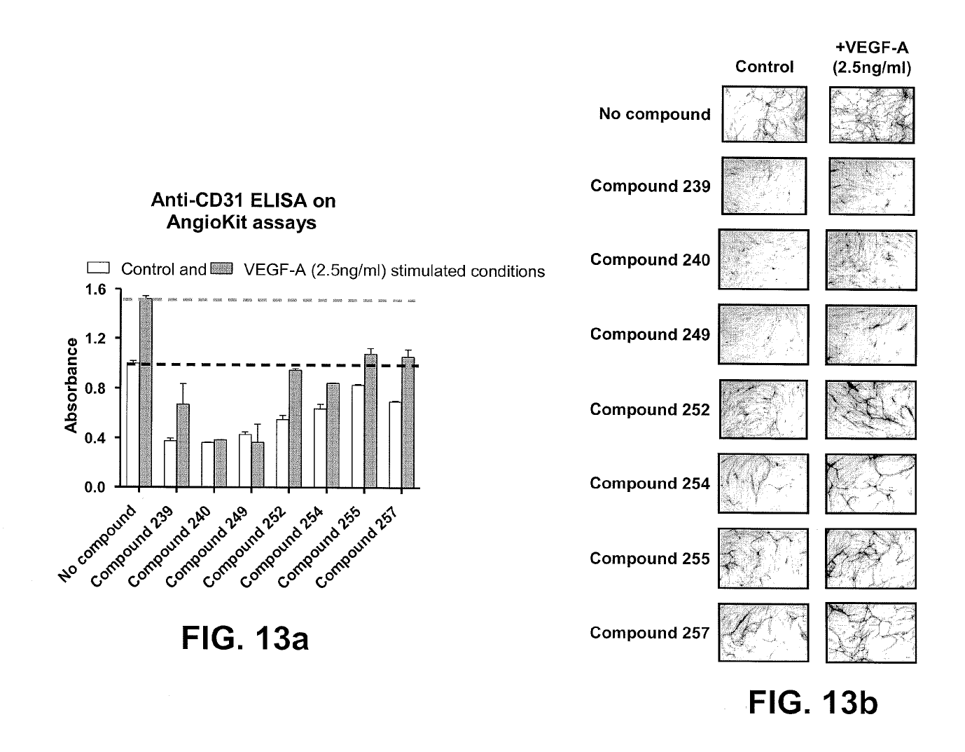

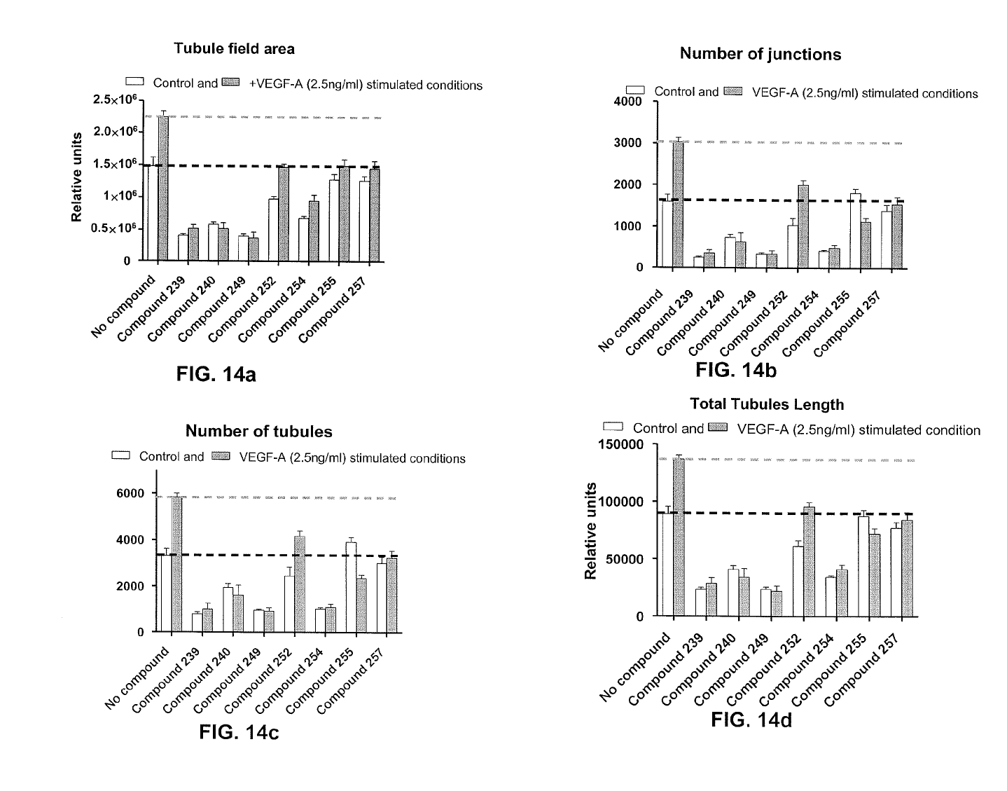

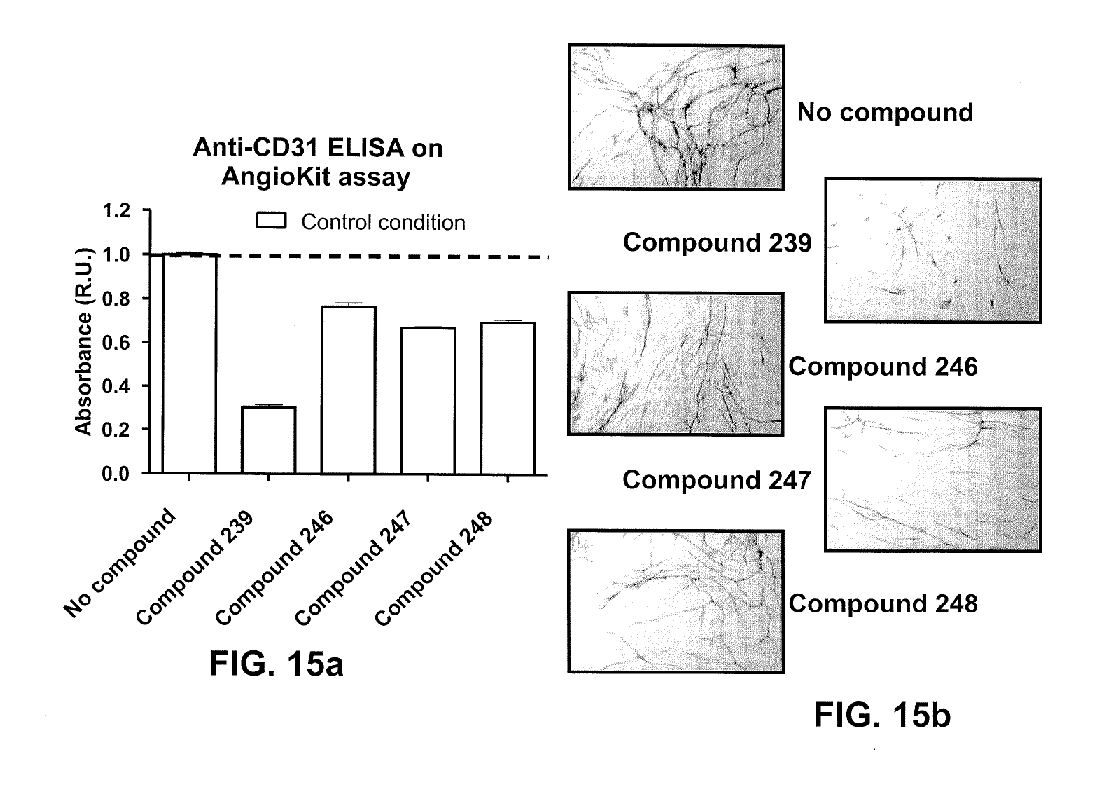

In the following assay the effects of compounds 239, 240, 246, 247, 248, 249, 252, 254, 255 and 257 on in vitro angiogenesis were measured (all compounds were added at the 30 μM concentration). The inhibition of control and/or VEGF-A-stimulated angiogenesis can be seen in FIGS. 13-15. For the data presented in FIG. 13, AngioSys image analyses are shown on FIG. 14. Photographs of endothelial tubules are shown on the side of the anti-CD31 ELISA (see FIGS. 13 and 15).

### Screening of Compounds by Growth Factor/Heparin Competition Assay Based on Surface Plasmon Resonance (SPR)

Heparin or low molecular weight heparin (6 kDa) from SIGMA were biotinylated at the reducing end and immobilized on a Biacore sensorchip. Different concentrations of the compounds of the present invention were co-incubated at a fixed concentration of targets: FGF-2, PDGF-β, VEGF-A or SDF-1α for 30 minutes. The mixture was then injected onto the streptavidin control (control reference) and HP surfaces. Only free growth factor (GF) or chemokine, i.e. the target molecules not bound to compounds of the present invention, were trapped on the heparin surface. From the binding of free targets on heparin, the percentages of inhibition were calculated and then reported in function of compound concentrations. The plot was fitted with a four-parameter model and IC₅₀ was calculated. The 0% inhibition value was obtained for the injection of the studied target in running-buffer, and the 100% inhibition value was obtained for the injection of the studied target co-incubated with 10 μM of low molecular weight heparin 6 kDa.

### FGF-2/Heparin Competition Assay by SPR

A FGF-2/heparin competition assay using the Biacore technology was performed in the following conditions: 10 nM FGF-2, biotinylated heparin, Reference Streptavidin, Sensorchip C1, PBS-T 0.02%, Regeneration NaCl 2M

### PDGF-β/Heparin Competition Assay by SPR

A PDGF-β/heparin competition assay using the Biacore technology was performed using the following conditions: 10 nM PDGF-β, biotinylated heparin, Reference Streptavidin, Sensorchip C1, HBS-P, Regeneration NaCl 2M.

### VEGF-A/Heparin Competition Assay by SPR

A VEGF-A/heparin competition assay using the Biacore technology was performed using the following conditions: 10 nM VEGF-A, biotinylated low molecular weight heparin, Reference Streptavidin, Sensorchip SA, HBS-P, Regeneration NaCl 2M.

### SDF-1α/Heparin Competition Assay by SPR

A SDF-1α/heparin competition assay using the Biacore technology was performed using the following conditions: 96 nM SDF-1α, biotinylated low molecular weight heparin (6 kDa), Reference Streptavidin, Sensorchip SA, HBS-P, Regeneration NaCl 2M

### Effects of Oligosaccharides on Growth Factor/Heparin Competition Assay by Surface Plasmon Resonance (SPR)

IC₅₀ values of compounds of the present invention were determined using the Biacore technology for the growth factor and chemokine/heparin competition assays that contained the following proteins: VEGF-A, SDF-1α, FGF-2 and PDGF-β. The results from the assays are show below.

The synthetic compounds' IC₅₀ for the target/heparin interaction range from 4.8 nM to 1,670 nM for VEGF-A, from 3.6 nM to >100,000 nM for FGF-2, from 23 nM to 22,600 nM for SDF-1α and from 10.9 nM to 28,000 nM for PDGF-β.

IC₅₀ (nM)

| Compound_Number | FGF-2 | PDGF-β | VEGF-A | SDF-1 |
|---|---|---|---|---|
| Compound 236 | 187 | 1,350 | 310 | 3,600 |
| Compound 239 | 4.5 | 85 | 5.2 | 43 |
| Compound 240 | 3.6 | 10.9 | 4.8 | 23 |
| Compound 246 | 167 | 194 | 59 | 865 |
| Compound 247 | >100,000 | 1,690 | 45 | 430 |
| Compound 248 | 1,660 | 28,000 | 1,670 | 22,600 |
| Compound 249 | 136 | 225 | ND* | ND* |
| Compound 252 | 121 | 171 | ND* | ND* |
| Compound 254 | 1,670 | 554 | ND* | ND* |
| Compound 255 | 36 | 122 | ND* | ND* |
| Compound 257 | 5,600 | 716 | ND* | ND* |

*Not Done

These data can be compared with a natural octasaccharide having the following IC₅₀ data
FGF-2 0.7 μM
PDGF-β 13 μM
VEGF-A 0.4 μM
SDF-1 3.6 μM

### Determination of the Compounds Anti-Heparanase Activity

To determine the compounds IC₅₀ values for the heparanase target, we adapted an assay based on the ability of heparanase to degrade fondaparinux (Sanofi patent No. 287 3377 FR), and the capacity of fondaparinux to inhibit factor Xa activity via AT III binding. This assay was carried out on a STA Compact robot (Diagnostica Stago). Briefly, different concentrations of compounds were added to a mixture containing the heparanase enzyme and fondaparinux and after a time-fixed incubation period, AT III, Factor Xa and a chromogenic substrate (CBS 31.39) were sequentially added to the reaction mix. Production of paranitroanilin resulting from the degradation of the chromogenic substrate CBS 31.39 was monitored at 405 nm. Data obtained for the different concentration points were plotted using a four-parameter fit model and IC₅₀ determined.

The heparanase inhibitions observed for the compounds were compared with the effects of suramin, a well-known inhibitor of the enzyme. The results for compounds 239 and 240 are shown below:

| Compounds | IC₅₀ (nM) |
|---|---|
| Suramin | 922 |
| Compound 239 | 9.2 |
| Compound 240 | 0.42 |

### Hematopoietic Stem Cell Mobilisation (HSC) Experiments

During cancer treatment, patients receive injections of a cytokine (G-CSF) mobilising stem cells. These stem cells are then collected from the peripheral blood using an apheresis machine. Apheresis is usually performed for several days until enough stem cells have been collected to support treatment with high-dose chemotherapy and/or radiotherapy. In the experiments of the present invention, different doses (5, 15 and 30 mg/kg body weight (bw)) and administration routes (intraperitoneal, intravenous and subcutaneous) were tested along time course experiments in mice (30 min, 1 h, 3 h and 5 h).

### Materials and Methods

### Animals

C57BL/6 mice (8-10 weeks old, obtained from Jackson Laboratory, USA) were housed 15 days in the CEA/DSV/iRCM animal facilities. Animal care was in accordance with French Government procedures (Services Veterinaires de la Sante et de la Production Animale, Ministere de l'Agriculture, France).

### Compound Administration

The compounds of the present invention were administered by the following routes:

i) intravenously: Anesthetized mice were placed on their left side. Injection of 100 μl of compound was performed with insulin syringe into the retro-orbital sinus of the right eye of mice.

ii) intraperitoneally: Anesthetized mice were manually restrained and 100 μl of compound were injected into the peritoneal cavity.

iii) subcutaneously: Anesthetized mice were injected with 100 μl of compound under the dorsal skin.

To maintain body temperature, mice are placed on a heating plate warmed at 38° C.

### Blood Sample Collection

At the following time points 30 min, 1 h, 3 h and 5 h, mice were anesthetized with isoflurane gas in a closed induction chamber. When the animals were asleep, 100 μl blood samples were collected with heparin-coated capillaries into tubes containing 20 mM EDTA solution. Blood formula was performed with a ABACUS JUNIOR VET cell numeration system (KITVIA) apparatus.

### Hematopoietic Stem Cells Phenotyping

Phenotyping was performed by flow cytometry from blood samples. After 5 nm centrifugation at 300 g, red blood cells were lyzed, washed and resuspended in PBS. Washing is necessary to remove EDTA solution which could interfere with antibody staining. To phenotype HSCs, white blood cells were stained with biotinylated anti-Lin antibody cocktail (Milteny), PE-anti-Sca-1 and APC anti-c-Kit (BD Bioscience) antibodies for 30 minutes at 4° C. The biotinylated antibodies were revealed with streptavidin (SA)-PE-Cy7 (BD Biosciences). Seven-parameter-color analysis were performed on a CYAN cytometer (Dako) equipped with argon ion (488 nm) and red (638 nm) lasers. Cells exhibiting Lin⁻ Sca⁺ c-kit⁺ (LSK) phenotype were identified as hematopoietic stem cells. At least 20,000 events were acquired per each tube and analysis was done with the FlowJo software.

### Sequential Administration of G-CSF, AMD3100 and Compound 240

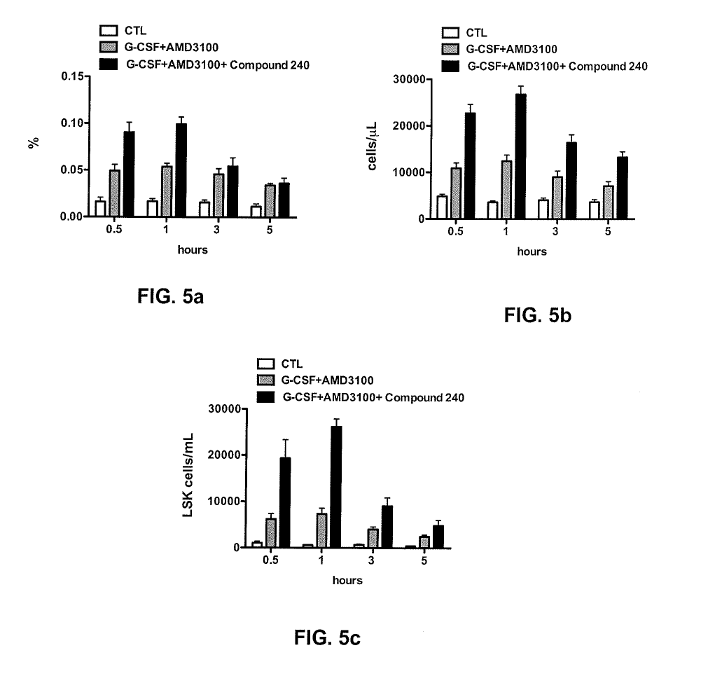

Two months aged-C57BL/6 mice were injected s.c. with 2.5 μg of G-CSF twice a day for 2 days. The third day, 1 hour after G-CSF injection, AMD3100 was administered s.c. at 5 mg/kg and for the group treated with the 3 compounds, compound 240 was given i.v. at 15 mg/kg 30 minutes after AMD3100 administration. Blood samples were collected at 0.5, 1, 3 and 5 hours after Compound 240 injection (mock-injection for Control and G-CSF+AMD3100 conditions). White blood cells were counted and labeled with lineage-PE-Cy7 (L), Sca-PE (S), and cKit-APC (K) antibodies for data acquisition by cytometry. The LSK labeling reveals hematopoietic stem cells (HSCs) mobilized for each condition (white: mock administration; grey: G-CSF+AMD3100; black: G-CSF+AMD3100+Compound 240). The results are indicated in FIG. 5. About 3 times more HSCs were mobilized when the compound 240 was added to the G-CSF+AMD3100 combination enhancing the effectiveness of HSC mobilization.

1.-16. (canceled)

17. A compound of the following formula or a salt or solvate thereof: 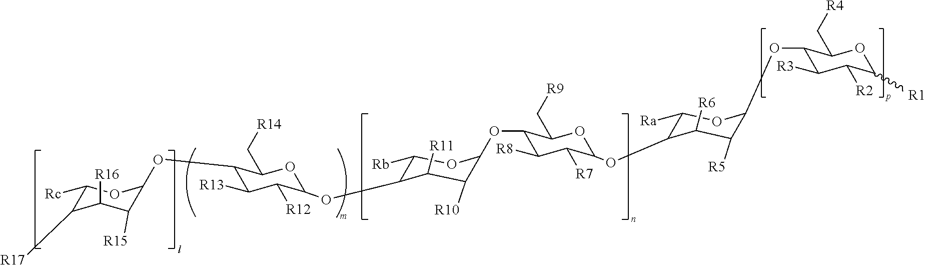
wherein:
Rₐ, R_{b} and R_{c} are selected from the group consisting of: COOH, COO—C₁₋₁₀alkyl. COO—C₃₋₁₀cycloalkyl and COO—C₁₋₁₀alkylC₃₋₁₀aryl, COO—C₃₋₁₀cycloalkylC₃₋₁₀aryl,
R₁ is selected from the group consisting of: hydrogen, C₁₋₁₀alkyl, C₃₋₁₀cycloalkyl, C₂₋₁₀alkenyl, C₃₋₁₀cycloakenyl, C₂₋₁₀alkynyl, O—C₁₋₁₀alkyl, O—C₃₋₁₀cycloalkyl, O—C₂₋₁₀alkenyl, O—C₃₋₁₀cycloalkenyl, O—C₂₋₁₀alkynyl, O—C₃₋₁₀aryl, OH and O—SO₃H.
R₂, R₇, R₁₂ are each independently selected from the group consisting of: hydrogen, NH—SO₃H, NH₂, NH—C(O)C₃₋₁₀aryl, NH—C(O)C₁₋₁₀alkylCOOH, NH—C(O)C₃₋₁₀cycloalkylCOOH, NH—C(O)C₃₋₁₀arylSO₃H, NH—C(O)C₃₋₁₀arylCOOH, O—C₁₋₁₀alkyl, O—C₃₋₁₀cycloalkyl, O—C₃₋₁₀cycloalkylC₃₋₁₀aryl, O—C₁₋₁₀alkylC₃₋₁₀aryl, O—C₁₋₁₀acyl, O—C₁₋₁₀acylC₃₋₁₀aryl, O—C(O)C₃₋₁₀arylSO₃H, O—C(O)C₃₋₁₀arylCOOH, O—SO₃H, O—C(O)C₃₋₁₀aryl, O—C(O)C₁₋₁₀alkylCOOH, O—C₁₋₁₀alkylOSO₃H, O—C₁₋₁₀alkylNHSO₃H, O—C(O)C₃₋₁₀cycloalkylCOOH, O—C₃₋₁₀cycloalkylOSO₃H, O—C₃₋₁₀cycloalkylNHSO₃H, and OH;
R₃, R₅, R₆, R₈, R₁₀, R₁₁, R₁₃, R₁₅ and R₁₆ are each independently selected from the group consisting of: hydrogen, O—C₁₋₁₀alkyl, O—C₁₋₁₀alkylC₃₋₁₀aryl, O—C₁₋₁₀alkylOSO₃H, O—C₁₋₁₀alkylNHSO₃H, O—C₃₋₁₀cycloalkyl, O—C₃₋₁₀cycloalkylC₃₋₁₀aryl, O—C₃₋₁₀cycloalkylOSO₃H, O—C₃₋₁₀cycloalkylNHSO₃H, O—C₁₋₁₀acyl, O—C₁₋₁₀acylC₃₋₁₀aryl, O—C(O)C₃₋₁₀aryl, O—C(O)C₁₋₁₀alkylCOOH, O—C(O)C₃₋₁₀cycloalkylCOOH, O—SO₃H, O—C(O)C₃₋₁₀arylSO₃H, O—C(O)C₃₋₁₀arylCOOH and OH;
R₄, R₉ and R₁₄ are each independently selected from the group consisting of: O—C₁₋₁₀alkyl, O—C₃₋₁₀cycloalkyl, O—C₁₋₁₀alkylC₃₋₁₀aryl, O—C₁₋₁₀alkylOSO₃H, O—C₃₋₁₀cycloalkylC₃₋₁₀aryl, O—C₃₋₁₀cycloalkylOSO₃H, O—C₃₋₁₀cycloalkylNHSO₃H, O—C₁₋₁₀alkylNHSO₃H, O—C₁₋₁₀acyl, O—C₁₋₁₀acylC₃₋₁₀aryl, O—C(O)C₃₋₁₀aryl, O—C(O)C₁₋₁₀alkylCOOH, O—C(O)C₃₋₁₀cycloalkylCOOH, O—SO₃H, OH, O—C(O)C₃₋₁₀arylSO₃H, O—C(O)C₃₋₁₀arylCOOH, NH—C₁₋₁₀acyl, NH—C₁₋₁₀acylC₃₋₁₀aryl, NH—C(O)C₃₋₁₀aryl, NH—C(O)C₁₋₁₀alkylCOOH, NH—C(O)C₃₋₁₀cycloalkylCOOH, NH—SO₃H, NH—C(O)C₃₋₁₀arylSO₃H, NH—C(O)C₃₋₁₀arylCOOH and NH₂.
R₁₇ is selected from the group consisting of: hydrogen, O—C₁₋₁₀alkyl, O—C₂₋₁₀alkenyl, O—C₃₋₁₀cycloalkenyl, O—C₂₋₁₀alkynyl, O—C₃₋₁₀cycloalkylC₃₋₁₀aryl, O—C₁₋₁₀alkylC₃₋₁₀aryl, O—C₃₋₁₀cycloalkyl, O—C₁₋₁₀acyl, O—C₁₋₁₀acylC₃₋₁₀aryl, O—SO₃H, O—C(O)C₃₋₁₀aryl, O—C(O)C₁₋₁₀alkylCOOH, O—C(O)C₃₋₁₀cycloalkylCOOH, O—C(O)C₃₋₁₀arylSO₃H, O—C(O)C₃₋₁₀arylCOOH and OH.
n is an integer selected from 0 to 4;
l, m and p are each an integer independently selected from 0 and 1;
provided at least one of the groups R₃, R₄, R₅, R₆, R₈, R₉, R₁₀, R₁₁, R₁₃, R₁₄, R₁₅ and R₁₆ does not represent O—SO₃H or OH when R₂, R₇ and R₁₂ represents independently of each other a group NH—SO₃H or NH—C₁₋₁₀acyl;
provided at least 20% of the groups R₂, R₃, R₄, R₅, R₆, R₇, R₈, R₉, R₁₀, R₁₁, R₁₂, R₁₃, R₁₄, R₁₅, R₁₆ and R₁₇ are O—SO₃H or NH—SO₃H;
provided at least 20% of the groups R₁, R₂, R₃, R₄, R₅, R₆, R₇, R₈, R₉, R₁₀, R₁₁, R₁₂, R₁₃, R₁₄, R₁₅, R₁₆ and R₁₇ are NH—C₁₋₁₀acyl, NH—C₁₋₁₀acylC₃₋₁₀aryl, NH—C(O)C₃₋₁₀aryl, NH—C(O)C₁₋₁₀alkylCOOH, NH—C(O)C₃₋₁₀cycloalkylCOOH, NH—C(O)C₃₋₁₀arylSO₃H, NH—C(O)C₃₋₁₀arylCOOH, O—C₁₋₁₀alkyl, O—C₃₋₁₀cycloalkyl, O—C₁₋₁₀alkylC₃₋₁₀aryl, O—C₃₋₁₀cycloalkylC₃₋₁₀aryl, O—C₁₋₁₀acyl, O—C₁₋₁₀acylC₃₋₁₀aryl, O—C₂₋₁₀alkenyl, O—C₂₋₁₀cycloalkenyl, O—C(O)C₃₋₁₀aryl, O—C(O)C₁₋₁₀alkylCOOH, O—C(O)C₃₋₁₀cycloalkylCOOH, O—C(O)C₃₋₁₀arylSO₃H, O—C(O)C₃₋₁₀arylCOOH, OH or O—C₂₋₁₀alkynyl; and
wherein any of R₁₋₁₇ are independently optionally substituted with one or more groups independently selected from C₁₋₁₀alkyl, C₃₋₁₀cycloalkyl, C₂₋₁₀alkenyl, C₃₋₁₀cycloalkenyl, O—C₁₋₁₀alkyl, O—C₃₋₁₀cycloalkyl, O—C₂₋₁₀alkenyl, O—C₃₋₁₀cycloalkenyl, O—C₁₋₁₀alkylC₃₋₁₀aryl, O—C₃₋₁₀cycloalkylC₃₋₁₀aryl, C₂₋₁₀alkynyl, C₃₋₁₀aryl, C₃₋₁₀arylSO₃H, C₃₋₁₀arylC₁₋₁₀alkyl, C₃₋₁₀arylC₃₋₁₀cycloalkyl, C₁₋₁₀alkylC₃₋₁₀aryl, COOH, C₁₋₁₀alkylCOOH, C₃₋₁₀cycloalkylC₃₋₁₀aryl, C₃₋₁₀cycloalkylCOOH, C₃₋₁₀arylCOOH, COOC₁₋₁₀alkyl, COO—C₃₋₁₀cycloalkyl, SH, S—C₁₋₁₀alkyl, S—C₃₋₁₀cycloalkyl, SO₂H, SO₂C₁₋₁₀alkyl, SO₂C₃₋₁₀cycloalkyl, SO₂C₃₋₁₀aryl, SO₂C₁₋₁₀alkylC₃₋₁₀aryl, SO₂C₃₋₁₀cycloalkylC₃₋₁₀aryl, O—SO₃H, O—P(O)(OH)₂, halo, C₁₋₁₀alkylhalo, C₃₋₁₀cycloalkylhalo, perhaloC₁₋₁₀alkyl, perhaloC₃₋₁₀cycloalkyl, OH, ═O, NH₂, ═NH, NH—C₁₋₁₀alkyl, N(C₁₋₁₀alkyl)₂, NH—C(O)C₁₋₁₀alkyl, NH—C₃₋₁₀cycloalkyl, N(C₃₋₁₀cycloalkyl)₂, N(C₁₋₁₀alkyl) (C₃₋₁₀cycloalkyl), ═N—C₃₋₁₀cycloalkyl, NH—C(O)C₃₋₁₀cycloalkyl, C(O)NH₂, C(O)NHC₁₋₁₀alkyl, C(O)N(C₁₋₁₀alkyl)₂, C(O)NHC₃₋₁₀cycloalkyl, C(O)N(C₃₋₁₀cycloalkyl)₂, C(O)N(C₁₋₁₀alkyl)(C₃₋₁₀cycloalkyl), C(O)NHC₃₋₁₀aryl, NO₂, ONO₂, CN, SO₂, SO₂NH₂, C(O)H and C(O)C₁₋₁₀alkyl, C(O)C₃₋₁₀cycloalkyl;
provided that when: R₁ is O—CH₂CH═CH₂; R₂, R₇ and R₁₂ are each NH—SO₃H; R₄, R₅, R₉, R₁₀, R₁₄ and R₁₅ are each O—SO₃H; R₃, R₆, R₈, R₁₁, R₁₃ and R₁₆ are each O-benzyl; and R₁₇ is not O-para-methoxybenzyl.

18. A compound or a salt or solvate of claim 17, wherein: R₂, R₇ and R₁₂ are each independently selected from the group consisting of: NH—C₁₋₁₀acyl, NH—C₁₋₁₀acylC₃₋₁₀aryl, NH—SO₃H and NH₂.

19. A compound or a salt or solvate of claim 17, wherein:
R₃, R₄, R₅, R₆, R₈, R₉, R₁₀, R₁₁, R₁₃, R₁₄, R₁₅, R₁₆ and R₁₇ are each independently selected from the group consisting of: O—C₁₋₁₀alkyl, O—C₁₋₁₀alkylC₃₋₁₀aryl, O—C₁₋₁₀acyl, O—C₁₋₁₀acylC₃₋₁₀-aryl, O—SO₃H and OH.

20. A compound or a salt or solvate of claim 17, wherein:
R₁ is selected from the group consisting of: O—C₁₋₁₀alkyl, O—C₂₋₁₀alkenyl and O—C₂₋₁₀alkynyl and OH; R₂, R₇ and R₁₂ are each independently selected from the group consisting of: NH—C₁₋₁₀acyl, NH—C₁₋₁₀acylC₁₋₁₀aryl, NH—SO₃H and NH₂; R₃, R₆, R₈, R₁₁, R₁₃ and R₁₆ are each independently selected from the group consisting of: O—C₁₋₁₀alkylC₃₋₁₀aryl, O—C₁₋₁₀alkyl and OH; R₄, R₅, R₉, R₁₀, R₁₄ and R₁₅ are each independently selected from the group consisting of: OH and O—SO₃H; R₁₇ is selected from the group consisting of: O—C₁₋₁₀alkylC₃₋₁₀aryl, O—C₁₋₁₀alkyl and OH; n is an integer selected from 0 to 4; l, m and p are each an integer independently selected from 0 and 1 and at least two of l, m and p are 1; wherein any of R₁, R₂, R₃, R₆, R₇, R₈, R₁₁, R₁₂, R₁₃, R₁₆ and R₁₇ are independently optionally substituted with one or more groups independently selected from C₁₋₁₀alkyl, C₂₋₄₀alkenyl, O—C₁₋₁₀alkyl, O—C₂₋₁₀alkenyl, O—C₁₋₁₀ alkylC₃₋₁₀aryl, C₂₋₁₀alkynyl, C₃₋₁₀aryl, C₃₋₁₀arylSO₃H, C₃₋₁₀aryl C₁₋₁₀alkyl, C₁₋₁₀alkyl C₃₋₁₀aryl, COOH, C₁₋₁₀alkylCOOH, C₃₋₁₀arylCOOH, COOC₁₋₁₀alkyl, SH, S—C₁₋₁₀alkyl, SO₂H, SO₂C₁₋₄₀alkyl, SO₂C₃₋₁₀aryl, SO₂C₁₋₁₀alkylC₃₋₄₀aryl, O—SO₃H, O—P(O)(OH)₂, halo, C₁₋₁₀alkylhalo, perhalo C₁₋₁₀alkyl, OH, ═O, NH₂, ═NH, NH—C₁₋₁₀alkyl, N(C₁₋₁₀alkyl)₂, ═NC₁₋₁₀alkyl, NH—C(O)C₁₋₁₀alkyl, C(O)NH₂, C(O)NHC₁₋₁₀alkyl, C(O)N(C₁₋₁₀alkyl)₂, C(O)NHC₃₋₁₀aryl, NO₂, ONO₂, CN, SO₂, SO₂NH₂, C(O)H, and C(O)C₁₋₁₀alkyl.

21. A compound or a salt, or solvate of claim 17, wherein l=m=1.

22. A compound or a salt or solvate of claim 17, wherein p is 1.

23. A compound or a salt or solvate of claim 17, wherein n is 1.

24. A compound or a salt or solvate of claim 17, wherein n is 2.

25. A compound or a salt or solvate of claim 17 wherein it is chosen from compounds 215-216, 236-241, 244-263, 266-268, 274-283, 288 and 294.

26. A pharmaceutical composition comprising a compound or a salt or solvate according to claim 17 and a compound of formula I in which R₁ is O—CH₂CH═CH₂; R₂, R₇ and R₁₂ are each NH—SO₃H; R₄, R₅, R₉, R₁₀, R₁₄ and R₁₅ are each O—SO₃H; R₃, R₆, R₈, R₁₁, R₁₃ and R₁₆ are each O-benzyl; and R₁₇ is O-para-methoxybenzyl or a salt or solvate thereof and a pharmaceutically acceptable diluent or carrier.

27. A pharmaceutical composition according to claim 26 wherein it further contain a cytokine, and/or other mobilising agents.

28. A method for the treatment of cancer comprising the administration of an effective amount of a compound or a salt or solvate of claim 17 to a patient in need thereof.

29. A method for the treatment of pathological angiogenesis comprising the administration of an effective amount of a compound or a salt or solvate of claim 17 to a patient in need thereof.

30. A method for interfering with the interaction of one or more heparin sulphate binding protein sulphate with heparan sulphate comprising the administration of a compound or a salt or solvate of claim 17 to a patient in need thereof.

31. A method for promoting the mobilisation of stem cells and/or for the treatment of diseases and conditions that are typically associated with patients suffering from blood and/or bone marrow cancers and/or solid tumours, and/or for the treatment of acquired or congenital diseases mediated by hematological disorders comprising the administration of an effective amount of a compound or a salt or solvate of claim 17 to a patient in need thereof.

32. A method according to claim 31 wherein the compound or salt or solvate is administered as a combined preparation for simultaneous, separate or sequential use with at least a cytokine and/or other mobilising agents.
